(19) United States
(12) Patent Application Publication
(10) Pub. No.: US 2018/0095067 A1
(43) Pub. Date: Apr. 5, 2018

(54) DEVICES AND METHODS FOR SAMPLE ANALYSIS

(71) Applicant: Abbott Laboratories, Abbott Park, IL (US)

(72) Inventors: Jeffrey B. Huff, Lincolnshire, IL (US); Mark A. Hayden, Ingleside, IL (US); Peter J. Karabatsos, Glencoe, IL (US); Andrew S. Schapals, Pleasant Prairie, WI (US); Anthony S. Muerhoff, Kenosha, WI (US); Felicia Bogdan, Gurnee, IL (US); Thomas Leary, Kenosha, WI (US); Shelley R. Holets-McCormack, Waukegan, IL (US); Sophie Laurenson, Basel-Land (CH); Andrew T. Fischer, Euless, TX (US); Richard Haack, Skokie, IL (US); Stefan Hershberger, Highland Park, IL (US); Dustin House, Carrollton, TX (US); Lei QIAO, Lake Bluff, IL (US); M. Shawn Murphy, Allen, TX (US); Mark R. Pope, Grayslake, IL (US); Edna M. Prieto-Ballengee, Dallas, TX (US); QiaoQiao Ruan, Kildeer, IL (US); Pathik Soni, Chicago, IL (US); Sergey Tetin, Lindenhurst, IL (US); Lyle Yarnell, Richardson, TX (US); John M. Robinson, Gurnee, IL (US)

(73) Assignee: Abbott Laboratories, Abbott Park, IL (US)

(21) Appl. No.: 15/724,200

(22) Filed: Oct. 3, 2017

Related U.S. Application Data
(63) Continuation-in-part of application No. PCT/US2016/025785, filed on Apr. 2, 2016.
Application No. PCT/US2016/025785 is a continuation-in-part of application No. PCT/US2016/025787, filed on Apr. 3, 2015.
(60) Provisional application No. 62/142,858, filed on Apr. 3, 2015.
(60) Provisional application No. 62/142,872, filed on Apr. 3, 2015.

(51) Int. Cl. G01N 33/487; G01N 33/53

(52) U.S. Cl. CPC G01N 33/48721; G01N 33/5302

(57) ABSTRACT

Integrated microfluidic and analyte detection devices are disclosed, along with methods of detecting target analytes. Digital microfluidic and analyte detection devices include a first substrate and a second substrate aligned generally parallel to each other to define a gap therebetween, the first substrate including a plurality of electrodes to generate electrical actuation forces on a liquid droplet disposed in the gap; at least one reagent disposed on at least one of the first substrate or the second substrate and configured to be carried by the liquid droplet; and an analyte detection device in fluid communication with the gap, wherein the plurality of electrodes are configured to move the liquid droplet towards the analyte detection device.

### CROSS-REFERENCE TO RELATED APPLICATIONS

This application is a continuation-in-part of International Application No. PCT/US16/025785, filed Apr. 2, 2016, which claims the benefit of U.S. Provisional Patent Application No. 62/142,858, filed Apr. 3, 2015, and a continuation-in-part of International Application No. PCT/US16/025787, filed Apr. 2, 2016, which claims the benefit of U.S. Provisional Application Nos. 62/142,872, filed Apr. 3, 2015, 62/278,303, filed Jan. 13, 2016, and 62/279,488, filed Jan. 15, 2016, each of which is incorporated herein by reference in its entirety.

### BACKGROUND

Methods and devices that can accurately analyze analyte(s) of interest in a sample are essential for diagnostics, prognostics, environmental assessment, food safety, detection of chemical or biological warfare agents and the like. Such methods and devices not only need to be accurate, precise and sensitive but are also advantageous when a minute sample is to be analyzed quickly and with minimal instrumentation.

Analyte analysis is usually performed by carrying out a sample preparation step that is either performed manually or using complicated robotics. After sample preparation, the assaying of an analyte in the prepared sample further involves use of expensive and complicated systems for transporting the prepared sample to a machine that then performs analysis of an analyte in the prepared sample.

Integrated devices that can be used to prepare a sample and assay the prepared sample are highly desirable in the field of analyte analysis. Such integrated devices would offer a low cost option and would considerably increase the ease of performing analyte analysis, especially in clinical applications, such as point-of-care applications.

As such, there is an interest in integrated devices for performing analyte analysis and with improved sample analysis capabilities.

### SUMMARY

Embodiments of the present disclosure relate to methods, systems, and devices for analysis of analyte(s) in a sample. For example, the present disclosure provides for the detection of analyte(s) in a sample. In certain embodiments, the sample may be a biological sample.

In certain aspects, the present disclosure provides a digital microfluidic and analyte detection device including a first substrate and a second substrate aligned generally parallel to each other to define a gap therebetween, the first substrate including a plurality of electrodes to generate electrical actuation forces on a liquid droplet disposed in the gap; at least one reagent disposed on at least one of the first substrate or the second substrate and configured to be carried by the liquid droplet; and an analyte detection device in fluid communication with the gap, wherein the plurality of electrodes are configured to move the liquid droplet towards the analyte detection device.

In certain other aspects, the present disclosure provides methods of detecting an analyte of interest. Such methods include introducing a liquid droplet including an analyte of interest into a device having a first substrate and a second substrate aligned generally parallel to each other to define a gap therebetween, the first substrate comprising a plurality of electrodes to generate electrical actuation forces on a liquid droplet disposed in the gap; at least one reagent disposed on at least one of the first substrate or the second substrate and configured to be carried by the liquid droplet; and an analyte detection device in fluid communication with the gap, wherein the plurality of electrodes are configured to move the liquid droplet towards the analyte detection device. The methods further include actuating at least one electrode to move the liquid droplet towards the analyte detection device, labeling the analyte of interest with a detectable label, and detecting the detectable label.

### BRIEF DESCRIPTION OF THE DRAWINGS

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1A illustrates a side view of an integrated digital microfluidic and analyte detection device according to one embodiment.

FIG. 1B illustrates a side view of the integrated digital microfluidic and analyte detection device according to another embodiment.

FIG. 2A illustrates a side view of an integrated digital microfluidic and analyte detection device according to an embodiment.

FIG. 2B illustrates a side view of the integrated digital microfluidic and analyte detection device according to another embodiment.

FIG. 3A illustrates a side view of the device of FIG. 2A with a liquid droplet being moved in the device.

FIG. 3B illustrate a side view of the device of FIG. 2B with of droplet being moved in the device.

FIG. 4A illustrates a side view of the device of FIG. 2A with a droplet containing particles/beads being moved onto an array of wells.

FIG. 4B illustrates a side view of the device of FIG. 2B with a droplet containing particles/beads being moved onto an array of wells with a droplet of an immiscible fluid.

FIG. 5 illustrates an aqueous droplet being moved over the array of wells using a hydrophilic capillary region of the device.

FIG. 6 illustrates an aqueous droplet being moved over the array of wells.

FIGS. 7A-7B illustrate various exemplary orientations of the sidewalls of the wells.

FIG. 8 illustrates an example of fabricating a second (e.g., bottom) substrate of the digital microfluidic and analyte detection device.

FIG. 9 illustrates an example of fabricating a first (e.g., top) substrate of the digital microfluidic and analyte detection device.

FIG. 10 illustrates an example of assembling the top and bottom substrates to manufacture a plurality of digital microfluidic and analyte detection devices.

FIG. 11A and FIG. 11B show a view from the top of a bottom substrate of exemplary digital microfluidic and analyte detection devices of the present disclosure.

FIGS. 12A-12D illustrate examples of fabricating the array of wells into the integrated digital microfluidic and analyte detection device.

FIG. 13A illustrates a side view of one embodiment of the surface acoustic component of the integrated microfluidic and analyte device and array of wells.

FIG. 13B illustrates a side view of another embodiment of the surface acoustic component of the integrated microfluidic and analyte device and array of wells.

FIGS. 14A-14B illustrate an example of fabricating the sample preparation component and well array component.

FIG. 15 depicts an exemplary method of the present disclosure.

FIG. 16 illustrates an exemplary method for removing beads not located in the wells of the depicted device.

FIG. 17 illustrates another exemplary method for removing beads not located in the wells of the depicted device.

FIG. 18 depicts a schematic of a fabrication process of a low-cost DMF chip.

FIG. 19 depicts a single flexible chip fabricated according to the schematic in FIG. 18.

FIG. 20 depicts actuation of droplets in a DMF chip, according to embodiments of the present disclosure.

FIGS. 21A-21E depicts performance of an immunoassay in a DMF chip, according to embodiments of the present disclosure.

FIGS. 22A and 22B are schematic diagrams showing a design and fabrication method of DMF top electrode chips and well array, according to embodiments of the present disclosure.

FIG. 23 shows a schematic diagram of a well design, according to embodiments of the present disclosure.

FIGS. 24A and 24B are schematic diagram showing well spacing formats, according to embodiments of the present disclosure.

FIG. 25 are a collection of magnified optical images of the array of wells, according to embodiments of the present disclosure.

FIG. 26 is a schematic diagram showing assembly of an integrated DMF-well device from a DMF top electrode chip and a well array, according to embodiments of the present disclosure.

FIGS. 27A-27G are a collection of schematic diagrams showing an immunoassay performed on a integrated DMF-well device, according to embodiments of the present disclosure.

FIG. 28 is a schematic diagram of an enzyme-linked immunosorbent assay (ELISA)-based sandwich immunoassay, coupled with digital fluorescence detection in a well array, according to embodiments of the present disclosure.

FIG. 29 is a schematic showing components for DMF-directed top loading of microparticles into a well array, according to embodiments of the present disclosure.

FIGS. 30A-30D are a collection of schematic diagrams showing steps of a thyroid stimulating hormone (TSH) immunoassay using an integrated DMF-well device, according to embodiments of the present disclosure.

FIG. 31A and FIG. 31B depict a microfluidics device used in conjunction with a nanopore device.

FIG. 32A and FIG. 32B depict a schematic of a reversibly integrated device having a microfluidics module combined with a nanopore module via a channel.

FIGS. 32C-32L depict schematics of exemplary integrated devices in which a microfluidics module is fluidically connected to a nanopore module. The nanopore module includes a nanopore in a layer physically separating two microfluidic channels at a location where the two microfluidic channels intersect.

FIG. 33 illustrates an exemplary integrated device which includes a microfluidics module and a nanopore module.

FIG. 34 provides an integrated device in which the digital microfluidics modules includes a built-in nanopore module.

FIG. 35A shows a top view of an integrated device.

FIG. 35B shows a side view of the integrated device of FIG. 35A.

FIG. 36 depicts an exemplary device and method of the present disclosure.

FIG. 37 depicts an exemplary device and method of the present disclosure.

FIG. 38 depicts a side view of an exemplary integrated device of the present disclosure.

FIG. 39 depicts an exemplary system of the present disclosure.

FIG. 40 depicts a schematic of a fabrication process of a low-cost DMF chip.

FIG. 41 depicts a single flexible DMF chip fabricated according to the schematic in FIG. 40.

FIG. 42 depicts actuation of droplets in a DMF chip, according to embodiments of the present disclosure.

FIGS. 43A-43E depict performance of an immunoassay in a DMF chip, according to embodiments of the present disclosure.

FIGS. 44A-44C depict fabrication and design of a nanopore module, according to embodiments of the present disclosure.

FIG. 45A shows a plot of leakage current measured in real-time.

FIG. 45B depicts a current-voltage (I-V) curve for a nanopore.

FIGS. 46A-46C show filling of a capillary channel in an integrated DMF-nanopore module device, according to embodiments of the present disclosure.

FIG. 47 shows a schematic diagram for droplet transfer between modules in an integrated DMF-nanopore module device, according to embodiments of the present disclosure.

FIG. 48 shows a schematic diagram of a nanopore module design, according to embodiments of the present disclosure.

FIG. 49 shows a schematic diagram of an integrated DMF-nanopore module device adapted to perform droplet transfer between the modules by passive transport, according to embodiments of the present disclosure.

FIG. 50 shows a schematic diagram of an integrated DMF-nanopore module device adapted to perform droplet transfer between the modules by passive transport, according to embodiments of the present disclosure.

FIG. 51 is a schematic diagram of a silicon microfluidic device containing silicon microchannels that allow passive movement of a liquid droplet by passive transport, according to embodiments of the present disclosure.

FIG. 52 is an image of a silicon microchannel of a silicon microfluidic device that allows passive movement of a liquid droplet by passive transport, according to embodiments of the present disclosure.

FIG. 53A and FIG. 53B show a schematic of a fabrication method for an integrated nanopore sensor, according to embodiments of the present disclosure.

FIGS. 54A-54C display the scatter plot (level duration versus level of blockage) for plots obtained using showing translocation events through: (FIG. 54A) nanopores comprised of regular double stranded DNA (“dsDNA”); (FIG. 54B) nanopores comprised of DBCO-modified dsDNA; and (FIG. 54C) nanopores comprised of dsDNA stars.

FIG. 55 shows a schematic of the thiol-mediated chemical cleavage.

FIG. 56A and FIG. 56B show a schematic of photocleavage experiments performed on magnetic microparticles.

FIG. 57 shows a schematic of the reagent placement on the DMF chip.

FIG. 58 displays a bar chart of sample versus nanopore flux (DMF cleavage) in sec-1.

FIG. 59 displays the means by which a threshold for digital signal counting is determined.

FIGS. 60A-60C show current blockages over different time periods for three standards of 94 nM (FIG. 60A), 182 nM (FIG. 60B), and 266 nM (FIG. 60C).

FIG. 61 shows a dose-response curve of number of events over a fixed amount of time (5 min).

FIG. 62 shows a dose-response curve of time required for fixed number of events.

FIG. 63 shows a dose-response curve of events per unit time.

FIG. 64 shows a dose-response curve of events per unit time using Seq31-SS-biotin.

FIG. 65 shows a schematic diagram of a nanopore chamber design in a silicon nanopore module, according to embodiments of the present disclosure.

FIG. 66 shows a table listing the physical parameters used for COMSOL electrical field simulations in a nanopore chamber of a silicon nanopore module, according to embodiments of the present disclosure.

FIG. 67 is a collection of images showing simulation results for counter ion concentration gradients near a nanopore in a silicon nanopore module, according to embodiments of the present disclosure.

FIG. 68 is a graph showing the effects of the diameter of a SiO2 via made over a nanopore membrane with a nanopore on the electroosmotic flow through the nanopore, according to embodiments of the present disclosure.

FIG. 69 is a graph showing the effects of the diameter of a SiO2 via made over a nanopore membrane with a nanopore on the conductance through the nanopore, according to embodiments of the present disclosure.

FIG. 70 shows a schematic diagram of an integrated DMF-nanopore module device with the nanopore module positioned on one side of the DMF module, according to embodiments of the present disclosure.

FIG. 71 is a collection of images showing movement of liquid from a DMF module through a hole in a DMF module substrate by capillary force, according to embodiments of the present disclosure.

FIG. 72 is a collection of images showing an integrated DMF-nanopore module device with the nanopore module positioned on one side of the DMF module and electrodes configured for nanopore fabrication, according to embodiments of the present disclosure.

FIG. 73 is a schematic diagram of an integrated DMF-nanopore module device with the nanopore module positioned on one side of the DMF module, according to embodiments of the present disclosure.

FIG. 74 is a schematic diagram of an integrated DMF-nanopore module device with the nanopore module positioned between two DMF modules, according to embodiments of the present disclosure.

FIG. 75 is a graph showing fabrication of a nanopore in a nanopore membrane (a transmission electron microscope (TEM) window) by applying a voltage across the nanopore membrane, and as evidenced by dielectric breakdown, according to embodiments of the present disclosure.

FIG. 76A and FIG. 76B are a collection of graphs showing current-voltage (I-V) curves of a nanopore formed in a membrane, before and after a conditioning process, according to embodiments of the present disclosure.

FIG. 77 shows a scatter plot of the averages of ratios plotted between counting label average diameter and nanopore size to the SNR (signal to noise ratio).

### DETAILED DESCRIPTION

An integrated microfluidic and analyte detection device is disclosed. The analyte detection device can be capable of detecting a target analyte in a sample, such as a biological sample. For example, the analyte detection device can include an array of wells and/or a nanopore module.

Also provided herein are exemplary methods for using an integrated microfluidic and analyte detection device and associated systems. Embodiments of the present disclosure relate to methods, systems, and devices for analysis of analyte(s) in a sample. In certain embodiments, the sample may be a biological sample.

It must be noted that as used herein and in the appended claims, the singular forms “a”, “an” and “the” include plural referents unless the context clearly dictates otherwise. Thus, for example, refer to “an electrode” includes plurality of such electrodes and reference to “the well” includes reference to one or more wells and equivalents thereof known to those skilled in the art, and so forth.

All publications mentioned herein are incorporated herein by reference to disclose and describe the methods and/or materials in connection with which the publications are cited. The present disclosure is controlling to the extent there is a contradiction between the present disclosure and a publication incorporated by reference.

### 1. Definitions

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting.

“Comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Affinity” and “binding affinity” as used interchangeably herein refer to the tendency or strength of binding of the binding member to the analyte. For example, the binding affinity may be represented by the equilibrium dissociation constant (K_{D}), the dissociation rate (k_{d}), or the association rate (kₐ).

“Analog” as used herein refers to a molecule that has a similar structure to a molecule of interest (e.g., nucleoside analog, nucleotide analog, sugar phosphate analog, analyte analog, etc.). An analyte analog is a molecule that is structurally similar to an analyte but for which the binding member has a different affinity.

“Aptamer” as used herein refers to an oligonucleotide or peptide molecule that can bind to pre-selected targets including small molecules, proteins, and peptides among others with high affinity and specificity. Aptamers may assume a variety of shapes due to their propensity to form helices and single-stranded loops. An oligonucleotide or nucleic acid aptamer can be a single-stranded DNA or RNA (ssDNA or ssRNA) molecule. A peptide aptamer can include a short variable peptide domain, attached at both ends to a protein scaffold.

“Bead” and “particle” are used herein interchangeably and refer to a substantially spherical solid support.

“Component,” “components,” or “at least one component,” refer generally to a capture antibody, a detection reagent or conjugate, a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, serum, whole blood, tissue aspirate, or plasma sample, in accordance with the methods described herein and other methods known in the art. Some components can be in solution or lyophilized for reconstitution for use in an assay.

“Control” as used herein refers to a reference standard for an analyte such as is known or accepted in the art, or determined empirically using acceptable means such as are commonly employed. A “reference standard” is a standardized substance which is used as a measurement base for a similar substance. For example, there are documented reference standards published in the U.S. Pharmacopeial Convention (USP-NF), Food Chemicals Codex, and Dietary Supplements Compendium (all of which are available at http://www.usp.org), and other well-known sources. Methods for standardizing references are described in the literature. Also well-known are means for quantifying the amounts of analyte present by use of a calibration curve for analyte or by comparison to an alternate reference standard. A standard curve can be generated using serial dilutions or solutions of known concentrations of analyte, by mass spectroscopy, gravimetric methods, and by other techniques known in the art. Alternate reference standards that have been described in the literature include standard addition (also known as the method of standard addition), or digital polymerase chain reaction.

“Digital microfluidics (DMF),” “digital microfluidic module (DMF module),” or “digital microfluidic device (DMF device)” as used interchangeably herein refer to a module or device that utilizes digital or droplet-based microfluidic techniques to provide for manipulation of discrete and small volumes of liquids in the form of droplets. Digital microfluidics uses the principles of emulsion science to create fluid-fluid dispersion into channels (principally water-in-oil emulsion). It allows the production of monodisperse drops/bubbles or with a very low polydispersity. Digital microfluidics is based upon the micromanipulation of discontinuous fluid droplets within a reconfigurable network. Complex instructions can be programmed by combining the basic operations of droplet formation, translocation, splitting, and merging.

Digital microfluidics operates on discrete volumes of fluids that can be manipulated by binary electrical signals. By using discrete unit-volume droplets, a microfluidic operation may be defined as a set of repeated basic operations, i.e., moving one unit of fluid over one unit of distance. Droplets may be formed using surface tension properties of the liquid. Actuation of a droplet is based on the presence of electrostatic forces generated by electrodes placed beneath the bottom surface on which the droplet is located. Different types of electrostatic forces can be used to control the shape and motion of the droplets. One technique that can be used to create the foregoing electrostatic forces is based on dielectrophoresis which relies on the difference of electrical permittivities between the droplet and surrounding medium and may utilize high-frequency AC electric fields. Another technique that can be used to create the foregoing electrostatic forces is based on electrowetting, which relies on the dependence of surface tension between a liquid droplet present on a surface and the surface on the electric field applied to the surface.

“Drag-tag” refers to a mobility modifier. The drag-tag may be genetically engineered, highly repetitive polypeptides (“protein polymers”) that are designed to be large, water-soluble, and completely monodisperse. Positively charged arginines may be deliberately introduced at regular intervals into the amino acid sequence to increase the hydrodynamic drag without increasing drag-tag length. Drag-tags are described in U.S. Patent Publication No. 20120141997, which is incorporated herein by reference.

“Enzymatic cleavable sequence” as used herein refers to any nucleic acid sequence that can be cleaved by an enzyme. For example, the enzyme may be a protease or an endonuclease, such as a restriction endonuclease (also called restriction enzymes). Restriction endonucleases are capable of recognizing and cleaving a DNA molecule at a specific DNA cleavage site between predefined nucleotides. Some endonucleases, such as for example Fokl, comprise a cleavage domain that cleaves the DNA unspecifically at a certain position regardless of the nucleotides present at this position. In some embodiments, the specific DNA cleavage site and the DNA recognition site of the restriction endonuclease are identical.

“Globular protein” refers to a water soluble protein that has a roughly spherical shape. Examples of globular proteins include but are not limited to ovalbumin, beta-globulin, C-reactive protein, fibrin, hemoglobin, IgG, IgM, and thrombin.

“Label” or “detectable label” as used interchangeably herein refers to a moiety attached to a specific binding member or analyte to render the reaction between the specific binding member and the analyte detectable, and the specific binding member or analyte so labeled is referred to as “detectably labeled.” A label can produce a signal that is detectable by visual or instrumental means. Various labels include: (i) a tag attached to a specific binding member or analyte by a cleavable linker; or (ii) signal-producing substance, such as chromagens, fluorescent compounds, enzymes, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

“Microparticle(s)(s)” and “microbead(s)” are used interchangeably herein and refer to a microbead or microparticle that is allowed to occupy or settle in an array of wells, such as, for example, in an array of wells in a detection module. The microparticle and microbead may contain at least one specific binding member that binds to an analyte of interest and at least one detectable label. Alternatively, the microparticle and microbead may containing a first specific binding member that binds to the analyte and a second specific binding member that also binds to the analyte and contains at least one detectable label.

“Nanoparticle(s)” and “nanobead(s)” are used interchangeably herein and refer to a nanobead or nanoparticle sized to translocate through or across a nanopore used for counting the number of nanobeads/nanoparticles traversing through it.

“Nucleobase” or “Base” means those naturally occurring and synthetic heterocyclic moieties commonly known in the art of nucleic acid or polynucleotide technology or peptide nucleic acid technology for generating polymers. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Nucleobases can be linked to other moieties to form nucleosides, nucleotides, and nucleoside/tide analogs.

“Nucleoside” refers to a compound consisting of a purine, deazapurine, or pyrimidine nucleobase, e.g., adenine, guanine, cytosine, uracil, thymine, 7-deazaadenine, 7-deazaguanosine, that is linked to the anomeric carbon of a pentose sugar at the 1′ position, such as a ribose, 2′-deoxyribose, or a 2′,3′-di-deoxyribose.

“Nucleotide’ as used herein refers to a phosphate ester of a nucleoside, e.g., a mono-, a di-, or a triphosphate ester, wherein the most common site of esterification is the hydroxyl group attached to the C-5 position of the pentose.

“Nucleobase polymer” or “nucleobase oligomer” refers to two or more nucleobases that are connected by linkages to form an oligomer. Nucleobase polymers or oligomers include, but are not limited to, poly- and oligonucleotides (e.g., DNA and RNA polymers and oligomers), poly- and oligo-nucleotide analogs and poly- and oligonucleotide mimics, such as polyamide or peptide nucleic acids. Nucleobase polymers or oligomers can vary in size from a few nucleobases to several hundred nucleobases or to several thousand nucleobases. The nucleobase polymers or oligomers may include from about 2 to 100 nucleobases or from about 8000 to 10000 nucleobases. For example, the nucleobase polymers or oligomers may have at least about 2 nucleobases, at least about 5 nucleobases, at least about 10 nucleobases, at least about 20 nucleobases, at least about 30 nucleobases, at least about 40 nucleobases, at least about 50 nucleobases, at least about 60 nucleobases, at least about 70 nucleobases, at least about 80 nucleobases, at least about 90 nucleobases, at least about 100 nucleobases, at least about 200 nucleobases, at least about 300 nucleobases, at least about 400 nucleobases, at least about 500 nucleobases, at least about 600 nucleobases, at least about 700 nucleobases, at least about 800 nucleobases, at least about 900 nucleobases, at least about 1000 nucleobases, at least about 2000 nucleobases, at least about 3000 nucleobases, at least about 4000 nucleobases, at least about 5000 nucleobases, at least about 6000 nucleobases, at least about 7000 nucleobases, at least about 8000 nucleobases, at least about 9000 nucleobases, or at least about 10000 nucleobases.

“One or more nanopores in a layer” means that in a single membrane structure or multiple membrane structures there is either one nanopore, or there are multiple nanopores (e.g., two or more) next to each other (e.g., side by side). When one or more nanopores are present (e.g., one, two, three, four, five, six, or other number of nanopores as technically feasible), optionally they are present side by side (e.g., next to each other) or in series (e.g., one nanopore in one layer present separate from or stacked onto (e.g., above or on top of) another nanopore in another layer, etc.), or in alternate structure such as would be apparent to one skilled in the art. Optionally, such nanopores are independently addressable, e.g., by each being within its own separate compartment (e.g., walled off from any other nanopore), or alternately can be addressed by an independent detection circuit.

“Polymer brush” refers to a layer of polymers attached with one end to a surface. The polymers are close together and form a layer or coating that forms its own environment. The brushes may be either in a solvent state, when the dangling chains are submerged into a solvent, or in a melt state, when the dangling chains completely fill up the space available. Additionally, there is a separate class of polyelectrolyte brushes, when the polymer chains themselves carry an electrostatic charge. The brushes may be characterized by the high density of grafted chains. The limited space then leads to a strong extension of the chains, and unusual properties of the system. Brushes may be used to stabilize colloids, reduce friction between surfaces, and to provide lubrication in artificial joints

“Polynucleotides” or “oligonucleotides” refer to nucleobase polymers or oligomers in which the nucleobases are connected by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof “Nucleic acid” encompasses “polynucleotide” and “oligonucleotides” and includes single stranded and double stranded polymers of nucleotide monomers.

“Polynucleotide analog” or “oligonucleotide analog” refers to nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. No. 6,013,785 and U.S. Pat. No. 5,696,253.

As used herein, a “pore” (alternately referred to herein as “nanopore”) or “channel” (alternately referred to herein as “nanopore” or a “nanochannel”) refers to an orifice, gap, conduit, or groove in a membrane/layer, where the pore or channel is of sufficient dimension that allows passage or analysis of a single molecule (e.g., a tag) at one time (e.g., one-by-one, as in a series).

“Receptor” as used herein refers to a protein-molecule that recognizes and responds to endogenous-chemical signals. When such endogenous-chemical signals bind to a receptor, they cause some form of cellular/tissue-response. Examples of receptors include, but are not limited to, neural receptors, hormonal receptors, nutrient receptors, and cell surface receptors.

As used herein, “spacer” refers to a chemical moiety that extends the cleavable group from the specific binding member, or which provides linkage between the binding member and the support, or which extends the label/tag from the photocleavable moiety. In some embodiments, one or more spacers may be included at the N-terminus or C-terminus of a polypeptide or nucleotide-based tag or label in order to distance optimally the sequences from the specific binding member. Spacers may include but are not limited to 6-aminocaproic acid, 6-aminohexanoic acid; 1,3-diamino propane; 1,3-diamino ethane; polyethylene glycol (PEG) polymer groups, short amino acid sequences, and such as polyglycine sequences, of 1 to 5 amino acids. In some embodiments, the spacer is a nitrobenzyl group, dithioethylamino, 6 carbon spacer, 12 carbon spacer, or 3-(9-((3-carboxypropyl)(tosyl)carbamoyl)acridin-10-ium-10-yl)propane-1-sulfonate.

“Specific binding partner” or “specific binding member” as used interchangeably herein refers to one of two or more different molecules that specifically recognize the other molecule compared to substantially less recognition of other molecules. The one of two different molecules has an area on the surface or in a cavity, which specifically binds to and is thereby defined as complementary with a particular spatial and polar organization of the other molecule. The molecules may be members of a specific binding pair. For example, a specific binding member may include, but is not limited to, a protein, such as a receptor, an enzyme, an antibody and an aptamer, a peptide a nucleotide, oligonucleotide, a polynucleotide and combinations thereof. As used herein, “tag” or “tag molecule” both refer to the molecule (e.g., cleaved from the second binding member or an aptamer dissociated from the target analyte) that is translocated through or across a nanopore, if provided, and provides an indication of the level of analyte in a sample. These terms refer to a single tag molecule or a plurality of the same tag molecule. Likewise “tags”, unless specified otherwise, refers to one or one or more tags.

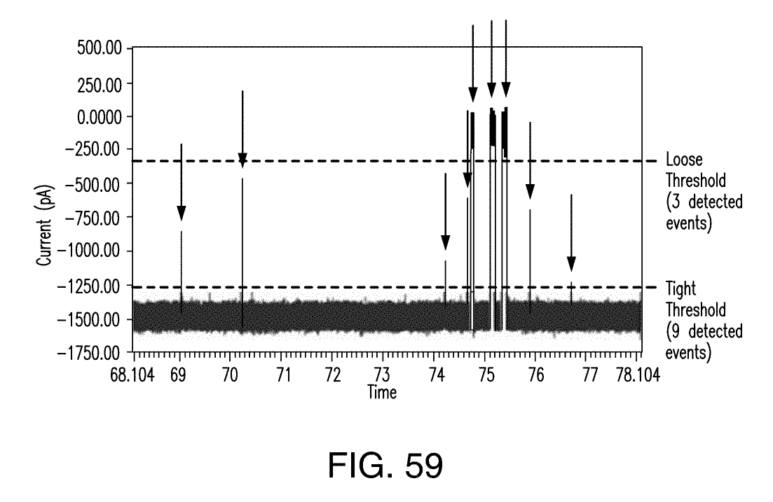

“Threshold” as used herein refers to an empirically determined and subjective cutoff level above which acquired data is considered “signal”, and below which acquired data is considered “noise”. The use of a threshold for digital signal counting is depicted in FIG. 59. A computer program based on CUSUM (Cumulative Sums Algorithm) is employed to process acquired data and detect events based on threshold input from the user. Variation between users is avoided by detection of any many events as possible followed by filtering the data afterwards for specific purposes. For example, as can be seen from this figure, events detected above the set threshold impact the population of events that are counted as signal. With a “loose” threshold a lesser number of events will be counted as signal. With a “tight” threshold a greater number of events will be counted as signal. Setting the threshold as loose or tight is a subjective choice based on the desired sensitivity or specificity for an assay, and whether in a given assessment false positives or false negatives would be preferred. Current blockade signatures from DNA translocations were calculated to be 1.2 nA, which was based on an empirical formula relating current change to the diameter of DNA and the thickness of the nanopore membrane (H. Kwok, et al., PLoS ONE, 9(3), 392880, 2014).

As used herein, reference to movement (e.g., of a nanoparticles, tag, tag molecule, or other) “through or across” a nanopore means alternately, through, or across, in other words, from one side to another of a nanopore, e.g., from the cis to the trans side, or vice versa.

“Tracer” as used herein refers to an analyte or analyte fragment conjugated to a tag or label, wherein the analyte conjugated to the tag or label can effectively compete with the analyte for sites on an antibody specific for the analyte. For example, the tracer may be an analyte or analog of the analyte, such as cyclosporine or its analog ISA247, vitamin D and its analogs, sex hormones and their analogs, etc.

“Translocation event” as used herein refers to an event in which a tag translocates through or across (e.g., from the cis to trans side or vice versa) the layer or nanopore.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety to disclose and describe the methods and/or materials in connection with which the publications are cited. The materials, methods, and examples disclosed herein are illustrative only and not intended to be limiting.

### 2. Methods for Analyte Analysis

Provided herein are methods for analyte analysis. The method may involve single molecule counting. In certain embodiments, a method for analyte analysis may involve assessing an analyte present in a sample. In certain embodiments, the assessing may be used for determining presence of and/or concentration of an analyte in a sample. In certain embodiments, the method may also be used for determining presence of and/or concentration of a plurality of different analytes present in a sample.

Provided herein are methods for detecting an analyte of interest in liquid droplet (wherein the analyte of interest is from a test or biological sample). The method includes providing a first liquid droplet containing an analyte of interest, providing a second liquid droplet containing at least one solid support (such as, for example, a magnetic solid support (such as a bead)) which contains a specific binding member that binds to the analyte of interest, using energy to exert a force to manipulate the first liquid droplet (which contains the analyte of interest) with the second liquid (containing the at least one solid support) to create a mixture, moving all or at least a portion of the mixture to an array of wells (where one or more wells of the array are of sufficient size to accommodate the at least one solid support), adding at least one detectable label to the mixture before, after or both before or after moving a portion of the mixture to the array of wells and detecting the analyte of interest in the wells. In certain embodiments, “using energy to exert a force to manipulate the first liquid droplet with the second liquid droplet” refers to the use of non-mechanical forces (namely, for example, energy created without the use of pumps and/or valves) to provide or exert a force that manipulates (such as merges or combines) at least the first and second liquid droplets (and optionally, additional droplets) into a mixture. Example of non-mechanical forces that can be used in the methods described herein include electric actuation force (such as droplet actuation, electrophoresis, electrowetting, dielectrophoresis, electrostatic actuation, electric field mediated, electrode mediated, capillary force, chromatography, centrifugation or aspiration) and/or acoustic force (such as surface acoustic wave (or “SAW”). In certain embodiments, the the electric actuation force generated is an alternating current. For example, the alternating current can have a root mean squared (rms) voltage of 10 V, 15 V, 20 V, 25 V, 30 V, 35V or more. For example, such alternating current can have a rms voltage of 10 V or more, 15 V or more, 20 V or more, 25 V or more, 30 V or more or 35 V or more. Alternatively, the alternating current can have a frequency in a radio frequency range.

In certain embodiments, if magnetic solid supports are used, an electric actuation force and a magnetic field can be applied and applied from opposition directions, relative to the at least a portion of the mixture. In certain other embodiments, the mixture is mixed by moving it: back and forth, in a circular pattern or by splitting it into two or more submixtures and then merging the submixtures. In certain other embodiments, an electric actuation force can be generated using a series or plurality of electrodes (namely, at least two or more, at least three or more, at least four or more, at least five or more, at least six or more, at least seven or more, at least eight or more, at least nine or more, at least ten or more, at least eleven or more, at least twelve or more, at least thirteen or more, at least fourteen or more, at least fifteen or more, etc.) to move the mixture to the array of wells in order to seal the wells (which are loaded with at least one solid support).

In certain embodiments, the moving of all or at least a portion of the mixture to an array of wells results in the loading (filling and/or placement) of the at least one solid support into the array of wells. In certain embodiments, a magnetic field is used to facilitate movement of the mixture and thus, at least one solid support, into one or more wells of the array. In certain embodiments, after the at least one solid supports are loaded into the wells, any solid supports that are not loaded into a well can be removed using routine techniques known in the art. For example, such removing can involve generating an electric actuation force (such as that described previously herein) with a series or plurality of electrodes to move a fluid droplet (such as a polarizable fluid droplet) to the array of wells to move at least a portion of the mixture to a distance (the length of which is not critical) from the array of wells. In certain embodiments, an aqueous washing liquid can be used to remove the solid supports not bound to any analyte of interest. In such embodiments, the removal involves generating an electric actuation force with a series or plurality of electrodes to move an aqueous wash (or washing) droplet (a third droplet) across the array of wells. The amount and type of aqueous liquid used for said washing is not critical.

In certain embodiments, the mixture in the method is an aqueous liquid. In other embodiments, the mixture is an immiscible liquid. In other embodiments, the liquid droplet is a hydrophobic liquid droplet. In other embodiments, the liquid droplet is a hydrophilic liquid droplet. In certain embodiments, the array of wells used in the method have a hydrophobic surface. In other embodiments, the array of wells has a hydrophilic surface.

In certain embodiments, the first liquid droplet used in the method is a polarizable liquid. In certain embodiments, the second liquid droplet used in the method is a polarizable liquid. In certain embodiments, the first and second liquid droplets used in the method are polarizable liquids. In certain embodiments, the mixture is a polarizable liquid. In certain embodiments one or more of the first droplet, second droplet and mixture is a polarizable liquid.

In certain embodiments, the at least one solid support comprises at least one binding member that specifically binds to the analyte of interest. In certain embodiments, the detectable label is added to the mixture before moving at least a portion of the mixture to the array of wells. In certain other embodiments, the detectable label is added to the mixture after the moving of at least a portion of the analyte of interest. In certain embodiments, the detectable label comprises at least one binding member that specifically binds to the analyte of interest. In certain embodiments, the detectable label comprises a chromagen, a florescent compound, an enzyme, a chemiluminescent compound or a radioactive compound. In certain embodiments, the binding member is a receptor, aptamer or antibody. In certain embodiments, the method further comprises positioning the at least a portion of the mixture over the array of wells using a capillary element configured to facilitate movement of the mixture to the array of wells.

In certain embodiments, the method described herein is performed using a microfluidics device. In certain embodiments, the method described herein is performed using a digital microfluidics device (DMF). In certain embodiments, method described herein is performed using a surface acoustic wave based microfluidics device (SAW). In certain embodiments, method described herein is performed using an integrated DMF and analyte detection device. In certain embodiments, method described herein is performed using an integrated surface acoustic wave based microfluidic device and analyte detection device. In certain embodiments, method described herein is performed using a Robotics based assay processing unit.

Provided herein are methods for detecting an analyte of interest in liquid droplet (wherein the analyte of interest is from a test or biological sample). The method includes providing a first liquid droplet containing an analyte of interest, providing a second liquid droplet containing at least one detectable label which contains a specific binding member that binds to the analyte of interest, using energy to exert a force to manipulate the first liquid droplet (which contains the analyte of interest) with the second liquid (containing the at least one solid support) to create a mixture (namely, an analyte/detectable label-specific binding member complex), moving all or at least a portion of the mixture to an array of wells (where one or more wells of the array are of sufficient size to accommodate the at least one solid support) and detecting the analyte of interest in the wells. In certain embodiments, “using energy to exert a force to manipulate the first liquid droplet with the second liquid droplet” refers to the use of non-mechanical forces (namely, for example, energy created without the use of pumps and/or valves) to provide or exert a force that manipulates (such as merges or combines) at least the first and second liquid droplets (and optionally, additional droplets) into a mixture. Example of non-mechanical forces that can be used in the methods described herein include electric actuation force (such as droplet actuation, electrophoresis, electrowetting, dielectrophoresis, electrostatic actuation, electric field mediated, electrode mediated, capillary force, chromatography, centrifugation or aspiration) and/or acoustic force (such as surface acoustic wave (or “SAW”). In certain embodiments, the the electric actuation force generated is an alternating current. For example, the alternating current can have a root mean squred (rms) voltage of 10 V, 15 V, 20 V, 25 V, 30 V, 35V or more. For example, such alternating current can have a rms voltage of 10 V or more, 15 V or more, 20 V or more, 25 V or more, 30 V or more or 35 V or more. Alternatively, the alternating current can have a frequency in a radio frequency range.

In certain embodiments, the mixture is mixed by moving it: back and forth, in a circular pattern or by splitting it into two or more submixtures and then merging the submixtures. In certain other embodiments, an electric actuation force can be generated using a series or plurality of electrodes (namely, at least two or more, at least three or more, at least four or more, at least five or more, at least six or more, at least seven or more, at least eight or more, at least nine or more, at least ten or more, at least eleven or more, at least twelve or more, at least thirteen or more, at least fourteen or more, at least fifteen or more, etc.) to move the mixture to the array of wells in order to seal the wells (which are loaded with at least one solid support).

In certain embodiments, the moving of all or at least a portion of the mixture to an array of wells results in the loading (filling and/or placement) of the an analyte/detectable label-specific binding member complex into the array of wells. In certain embodiments, a magnetic field is used to facilitate movement of the mixture and thus, at least one an analyte/detectable label-specific binding member complex into one or more wells of the array. For example, such removing can involve generating an electric actuation force (such as that described previously herein) with a series or plurality of electrodes to move a fluid droplet (such as a polarizable fluid droplet) to the array of wells to move at least a portion of the mixture to a distance (the length of which is not critical) from the array of wells. In certain embodiments, an aqueous washing liquid can be used to remove any detectable label-specific binding members not bound to any analyte. In such embodiments, the removal involves generating an electric actuation force with a series or plurality of electrodes to move an aqueous wash (or washing) droplet (a third droplet) across the array of wells. The amount and type of aqueous liquid used for said washing is not critical.

In certain embodiments, the mixture in the method is an aqueous liquid. In other embodiments, the mixture is an immiscible liquid. In other embodiments, the liquid droplet is a hydrophobic liquid droplet. In other embodiments, the liquid droplet is a hydrophilic liquid droplet. In certain embodiments, the array of wells used in the method have a hydrophobic surface. In other embodiments, the array of wells has a hydrophilic surface.

In certain embodiments, the first liquid droplet used in the method is a polarizable liquid. In certain embodiments, the second liquid droplet used in the method is a polarizable liquid. In certain embodiments, the first and second liquid droplets used in the method are polarizable liquids. In certain embodiments, the mixture is a polarizable liquid. In certain embodiments one or more of the first droplet, second droplet and mixture is a polarizable liquid.

In certain embodiments, the detectable label is bound to at least one solid support. In certain embodiments, the detectable label comprises a chromagen, a florescent compound, an enzyme, a chemiluminescent compound or a radioactive compound. In certain embodiments, the binding member is a receptor, aptamer or antibody. In certain embodiments, the method further comprises positioning the at least a portion of the mixture over the array of wells using a capillary element configured to facilitate movement of the mixture to the array of wells.

In certain embodiments, the method described herein is performed using a microfluidics device. In certain embodiments, the method described herein is performed using a digital microfluidics device (DMF). In certain embodiments, method described herein is performed using a surface acoustic wave based microfluidics device (SAW). In certain embodiments, method described herein is performed using an integrated DMF and analyte detection device. In certain embodiments, method described herein is performed using an integrated surface acoustic wave based microfluidic device and analyte detection device. In certain embodiments, method described herein is performed using a Robotics based assay processing unit.

Provided herein are methods for measuring an analyte of interest in liquid droplet (wherein the analyte of interest is from a test or biological sample). The method includes providing a first liquid droplet containing an analyte of interest, providing a second liquid droplet containing at least one solid support (such as, for example, a magnetic solid support (such as a bead)) which contains a specific binding member that binds to the analyte of interest, using energy to exert a force to manipulate the first liquid droplet (which contains the analyte of interest) with the second liquid (containing the at least one solid support) to create a mixture, moving all or at least a portion of the mixture to an array of wells (where one or more wells of the array are of sufficient size to accommodate the at least one solid support), adding at least one detectable label to the mixture before, after or both before or after moving a portion of the mixture to the array of wells and measuring the analyte of interest in the wells. In certain embodiments, “using energy to exert a force to manipulate the first liquid droplet with the second liquid droplet” refers to the use of non-mechanical forces (namely, for example, energy created without the use of pumps and/or valves) to provide or exert a force that manipulates (such as merges or combines) at least the first and second liquid droplets (and optionally, additional droplets) into a mixture. Example of non-mechanical forces that can be used in the methods described herein include electric actuation force (such as droplet actuation, electrophoresis, electrowetting, dielectrophoresis, electrostatic actuation, electric field mediated, electrode mediated, capillary force, chromatography, centrifugation or aspiration) and/or acoustic force (such as surface acoustic wave (or “SAW”). In certain embodiments, the the electric actuation force generated is an alternating current. For example, the alternating current can have a root mean squred (rms) voltage of 10 V, 15 V, 20 V, 25 V, 30 V, 35V or more. For example, such alternating current can have a rms voltage of 10 V or more, 15 V or more, 20 V or more, 25 V or more, 30 V or more or 35 V or more. Alternatively, the alternating current can have a frequency in a radio frequency range.

In certain embodiments, if magnetic solid supports are used, an electric actuation force and a magnetic field can be applied and applied from opposition directions, relative to the at least a portion of the mixture. In certain other embodiments, the mixture is mixed by moving it: back and forth, in a circular pattern or by splitting it into two or more submixtures and then merging the submixtures. In certain other embodiments, an electric actuation force can be generated using a series or plurality of electrodes (namely, at least two or more, at least three or more, at least four or more, at least five or more, at least six or more, at least seven or more, at least eight or more, at least nine or more, at least ten or more, at least eleven or more, at least twelve or more, at least thirteen or more, at least fourteen or more, at least fifteen or more, etc.) to move the mixture to the array of wells in order to seal the wells (which are loaded with at least one solid support).

In certain embodiments, the moving of all or at least a portion of the mixture to an array of wells results in the loading (filling and/or placement) of the at least one solid support into the array of wells. In certain embodiments, a magnetic field is used to facilitate movement of the mixture and thus, at least one solid support, into one or more wells of the array. In certain embodiments, after the at least one solid supports are loaded into the wells, any solid supports that are not loaded into a well can be removed using routine techniques known in the art. For example, such removing can involve generating an electric actuation force (such as that described previously herein) with a series or plurality of electrodes to move a fluid droplet (such as a polarizable fluid droplet) to the array of wells to move at least a portion of the mixture to a distance (the length of which is not critical) from the array of wells. In certain embodiments, an aqueous washing liquid can be used to remove the solid supports not bound to any analyte of interest. In such embodiments, the removal involves generating an electric actuation force with a series or plurality of electrodes to move an aqueous wash (or washing) droplet (a third droplet) across the array of wells. The amount and type of aqueous liquid used for said washing is not critical.

In certain embodiments, the mixture in the method is an aqueous liquid. In other embodiments, the mixture is an immiscible liquid. In other embodiments, the liquid droplet is a hydrophobic liquid droplet. In other embodiments, the liquid droplet is a hydrophilic liquid droplet. In certain embodiments, the array of wells used in the method have a hydrophobic surface. In other embodiments, the array of wells has a hydrophilic surface.

In certain embodiments, the first liquid droplet used in the method is a polarizable liquid. In certain embodiments, the second liquid droplet used in the method is a polarizable liquid. In certain embodiments, the first and second liquid droplets used in the method are polarizable liquids. In certain embodiments, the mixture is a polarizable liquid. In certain embodiments one or more of the first droplet, second droplet and mixture is a polarizable liquid.

In certain embodiments, the at least one solid support comprises at least one binding member that specifically binds to the analyte of interest. In certain embodiments, the detectable label is added to the mixture before moving at least a portion of the mixture to the array of wells. In certain other embodiments, the detectable label is added to the mixture after the moving of at least a portion of the analyte of interest to the array of wells. In certain embodiments, the detectable label comprises at least one binding member that specifically binds to the analyte of interest. In certain embodiments, the detectable label comprises a chromagen, a florescent compound, an enzyme, a chemiluminescent compound or a radioactive compound. In certain embodiments, the binding member is a receptor, aptamer or antibody. In certain embodiments, the method further comprises positioning the at least a portion of the mixture over the array of wells using a capillary element configured to facilitate movement of the mixture to the array of wells.

In certain embodiments, the method described herein is performed using a microfluidics device. In certain embodiments, the method described herein is performed using a digital microfluidics device (DMF). In certain embodiments, method described herein is performed using a surface acoustic wave based microfluidics device (SAW). In certain embodiments, method described herein is performed using an integrated DMF and analyte detection device. In certain embodiments, method described herein is performed using an integrated surface acoustic wave based microfluidic device and analyte detection device. In certain embodiments, method described herein is performed using a Robotics based assay processing unit.

In certain embodiments, the measuring first involves determining the total number of solid supports in the well of the array (“total solid support number”). Next, the number of solid supports in the wells of the array that contain the detectable label are determined, such as, for example, determining the intensity of the signal produced by the detectable label (“positives”). The positives are subtracted from the total solid support number to provide the number of solid supports in the array of wells that do not contain a detectable label or are not detected (“negatives”). Then, the ratio of positives to negatives in the array of wells can be determined and then compared to a calibration curve. Alternatively, digital quantitation using the Poission equation P(x; μ) as shown below:

P(x;μ)=(e−μ)(μx)/x!

where:

e: A is a constant equal to approximately 2.71828,

μ: ix ghd mean number of successes that occur in a specified region, and

x: is the tactual number of successes that occur in a specified region.

Provided herein are methods for measuring or detecting an analyte present in a biological sample. The method includes contacting the sample with a first binding member, wherein the first binding member is immobilized on a solid support and wherein the first binding member specifically binds to the analyte; contacting the analyte with a second binding member, wherein the second binding member specifically binds to the analyte and wherein the second binding member includes a cleavable tag attached thereto; removing second binding member not bound to the analyte bound to the first binding member; cleaving the tag attached to the second binding member that is bound to the analyte bound to the first binding member; translocating the cleaved tag through or across one or more nanopores in a layer; detecting or measuring tags translocating through the layer; and assessing the tag translocating through the layer, wherein measuring the number of tags translocating through the layer measures the amount of analyte present in the sample, or wherein detecting tags translocating through the layer detects that the analyte is present in the sample. In some embodiments, measuring the tags translocating through the layer is assessed, wherein the number of tags translocating through the layer measures the amount of analyte present in the sample. In some embodiments, detecting the tags translocating through the layer is assessed, wherein detecting tags translocating through the layer detects that the analyte is present in the sample.

Provided herein are methods for measuring or detecting an analyte present in a biological sample. The method includes contacting the sample with a first binding member, wherein the first binding member is immobilized on a solid support and wherein the first binding member specifically binds to the analyte; contacting the analyte with a second binding member, wherein the second binding member specifically binds to the analyte and wherein the second binding member includes an aptamer; removing aptamer not bound to the analyte bound to the solid substrate; dissociating the aptamer bound to the analyte and translocating the dissociated aptamer through or across one or more nanopores in a layer; and assessing the aptamer translocating through the layer, wherein measuring the number of aptamers translocating through the layer measures the amount of analyte present in the sample, or detecting aptamers translocating through the layer detects that the analyte is present in the sample. In some embodiments, measuring the aptamers translocating through the layer is assessed, wherein the number of aptamers translocating through the layer measures the amount of analyte present in the sample. In some embodiments, detecting the aptamers translocating through the layer is assessed, wherein detecting tags translocating through the layer detects that the analyte is present in the sample.

In some embodiments, each tag, such as an aptamer, translocating through the layer is a translocation event. Measuring the number of translocation events measures the amount of analyte present in the sample. In some embodiments, the amount of analyte present in the sample can be determined by counting the number of translocation events during a set period of time and correlating the number of translocation events to a control. The standard curve can be determined by measuring the number of translocation events for control concentrations of analyte during a set period of time. In some embodiments, the amount of analyte present in the sample can be determined by measuring the amount of time for a set number of translocation events to occur and correlating to a control. The standard curve can be determined by measuring the time it takes for a set number of translocation events to occur for control concentrations of analyte. In some embodiments, the amount of analyte present in the sample can be determined by measuring the average time between translocation events to occur and correlating to a control. The standard curve can be determined by measuring the average time between translocation events to occur for control concentrations of analyte. In some embodiments, the control can be a reference standard comprising a calibration curve, standard addition, or digital polymerase chain reaction.

In exemplary cases, the method may include contacting the sample with a first binding member (“binding members” alternately referred to as “specific binding members,” and as described in section c) below), where the first binding member is immobilized on a solid support and where the first binding member specifically binds to the analyte; contacting the analyte with a second binding member, which second binding member specifically binds to the analyte and which second binding member includes a cleavable tag (“tag” as defined herein and described in section d) below) attached thereto; removing second binding member not bound to the analyte bound to the first binding member; cleaving the tag attached to the second binding member that is bound to the analyte bound to the first binding member; translocating the tag through nanopores in a layer; determining the number of tags translocating through the layer; determining concentration of the analyte in the sample based on the number of tags translocating through the layer. In certain embodiments, the concentration of the analyte may be determined by counting the number of tags translocating through the layer per unit time. In other embodiments, the concentration of the analyte may be determined by determining the time at which the number of tags translocating through the layer reaches a threshold.

The sample may be any test sample containing or suspected of containing an analyte of interest. As used herein, “analyte”, “target analyte”, “analyte of interest” are used interchangeably and refer to the analyte being measured in the methods and devices disclosed herein. Analytes of interest are further described below.

“Contacting” and grammatical equivalents thereof as used herein refer to any type of combining action which brings a binding member into sufficiently close proximity with the analyte of interest in the sample such that a binding interaction will occur if the analyte of interest specific for the binding member is present in the sample. Contacting may be achieved in a variety of different ways, including combining the sample with a binding member, exposing a target analyte to a binding member by introducing the binding member in close proximity to the analyte, and the like.

In certain cases, the first binding member may be immobilized on a solid support. As used herein, “immobilized” refers to a stable association of the first binding member with a surface of a solid support. By “stable association” is meant a physical association between two entities in which the mean half-life of association is one day or more, e.g., under physiological conditions. In certain aspects, the physical association between the two entities has a mean half-life of two days or more, one week or more, one month or more, including six months or more, e.g., 1 year or more, in PBS at 4° C. According to certain embodiments, the stable association arises from a covalent bond between the two entities, a non-covalent bond between the two entities (e.g., an ionic or metallic bond), or other forms of chemical attraction, such as hydrogen bonding, Van der Waals forces, and the like.

The solid support having a surface on which the binding reagent is immobilized may be any convenient surface in planar or non-planar conformation, such as a surface of a microfluidic chip, an interior surface of a chamber, an exterior surface of a bead (as defined herein), or an interior and/or exterior surface of a porous bead. For example, the first binding member may be attached covalently or non-covalently to a bead, e.g., latex, agarose, sepharose, streptavidin, tosylactivated, epoxy, polystyrene, amino bead, amine bead, carboxyl bead, or the like. In certain embodiments, the bead may be a particle, e.g., a microparticle. In some embodiments, the microparticle may be between about 0.1 nm and about 10 microns, between about 50 nm and about 5 microns, between about 100 nm and about 1 micron, between about 0.1 nm and about 700 nm, between about 500 nm and about 10 microns, between about 500 nm and about 5 microns, between about 500 nm and about 3 microns, between about 100 nm and 700 nm, or between about 500 nm and 700 nm. For example, the microparticle may be about 4-6 microns, about 2-3 microns, or about 0.5-1.5 microns. Particles less than about 500 nm are sometimes considered nanoparticles. Thus, the microparticle optionally may be a nanoparticle between about 0.1 nm and about 500 nm, between about 10 nm and about 500 nm, between about 50 nm and about 500 nm, between about 100 nm and about 500 nm, about 100 nm, about 150 nm, about 200 nm, about 250 nm, about 300 nm, about 350 nm, about 400 nm, about 450 nm, or about 500 nm.

In certain embodiments, the bead may be a magnetic bead or a magnetic particle. In certain embodiments, the bead may be a magnetic nanobead, nanoparticle, microbead or microparticle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, NiO/Fe. Examples of ferrimagnetic materials include NiFe₂O₄, CoFe₂O₄, Fe₃O₄ (or FeO.Fe₂O₃). Beads can have a solid core portion that is magnetic and is surrounded by one or more non-magnetic layers. Alternately, the magnetic portion can be a layer around a non-magnetic core. The solid support on which the first binding member is immobilized may be stored in dry form or in a liquid. The magnetic beads may be subjected to a magnetic field prior to or after contacting with the sample with a magnetic bead on which the first binding member is immobilized.

After the contacting step, the sample and the first binding member may be incubated for a sufficient period of time to allow for the binding interaction between the binding member and analyte to occur. In addition, the incubating may be in a binding buffer that facilitates the specific binding interaction. The binding affinity and/or specificity of the first binding member and/or the second binding member may be manipulated or altered in the assay by varying the binding buffer. In some embodiments, the binding affinity and/or specificity may be increased by varying the binding buffer. In some embodiments, the binding affinity and/or specificity may be decreased by varying the binding buffer.

The binding affinity and/or specificity of the first binding member and/or the second binding member may be measured using the disclosed methods and device described below. In some embodiments, the one aliquot of sample is assayed using one set of conditions and compared to another aliquot of sample assayed using a different set of conditions, thereby determining the effect of the conditions on the binding affinity and/or specificity. For instance, changing or altering the condition can be one or more of removing the target analyte from the sample, adding a molecule that competes with the target analyte or the ligand for binding, and changing the pH, salt concentration, or temperature. Additionally or alternatively, a duration of time can be the variable and changing the condition may include waiting for a duration of time before again performing the detection methods.

In some embodiments, after the tag or aptamer passes through the pore of a nanopore device, if provided, the device can be reconfigured to reverse the movement direction of the tag or aptamer such that the tag or aptamer can pass through the pore again and be re-measured or re-detected, for example, in a confirmatory assay on an infectious disease assay to confirm the measured results.

The binding buffer may include molecules standard for antigen-antibody binding buffers such as, albumin (e.g., BSA), non-ionic detergents (Tween-20, Triton X-100), and/or protease inhibitors (e.g., PMSF). In certain cases, the binding buffer may be added to the microfluidic chip, chamber, etc., prior to or after adding the sample. In certain cases, the first binding member may be present in a binding buffer prior to contacting with the sample. The length of time for binding interaction between the binding member and analyte to occur may be determined empirically and may depend on the binding affinity and binding avidity between the binding member and the analyte. In certain embodiments, the contacting or incubating may be for a period of 5 sec to 1 hour, such as, 10 sec-30 minutes, or 1 minute-15 minutes, or 5 minutes-10 minutes, e.g., 10 sec, 15 sec, 30 sec, 1 minute, 5 minutes, 10 minutes, 15 minutes, 30 minutes, 45 minutes, 1 hour or 2 hours. Other conditions for the binding interaction, such as, temperature, salt concentration, may also be determined empirically or may be based on manufacturer's instructions. For example, the contacting may be carried out at room temperature (21° C.-28° C., e.g., 23° C.-25° C.), 37° C., or 4° C. In certain embodiments, an optional mixing of the sample with the first binding member may be carried out during the contacting step.

Following complex formation between the immobilized first binding member and the analyte, any unbound analyte may be removed from the vicinity of the first binding member along with the sample while the complex of the first binding member and the analyte may be retained due to its association with the solid support. Optionally, the solid support may be contacted with a wash buffer to remove any molecules non-specifically bound to the solid support.

After the first contacting step, and the optional removal of sample and/or optional wash steps, the complex of the first binding member and the analyte may be contacted with a second binding member, thereby leading to the formation of a sandwich complex in which the analyte is bound by the two binding members. An optional mixing of the second member with the first binding member-analyte complex may be carried out during the second contacting step. In some embodiments, immobilization of the analyte molecules with respect to a surface may aid in removal of any excess second binding members from the solution without concern of dislodging the analyte molecule from the surface. In some embodiments, the second binding member may include a detectable label comprising one or more signal-producing substances, such as chromagens, fluorescent compounds, chemiluminescent compounds, enzymes, radioactive compounds, and the like. In some embodiments, the second binding member may include a tag, such as a cleavable tag, attached thereto.

As noted above, the second contacting step may be carried out in conditions sufficient for binding interaction between the analyte and the second binding member. Following the second contacting step, any unbound second binding member may be removed, followed by an optional wash step. Any unbound second binding member may be separated from the complex of the first binding member-analyte-second binding member by a suitable means such as, droplet actuation, electrophoresis, electrowetting, dielectrophoresis, electrostatic actuation, electric field mediated, electrode mediated, capillary force, chromatography, centrifugation, aspiration or SAW. Upon removal of any unbound second binding member from the vicinity of the complex of the first binding member-analyte-second binding member, the detectable label or tag attached to the second binding member present in the complex of the first binding member-analyte-second binding member may be separated by a suitable means or may be detected using techniques known in the art. In some embodiments, the detectable label comprises a detectable label comprising one or more signal-producing substances, such as chromagens, fluorescent compounds, enzymes, chemiluminescent compounds, radioactive compounds, and the like. Alternatively, in some embodiments, if the detectable label comprises a tag, the tag can be cleaved or disassociated from the complex which remains after removal of unbound reagents. For example, the tag may be attached to the second binding member via a cleavable linker (e.g., “cleavable linker” as described in section f) below). The complex of the first binding member-analyte-second binding member may be exposed to a cleavage agent that mediates cleavage of the cleavable linker.

In certain embodiments, the separation of the tag from the first binding member-analyte-second binding member complex is carried out under conditions that do not result in disruption of the complex, resulting in release of only the tag from the complex. In other cases, the separation of the tag from the first binding member-analyte-second binding member complex is carried out under conditions that may result in disruption of the complex, resulting in release of the tag, as well as one or more of the second binding member, the analyte, the first binding member from the complex. In certain embodiments, the size of the nanopore used for counting the tag may prevent the second binding member, the analyte, the first binding member from translocating through the nanopore. In other embodiments, where the complex of second binding member, the analyte, the first binding member is retained on the solid support, the nanopore may not be sized to exclude the second binding member, the analyte, and the first binding member.

The separation step results in the generation of a free tag that can be caused to translocate through or across a nanopore or nanopore layer (as described in section f) below) under the influence of an electric field. In certain cases, the cleavage step may result in separation of substantially all the tag molecule(s) attached to each of the second binding member in the first binding member-analyte-second binding member complex. The number of tag molecules can be correlated to the number of analyte molecules in the complex which are proportional to the concentration of the analyte in the sample. In certain embodiments, the correlation between the counted tag and the analyte concentration may be direct (higher number of tag molecules relates to higher analyte concentration). In embodiments where a tagged competitor or tagged analyte, such as a tracer (as defined herein), is combined with the sample, which tagged competitor or tagged analyte competes with the analyte in the sample for binding to the first binding member, the correlation between the counted tag and the analyte concentration may be inverse (lower number of tag molecules relates to higher analyte concentration). The correlation between the number of tag molecules and analyte concentration, whether direct or inverse, may be linear or logarithmic. Thus, the number of tag molecules translocating through the nanopore may be used to determine analyte concentration in the sample. In certain embodiments, the concentration of the analyte may be determined by counting the number of tags translocating through the layer per unit time. In other embodiments, the concentration of the analyte may be determined by determining the time at which the number of tags translocating through the layer reaches a threshold. In certain embodiments, the number of tag molecules translocating through or across a nanopore may be determined by the frequency of current blockage at the nanopore per unit time. Signal detection is further described in section g) below. As described in section d) below, the tag molecule may be a nanoparticle or a nanobead (“nanoparticle” and “nanobead” as defined herein).

The number of tags incorporated in the second binding member (i.e., the number of tags in the tag/second binding member conjugate) provides a defined stoichiometry with the analyte. In certain embodiments, a tag may be attached to the second binding member using a procedure that yields a consistent number of tag(s) attached to each second binding member. The number of tags may be optimized based on the speed of counting. A faster read rate may be obtained by including more tags on the binding member as the count rate is dependent on the concentration. The number of tags may be optimized based on the stoichiometry of tag incorporation, for example 1:1 or 1:4 incorporation rate. In some embodiments, there is a 1:5 incorporation rate. For example, one second binding member may have 1 tag molecule, 2 tag molecules, 3 tag molecules, 4 tag molecules, or up to 10 tag molecules attached thereto. In some embodiments, one second binding member may have 5 tag molecules attached thereto. A number of conjugation methods for conjugating a tag to a second binding member (e.g., a peptide, a polypeptide, a nucleic acid) are known, any of which may be used to prepare tagged second binding members for use in the present methods and devices. For example, site specific conjugation of a tag to an analyte specific antibody may be carried out using thiol-maleimide chemistry, amine-succinimidyl chemistry, THIOBRIDGE™ technology, using antibodies with a C- or N-terminal hexahistidine tag, antibodies with an aldehyde tag, copper-free click reaction, and the like.

In some embodiments, the methods can measure the amount of analyte by determining the number of translocation events. In some embodiments, one or more translocation event(s) can correspond to a binding event between a binding member and an analyte depending on the stoichiometry of tag incorporation into the specific binding member. For example, if one tag is incorporated per binding member, then one translocation event represents the binding of the binding member to the analyte; if two tags are incorporated per binding member, then two translocation events represents the binding of the binding member to the analyte; if three tags are incorporated per binding member, then three translocation events represents the binding of the binding member to the analyte, etc.

In another embodiment, the second binding member may be an aptamer that specifically binds to the analyte. In this embodiment, a tag may not be attached to the aptamer. Rather, the aptamer is counted as it translocates through or across a nanopore, i.e., the aptamer serves a dual function of being the second binding member and being the tag. In these embodiments, the aptamer in the complex of first binding member-analyte-aptamer complex may be dissociated from the complex by any suitable method. For example, prior to translocation through or across a nanopore, the aptamer bound to the complex of first binding member-analyte may be dissociated via a denaturation step. The denaturation step may involve exposure to a chaotropic reagent, a high salt solution, an acidic reagent, a basic reagent, solvent, or a heating step. The aptamer may then be translocated through or across a nanopore and the number of aptamer molecules translocating through or across a nanopore may be used to determine concentration of the analyte in the sample.

As noted herein, the tag or aptamer may include a nucleic acid. In certain embodiments, the quantification of the analyte, or counting step using a nanopore, if provided, does not include determining the identity of the tag or the aptamer by determining identity of at least a portion of the nucleic acid sequence present in the tag/aptamer. For example, the counting step may not include determining a sequence of the tag/aptamer. In other embodiments, the tag/aptamer may not be sequenced, however, identity of the tag/aptamer may be determined to the extent that one tag/aptamer may be distinguished from another tag/aptamer based on a differentiable signal associated with the tag/aptamer due its size, conformation, charge, amount of charge and the like. Identification of tag/aptamer may be useful in methods involving simultaneous analysis of a plurality of different analytes in a sample, for example, two, three, four, or more different analytes in a sample.

In certain embodiments, the simultaneous analysis of multiple analytes in a single sample may be performed by using a plurality of different first and second binding members where a pair of first and second binding members is specific to a single analyte in the sample. In these embodiments, the detectable label or tag associated with the second binding member of a first pair of first and second binding members specific to a single analyte may be distinguishable from the detectable label or tag associated with the second binding member of a second pair of first and second binding members specific to a different analyte. As noted above, a first detectable label or tag may be distinguishable from second detectable label or tag based on difference in signal-producing substances, dimensions, and/or charge, etc.

In some embodiments, the concentration of an analyte in the fluid sample that may be substantially accurately determined is less than about 5000 fM (femtomolar), less than about 3000 fM, less than about 2000 fM, less than about 1000 fM, less than about 500 fM, less than about 300 fM, less than about 200 fM, less than about 100 fM, less than about 50 fM, less than about 25 fM, less than about 10 fM, less than about 5 fM, less than about 2 fM, less than about 1 fM, less than about 500 aM (attomolar), less than about 100 aM, less than about 10 aM, less than about 5 aM, less than about 1 aM, less than about 0.1 aM, less than about 500 zM (zeptomolar), less than about 100 zM, less than about 10 zM, less than about 5 zM, less than about 1 zM, less than about 0.1 zM, or less.

In some cases, the limit of detection (e.g., the lowest concentration of an analyte which may be determined in solution) is about 100 fM, about 50 fM, about 25 fM, about 10 fM, about 5 fM, about 2 fM, about 1 fM, about 500 aM (attomolar), about 100 aM, about 50 aM, about 10 aM, about 5 aM, about 1 aM, about 0.1 aM, about 500 zM (zeptomolar), about 100 zM, about 50 zM, about 10 zM, about 5 zM, about 1 zM, about 0.1 zM, or less. In some embodiments, the concentration of analyte in the fluid sample that may be substantially accurately determined is between about 5000 fM and about 0.1 fM, between about 3000 fM and about 0.1 fM, between about 1000 fM and about 0.1 fM, between about 1000 fM and about 0.1 zM, between about 100 fM and about 1 zM, between about 100 aM and about 0.1 zM, or less.

The upper limit of detection (e.g., the upper concentration of an analyte which may be determined in solution) is at least about 100 fM, at least about 1000 fM, at least about 10 pM (picomolar), at least about 100 pM, at least about 100 pM, at least about 10 nM (nanomolar), at least about 100 nM, at least about 1000 nM, at least about 10 μM, at least about 100 μM, at least about 1000 μM, at least about 10 mM, at least about 100 mM, at least about 1000 mM, or greater.

In some cases, the presence and/or concentration of the analyte in a sample may be detected rapidly, usually in less than about 1 hour, e.g., 45 minutes, 30 minutes, 15 minutes, 10 minutes, 5 minutes, 1 minute, or 30 seconds.

In certain embodiments, at least some steps of the methods described herein may be carried out on a digital microfluidics device, such as the device described in section 3, below. In certain embodiments, the methods of the present disclosure are carried out using a digital microfluidics device in conjunction with an analyte detection device. For example, the digital microfluidics device and the analyte detection device may be separate devices and a droplet containing the detectable label, cleaved tag(s) or the dissociated aptamer(s) may be generated in the microfluidics device and transported to the analyte detection device. In certain embodiments, a droplet containing the cleaved tag(s) or the dissociated aptamer(s) may be aspirated from the microfluidics device and transported to the analyte detection device, which can be a nanopore device, using pipette operated by a user or a robot.

In certain embodiments, the methods of the present disclosure are carried out using a device in which a digital microfluidics module is integrated with an analyte detection device, such as the devices described below. In certain embodiments, the digital integrated microfluidics module and the analyte detection device may be reversibly integrated. For example, the two modules may be combined physically to form the integrated device and which device could then be separated into the individual modules. In certain embodiments, the methods of the present disclosure are carried out using a disposable cartridge that includes a microfluidics module with a built-in analyte detection device. Exemplary embodiments of the devices used for performing the methods provided herein are described further in the next section.

In certain cases, the microfluidics device or the microfluidics module of the device integrated (reversibly or fully) with the nanopore module, if provided, may include a first substrate and a second substrate arranged in a spaced apart manner, where the first substrate is separated from the second substrate by a gap/space, and where at least the steps of contacting the sample with a first binding member, contacting the analyte with a second binding member, removing second binding member not bound to the analyte bound to the first binding member, and cleaving the tag attached to the second binding member (that remains bound to the analyte bound to the first binding member) is carried out in the space/gap between the first and second substrates.

Exemplary embodiments of the present method include merging a sample droplet containing an analyte of interest with a droplet containing a first binding member that binds to the analyte of interest and that may be immobilized on a solid support (such as magnetic particles or beads). The single merged droplet can be incubated for a period of time sufficient to allow binding of the first binding member to the analyte of interest. Optionally, the single droplet may be agitated to facilitate mixing of the sample with the first binding member. Mixing may be achieved by moving the single droplet back and forth, moving the single droplet around over a plurality of electrodes, splitting a droplet and then merging the droplets, or using SAWs, and the like. Next, the single droplet may be subjected to a magnetic force to retain the beads at a location in the device while the droplet may be moved away and replaced with a droplet containing a second binding member, which second binding member can optionally contain a detectable label. An optional wash step may be performed, prior to adding the second binding member, by moving a droplet of wash buffer to the location at which the beads are retained using the magnetic force. After a period of time sufficient for the second binding member to bind the analyte bound to the first binding member, the droplet containing the second binding member may be moved away while the beads are retained at the first location. The beads may be washed using a droplet of wash buffer. Following the wash step, the magnetic force may be removed and the droplet containing labeled beads (containing the first specific binding member/analyte/second specific binding member—an optional detectable label) are moved to a detection module such as that described herein. The labeled beads are allowed to settle into an array of wells in the detection module. The beads may settle via gravitational force or by applying electric or magnetic force. Following a wash step to remove any beads not located inside the wells, the wells may be sealed using a hydrophobic liquid. In the above embodiments, optionally, after the combining, a droplet may be manipulated (e.g., moved back and forth, moved in a circular direction, oscillated, split/merged, exposed to SAW, etc.) to facilitate mixing of the sample with the assay reagents, such as, the first binding member, second binding member, etc. In embodiments where the detectable label is an enzyme, a substrate can be added either before or after moving the complex is moved to the array of wells.

The moving of the droplets in the integrated microfluidic and analyte detection device may be carried out using electrical force (e.g., electrowetting, dielectrophoresis, electrode-mediated, opto-electrowetting, electric-field mediated, and electrostatic actuation) pressure, surface acoustic waves and the like. The force used for moving the droplets may be determined based on the specifics of the device, which are described in the following sections, and for the particular device described herein.

Exemplary embodiments of the present method include generating a droplet of the sample and combining the droplet of the sample with a droplet containing the first binding member to generate a single droplet. The first binding member may be immobilized on a solid substrate, such as, a bead (e.g., a magnetic bead). The single droplet may be incubated for a time sufficient to allow binding of the first binding member to an analyte present in the sample droplet. Optionally, the single droplet may be agitated to facilitate mixing of the sample with the first binding member. Mixing may be achieved by moving the single droplet back and forth, moving the single droplet around over a plurality of electrodes, splitting a droplet and then merging the droplets, or using SAWs, and the like. Next, the single droplet may be subjected to a magnetic force to retain the beads at a location in the device while the droplet may be moved away and replaced with a droplet containing a second binding member. An optional wash step may be performed, prior to adding the second binding member, by moving a droplet of wash buffer to the location at which the beads are retained using the magnetic force. After a period of time sufficient for the second binding member to bind the analyte bound to the first binding member, the droplet containing the second binding member may be moved away while the beads are retained at the first location. The beads may be washed using a droplet of wash buffer followed by contacting the beads with a droplet containing a cleavage reagent to cleave the tag attached to the second binding member. In embodiments where the tag is attached to the second binding member via a photocleavable linker, the beads may be exposed to light of the appropriate wavelength to cleave the linker. In certain cases, the beads may be exposed to a droplet of buffer prior to cleavage of the photocleavable linker. Optionally, after the washing step to remove any unbound second binding member, a droplet containing buffer may be left covering the beads, the magnetic force retaining the beads at the first location may be removed and the buffer droplet containing the beads may be moved to a second location at which the photocleavage may be carried out. The droplet containing the cleaved tags may then be moved to the nanopore device or the nanopore module portion of the integrated device. In embodiments using aptamer as the second binding member, after the washing step to remove any unbound aptamer, a droplet containing buffer may be left covering the beads, the magnetic force retaining the beads at the first location may be removed and the buffer droplet containing the beads may be moved to a second location at which the dissociation of the aptamer may be carried out. In other embodiments, after the washing step, the beads may be exposed to a droplet of a reagent for dissociating aptamer bound to the analyte. A droplet containing the dissociated aptamer may be moved to the nanopore while the beads may be retained in place using a magnet. The droplet containing the dissociated aptamer may be moved to the nanopore device or the nanopore module portion of the integrated device.

In an alternate embodiment, the first binding member may be immobilized on a surface of the first or the second substrate at a location in the gap/space. The step of contacting a sample with the first binding member may include moving a droplet of the sample to the location in the gap/space at which the first binding member is immobilized. The subsequent steps may be substantially similar to those described above for first binding member immobilized on magnetic beads.

After the cleaving/dissociating step, the droplet containing the cleaved tag(s)/dissociated aptamer(s) may be moved to the nanopore device or the nanopore module of the integrated device. As noted above, the droplet(s) may be moved using a liquid transfer system, such as a pipette. In certain cases, the microfluidic module may be fluidically connected to the nanopore module. Fluidic connection may be achieved by connecting the microfluidics module to the nanopore module via a channel or by placing the nanopore module within the microfluidics module, either reversibly or during the manufacturing process of the integrated device. Such devices are further described in the following section.

In the above embodiments, optionally, after the combining, a droplet may be manipulated (e.g., moved back and forth, moved in a circular direction, oscillated, split/merged, exposed to SAW, etc.) to facilitate mixing of the sample with the assay reagents, such as, the first binding member, second binding member, etc.

The moving of the droplets in the integrated microfluidics nanopore device may be carried out using electrical force (e.g., electrowetting, dielectrophoresis, electrode-mediated, opto-electrowetting, electric-field mediated, and electrostatic actuation) pressure, surface acoustic waves and the like. The force used for moving the droplets may be determined based on the specifics of the device, which are described in the following sections a) through g) below, and for the particular device described in section 3.

i. Multiplexing

The methods may include one or more (or alternately two or more) specific binding members to detect one or more (or alternately two or more) target analytes in the sample in a multiplexing assay. Each of the one or more (or alternately two or more) specific binding members binds to a different target analyte and each specific binding member is labeled with a different detectable label, tag and/or aptamer. For example, a first specific binding member binds to a first target analyte, a second specific binding member binds to a second target analyte, a third specific binding member binds to a third target analyte, etc. and the first specific binding member is labeled with a detectable label, the second specific binding member is labeled with a second detectable label, the third specific binding member is labeled with a third detectable label, etc. For example the first, second and third detectable labels can each have a different color. Alternatively, different types of labels can be used, such as, for example, the first label is an enzymatic label, the second label is a chromagen and the third label is a chemiluminescent compound. In another example, a first specific binding member binds to a first target analyte, a second specific binding member binds to a second target analyte, a third specific binding member binds to a third target analyte, etc. and the first specific binding member is labeled with a first tag and/or aptamer, the second specific binding member is labeled with a second tag and/or aptamer, the third specific binding member is labeled with a third tag and/or aptamer, etc. In some embodiments, a first condition causes the cleavage or release of the first tag if the first specific binding member is labeled with a tag or the dissociation or release of the first aptamer if the first specific binding member is labeled with an aptamer, a second condition causes the cleavage or release of the second tag if the second specific binding member is labeled with a tag or the dissociation or release of the second aptamer if the second specific binding member is labeled with an aptamer, a third condition causes the cleavage or release of the third tag if the third specific binding member is labeled with a tag or the dissociation or release of the third aptamer if the third specific binding member is labeled with an aptamer, etc. In some embodiments, the conditions of the sample can be changed at various times during the assay, allowing detection of the first tag or aptamer, the second tag or aptamer, the third tag or aptamer, etc., thereby detecting one or more (or alternately two or more) target analytes. In some embodiments, the one or more (or alternately two or more) cleaved tags and/or dissociated aptamers are detected simultaneously through the pore based on the residence duration in the nanopore, magnitude of current impedance, or a combination thereof.

ii. Exemplary Target Analytes

As will be appreciated by those in the art, any analyte that can be specifically bound by a first binding member and a second binding member may be detected and, optionally, quantified using methods and devices of the present disclosure.

In some embodiments, the analyte may be a biomolecule. Non-limiting examples of biomolecules include macromolecules such as, proteins, lipids, and carbohydrates. In certain instances, the analyte may be hormones, antibodies, growth factors, cytokines, enzymes, receptors (e.g., neural, hormonal, nutrient, and cell surface receptors) or their ligands, cancer markers (e.g., PSA, TNF-alpha), markers of myocardial infarction (e.g., troponin, creatine kinase, and the like), toxins, drugs (e.g., drugs of addiction), metabolic agents (e.g., including vitamins), and the like. Non-limiting embodiments of protein analytes include peptides, polypeptides, protein fragments, protein complexes, fusion proteins, recombinant proteins, phosphoproteins, glycoproteins, lipoproteins, or the like.

In certain embodiments, the analyte may be a post-translationally modified protein (e.g., phosphorylated, methylated, glycosylated protein) and the first or the second binding member may be an antibody specific to a post-translational modification. A modified protein may be bound to a first binding member immobilized on a solid support where the first binding member binds to the modified protein but not the unmodified protein. In other embodiments, the first binding member may bind to both the unmodified and the modified protein, and the second binding member may be specific to the post-translationally modified protein.

In some embodiments, the analyte may be a cell, such as, circulating tumor cell, pathogenic bacteria, viruses (including retroviruses, herpesviruses, adenoviruses, lentiviruses, Filoviruses (ebola), hepatitis viruses (e.g., A, B, C, D, and E); HPV, etc.; spores, etc.

A non-limiting list of analytes that may be analyzed by the methods presented herein include Aβ42 amyloid beta-protein, fetuin-A, tau, secretogranin II, prion protein, Alpha-synuclein, tau protein, neurofilament light chain, parkin, PTEN induced putative kinase 1, DJ-1, leucine-rich repeat kinase 2, mutated ATP13A2, Apo H, ceruloplasmin, Peroxisome proliferator-activated receptor gamma coactivator-1 alpha (PGC-1α), transthyretin, Vitamin D-binding Protein, proapoptotic kinase R (PKR) and its phosphorylated PKR (pPKR), CXCL13, IL-12p40, CXCL13, IL-8, Dkk-3 (semen), p14 endocan fragment, Serum, ACE2, autoantibody to CD25, hTERT, CAI25 (MUC 16), VEGF, sIL-2, Osteopontin, Human epididymis protein 4 (HE4), Alpha-Fetoprotein, Albumin, albuminuria, microalbuminuria, neutrophil gelatinase-associated lipocalin (NGAL), interleukin 18 (IL-18), Kidney Injury Molecule-1 (KIM-1), Liver Fatty Acid Binding Protein (L-FABP), LMP1, BARF1, IL-8, carcinoembryonic antigen (CEA), BRAF, CCNI, EGRF, FGF19, FRS2, GREB1, and LZTS1, alpha-amylase, carcinoembryonic antigen, CA 125, IL8, thioredoxin, beta-2 microglobulin levels—monitor activity of the virus, tumor necrosis factor-alpha receptors—monitor activity of the virus, CA15-3, follicle-stimulating hormone (FSH), leutinizing hormone (LH), T-cell lymphoma invasion and metastasis 1 (TIAM1), N-cadherin, EC39, amphiregulin, dUTPase, secretory gelsolin (pGSN), PSA (prostate specific antigen), thymosin βl5, insulin, plasma C-peptide, glycosylated hemoglobin (HBA1c), C-Reactive Protein (CRP), Interleukin-6 (IL-6), ARHGDIB (Rho GDP-dissociation inhibitor 2), CFL1 (Cofilin-1), PFN1 (profilin-1), GSTP1 (Glutathione S-transferase P), S100A11 (Protein S100-A11), PRDX6 (Peroxiredoxin-6), HSPE1 (10 kDa heat shock protein, mitochondrial), LYZ (Lysozyme C precursor), GPI (Glucose-6-phosphate isomerase), HIST2H2AA (Histone H2A type 2-A), GAPDH (Glyceraldehyde-3-phosphate dehydrogenase), HSPG2 (Basement membrane-specific heparan sulfate proteoglycan core protein precursor), LGALS3BP (Galectin-3-binding protein precursor), CTSD (Cathepsin D precursor), APOE (Apolipoprotein E precursor), IQGAP1 (Ras GTPase-activating-like protein IQGAP1), CP (Ceruloplasmin precursor), and IGLC2 (IGLC1 protein), PCDGF/GP88, EGFR, HER2, MUC4, IGF-IR, p27(kip1), Akt, HER3, HER4, PTEN, PIK3CA, SHIP, Grb2, Gab2, PDK-1 (3-phosphoinositide dependent protein kinase-1), TSC1, TSC2, mTOR, MIG-6 (ERBB receptor feedback inhibitor 1), S6K, src, KRAS, MEK mitogen-activated protein kinase 1, cMYC, TOPO II topoisomerase (DNA) II alpha 170 kDa, FRAP1, NRG1, ESR1, ESR2, PGR, CDKN1B, MAP2K1, NEDD4-1, FOXO3A, PPP1R1B, PXN, ELA2, CTNNB1, AR, EPHB2, KLF6, ANXA7, NKX3-1, PITX2, MKI67, PHLPP, adiponectin (ADIPOQ), fibrinogen alpha chain (FGA), leptin (LEP), advanced glycosylation end product-specific receptor (AGER aka RAGE), alpha-2-HS-glycoprotein (AHSG), angiogenin (ANG), CD14 molecule (CD14), ferritin (FTH1), insulin-like growth factor binding protein 1 (IGFBP1), interleukin 2 receptor, alpha (IL2RA), vascular cell adhesion molecule 1 (VCAM1) and Von Willebrand factor (VWF), myeloperoxidase (MPO), IL1α, TNFα, perinuclear anti-neutrophil cytoplasmic antibody (p-ANCA), lactoferrin, calprotectin, Wilm's Tumor-1 protein, Aquaporin-1, MLL3, AMBP, VDAC1, E. coli enterotoxins (heat-labile exotoxin, heat-stable enterotoxin), influenza HA antigen, tetanus toxin, diphtheria toxin, botulinum toxins, Shiga toxin, Shiga-like toxin I, Shiga-like toxin II, Clostridium difficile toxins A and B, etc.

Exemplary targets of nucleic acid aptamers that may be measured in a sample such as an environmental sample, a biological sample obtained from a patient or subject in need using the subject methods and devices include: drugs of abuse (e.g. cocaine), protein biomarkers (including, but not limited to, Nucleolin, nuclear factor-kB essential modulator (NEMO), CD-30, protein tyrosine kinase 7 (PTK7), vascular endothelial growth factor (VEGF), MUC1 glycoform, immunoglobulin μ Heavy Chains (IGHM), Immunoglobulin E, αvβ3 integrin, α-thrombin, HIV gp120, NF-κB, E2F transcription factor, HER3, Plasminogen activator inhibitor, Tenascin C, CXCL12/SDF-1, prostate specific membrane antigen (PSMA), gastric cancer cells, HGC-27); cells (including, but not limited to, non-small cell lung cancer (NSCLC), colorectal cancer cells, (DLD-1), H23 lung adenocarcinoma cells, Ramos cells, T-cell acute lymphoblastic leukemia (T-ALL) cells, CCRF-CEM, acute myeloid leukemia (AML) cells (HL60), small-cell lung cancer (SCLC) cells, NCIH69, human glioblastoma cells, U118-MG, PC-3 cells, HER-2-overexpressing human breast cancer cells, SK-BR-3, pancreatic cancer cell line (Mia-PaCa-2)); and infectious agents (including, but not limited to, Mycobacterium tuberculosis, Staphylococcus aureus, Shigella dysenteriae, Escherichia coli O157:H7, Campylobacter jejuni, Listeria monocytogenes, Pseudomonas aeruginosa, Salmonella O8, Salmonella enteritidis).

Exemplary targets of protein or peptide aptamers that may be measured in a sample obtained from a patient or subject in need using the subject methods and devices include, but are not limited to: HBV core capsid protein, CDK2, E2F transcription factor, Thymidylate synthase, Ras, EB1, and Receptor for Advanced Glycated End products (RAGE). Aptamers, and use and methods of production thereof are reviewed in e.g., Shum et al., J Cancer Ther. 2013 4:872; Zhang et al., Curr Med Chem. 2011; 18:4185; Zhu et al., Chem Commun (Camb). 2012 48:10472; Crawford et al., Brief Funct Genomic Proteomic. 2003 2:72; Reverdatto et al., PLoS One. 2013 8:e65180.

iii. Samples

As used herein, “sample”, “test sample”, “biological sample” refer to fluid sample containing or suspected of containing an analyte of interest. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing the analyte may be assayed directly. The source of the analyte molecule may be synthetic (e.g., produced in a laboratory), the environment (e.g., air, soil, fluid samples, e.g., water supplies, etc.), an animal, e.g., a mammal, a plant, or any combination thereof. In a particular example, the source of an analyte is a human bodily substance (e.g., bodily fluid, blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL.

In some cases, the fluid sample may be diluted prior to use in an assay. For example, in embodiments where the source of an analyte molecule is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use.

In some cases, the sample may undergo pre-analytical processing. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art. In some cases, the fluid sample may be concentrated prior to use in an assay. For example, in embodiments where the source of an analyte molecule is a human body fluid (e.g., blood, serum), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

In certain embodiments, the analyte is not amplified (i.e., the copy number of the analyte is not increased) prior to the measurement of the analyte. For example, in cases where the analyte is DNA or RNA, the analyte is not replicated to increase copy numbers of the analyte. In certain cases, the analyte is a protein or a small molecule.

iv. Specific Binding Members

As will be appreciated by those in the art, the binding members will be determined by the analyte to be analyzed. Binding members for a wide variety of target molecules are known or can be readily found or developed using known techniques. For example, when the target analyte is a protein, the binding members may include proteins, particularly antibodies or fragments thereof (e.g., antigen-binding fragments (Fabs), Fab′ fragments, F(ab′)₂ fragments, recombinant antibodies, chimeric antibodies, single-chain Fvs (“scFv”), single chain antibodies, single domain antibodies, such as variable heavy chain domains (“VHH”; also known as “VHH fragments”) derived from animals in the Camelidae family (VHH and methods of making them are described in Gottlin et al., Journal of Biomolecular Screening, 14:77-85 (2009)), recombinant VHH single-domain antibodies, and V_{NAR} fragments, disulfide-linked Fvs (“sdFv”), and anti-idiotypic (“anti-Id”) antibodies, and functionally active epitope-binding fragments of any of the above, full-length polyclonal or monoclonal antibodies, antibody-like fragments, etc.), other proteins, such as receptor proteins, Protein A, Protein C, or the like. In case where the analyte is a small molecule, such as, steroids, bilins, retinoids, and lipids, the first and/or the second binding member may be a scaffold protein (e.g., lipocalins) or a receptor. In some cases, binding member for protein analytes may be a peptide. For example, when the target analyte is an enzyme, suitable binding members may include enzyme substrates and/or enzyme inhibitors which may be a peptide, a small molecule and the like. In some cases, when the target analyte is a phosphorylated species, the binding members may comprise a phosphate-binding agent. For example, the phosphate-binding agent may comprise metal-ion affinity media such as those describe in U.S. Pat. No. 7,070,921 and U.S. Patent Application No. 20060121544.

In certain cases, at least one of the binding members may be an aptamer, such as those described in U.S. Pat. Nos. 5,270,163, 5,475,096, 5,567,588, 5,595,877, 5,637,459, 5,683,867, 5,705,337. Nucleic acid aptamers (e.g., single-stranded DNA molecules or single-stranded RNA molecules) may be developed for capturing virtually any target molecule. Aptamers bind target molecules in a highly specific, conformation-dependent manner, typically with very high affinity, although aptamers with lower binding affinity can be selected. Aptamers may distinguish between target analyte molecules based on very small structural differences such as the presence or absence of a methyl or hydroxyl group and certain aptamers can distinguish between D- and L-enantiomers and diastereomers. Aptamers may bind small molecular targets, including drugs, metal ions, and organic dyes, peptides, biotin, and proteins. Aptamers can retain functional activity after biotinylation, fluorescein labeling, and when attached to glass surfaces and microspheres.

Nucleic acid aptamers are oligonucleotides that may be single stranded oligodeoxynucleotides, oligoribonucleotides, or modified oligodeoxynucleotide or oligoribonucleotides. “Modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2-azido-ribose, carbocyclic sugar analogues a-anomeric sugars; epimeric sugars such as arabinose, xyloses or lyxoses, pyranose sugars, furanose sugars, and sedoheptulose. In some embodiments, the binding member comprises a nucleic acid comprising a nucleotide sequence set forth in any one of SEQ ID NOs: 1-11.

Peptide aptamers may be designed to interfere with protein interactions. Peptide aptamers may be based on a protein scaffold onto which a variable peptide loop is attached, thereby constraining the conformation of the aptamer. In some cases, the scaffold portion of the peptide aptamer is derived from Bacterial Thioredoxin A (TrxA).

When the target molecule is a carbohydrate, potentially suitable capture components (as defined herein) include, for example, antibodies, lectins, and selectins. As will be appreciated by those of ordinary skill in the art, any molecule that can specifically associate with a target molecule of interest may potentially be used as a binding member.

For certain embodiments, suitable target analyte/binding member complexes can include, but are not limited to, antibodies/antigens, antigens/antibodies, receptors/ligands, ligands/receptors, proteins/nucleic acid, enzymes/substrates and/or inhibitors, carbohydrates (including glycoproteins and glycolipids)/lectins and/or selectins, proteins/proteins, proteins/small molecules, etc.

In a particular embodiment, the first binding member may be attached to a solid support via a linkage, which may comprise any moiety, functionalization, or modification of the support and/or binding member that facilitates the attachment of the binding member to the support. The linkage between the binding member and the support may include one or more chemical or physical (e.g., non-specific attachment via van der Waals forces, hydrogen bonding, electrostatic interactions, hydrophobic/hydrophilic interactions; etc.) bonds and/or chemical spacers providing such bond(s).

In certain embodiments, a solid support may also comprise a protective, blocking, or passivating layer that can eliminate or minimize non-specific attachment of non-capture components (e.g., analyte molecules, binding members) to the binding surface during the assay which may lead to false positive signals during detection or to loss of signal. Examples of materials that may be utilized in certain embodiments to form passivating layers include, but are not limited to: polymers, such as poly(ethylene glycol), that repel the non-specific binding of proteins; naturally occurring proteins with this property, such as serum albumin and casein; surfactants, e.g., zwitterionic surfactants, such as sulfobetaines; naturally occurring long-chain lipids; polymer brushes, and nucleic acids, such as salmon sperm DNA.

Certain embodiments utilize binding members that are proteins or polypeptides. As is known in the art, any number of techniques may be used to attach a polypeptide to a wide variety of solid supports. A wide variety of techniques are known to add reactive moieties to proteins, for example, the method outlined in U.S. Pat. No. 5,620,850. Further, methods for attachment of proteins to surfaces are known, for example, see Heller, Acc. Chem. Res. 23:128 (1990).

As explained herein, binding between the binding members and the analyte, is specific, e.g., as when the binding member and the analyte are complementary parts of a binding pair. In certain embodiments, the binding member binds specifically to the analyte. By “specifically bind” or “binding specificity,” it is meant that the binding member binds the analyte molecule with specificity sufficient to differentiate between the analyte molecule and other components or contaminants of the test sample. For example, the binding member, according to one embodiment, may be an antibody that binds specifically to an epitope on an analyte. The antibody, according to one embodiment, can be any antibody capable of binding specifically to an analyte of interest. For example, appropriate antibodies include, but are not limited to, monoclonal antibodies, bispecific antibodies, minibodies, domain antibodies (dAbs) (e.g., such as described in Holt et al. (2014) Trends in Biotechnology 21:484-490), and including single domain antibodies sdAbs that are naturally occurring, e.g., as in cartilaginous fishes and camelid, or which are synthetic, e.g., nanobodies, VHH, or other domain structure), synthetic antibodies (sometimes referred to as antibody mimetics), chimeric antibodies, humanized antibodies, antibody fusions (sometimes referred to as “antibody conjugates”), and fragments of each, respectively. As another example, the analyte molecule may be an antibody and the first binding member may be an antigen and the second binding member may be a secondary antibody that specifically binds to the target antibody or the first binding member may be a secondary antibody that specifically binds to the target antibody and the second binding member may be an antigen.

In some embodiments, the binding member may be chemically programmed antibodies (cpAbs) (described in Rader (2014) Trends in Biotechnology 32:186-197), bispecific cpAbs, antibody-recruiting molecules (ARMs) (described in McEnaney et al. (2012) ACS Chem. Biol. 7:1139-1151), branched capture agents, such as a triligand capture agent (described in Millward et al. (2011) J. Am. Chem. Soc. 133:18280-18288), engineered binding proteins derived from non-antibody scaffolds, such as monobodies (derived from the tenth fibronectin type III domain of human fibronectin), affibodies (derived from the immunoglobulin binding protein A), DARPins (based on Ankyrin repeat modules), anticalins (derived from the lipocalins bilin-binding protein and human lipocalin 2), and cysteine knot peptides (knottins) (described in Gilbreth and Koide, (2012) Current Opinion in Structural Biology 22:1-8; Banta et al. (2013) Annu. Rev. Biomed. Eng. 15:93-113), WW domains (described in Patel et al. (2013) Protein Engineering, Design & Selection 26(4):307-314), repurposed receptor ligands, affitins (described in Béhar et al. (2013) 26:267-275), and/or Adhirons (described in Tiede et al. (2014) Protein Engineering, Design & Selection 27:145-155).

According to one embodiment in which an analyte is a biological cell (e.g., mammalian, avian, reptilian, other vertebrate, insect, yeast, bacterial, cell, etc.), the binding members may be ligands having specific affinity for a cell surface antigen (e.g., a cell surface receptor). In one embodiment, the binding member may be an adhesion molecule receptor or portion thereof, which has binding specificity for a cell adhesion molecule expressed on the surface of a target cell type. In use, the adhesion molecule receptor binds with an adhesion molecule on the extracellular surface of the target cell, thereby immobilizing or capturing the cell, the bound cell may then be detected by using a second binding member that may be the same as the first binding member or may bind to a different molecule expressed on the surface of the cell.

In some embodiments, the binding affinity between analyte molecules and binding members should be sufficient to remain bound under the conditions of the assay, including wash steps to remove molecules or particles that are non-specifically bound. In some cases, for example in the detection of certain biomolecules, the binding constant of the analyte molecule to its complementary binding member may be between at least about 10⁴ and about 10⁶ M⁻¹, at least about 10⁵ and about 10⁹ M⁻¹, at least about 10⁷ and about 10⁹ M⁻¹, greater than about 10⁹ M⁻¹, or greater.

v. Tag or Label

The methods described herein may include a specific binding member bound to a detectable label or tag to analyze an analyte. The incorporated tags or labels do not substantially interfere with the conduct of the reaction scheme. For example, the incorporated tag or label does not interfere with the binding constant of or the interaction between the analyte and its complementary binding member. The size and number of incorporated tags or labels may be related to the speed of capture and read rate. The speed of capture and read rate may be increased by increasing the size and/or number of incorporated tags or labels. For example, the size and number of incorporated tags or labels may increase the charge and increase the capture zone of the nanopore, if provided. The incorporated tag or labels do not alter the binding member kinetics, for example, antibody kinetics, or the reaction scheme. Exemplary tags include polymers such as, an anionic polymer or a cationic polymer (e.g., a polypeptide with a net positive charge, such as, polyhistidine or polylysine), where the polymer is about 5-1000 residues in length; a protein (e.g., a globular protein) which does not cross react with the binding member and/or interfere with the assay, a dendrimer, e.g., a DNA dendrimer; and a charged particle or nanoparticle, e.g., a bead or nanobead. A polymer tag may include a nucleic acid, such as, a deoxyribonucleic acid or a ribonucleic acid. A polymer tag may include a nucleobase polymer. In certain cases, the tag may be DNA or a RNA aptamer, where the aptamer does not bind to the analyte. In cases, where the tag is an aptamer, it may be optionally denatured prior to the translocation through the nanopore. A polymer tag or a particle or nanoparticle (e.g., a bead or nanobead) may be sufficiently large to generate a reproducible signal as it translocates through or across a nanopore, if provided. Aptamers may be 20-220 bases in length, e.g., 20-60 bases long. The size of the particle or nanoparticle (e.g., a bead, nanobead or a dendrimer) may range from about 1 nm to about 950 nm in diameter for example, 10 nm-900 nm, 20 nm-800 nm, 30 nm-700 nm, 50 nm-600 nm, 80 nm-500 nm, 100 nm-500 nm, 200 nm-500 nm, 300 nm-500 nm, or 400 nm-500 nm in diameter, e.g., 10 nm, 20 nm, 30 nm, 50 nm, 100 nm, 200 nm, 300 nm, 400 nm, 500 nm, 600 nm, 700 nm, 800 nm, or 900 nm. When used as a tag, a preferred size for nanoparticle is one that can pass through or across a nanopore (as further described herein, if provided). In certain cases, the bead/particle or nanobead/nanoparticle may be made of a material that has a net negative or positive charge or can be treated to have a net negative or positive charge. Exemplary beads/particles or nanobeads/nanoparticles include those made from organic or inorganic polymers. Organic polymers include polymers such as, polystyrene, carbon, polyacrylamide, etc. Inorganic polymers include silicon or metal beads/particles or nanobeads/nanoparticles. In certain cases, the beads/particles or nanobeads/nanoparticles may not be magnetic.

In certain cases, the tag may be a single stranded DNA or RNA. The single stranded DNA or RNA may be hybridized to a probe molecule prior to translocation through or across a nanopore, if provided. In certain cases, the method may include analysis of multiple analytes in a single sample. The second binding members that bind to the different analytes in a sample may include different single stranded DNA or RNA attached thereto as tags and the different single stranded DNA or RNA may be hybridized to different probes that further distinguish the different single stranded DNA or RNA from each other, e.g., as they traverse though the nanopores, if provided. In other embodiments, the tags attached to the different second binding members may have different hairpin structures (e.g., length of the hairpin structure) that are distinguishable when the tags pass through or across a nanopore, if provided. In yet another embodiment, the tags attached to the different second binding members may have different lengths that are distinguishable when the tags traverse through or across the nanopores, if provided—for example, the tags may be double stranded DNA of different lengths (e.g., 25 bp, 50 bp, 75 bp, 100 bp, 150 bp, 200 bp, or more). In certain cases, the tags attached to the different second binding members may have different lengths of polyethylene glycol (PEG) or may be DNA or RNA modified differentially with PEG.

It is noted that reference to a tag or a tag molecule encompasses a single tag or a single tag molecule as well as multiple tags (that all may be identical). It is further noted that the nanopore, if provided, encompasses a single nanopore as well as multiple nanopores present in a single layer, such as, a substrate, a membrane, and the like. As such, counting the number of tags translocating through or across a nanopore in a layer/sheet/membrane refers to counting multiple tags translocating through or across one or more nanopores in a layer/sheet/membrane. Nanopores, if provided, may be present in a single layer, such as a substrate or a membrane, the layer may be made of any suitable material that is electrically insulating or has a high electrical resistance, such as a lipid bilayer, a dielectric material, e.g., silicon nitride and silica, atomically thin membrane such as graphene, silicon, silicene, molybdenum disulfide (MoS₂), etc., or a combination thereof.

The tag may be any size or shape. In some embodiments, the tag may be a nanoparticle or a nanobead about 10 and 950 nm in diameter, e.g., 20-900 nm, 30-800 nm, 40-700 nm, 50-600 nm, 60-500 nm, 70-400 nm, 80-300 nm, 90-200 nm, 100-150 nm, 200-600 nm, 400-500 nm, 2-10 nm, 2-4 nm, or 3-4 nm in diameter. The tag may be substantially spherical, for example a spherical bead or nanobead, or hemi-spherical. The tag may be a protein about 0.5 kDa to about 50 kDa in size, e.g., about 0.5 kDa to about 400 kDa, about 0.8 kDa to about 400 kDa, about 1.0 kDa to about 400 kDa, about 1.5 kDa to about 400 kDa, about 2.0 kDa to about 400 kDa, about 5 kDa to about 400 kDa, about 10 kDa to about 400 kDa, about 50 kDa to about 400 kDa, about 100 kDa to about 400 kDa, about 150 kDa to about 400 kDa, about 200 kDa to about 400 kDa, about 250 kDa to about 400 kDa, about 300 kDa to about 400 kDa, about 0.5 kDa to about 300 kDa, about 0.8 kDa to about 300 kDa, about 1.0 kDa to about 300 kDa, about 1.5 kDa to about 300 kDa, about 2.0 kDa to about 300 kDa, about 5 kDa to about 300 kDa, about 10 kDa to about 300 kDa, about 50 kDa to about 300 kDa, about 100 kDa to about 300 kDa, about 150 kDa to about 300 kDa, about 200 kDa to about 300 kDa, about 250 kDa to about 300 kDa, about 0.5 kDa to about 250 kDa, about 0.8 kDa to about 250 kDa, about 1.0 kDa to about 250 kDa, about 1.5 kDa to about 250 kDa, about 2.0 kDa to about 250 kDa in size, about 5 kDa to about 250 kDa, about 10 kDa to about 250 kDa, about 50 kDa to about 250 kDa, about 100 kDa to about 250 kDa, about 150 kDa to about 250 kDa, about 200 kDa to about 250 kDa, about 0.5 kDa to about 200 kDa, about 0.8 kDa to about 200 kDa, about 1.0 kDa to about 200 kDa, about 1.5 kDa to about 200 kDa, about 2.0 kDa to about 200 kDa in size, about 5 kDa to about 200 kDa, about 10 kDa to about 200 kDa, about 50 kDa to about 200 kDa, about 100 kDa to about 200 kDa, about 150 kDa to about 200 kDa, about 0.5 kDa to about 100 kDa, about 0.8 kDa to about 100 kDa, about 1.0 kDa to about 100 kDa, about 1.5 kDa to about 100 kDa, about 2.0 kDa to about 100 kDa, about 5 kDa to about 100 kDa, about 10 kDa to about 100 kDa, about 50 kDa to about 100 kDa, about 0.5 kDa to about 50 kDa, about 0.8 kDa to about 50 kDa, about 1.0 kDa to about 50 kDa, about 1.5 kDa to about 50 kDa, about 2.0 kDa to about 50 kDa, about 5 kDa to about 50 kDa, about 10 kDa to about 50 kDa. about 10 kDa to about 90 kDa, about 10 kDa to about 80 kDa, about 10 kDa to about 70 kDa, about 10 kDa to about 60 kDa, about 20 kDa to about 90 kDa, about 20 kDa to about 80 kDa, about 20 kDa to about 70 kDa, about 20 kDa to about 60 kDa, about 40 kDa to about 90 kDa, about 40 kDa to about 80 kDa, about 40 kDa to about 70 kDa, or about 40 kDa to about 60 kDa.

In certain embodiments, the tag may be a nanoparticle or nanobead. As noted herein, the nanoparticle may be reversibly (e.g., cleavably) attached to the second binding member. In certain aspects, the nanoparticle may be a nanobead of a defined diameter which may the property of the nanobead measured by the nanopore layer. In certain cases, the methods, systems, and devices of the present disclosure may be used to simultaneously analyze a plurality of different analytes in a sample. For such analysis a plurality of second binding members that each specifically bind to a cognate analyte may be used. Each of the different second binding member may be attached to a different sized nanobead that may be used to identify the second binding member. For example, the different nanobead tags may have different diameters, such as, 1 nm, 2 nm, 4 nm, 6 nm, 8 nm, 10 nm, 12 nm, 14 nm, or larger, such as up to 20 nm, 30 nm, 50 nm, 100 nm, 200 nm, 300 nm, 400 nm, 500 nm, 600 nm, 700 nm, 800 nm, 900 nm, 950 nm, or 990 nm.

In certain embodiments, the nanobeads of different diameters may all translocate through a nanopore layer having nanopores of a single diameter, where the different sized nanobeads may be identified based on the residence duration in the nanopore, magnitude of current impedance, or a combination thereof. In certain cases, a stacked nanopore layer device containing multiple nanopore layers, where a first layer may have nanopores of a first diameter and the second layer may have nanopores of a second diameter may be used to detect and count the nanobeads translocating through or across the nanopores. The multiple nanopore layers may be arranged in a manner such that layer with nanopores of a larger diameter is placed upstream to layer having nanopores of a smaller diameter. Exemplary stacked nanopore layers are disclosed in US20120080361.

Exemplary nanoparticles that may be used as tags in the present methods include gold nanoparticles or polystyrene nanoparticles ranging in diameter from 5 nm-950 nm.

In certain cases, the tag may be a polymer, such as, a nucleic acid. The presence of the tag may be determined by detecting a signal characteristic of the tag, such as a signal related to the size or length of the polymer tag. The size or length of the polymer tag can be determined by measuring its residence time in the pore or channel, e.g., by measuring duration of transient blockade of current.

Elements which can be part of, all of, associated with, or attached to the tag or label include: a nanoparticle; gold particle; silver particle; silver, copper, zinc, or other metal coating or deposit; polymer; drag-tag (as defined herein); magnetic particle; buoyant particle; metal particle; charged moiety; dielectrophoresis tag, silicon dioxide, with and without impurities (e.g., quartz, glass, etc.); poly(methylmethacrylate) (PMMA); polyimide; silicon nitride; gold; silver; quantum dot (including CdS quantum dot); carbon dot; a fluorophore; a quencher; polymer; polystyrene; Janus particle; scattering particle; fluorescent particle; phosphorescent particle; sphere; cube; insulator; conductor; bar-coded or labeled particle; porous particle; solid particle; nanoshell; nanorod; microsphere; analyte such as a virus, cell, parasite and organism; nucleic acid; protein; molecular recognition element; spacer; PEG; dendrimer; charge modifier; magnetic material; enzyme; DNA including aptamer sequence; amplifiable DNA; repeated sequence of DNA; fusion or conjugate of detectable elements with molecular recognition elements (e.g., engineered binding member); anti-antibody aptamer; aptamer directed to antibody-binding protein; absorbed or adsorbed detectable compound; heme; luciferin; a phosphor; an azido, or alkyne (e.g., terminal or non-terminal alkyne) or other click chemistry participant.

In certain embodiments, the tag may be chosen to provide a rate of capture that is sufficiently high to enable a rapid analysis of a sample. In certain embodiments, the capture rate of the tag may be about 1 event per 10 seconds, 1 event per 5 seconds, 1 event per second or higher. In certain embodiments, linear polymer tags, such as, ribose polymers, deoxyribose polymers, oligonucleotides, DNA, or RNA may be used. Typically for 1 nM solution of DNA, capture rates are approximately 1 event sec⁻¹ using a solid-state nanopore (Si₃N₄), with no salt gradient, a voltage of 200-800 mV, and a salt (KCl) concentration of 1 M.

In certain cases, linear polymer tags, such as, ribose polymers, deoxyribose polymers, oligonucleotides, DNA, or RNA may not be used as the capture rate for these tags may be too low for certain applications. Tags that are hemispherical, spherical or substantially spherical in shape rapidly translocate through the nanopores, if provided, and thus shorten the assay duration may be used in applications requiring faster tag counting. In certain cases, the size of the spherical or hemispherical tag may be chosen based on the capture rate needed for the assay. For example, for a higher capture rate, spherical or hemispherical tags of larger size may be selected. In certain cases, the tag may be spherical tag, such as, a nanoparticle/nanobead that has a capture rate about a 10 times, 30 times, 50 times, 100 times, 300 times, 500 times, or a 1000 times faster than capture rate for a linear tag, such as, a DNA tag, under the same measurement conditions.

In some embodiments, the tag may be conjugated to an antibody, for example, a CPSP antibody conjugate. In some embodiments, the tag may be conjugated to an antibody with a spacer, for example, a CPSP antibody conjugate with a spacer. In some embodiments, the tag may be may be conjugated to an oligonucleotide and an antibody, for example, a CPSP oligonucleotide-antibody conjugate. In some embodiments, the tag may be may be conjugated to an oligonucleotide and an antibody with a spacer, for example, a CPSP oligonucleotide-antibody conjugate with spacer. In some embodiments, the tag may be may be conjugated to an oligonucleotide, for example, a CPSP oligonucleotide conjugate. In some embodiments, the spacer includes a nitrobenzyl group, dithioethylamino, 6 carbon spacer, 12 carbon spacer, or 3-(9-((3-carboxypropyl)(tosyl)carbamoyl)acridin-10-ium-10-yl)propane-1-sulfonate. In some embodiments, the spacer comprises a nitrobenzyl group, and the tag is a DNA molecule. In some embodiments, the spacer is dithioethylamino and the tag is a carboxylated nanoparticle. In some embodiments, the spacer is 3-(9-((3-carboxypropyl)(tosyl)carbamoyl)acridin-10-ium-10-yl)propane-1-sulfonate and the tag is an oligonucleotide. In some embodiments, the spacer comprises a 6 carbon spacer or a 12 carbon spacer and the tag is biotin.

In certain embodiments methods described herein may include a specific binding member bound to a detectable label, such as a signal-producing substance, such as chromagens, fluorescent compounds, enzymes, chemiluminescent compounds, radioactive compounds, particles (provided that they have fluorescent properties) and the like. Examples of labels that include moieties that produce light, e.g., acridinium compounds, and moieties that produce fluorescence, e.g., fluorescein.

Any suitable signal-producing substance known in the art can be used as a detectable label. For example, the detectable label can be a radioactive label (such as 3H, 14C, 32P, 33P, 35S, 90Y, 99Tc, 111In, 125I, 131I, 177Lu, 166Ho, and 153Sm), an enzymatic label (such as horseradish peroxidase, alkaline peroxidase, glucose 6-phosphate dehydrogenase, and the like (if enzymes are used then a corresponding enzymatic substrate must also be added)), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

In one aspect, the acridinium compound is an acridinium-9-carboxamide. Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 11: 714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl)acridinium fluorosulfonate (available from Cayman Chemical, Ann Arbor, Mich.). Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

vi. Cleavable Linker

The tags used in the methods described herein may be attached to specific binding member by a generic linker. The cleavable linker ensures that the tag can be removed. The generic linker may be a cleavable linker. For example, the tag may be attached to the second binding member via a cleavable linker. The complex of the first binding member-analyte-second binding member may be exposed to a cleavage agent that mediates cleavage of the cleavable linker. The linker can be cleaved by any suitable method, including exposure to acids, bases, nucleophiles, electrophiles, radicals, metals, reducing or oxidizing agents, light, temperature, enzymes etc. Suitable linkers can be adapted from standard chemical blocking groups, as disclosed in Greene & Wuts, Protective Groups in Organic Synthesis, John Wiley & Sons. Further suitable cleavable linkers used in solid-phase synthesis are disclosed in Guillier et al. (Chem. Rev. 100:2092-2157, 2000). The linker may be acid-cleavable, base-cleavable or photocleavable. A redox reaction may be part of the cleavage scheme. The cleavable linker may be a charged polymer.

The linker may be a photocleavable linker, a chemically cleavable linker, or a thermally cleavable linker. In embodiments, the linker may be thermal-sensitive cleavable linker. Where the linker is a photocleavable group, the cleavage agent may be light of appropriate wavelength that disrupts or cleaves the photocleavable group. In many embodiments, the wavelength of light used to cleave the photocleavable linking group ranges from about 180 nm to 400 nm, e.g., from about 250 nm to 400 nm, or from about 300 nm to 400 nm. It is preferable that the light required to activate cleavage does not affect the other components of the analyte. Suitable linkers include those based on O-nitrobenzyl compounds and nitroveratryl compounds. Linkers based on benzoin chemistry can also be used (Lee et al., J. Org. Chem. 64:3454-3460, 1999). In some embodiments, the photocleavable linker may be derived from the following moiety:

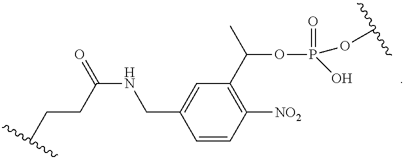 [SMILES: *CCC(=O)NCc1ccc([N+](=O)[O-])c(C(C)OP(=O)(O)O*)c1]

Alternatively, where the cleavage linker is a chemically cleavable group, the cleavage agent may be a chemical agent capable of cleaving the group. A chemically cleavable linker may be cleaved by oxidation/reduction-based cleavage, acid-catalyzed cleavage, base-catalyzed cleavage, or nucleophilic displacement. For example, where the linking group is a disulfide, thiol-mediated cleavage with dithiothreitol or betamercaptoethanol may be used to release the tag. In yet other embodiments where the linking group is a restriction site, the agent is a catalytic agent, such as an enzyme which may be a hydrolytic enzyme, a restriction enzyme, or another enzyme that cleaves the linking group. For example, the restriction enzyme may be a type I, type II, type IIS, type III and type IV restriction enzyme.

In some embodiments, the cleavage linker is an enzymatic cleavable sequence. In one aspect of any of the embodiments herein, an enzymatic cleavable sequence is a nucleic acid sequence of 2, 3, 4, 5, 6, 7, 8, 9 or 10 nucleotides in length. In one embodiment, the enzymatic cleavable sequence comprises a sequence of at least 10 nucleotides. In one embodiment, the enzymatic cleavable sequence comprises a sequence of between 2 and 20 nucleotides. In one embodiment, the enzymatic cleavable sequence comprises a sequence of between 2 and 15 nucleotides. In one embodiment, the enzymatic cleavable sequence comprises a sequence of between 4 and 10 nucleotides. In one embodiment, the enzymatic cleavable sequence comprises a sequence of between 4 and 15 nucleotides.

For example, the cleavable linker may be an acridinium, ethers such as substituted benzyl ether or derivatives thereof (e.g., benzylhydryl ether, indanyl ether, etc.) that can be cleaved by acidic or mild reductive conditions (e.g., hydrogen peroxide to produce an acridone and a sulfonamide), a charged polymer generated using P-elimination, where a mild base can serve to release the product, acetals, including the thio analogs thereof, where detachment is accomplished by mild acid, particularly in the presence of a capturing carbonyl compound, photolabile linkages (e.g., O-nitrobenzoyl, 7-nitroindanyl, 2-nitrobenzhydryl ethers or esters, etc.), or peptide linkers, which are subject to enzymatic hydrolysis (e.g., enzymatic cleavable linkers), particularly where the enzyme recognizes a specific sequence, such as a peptide for Factor Xa or enterokinase. Examples of linkers include, but are not limited to, disulfide linkers, acid labile linkers (including dialkoxybenzyl linkers), Sieber linkers, indole linkers, t-butyl Sieber linkers, electrophilically cleavable linkers, nucleophilically cleavable linkers, photocleavable linkers, cleavage under reductive conditions, oxidative conditions, cleavage via use of safety-catch linkers, and cleavage by elimination mechanisms.

Electrophilically cleaved linkers are typically cleaved by protons and include cleavages sensitive to acids. Suitable linkers include the modified benzylic systems such as trityl, p-alkoxybenzyl esters and p-alkoxybenzyl amides. Other suitable linkers include tert-butyloxycarbonyl (Boc) groups and the acetal system. The use of thiophilic metals, such as nickel, silver or mercury, in the cleavage of thioacetal or other sulphur-containing protecting groups can also be considered for the preparation of suitable linker molecules.

For nucleophilic cleavage, groups such as esters that are labile in water (i.e., can be cleaved simply at basic pH) and groups that are labile to non-aqueous nucleophiles, can be used. Fluoride ions can be used to cleave silicon-oxygen bonds in groups such as triisopropyl silane (TIPS) or t-butyldimethyl silane (TBDMS).

A linker susceptible to reductive cleavage may be used such as with disulphide bond reduction. Catalytic hydrogenation using palladium-based catalysts has been used to cleave benzyl and benzyloxycarbonyl groups.

Oxidation-based approaches are well known in the art. These include oxidation of p-alkoxybenzyl groups and the oxidation of sulphur and selenium linkers. Aqueous iodine to cleave disulphides and other sulphur or selenium-based linkers may also be used.

Safety-catch linkers are those that cleave in two steps. In a preferred system the first step is the generation of a reactive nucleophilic center followed by a second step involving an intra-molecular cyclization that results in cleavage. For example, levulinic ester linkages can be treated with hydrazine or photochemistry to release an active amine, which can then be cyclised to cleave an ester elsewhere in the molecule (Burgess et al., J. Org. Chem. 62:5165-5168, 1997).

Elimination reactions may also be used. For example, the base-catalysed elimination of groups such as Fmoc and cyanoethyl, and palladium-catalysed reductive elimination of allylic systems, may be used.

vii. Nanopore Layer

In the present disclosure, detecting and/or counting the tag (e.g., polymer, aptamer, nanoparticle) may be carried out by translocating the tag through or across a nanopore or nanochannel. In some embodiments, detecting and/or counting the tag (e.g., polymer, aptamer, nanoparticle) may be carried out by translocating the tag through or across at least one or more nanopores or nanochannels. In some embodiments, at least to or more nanopores or nanochannels are presented side by side or in series. In some embodiments, the nanopore or nanochannel is dimensioned for translocation of not more than one tag at a time. Thus, the dimensions of the nanopore in some embodiments will typically depend on the dimensions of the tag to be examined. A tag with a double-stranded region can require a nanopore dimension greater than those sufficient for translocation of a tag which is entirely single-stranded. In addition, a nanoparticle tag such as a nanobead tag can require larger pores or channels than oligomer tags. Typically, a pore of about 1 nm diameter can permit passage of a single stranded polymer, while pore dimensions of 2 nm diameter or larger will permit passage of a double-stranded nucleic acid molecule. In some embodiments, the nanopore or nanochannel is selective for a single stranded tag (e.g., from about 1 nm to less than 2 nm diameter) while in other embodiments, the nanopore or nanochannel is of a sufficient diameter to permit passage of double stranded polynucleotides (e.g., 2 nm or larger). The chosen pore size provides an optimal signal-to noise ratio for the analyte of interest.

In some embodiments, the pore may be between about 0.1 nm and about 1000 nm in diameter, between about 50 nm and about 1000 nm, between about 100 nm and 1000 nm, between about 0.1 nm and about 700 nm, between about 50 nm and about 700 nm, between about 100 nm and 700 nm, between about 0.1 nm and about 500 nm, between about 50 nm and about 500 nm, or between about 100 nm and 500 nm. For example, the pore may be about 0.1 nm, about 0.2 nm, about 0.3 nm, about 0.4 nm, about 0.5 nm, about 0.6 nm, about 0.7 nm, about 0.8 nm, about 0.9 nm, about 1.0 nm, about 1.5 nm, about 2.0 nm, about 2.5 nm, about 3.0 nm, about 3.5 nm, about 4.0 nm, about 4.5 nm, about 5.0 nm, about 7.5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 150 nm, about 200 nm, about 250 nm, about 300 nm, about 3500 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm in diameter.

In general, nanopores are shorter in length than nanochannels. A nanochannel is substantially longer than a nanopore and may be useful in applications where increasing the time it takes for a molecule to translocate through it (as compared to the time for translocating through or across a nanopore of the same diameter) is desirable. Length of a nanopore may range from about 0.1 nm to less than about 200 nm. Length of a nanochannel may range from about 500 nm to about 100 μm, or longer. The diameter of a nanopore and a nanochannel may be similar.

Various types of nanopores may be used for analyzing the tags/aptamer. These include, among others, biological nanopores that employ a biological pore or channel embedded in a membrane. Another type of nanopore layer is a solid state nanopore in which the channel or pore is made whole or in part from a fabricated or sculpted solid state component, such as silicon. In some embodiments, the nanopore is a solid state nanopore produced using controlled dielectric breakdown. In some embodiments, the nanopore is a solid state nanopore produced by a method other than controlled dielectric breakdown.

In certain embodiments, the length of a nanopore may be up to about 200 nm, e.g., from about 0.1 nm to about 30 nm, from about 10 to about 80 nm, from about 1 to about 50 nm, from about 0.1 nm to about 0.5 nm, from about 0.3 nm to about 1 nm, from about 1 nm to about 2 nm, from about 0.3 nm to about 10 nm, or from about 10 to about 30 nm. The number of nanopores in a nanopore layer may be about 1, 2, 3, 4, 5, 10, 30, 100, 300, 1000, 3000, 10000, 30000, 100000, 300000 or more. The distance between nanopores in a layer between center to center may be about 100 nm to about 300 nm, about 300 nm to about 500 nm, about 500 nm to about 1000 nm, for example, 100 nm, 150 nm, 200 nm, or 300 nm.

In certain embodiments, multiple nanopore layers, each containing on or more nanopores, can be arranged in series with with each other, for detecting and/or counting the tag (e.g., polymer, aptamer, nanoparticle). In this case, detecting and/or counting the tag may be carried out by translocating the tag through or across each nanopore layer. As such, counting the number of tags translocating through or across a nanopore in a layer/sheet/membrane refers to counting multiple tags translocating through or across one or more nanopores in one or more layer/sheet/membrane. In certain embodiments, when more than one nanopore layers are present (e.g., one, two, three, four, five, six, or other number of nanopore layers as technically feasible), optionally they are present in series wherein at least one nanopore in one layer is separate from or stacked onto (e.g., above or on top of) another nanopore in another layer, etc.). Where the nanopore layers are in series, at least two electrodes can be used to create an alectric field to drive tags through the pores and, optionally, additional electrodes positioned between the nanopore layers can further provide driving current.

i) Biological Pores

For detecting and, optionally, counting the tags/aptamer, any biological pore with channel dimensions that permit translocation of the tags can be used. Two broad categories of biological channels are suitable for the methods disclosed herein. Non-voltage gated channels allow passage of molecules through the pore without requiring a change in the membrane potential to activate or open the channel. On the other hand, voltage gated channels require a particular range of membrane potential to activate channel opening. Most studies with biological nanopores have used α-hemolysin, a mushroom-shaped homo-oligomeric heptameric channel of about 10 nm in length found in Staphylococcus aureus. Each subunit contributes two beta strands to form a 14 strand anti-parallel beta barrel. The pore formed by the beta barrel structure has an entrance with a diameter of approximately 2.6 nm that contains a ring of lysine residues and opens into an internal cavity with a diameter of about 3.6 nm. The stem of the hemolysin pore, which penetrates the lipid bilayer, has an average inside diameter of about 2.0 nm with a 1.5 nm constriction between the vestibule and the stem. The dimensions of the stem are sufficient for passage of single-stranded nucleic acids but not double-stranded nucleic acids. Thus, α-hemolysin pores may be used as a nanopore selective for single-stranded polynucleotides and other polymers of similar dimensions.

In other embodiments, the biological nanopore is of a sufficient dimension for passage of polymers larger than a single-stranded nucleic acid. An exemplary pore is mitochondrial porin protein, a voltage dependent anion channel (VDAC) localized in the mitochondrial outer membrane. Porin protein is available in purified form and, when reconstituted into artificial lipid bilayers, generates functional channels capable of permitting passage of double-stranded nucleic acids (Szabo et al., 1998, FASEB J. 12:495-502). Structural studies suggest that porin also has a beta-barrel type structure with 13 or 16 strands (Rauch et al., 1994, Biochem Biophys Res Comm 200:908-915). Porin displays a larger conductance compared conductance of pores formed by α-hemolysin, maltoporin (LamB), and gramicidin. The larger conductance properties of porin support studies showing that the porin channel is sufficiently dimensioned for passage of double-stranded nucleic acids. Pore diameter of the porin molecule is estimated at 4 nm. The diameter of an uncoiled double-stranded nucleic acid is estimated to be about 2 nm.

Another biological channel that may be suitable for scanning double stranded polynucleotides are channels found in B. subtilis (Szabo et al., 1997, J. Biol. Chem. 272:25275-25282). Plasma membrane vesicles made from B. subtilis and incorporated into artificial membranes allow passage of double-stranded DNA across the membrane. Conductance of the channels formed by B. subtilis membrane preparations is similar to those of mitochondrial porin. Although there is incomplete characterization (e.g., purified form) of these channels, it is not necessary to have purified forms for the purposes herein. Diluting plasma membrane preparations, either by solubilizing in appropriate detergents or incorporating into artificial lipid membranes of sufficient surface area, can isolate single channels in a detection apparatus. Limiting the duration of contact of the membrane preparations (or protein preparations) with the artificial membranes by appropriately timed washing provides another method for incorporating single channels into the artificial lipid bilayers. Conductance properties may be used to characterize the channels incorporated into the bilayer.

In certain cases, the nanopores may be hybrid nanopores, where a biological pore is introduced in a solid state nanopore, e.g., a nanopore fabricated in a non-biological material. For example, α-haemolysin pore may be inserted into a solid state nanopore. In certain cases, the nanopores may be a hybrid nanopore described in Hall et al., Nature Nanotechnology, 28 Nov. 2010, vol. 5, pg. 874-877.

ii) Solid State Pores

In other embodiments, analysis of the tags is carried out by translocating the tag through or across a nanopore or nanochannel fabricated from non-biological materials. Nanopores or nanochannels can be made from a variety of solid state materials using a number of different techniques, including, among others, chemical deposition, electrochemical deposition, electroplating, electron beam sculpting, ion beam sculpting, nanolithography, chemical etching, laser ablation, focused ion beam, atomic layer deposition, and other methods well known in the art (see, e.g., Li et al., 2001, Nature 412:166-169; and WO 2004/085609).

In particular embodiments, the nanopores may be the nanopores described in WO13167952A1 or WO13167955A1. As described in WO13167952A1 or WO13167955A1, nanopores having an accurate and uniform pore size may be formed by precisely enlarging a nanopore formed in a membrane. The method may involve enlarging a nanopore by applying a high electric potential across the nanopore; measuring current flowing through the nanopore; determining size of the nanopore based in part on the measured current; and removing the electric potential applied to the nanopore when the size of the nanopore corresponds to a desired size. In certain cases, the applied electric potential may have a pulsed waveform oscillating between a high value and a low value, the current flowing through the nanopore may be measured while the electric potential is being applied to the nanopore at a low value.

Solid state materials include, by way of example and not limitation, any known semiconductor materials, insulating materials, and metals coated with insulating material. Thus, at least part of the nanopore(s) may comprise without limitation silicon, silica, silicene, silicon oxide, graphene, silicon nitride, germanium, gallium arsenide, or metals, metal oxides, and metal colloids coated with insulating material.

To make a pore of nanometer dimensions, various feedback procedures can be employed in the fabrication process. In embodiments where ions pass through a hole, detecting ion flow through the solid state material provides a way of measuring pore size generated during fabrication (see, e.g., U.S. Published Application No. 2005/0126905). In other embodiments, where the electrodes define the size of the pore, electron tunneling current between the electrodes gives information on the gap between the electrodes. Increases in tunneling current indicate a decrease in the gap space between the electrodes. Other feedback techniques will be apparent to the skilled artisan.

In some embodiments, the nanopore is fabricated using ion beam sculpting, as described in Li et al., 2003, Nature Materials 2:611-615. In some embodiments, the nanopore is fabricated using high current, as described in WO13167952A1 or WO13167955A1. In other embodiments, the nanopores may be made by a combination of electron beam lithography and high energy electron beam sculpting (see, e.g., Storm et al., 2003, Nature Materials 2:537-540). A similar approach for generating a suitable nanopore by ion beam sputtering technique is described in Heng et al., 2004, Biophy J 87:2905-2911. The nanopores are formed using lithography with a focused high energy electron beam on metal oxide semiconductor (CMOS) combined with general techniques for producing ultrathin films. In other embodiments, the nanopore is constructed as provided in U.S. Pat. Nos. 6,627,067; 6,464,842; 6,783,643; and U.S. Publication No. 2005/0006224 by sculpting of silicon nitride.

In some embodiments, the nanochannels can be constructed as a gold or silver nanotube. These nanochannels are formed using a template of porous material, such as polycarbonate filters prepared using a track etch method, and depositing gold or other suitable metal on the surface of the porous material. Track etched polycarbonate membranes are typically formed by exposing a solid membrane material to high energy nuclear particles, which creates tracks in the membrane material. Chemical etching is then employed to convert the etched tracks to pores. The formed pores have a diameter of about 10 nm and larger. Adjusting the intensity of the nuclear particles controls the density of pores formed in the membrane. Nanotubes are formed on the etched membrane by depositing a metal, typically gold or silver, into the track etched pores via an electroless plating method (Menon et al., 1995, Anal Chem 67:1920-1928). This metal deposition method uses a catalyst deposited on the surface of the pore material, which is then immersed into a solution containing Au(I) and a reducing agent. The reduction of Au(I) to metallic Au occurs on surfaces containing the catalyst. Amount of gold deposited is dependent on the incubation time such that increasing the incubation time decreases the inside diameter of the pores in the filter material. Thus, the pore size may be controlled by adjusting the amount of metal deposited on the pore. The resulting pore dimension is measured using various techniques, for instance, gas transport properties using simple diffusion or by measuring ion flow through the pores using patch clamp type systems. The support material is either left intact, or removed to leave gold nanotubes. Electroless plating technique is capable of forming pore sizes from less than about 1 nm to about 5 nm in diameter, or larger as required. Gold nanotubes having pore diameter of about 0.6 nm appears to distinguish between Ru(bpy)2+2 and methyl viologen, demonstrating selectivity of the gold nanopores (Jirage et al., 1997, Science 278:655-658). Modification of a gold nanotube surface is readily accomplished by attaching thiol containing compounds to the gold surface or by derivatizing the gold surface with other functional groups. This features permits attachment of pore modifying compounds as well as sensing labels, as discussed herein. Devices, such as the cis/trans apparatuses used for biological pores described herein, can be used with the gold nanopores to analyze single coded molecules.

Where the mode of detecting the tag involves current flow through the tag (e.g., electron tunneling current), the solid state membrane may be metalized by various techniques. The conductive layer may be deposited on both sides of the membrane to generate electrodes suitable for interrogating the tag along the length of the chain, for example, longitudinal electron tunneling current. In other embodiments, the conductive layer may be deposited on one surface of the membrane to form electrodes suitable for interrogating tag across the pore, for example, transverse tunneling current. Various methods for depositing conductive materials are known, including, sputter deposition (i.e., physical vapor deposition), non-electrolytic deposition (e.g., colloidal suspensions), and electrolytic deposition. Other metal deposition techniques are filament evaporation, metal layer evaporation, electron-beam evaporation, flash evaporation, and induction evaporation, and will be apparent to the skilled artisan.

In some embodiments, the detection electrodes are formed by sputter deposition, where an ion beam bombards a block of metal and vaporizes metal atoms, which are then deposited on a wafer material in the form of a thin film. Depending on the lithography method used, the metal films are then etched by means of reactive ion etching or polished using chemical-mechanical polishing. Metal films may be deposited on preformed nanopores or deposited prior to fabrication of the pore.

In some embodiments, the detection electrodes are fabricated by electrodeposition (see, e.g., Xiang et al., 2005, Angew. Chem. Int. Ed. 44:1265-1268; Li et al., Applied Physics Lett. 77(24):3995-3997; and U.S. Publication Application No. 2003/0141189). This fabrication process is suitable for generating a nanopore and corresponding detection electrodes positioned on one face of the solid state film, such as for detecting transverse electron tunneling. Initially, a conventional lithographic process is used to form a pair of facing electrodes on a silicon dioxide layer, which is supported on a silicon wafer. An electrolyte solution covers the electrodes, and metal ions are deposited on one of the electrodes by passing current through the electrode pair. Deposition of metal on the electrodes over time decreases the gap distance between the electrodes, creating not only detection electrodes but a nanometer dimensioned gap for translocation of coded molecules. The gap distance between the electrodes may be controlled by a number of feedback processes.

Where the detection is based on imaging of charge induced field effects, a semiconductor can be fabricated as described in U.S. Pat. No. 6,413,792 and U.S. published application No. 2003/0211502. The methods of fabricating these nanopore devices can use techniques similar to those employed to fabricate other solid state nanopores.

Detection of the tag, such as a polynucleotide, is carried out as further described below. For analysis of the tag, the nanopore may be configured in various formats. In some embodiments, the device comprises a membrane, either biological or solid state, containing the nanopore held between two reservoirs, also referred to as cis and trans chambers (see, e.g., U.S. Pat. No. 6,627,067). A conduit for electron migration between the two chambers allows electrical contact of the two chambers, and a voltage bias between the two chambers drives translocation of the tag through the nanopores. A variation of this configuration is used in analysis of current flow through nanopores, as described in U.S. Pat. Nos. 6,015,714 and 6,428,959; and Kasianowiscz et al., 1996, Proc Natl Acad Sci USA 93:13770-13773, the disclosures of which are incorporated herein by reference.

Variations of above the device are disclosed in U.S. application publication no. 2003/0141189. A pair of nanoelectrodes, fabricated by electrodeposition, is positioned on a substrate surface. The electrodes face each other and have a gap distance sufficient for passage of a single nucleic acid. An insulating material protects the nanoelectrodes, exposing only the tips of the nanoelectrodes for the detection of the nucleic acid. The insulating material and nanoelectrodes separate a chamber serving as a sample reservoir and a chamber to which the polymer is delivered by translocation. Cathode and anode electrodes provide an electrophoresis electric field for driving the tag from the sample chamber to the delivery chamber.

The current bias used to drive the tag through the nanopore can be generated by applying an electric field directed through the nanopore. In some embodiments, the electric field is a constant voltage or constant current bias. In other embodiments, the movement of the tag is controlled through a pulsed operation of the electrophoresis electric field parameters (see, e.g., U.S. Patent Application No. 2003/141189 and U.S. Pat. No. 6,627,067). Pulses of current may provide a method of precisely translocating one or only a few bases of an oligonucleotide tag for a defined time period through the pore and to briefly hold the tag within the pore, and thereby provide greater resolution of the electrical properties of the tag.

The nanopore devices may further comprise an electric or electromagnetic field for restricting the orientation of the oligonucleotide tag as it passes through the nanopore. This holding field can be used to decrease the movement of the oligonucleotide tag within the pore. In some embodiments, an electric field that is orthogonal to the direction of translocation is provided to restrict the movement of the tag molecule within the nanopore. This is illustrated in U.S. Application Publication No. 2003/0141189 through the use of two parallel conductive plates above and beneath the sample plate. These electrodes generate an electric field orthogonal to the direction of translocation of a tag molecule, and thus holding the tag molecule to one of the sample plates. A negatively charged backbone of a DNA, or nucleic acid modified to have negative charges on one strand, will be oriented onto the anodic plate, thereby limiting the motion of the tag molecule.

In still other embodiments, controlling the position of the tag is carried out by the method described in U.S. Application Publication No. 2004/0149580, which employs an electromagnetic field created in the pore via a series of electrodes positions near or on the nanopore. In these embodiments, one set of electrodes applies a direct current voltage and radio frequency potential while a second set of electrodes applies an opposite direct current voltage and a radio frequency potential that is phase shifted by 180 degrees with respect to the radio frequency potential generated by the first set of electrodes. This radio frequency quadrupole holds a charged particle (e.g., nucleic acid) in the center of the field (i.e., center of the pore).

In exemplary embodiments, the nanopore membrane may be a multilayer stack of conducting layers and dielectric layers, where an embedded conducting layer or conducting layer gates provides well-controlled and measurable electric field in and around the nanopore through which the tag translocates. In an aspect, the conducting layer may be graphene. Examples of stacked nanopore membranes are found in US20080187915 and US20140174927, for example.

It is understood that the nanopore may be located in a membrane, layer or other substrate, which terms have been used interchangeably to describe a two-dimensional substrate comprising a nanopore.

In certain embodiments, the nanopore may be formed as part of the assay process for detecting and/or determining concentration of an analyte using the nanopore. Specifically, a device for detecting and/or determining concentration of an analyte using a nanopore may initially be provided without a nanopore formed in a membrane or layer. The device may include a membrane separating two chambers on the opposite sides of the membrane (a cis and a trans chamber). The cis and the trans chambers may include a salt solution and may be connected to a source of electricity. When a nanopore is to be created in the membrane, a voltage is applied to the salt solution in the cis and trans chamber and conductance through the membrane measured. Prior to the creation of a nanopore, there is no or minimal current measured across the membrane. Following creation of a nanopore, the current measured across the membrane increases. The voltage may be applied for an amount of time sufficient to create a nanopore of the desired diameter. Following the creation of a nanopore, an analyte or tag may be translocated through the nanopore and the translocation event detected. In certain embodiments, the same salt solution may be used for nanopore creation as well as for detection of translocation of an analyte or tag through the nanopore. Any suitable salt solution may be utilized for nanopore creation and/or translocation of an analyte or tag through the nanopore. Any salt solution that does not damage the counting label can be used. Exemplary salt solutions include lithium chloride, potassium chloride, sodium chloride, calcium chloride, magnesium chloride and the like. The concentration of the salt solution may be selected based on the desired conductivity of the salt solution. In certain embodiments, the salt solution may have a concentration ranging from 1 mM to 10 M, e.g., 10 mM-10 M, 30 mM-10 M, 100 mM-10 M, 1 M-10 M, 10 mM-5 M, 10 mM-3 M, 10 mM-1 M, 30 mM-5 M, 30 mM-3 M, 30 mM-1 M, 100 mM-5 M, 100 mM-3 M, 100 mM-1 M, 500 mM-5 M, 500 mM-3 M, or 500 mM-1 M, such as, 10 mM, 30 mM, 100 mM, 500 mM, 1 M, 3 M, 5 M, or 10 M.

In some embodiments, the nanopore may become blocked, and the blocked nanopore is cleared by modulating the pattern of voltage applied by the electrodes across the nanopore layer or membrane. In some cases, a blocked nanopore is cleared by reversing polarity of the voltage across the nanopore layer or membrane. In some cases, a blocked nanopore is cleared by increasing the magnitude of the voltage applied across the nanopore layer or membrane. The increase in voltage may be transitory increase, lasting 10 seconds (s) or less, e.g., 8 s or less, 6 s or less, 5 s or less, 4 s or less, 3 s or less, 2 s or less, 1 s or less, 0.5 s or less, 0.4 s or less, 0.3 s or less, 0.2 s or less, including 0.1 s or less.

viii. Signal Detection

Interrogating the tag/aptamer by translocation through or across a nanopore and detecting the detectable property generates a signal that can be used to count (i.e., determine the quantity or concentration) and/or identify (i.e., determine the presence of) the tag/aptamer. The type of detection method employed may correspond to the property being detected for the tags.

In some embodiments, the detectable property is the effect of the tag on the electrical properties of the nanopore as the tag translocates through the pore. Electrical properties of the nanopore include among others, current amplitude, impedance, duration, and frequency. In certain cases, the tag may be identified by using nanopore force spectroscopy (see e.g., Tropini C. and Marziali A., Biophysical Journal, 2007, Vol. 92, 1632-1637). Devices for detecting the pore's electrical properties may include a nanopore incorporated into a layer such as, a thin film or a membrane, where the film or membrane separates a cis chamber and a trans chamber connected by a conducting bridge. The tag to be analyzed may be present on the cis side of the nanopore in an aqueous solution typically comprising one or more dissolved salts, such as potassium chloride. Application of an electric field across the pore using electrodes positioned in the cis and trans side of the nanopore causes translocation of the tag through the nanopore, which affects the migration of ions through the pore, thereby altering the pore's electrical properties. Current may be measured at a suitable time frequency to obtain sufficient data points to detect a current signal pattern. The generated signal pattern can then be compared to a set of reference patterns in which each reference pattern is obtained from examination of a single population of known tags bound to analyte in a sample with a known analyte concentration. As previously noted, the number of tags of the same type translocating though a nanopore(s) may be counted per unit time, such as, the number of tags of the same type translocating through or across nanopore(s) per 15 min, 13 min, 10 min, 8 min, 6 min, 4 min, 2 min, 1 min, 30 sec, per 20 sec, per 15 sec, per 10 sec, per 5 sec, per 1 sec, per 100 millisec, per 10 millisec, or per 1 millisec. In some cases, the number of tags of the same type translocating though a nanopore(s) may be counted for a certain period of time to determine the amount of time to reach a threshold count. Shifts in current amplitude, current duration, current frequency, and current magnitude may define a signal pattern for the tag and may be used to distinguish different tags from each other. Measurement of current properties of a nanopore, such as by patch clamp techniques, is described in publications discussed above and in various reference works, for example, Hille, B, 2001, Ion Channels of Excitable Membranes, 3rd Ed., Sinauer Associates, Inc., Sunderland, Mass. The number of counts measured over a time period (counts/time) is proportional to the concentration of the molecule (e.g., tag) translocating through or across the nanopore. The concentration of the tag may be determined by generating a standard curve. For example, a series of different concentrations of a standard molecule may be translocated through a nanopore and the counts/time measured to calculate a count rate for each concentration. The count rate of the tag being measured would be compared to the standard curve to calculate the concentration of the tag.

In some embodiments, the detectable property of the tag may be quantum tunneling of electrons. Quantum tunneling is the quantum-mechanical effect of transitioning through a classically-forbidden energy state via a particle's quantum wave properties. Electron tunneling occurs where a potential barrier exists for movement of electrons between a donor and an acceptor. To detect electron tunneling, a microfabricated electrode tip may be positioned about 2 nanometers from the specimen. At an appropriate separation distance, electrons tunnel through the region between the tip and the sample, and if a voltage is applied between the tip and the sample, a net current of electrons (i.e., tunneling current) flows through the gap in the direction of the voltage bias. Where the nanodevice uses detection electrodes for measuring tunneling current, the electrodes are positioned proximately to the translocating tag such that there is electron tunneling between the detection electrodes and tag. As further discussed below, the arrangement of the electrodes relative to the translocating tag may dictate the type of electron transport occurring through the tag.

In some embodiments, analysis of the tag may involve detecting current flow occurring through the nucleic acid chain (i.e., longitudinally along the nucleic acid chain) (Murphy et al., 1994, Proc Natl Acad Sci USA 91(12):5315-9). The exact mechanism of electron transfer is unknown, although electron tunneling is given as one explanation for DNA's transport properties. However, the physics underlying electron transport through a double-stranded nucleic acid is not limiting for the purposes herein, and detection of current flowing through the nucleic acid serves to distinguish one polymer tag from another polymer tag. For detection of electron flow occurring longitudinally through the tag molecule chain, the detection electrodes may be positioned longitudinally to the direction of tag molecule translocation such that there is a gap between the electrodes parallel to the chain of an extended tag molecule. In various embodiments, the detection electrodes may be placed on opposite sides of a layer(s) (e.g., membrane) separating the two sides of the nanopore, while in other embodiments, the detection electrodes may be positioned within the layer(s) that separate the two sides of the nanopore.

Another mode of electron flow in a nucleic acid is that occurring across the nucleic acid, for example, a direction transverse to an extended nucleic acid chain (e.g., across the diameter of a double-stranded nucleic acid). In a double-stranded nucleic acid, electron transport may occur through the paired bases while in a single-stranded nucleic acid, electron transport may occur through a single unpaired base. Furthermore, differences in the chemical compositions, hydration structures, interactions with charged ions, spatial orientation of each base, and different base pairing combinations may alter the transverse electron transport characteristics, and thus provide a basis for distinguishing tag molecules that differ in sequence and/or polymer backbone. For detection of electron flow across a tag molecule (i.e., transverse to an extended nucleic acid chain), the detection electrodes are positioned on one side of the nanopore to interrogate the tag molecule across rather than through the nanopore.

In embodiments of longitudinal or transverse detection, the thickness of the electrodes may determine the total number of bases interrogated by the electrodes. For transverse detection, the tips of the detection electrodes may be dimensioned to interrogate a single nucleobase (as defined herein), and thereby obtain single base resolution. In other embodiments, the dimensions of the detection electrode are arranged to interrogate more than one nucleobase. Thus, in some embodiments, the number of nucleobases interrogated at any one time may be about 2 or more, about 5 or more, about 10 or more, or about 20 or more depending on the resolution required to detect differences in the various polymer sequences of the tag molecule.

In other embodiments, differences in the structure of a tag may be detected as differences in capacitance. This type of measurement is illustrated in US2003/0141189. Capacitance causes a phase shift in an applied ac voltage at a defined applied frequency and impedance. Phase shift characteristics for each nucleobase is determined for nucleic acids of known sequence and structure, and used as reference standards for identifying individual base characteristics. Nearest neighbor analysis may permit capacitance measurements extending to more than a single nucleobase.

In other embodiments, the detection technique may be based on imaging charge-induced fields, as described in U.S. Pat. No. 6,413,792 and U.S. published application No. 2003/0211502, the disclosures of which are incorporated herein by reference. For detecting a tag based on charge induced fields, a semiconductor device described above is used. Application of a voltage between a source region and a drain region results in flow of current from the source to the drain if a channel for current flow forms in the semi-conductor. Because each nucleobase has an associated charge, passage of a tag molecule through the semiconductor pore induces a change in the conductivity of the semiconductor material lining the pore, thereby inducing a current of a specified magnitude and waveform. Currents of differing magnitude and waveform are produced by different bases because of differences in charge, charge distribution, and size of the bases. In the embodiments disclosed in U.S. Pat. No. 6,413,792, the polymer passes through a pore formed of a p-type silicon layer. Translocation of the tag molecule is achieved by methods similar to those used to move a polymer through other types of channels, as described above. The magnitude of the current is expected to be on the order of microampere range, which is much higher than the expected picoampere currents detected by electron tunneling. Because the polymer block regions in the tag molecule comprise more than a single nucleobase, these block polymer regions should produce distinctive signals reflective of the charge and charge distribution of the block polymer regions.

It is to be understood that although descriptions above relate to individual detection techniques, in some embodiments, a plurality of different techniques may be used to examine a single tag molecule (see, e.g., Kassies et al., 2005, J Microsc 217:109-16). Examples of multiple detection modes include, among others, current blockade in combination with electron tunneling current, and current blockage in combination with imaging charge induced fields. Concurrent detection with different detection modes may be used to identity a tag molecule by correlating the detection time of the resulting signal between different detection modes.

In some embodiments, measuring the number of tags translocating through the layer or detecting tags translocating through the layer includes observing a current blockade effect of the tags on the nanopores. In some embodiments, an analyte is present in the sample when the current blockade effect is above a threshold level.

### 3. Devices for Analyte Analysis

Systems, devices, and method are described herein that relate to an integrated digital microfluidic and analyte detection device.

In certain embodiments, the integrated digital microfluidic and analyte detection device may have two modules: a sample preparation module and an analyte detection module. In certain embodiments, the sample preparation module and the analyte detection module are separate or separate and adjacent. In certain embodiments, the sample preparation module and the analyte detection module are co-located, comingled or interdigitated. The sample preparation module may include a series or plurality of electrodes for moving, merging, diluting, mixing, separating droplets of samples and reagents. The analyte detection module may include an array of wells in which an analyte related signal is detected. In certain cases, the detection module may also include the series or plurality of electrodes for moving a droplet of prepared sample to the array of wells. In certain embodiments, the detection module may include an array of wells in a first substrate (e.g., upper substrate) which is disposed over a second substrate (e.g., lower substrate) separated by a gap. In these embodiments, the array of wells is in an upside-down orientation. In certain embodiments, the detection module may include an array of wells in a second substrate (e.g., lower substrate) which is disposed below a first substrate (e.g., upper substrate) separated by a gap. In such embodiments, the first substrate and the second substrate are in a facing arrangement. A droplet may be moved (e.g., by electrical actuation) to the array of wells using electrode(s) present in the first substrate and/or the second substrate. In certain embodiments, the array of wells including the region in between the wells may be hydrophobic. In other embodiments, the series or plurality of electrodes may be limited to the sample preparation module and a droplet of prepared sample (and/or a droplet of immiscible fluid) may be moved to the detection module using other means.

In certain embodiments, the sample preparation module may be used for performing steps of an immunoassay. Any immunoassay format may be used to generate a detectable signal which signal is indicative of presence of an analyte of interest in a sample and is proportional to the amount of the analyte in the sample. Exemplary immunoassays are described herein.

In certain cases, the detection module includes the array of wells that are optically interrogated to measure a signal related to the amount of analyte present in the sample. The array of wells may have sub-femtoliter volume, femtoliter volume, sub-nanoliter volume, nanoliter volume, sub-microliter volume, or microliter volume. For example the array of wells may be array of femtoliter wells, array of nanoliter wells, or array of microliter wells. In certain embodiments, the wells in an array may all have substantially the same volume. The array of wells may have a volume up to 100 μl, e.g., about 0.1 femtoliter, 1 femtoliter, 10 femtoliter, 25 femtoliter, 50 femtoliter, 100 femtoliter, 0.1 pL, 1 pL, 10 pL, 25 pL, 50 pL, 100 pL, 0.1 nL, 1 nL, 10 nL, 25 nL, 50 nL, 100 nL, 0.1 microliter, 1 microliter, 10 microliter, 25 microliter, 50 microliter, or 100 microliter.

In certain embodiments, the sample preparation module and the detection module may both be present on a single base substrate and both the sample preparation module and the detection module may include a series or plurality of electrodes for moving liquid droplets. In certain embodiments, such a device may include a first substrate and a second substrate, where the second substrate is positioned over the first substrate and separated from the first substrate by a gap. The first substrate may include a first portion (e.g., proximal portion) at which the sample preparation module is located, where a liquid droplet is introduced into the device, and a second portion (e.g., distal portion) towards which the liquid droplet moves, at which second portion the detection module is located. It will be understood by one skilled in the art that the use of “proximal” in view of “distal” and “first” in view of “second” are relative terms and are interchangeable with respect to each other. In certain embodiments, first portion and the second portion are separate or separate and adjacent. In certain embodiments, the first portion and the second portion are co-located, comingled or interdigitated. The first substrate may include a series or plurality of electrodes overlayed on an upper surface of the first substrate and extending from the first portion to the second portion. The first substrate may include a layer disposed on the upper surface of the first substrate, covering the series or plurality of electrodes, and extending from the first portion to the second portion. The first layer may be made of a material that is a dielectric and a hydrophobic material. Examples of a material that is dielectric and hydrophobic include polytetrafluoroethylene material (e.g., Teflon®) or a fluorosurfactant (e.g., FluoroPel™). The first layer may be deposited in a manner to provide a substantially planar surface. An array of wells may be positioned in the second portion of the first substrate and overlying a portion of the series or plurality of electrodes, and form the detection module. The array of wells may be positioned in the first layer. In certain embodiments, prior to or after fabrication of the array of wells in the first layer, a hydrophilic layer may be disposed over the first layer in the second portion of the first substrate to provide an array of wells that have a hydrophilic surface. The space/gap between the first and second substrates may be filled with air or an immiscible fluid. In certain embodiments, the space/gap between the first and second substrates may be filled with air.

In certain embodiments, the sample preparation module and the detection module may both be fabricated using a single base substrate but a series or plurality of electrodes for moving liquid droplets may only be present only in the sample preparation module. In such an embodiment, the first substrate may include a series or plurality of electrodes overlayed on an upper surface of the first substrate at the first portion of the first substrate, where the series or plurality of electrodes do not extend to the second portion of the first substrate. In such embodiments, the the series or plurality of electrodes are only positioned in the first portion. A first layer of a dielectric/hydrophobic material (e.g., Teflon), as described above, may be disposed on the upper surface of the first substrate and may cover the series or plurality of electrodes. In certain embodiments, the first layer may be disposed only over a first portion of the first substrate. In other embodiments, the first layer may be disposed over the upper surface of the first substrate over the first portion as well as the second portion. An array of wells may be positioned in the first layer in the second portion of the first substrate, forming the detection module that does not include a series or plurality of electrodes present under the array of wells.

In certain cases, the first layer may be a dielectric layer and a second layer of a hydrophobic material may be disposed over the dielectric layer. The array of wells may be positioned in the hydrophobic layer. Prior to or after fabrication of the array of wells in the hydrophobic layer, a hydrophilic layer may be disposed over the hydrophobic layer in the second portion of the first substrate.

In certain embodiments, the second substrate may extend over the first and second portions of the first substrate. In such an embodiment, the second substrate may be substantially transparent, at least in region overlaying the array of wells. In other cases, the second substrate may be disposed in a spaced apart manner over the first portion of the first substrate and may not be disposed over the second portion of the first substrate. Thus, in certain embodiments, the second substrate may be present in the sample preparation module but not in the detection module.

In certain cases, the second substrate may include a conductive layer that forms an electrode. The conductive layer may be disposed on a lower surface of the second substrate. The conductive layer may be covered by a first layer made of a dielectric/hydrophobic material, as described above. In certain cases, the conductive layer may be covered by a dielectric layer. The dielectric layer may be covered by a hydrophobic layer. The conductive layer and any layer(s) covering it may be disposed across the lower surface of the second substrate or may only be present on the first portion of the second substrate. In certain embodiments, the second substrate may extend over the first and second portions of the first substrate. In such an embodiment, the second substrate and any layers disposed thereupon (e.g., conductive layer, dielectric layer, etc.) may be substantially transparent, at least in region overlaying the array of wells.

In other cases, the series or plurality of electrodes on the first substrate may be configured as co-planar electrodes and the second substrate may not include an electrode.

In certain cases, the electrodes present in the first layer and/or the second layer may be fabricated from a substantially transparent material, such as indium tin oxide, fluorine doped tin oxide (FTO), doped zinc oxide, and the like.

In some embodiments, the sample preparation module and the detection module may be fabricated on a single base substrate. In other embodiments, the sample preparation module and the detection modules may be fabricated on separate substrates that may subsequently be joined to form an integrated microfluidic and analyte detection device. In certain embodiments, the first and second substrates may be spaced apart using a spacer that may be positioned between the substrates.

The devices described herein may be planar and may have any shape, such as, rectangular or square, rectangular or square with rounded corners, circular, triangular, and the like.

Droplet-based microfluidics refer to generating and actuating (such as moving, merging, splitting, etc.) liquid droplets via active or passive forces. Examples of active forces include, but are not limited to, electric field. Exemplary active force techniques include electrowetting, dielectrophoresis, opto-electrowetting, electrode-mediated, electric-field mediated, electrostatic actuation, and the like or a combination thereof. In some examples, the device may actuate liquid droplets across the upper surface of the first layer (or upper surface of the second layer, when present) in the gap via droplet-based microfluidics, such as, electrowetting or via a combination of electrowetting and continuous fluid flow of the liquid droplets. In other examples, the device may include micro-channels to deliver liquid droplets from the sample preparation module to the detection module. In other examples, the device may rely upon the actuation of liquid droplets across the surface of the hydrophobic layer in the gap via droplet based microfluidics. Electrowetting may involve changing the wetting properties of a surface by applying an electrical field to the surface, and affecting the surface tension between a liquid droplet present on the surface and the surface. Continuous fluid flow may be used to move liquid droplets via an external pressure source, such as an external mechanical pump or integrated mechanical micropumps, or a combination of capillary forces and electrokinetic mechanisms. Examples of passive forces include, but are not limited to, T-junction and flow focusing methods. Other examples of passive forces include use of denser immiscible liquids, such as, heavy oil fluids, which can be coupled to liquid droplets over the surface of the first substrate and displace the liquid droplets across the surface. The denser immiscible liquid may be any liquid that is denser than water and does not mix with water to an appreciable extent. For example, the immiscible liquid may be hydrocarbons, halogenated hydrocarbons, polar oil, non-polar oil, fluorinated oil, chloroform, dichloromethane, tetrahydrofuran, 1-hexanol, etc.

The space between the first and second substrates may be up to 1 mm in height, e.g., 0.1 μm, 0.5 μm, 1 μm, 5 μm, 10 μm, 20 μm, 50 μm, 100 μm, 140 μm, 200 μm, 300 μm, 400 μm, 500 μm, 1 μm-500 μm, 100 μm-200 μm, etc. The volume of the droplet generated and moved in the devices described herein may range from about 10 μl to about 5 picol, such as, 10 μl-1 picol, 7.5 μl-10 picol, 5 μl-1 nL, 2.5 μl-10 nL, or 1 μl-100 nL, 800-200 nL, 10 nL-0.5 μl e.g., 10 μl, 1 μl, 800 nL, 100 nL, 10 nL, 1 nL, 0.5 nL, 10 picol, or lesser.

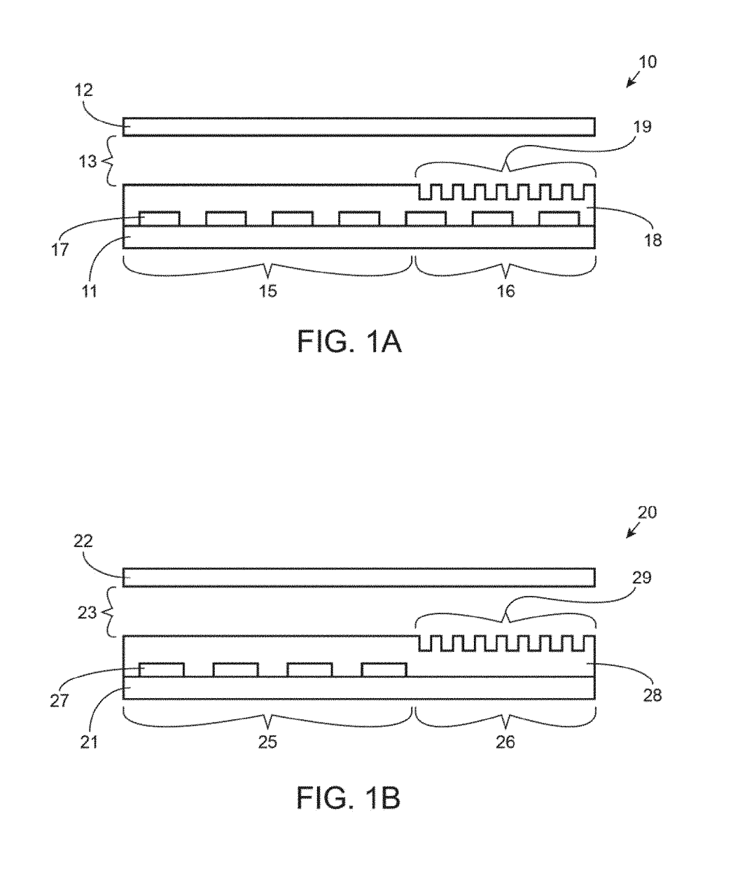

FIG. 1A illustrates an exemplary integrated digital microfluidic and analyte detection device 10. The device 10 includes a first substrate 11 and a second substrate 12, where the second substrate 12 is positioned over the first substrate 11 and separated from the first substrate by a gap 13. As illustrated in FIG. 1A, the second substrate 12 is the same length as the first substrate 11. However, in other exemplary devices, the first substrate 11 and the second substrate 12 may be of different lengths. The second substrate may or may not include an electrode. The first substrate 11 includes a first portion 15, where liquid droplet, such as, a sample droplet, reagent droplet, etc., is introduced onto the first substrate 11. The first substrate 11 includes a second portion 16, towards which a liquid droplet is moved. The first portion 15 may also be referred to as the sample preparation module and the second portion 16 may be referred to as the analyte detection module. The first substrate 11 includes a series or plurality of electrodes 17 positioned on the upper surface of the first substrate 11. A layer 18 of dielectric/hydrophobic material (e.g., Teflon which is both dielectric and hydrophobic) is disposed on the upper surface of the first substrate and covers the series or plurality of electrodes 17. An array of wells 19 is positioned in the layer 18 on the second portion 16 of the first substrate.

FIG. 1B illustrates another example of an integrated digital microfluidic and analyte detection device 20 that includes a first substrate 21 and a second substrate 22, where the second substrate 22 is positioned over the first substrate 20 and separated from an upper surface of the first substrate by a gap 23. The first substrate 21 includes a first portion 25, where a liquid is introduced onto the first substrate 21, and a second portion 26, towards which liquid is directed for detection of an analyte related signal. The first substrate 21 includes a series or plurality of electrodes 27 positioned on the upper surface of the first substrate. A layer 28 of dielectric material is positioned on the upper surface of the first substrate 21 and covers the series or plurality of electrodes 27. In this exemplary device, the series or plurality of electrodes 27 is positioned on only the first portion of the first substrate 21. The second substrate 22 may or may not include an electrode.

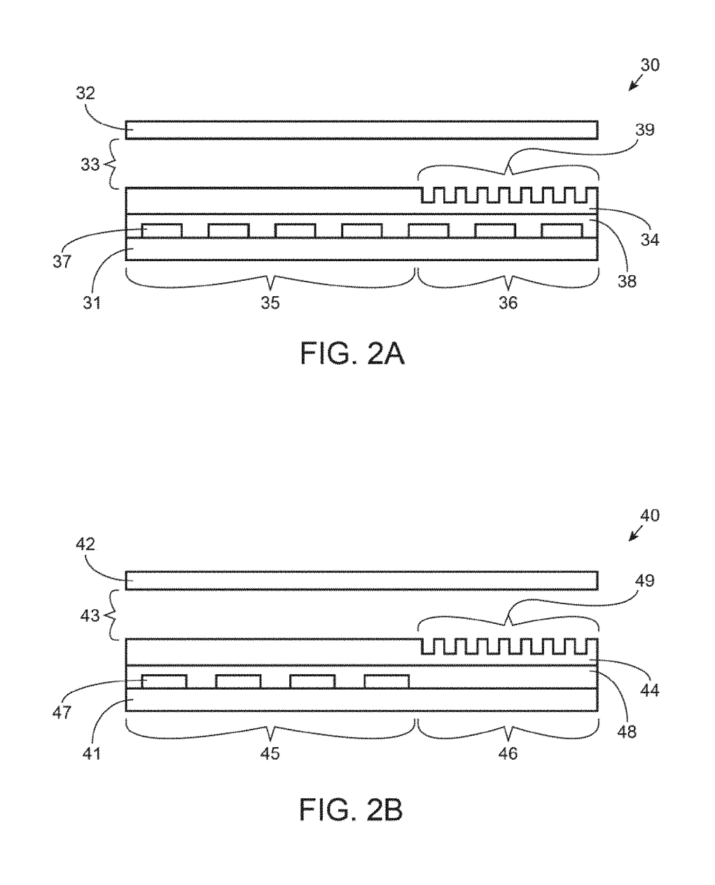

FIG. 2A illustrates another exemplary integrated digital microfluidic and analyte detection device 30. The device 30 includes a first substrate 31 and a second substrate 32, where the second substrate 32 is positioned over the first substrate 31 and separated from an upper surface of the first substrate by a gap 33. The first substrate 31 includes a first portion 35, where liquid droplet, such as, a sample droplet, reagent droplet, etc., is introduced onto the first substrate 31. The first substrate 31 includes a second portion 36, towards which a liquid droplet is moved. The first portion may also be referred to as the sample preparation module and the second portion may be referred to as the detection module. The first substrate 31 includes a series or plurality of electrodes 37 positioned on the upper surface of the first substrate. A layer 38 of dielectric material is disposed on the upper surface of the first substrate and covers the series or plurality of electrodes 37. A layer 34 of hydrophobic material is overlayed on the dielectric layer 38. An array of wells 39 is positioned in the hydrophobic layer 34 on the second portion of the first substrate 31. The array of wells may have a hydrophilic or hydrophobic surface.

FIG. 2B illustrates another example of an integrated digital microfluidic and analyte detection device 40 that includes a first substrate 41 and a second substrate 42, where the second substrate 42 is positioned over the first substrate 40 and separated from an upper surface of the first substrate by a gap 43. The first substrate includes a first portion 45, where a liquid is introduced onto the first substrate 41, and a second portion 46, towards which liquid is directed for detection of an analyte related signal. The first substrate 41 includes a series or plurality of electrodes 47 positioned on the upper surface of the first substrate. A layer 48 of dielectric material is positioned on the upper surface of the first substrate 41 and covers the series or plurality of electrodes 47. In this exemplary device, the series or plurality of electrodes 47 is positioned on only the first portion 45 of the first substrate 41. The dielectric layer 48 covers the entire upper surface of the first substrate 41 and the hydrophobic layer 44 covers the entire upper surface of the dielectric layer. An array of wells 49 is positioned in the hydrophobic layer 44, and the array of wells 49 are positioned at only a portion of the hydrophobic layer overlaying the second portion 46 of the first substrate 41. In this example, the dielectric layer 48 is shown as extending over the entire upper surface of the first substrate 41. In other examples, the dielectric layer and the hydrophobic layer may be limited to the first portion and the wells may be positioned in a hydrophilic layer positioned on the second portion of the first substrate.

In some examples, liquid may be introduced into the gap via a droplet actuator (not illustrated). In other examples, liquid may be into the gap via a fluid inlet, port, or channel. Additional associated components of the device are not illustrated in the figures. Such figures may include chambers for holding sample, wash buffers, binding members, enzyme substrates, waste fluid, etc. Assay reagents may be contained in external reservoirs as part of the integrated device, where predetermined volumes may be moved from the reservoir to the device surface when needed for specific assay steps. Additionally, assay reagents may be deposited on the device in the form of dried, printed, or lyophilized reagents, where they may be stored for extended periods of time without loss of activity. Such dried, printed, or lyophilized reagents may be rehydrated prior or during analyte analysis.

In some examples, the first substrate can be made from a flexible material, such as paper (with ink jet printed electrodes) or polymers. In other examples, the first substrate can be made from a non-flexible material, such as for example, printed circuit board, plastic or glass or silicon. In some examples, the first substrate is made from a single sheet, which then may undergo subsequent processing to create the series or plurality of electrodes. In some examples, multiple series or plurality of electrodes may be fabricated on a first substrate which may be cut to form a plurality of first substrates overlayed with a series or plurality of electrodes. In some examples, the electrodes may be bonded to the surface of the conducting layer via a general adhesive agent or solder. The second substrate may be made from any suitable material including but not limited to a flexible material, such as paper (with or without ink jet printed electrodes), polymers, printed circuit board, and the like.

In some examples, the electrodes are comprised of a metal, metal mixture or alloy, metal-semiconductor mixture or alloy, or a conductive polymer. Some examples of metal electrodes include copper, gold, indium, tin, indium tin oxide, and aluminum. In some examples, the dielectric layer comprises an insulating material, which has a low electrical conductivity or is capable of sustaining a static electrical field. In some examples, the dielectric layer may be made of porcelain (e.g., a ceramic), polymer or a plastic. In some examples, the hydrophobic layer may be made of a material having hydrophobic properties, such as for example Teflon and generic fluorocarbons. In another example, the hydrophobic material may be a fluorosurfactant (e.g., FluoroPel). In embodiments including a hydrophilic layer deposited on the dielectric layer, it may be a layer of glass, quartz, silica, metallic hydroxide, or mica.

One having ordinary skill in the art would appreciate that the array (e.g., series) of electrodes may include a certain number of electrodes per unit area of the first substrate, which number may be increased or decreased based on size of the electrodes and a presence or absence of inter-digitated electrodes. Electrodes may be fabricated using a variety of processes including, photolithography, atomic layer deposition, laser scribing or etching, laser ablation, flexographic printing and ink-jet printing of electrodes.

In some examples, a special mask pattern may be applied to a conductive layer disposed on an upper surface of the first substrate followed by laser ablation of the exposed conductive layer to produce a series or plurality of electrodes on the first substrate.

In some examples, the electrical potential generated by the series or plurality of electrodes transfer liquid droplets formed on an upper surface of the first layer (or the second layer when present) covering the series or plurality of electrodes, across the surface of the digital microfluidic device to be received by the array of wells. Each electrode may be capable of independently moving the droplets across the surface of the digital microfluidic device.

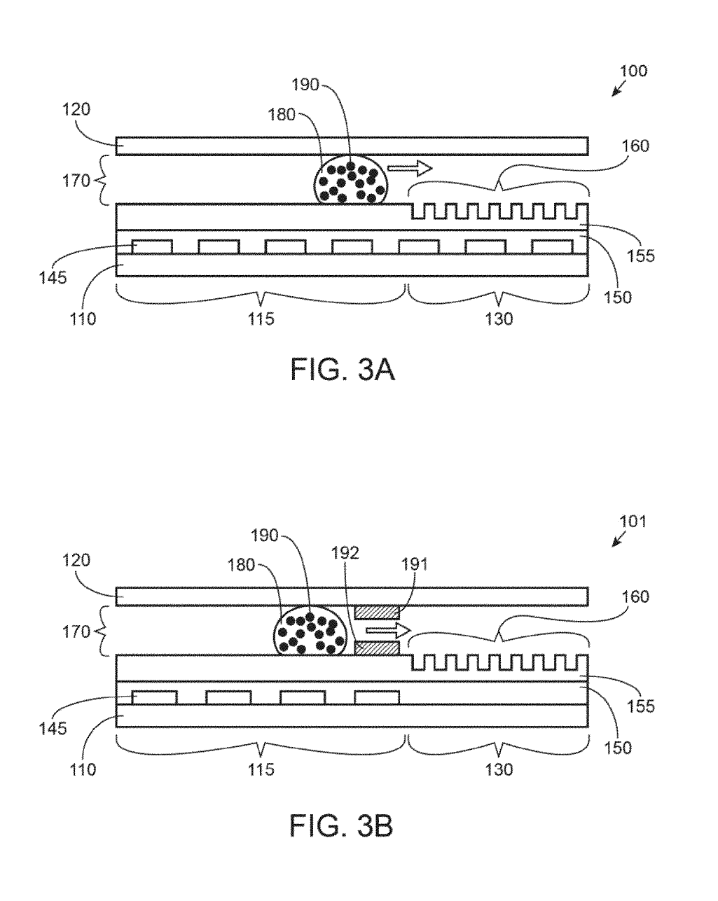

FIG. 3A illustrates a side view of an exemplary integrated digital microfluidic and analyte detection device 100 with a liquid droplet being moved in the gap 170. The device 100 includes a first substrate 110 and a second substrate 120, where the second substrate 120 is positioned over the first substrate 110 and separated from an upper surface of the first substrate by a gap 170. The first substrate 110 includes a first portion 115, where liquid droplet, such as, a sample droplet, reagent droplet, etc., is introduced onto the first substrate 110. The first substrate 110 includes a second portion 130, towards which a liquid droplet is moved. The first portion may also be referred to as the sample preparation module and the second portion may be referred to as the detection module. The first substrate 110 includes a series or plurality of electrodes 145 positioned on the upper surface of the first substrate. A layer 150 of dielectric material is disposed on the upper surface of the first substrate and covers the series or plurality of electrodes 145. A layer 155 of hydrophobic material is overlayed on the dielectric layer 150. An array of wells 160 is positioned in the hydrophobic layer 155 on the second portion of the first substrate 110. The array of wells may have a hydrophilic or hydrophobic surface. As illustrated in FIG. 3A, a liquid droplet is illustrated as being actuated from the first portion 115 to the second portion 130 containing the array of wells 160. A liquid droplet 180 containing a plurality of beads or particles 190 is being moved across the first portion 115 and over to the second portion 130 via active directional movement using the series or plurality of electrodes 145. The arrow indicates the direction of movement of the liquid droplet. Although not shown here, polarizable oil may be used to move the droplet and seal the wells. Although beads/particles are illustrated here, the droplet may include analyte molecules instead of or in addition to the solid supports.

FIG. 3B illustrates a side view of an exemplary integrated digital microfluidic and analyte detection device 101 with a droplet 180 being moved in the gap 170 from the first portion 115 to the second portion 130 that includes the array of wells 160. The device 101 includes a first substrate 110 and a second substrate 120, where the second substrate 120 is positioned over the first substrate 110 and separated from an upper surface of the first substrate by a gap 170. The first substrate 110 includes a first portion 115, where liquid droplet, such as, a sample droplet, reagent droplet, etc., is introduced onto the first substrate 110. The first substrate 110 includes a second portion 130, towards which a liquid droplet is moved. The first portion may also be referred to as the sample preparation module and the second portion may be referred to as the detection module. The first substrate 110 includes a series or plurality of electrodes 145 positioned on the upper surface at the first portion 115 of the first substrate. A layer 150 of dielectric material is disposed on the upper surface of the first substrate and covers the series or plurality of electrodes 145. A layer 155 of hydrophobic material is overlayed on the dielectric layer 150. An array of wells 160 is positioned in the hydrophobic layer 155 on the second portion of the first substrate 110. The array of wells may have a hydrophilic or hydrophobic surface. Movement across the surface of the first portion of the device is via the electrodes 145 and then the droplet 180 is moved to the second portion using passive fluid force, such as capillary movement through capillary element formed by 191 and 192. In some examples, the capillary element may include a hydrophilic material for facilitating movement of the aqueous droplet from the first portion to the second portion in the absence of an applied electric field generated by the series or plurality of electrodes. In some examples, a striping of a hydrophobic material may be disposed next to the hydrophilic capillary space. The striping of hydrophobic material may be used to move a droplet of immiscible fluid over to the array of wells in absence of the digital microfluidics electrodes. Some examples of liquids that may flow through a hydrophobic capillary element includes heavy oil fluids, such as fluorinated oils, can be used to facilitate liquid droplet movement over the array of wells. In other examples, oil droplets may also be utilized to remove excess droplets.

In addition to moving aqueous-based fluids, immiscible fluids, such as organic based immiscible fluids, may also be moved by electrical-mediated actuation. It is understood that droplet actuation is correlated with dipole moment and dielectric constant, which are interrelated, as well as with conductivity. In certain embodiments, the immiscible liquid may have a molecular dipole moment greater than about 0.9 D, dielectric constant greater than about 3 and/or conductivities greater than about 10⁻⁹ S m⁻¹. Examples of movable immiscible liquids and characteristics thereof are discussed in Chatterjee, et al. Lab on Chip, 6, 199-206 (2006). Examples of use of the immiscible liquid in the analyte analysis assays disclosed herein include aiding aqueous droplet movement, displacing aqueous fluid positioned above the wells, displacing undeposited beads/particles/analyte molecules from the wells prior to optical interrogation of the wells, sealing of the wells, and the like. Some examples of organic-based immiscible fluids that are moveable in the devices disclosed herein include 1-hexanol, dichloromethane, dibromomethane, THF and chloroform. Organic-based oils that satisfy the above mentioned criteria would also be expected to be moveable under similar conditions. In some embodiments using immiscible fluid droplets, the gap/space in the device may be filled with air.

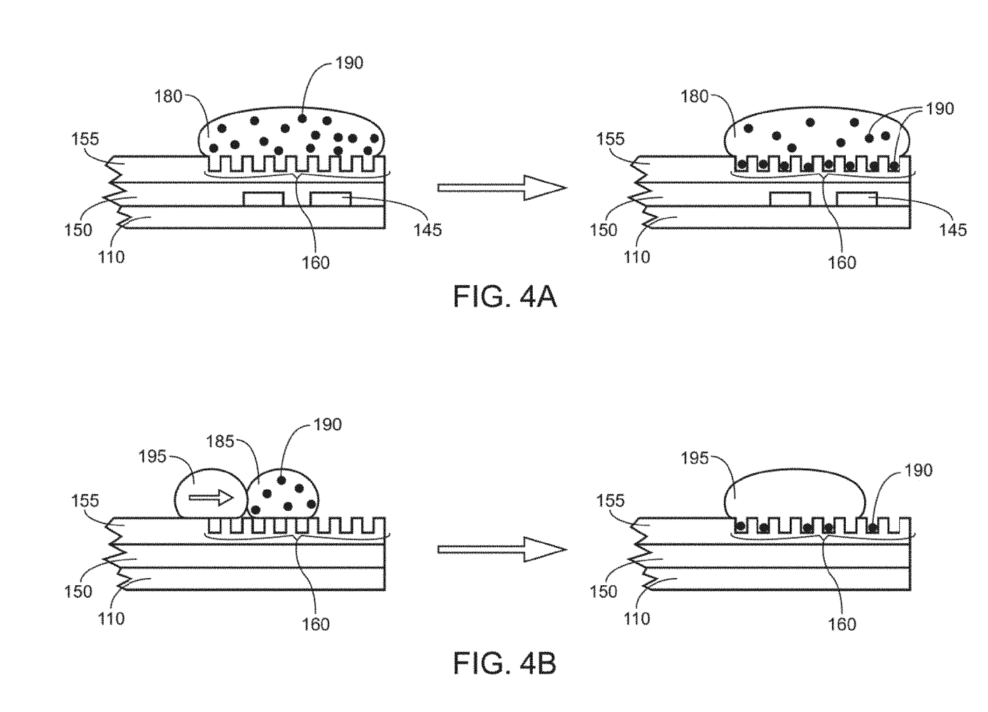

FIG. 4A illustrates a liquid droplet 180 containing beads or particles 190 that has been moved to the second portion of the integrated device of FIG. 3A and is positioned over the array of wells 160. The droplet may be continuously moved over the array of wells in linear or reciprocating motion or movement and may be paused over the array of wells. Moving of the droplet and/or pausing the droplet over the array of wells facilitates the deposition of the particles or beads 190 into the array of wells 160. The wells are dimensioned to include one bead/particle. In the device illustrated in FIG. 4A, the droplet is moved over the array of wells using the series or plurality of electrodes 145. Although beads/particles are depicted here, droplets contain analyte molecules may also be moved in a similar manner, and by pausing the droplet containing the analyte molecules above the wells for a sufficient period of time to allow for the analyte molecules to diffuse into the wells before the immiscible fluid seals the wells. The wells are dimensioned to include one bead/particle. The wells can also be dimensioned to include one analyte molecule per well.

FIG. 4B illustrates a liquid droplet 185 containing beads or particles 190 that has been moved to the second portion of the integrated device of FIG. 3B and is positioned over the array of wells without using a series or plurality of electrodes. In FIG. 4B, a droplet of hydrophobic liquid 195 (such as an immiscible fluid) is being used to move the liquid droplet over the well array to facilitate deposition of the beads/particles 190 into the wells 160. The direction of the arrow indicates the direction in which the droplet 185 is being moved.

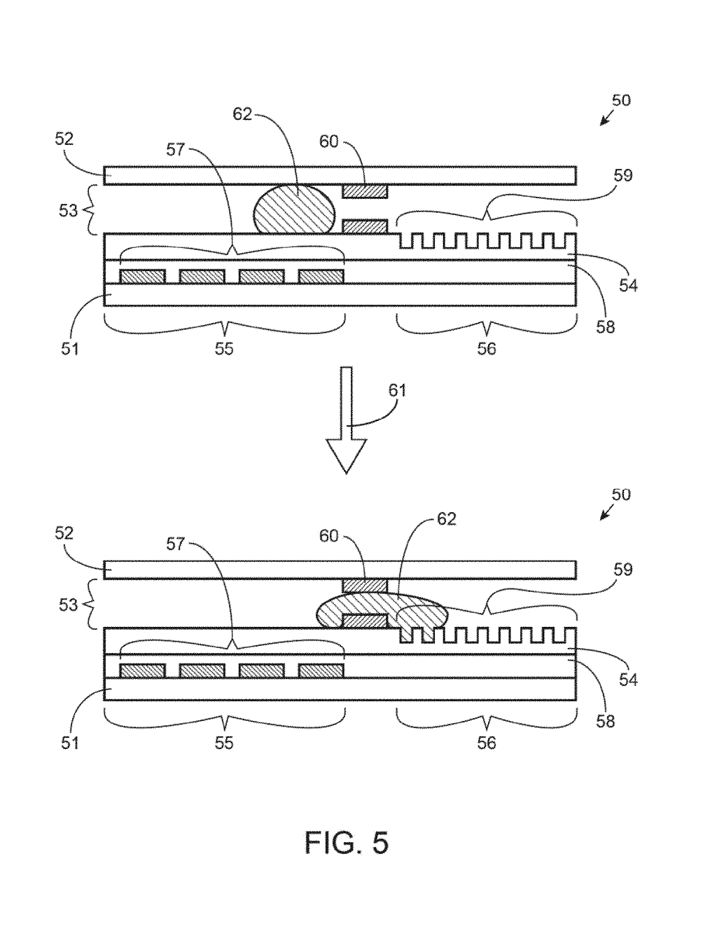

FIG. 5 shows a hydrophobic fluid droplet 62 (e.g., polarizable fluid) being moved over the first portion 55 using the series or plurality of electrodes 57. The depicted device 50 includes a a first substrate 51 and a second substrate 52, where the second substrate 52 is positioned over the first substrate 51 and separated from an upper surface of the first substrate by a gap 53. The first substrate 51 includes a first portion 55, where liquid droplet, such as, a sample droplet, reagent droplet, etc., is introduced onto the first substrate 51. The first substrate 51 includes a second portion 56, towards which a liquid droplet is moved. The first substrate 51 includes a series or plurality of electrodes 57 positioned on the upper surface at the first portion 55 of the first substrate. A layer 58 of dielectric material is disposed on the upper surface of the first substrate and covers the series or plurality of electrodes 57. A layer 54 of hydrophobic material is overlayed on the dielectric layer 58. An array of wells 59 is positioned in the hydrophobic layer 54 on the second portion of the first substrate 51. The array of wells may have a hydrophilic or hydrophobic surface. A capillary element 60 is formed by deposition of two stripes of a hydrophobic material on the first 51 and second substrates 52. The hydrophobic capillary facilitates movement of the hydrophobic fluid droplet 62 to the array of wells 59, in absence of the series or plurality of electrodes in the second portion 56. In other embodiments, the capillary element may be formed by deposition of two stripes of a hydrophilic material on the first 51 and second substrates 52. The hydrophilic material facilitates movement of an aqueous droplet to the array of wells 59, in absence of the series or plurality of electrodes in the second portion 56. In certain embodiments, the capillary element may include a pair of stripes of hydrophilic material alternating with a pair of stripes of hydrophobic material. An aqueous droplet may be directed to the region at which a pair of hydrophilic stripes is positioned, while a droplet of immiscible fluid may be directed to the region at which a pair of hydrophobic stripes is positioned.

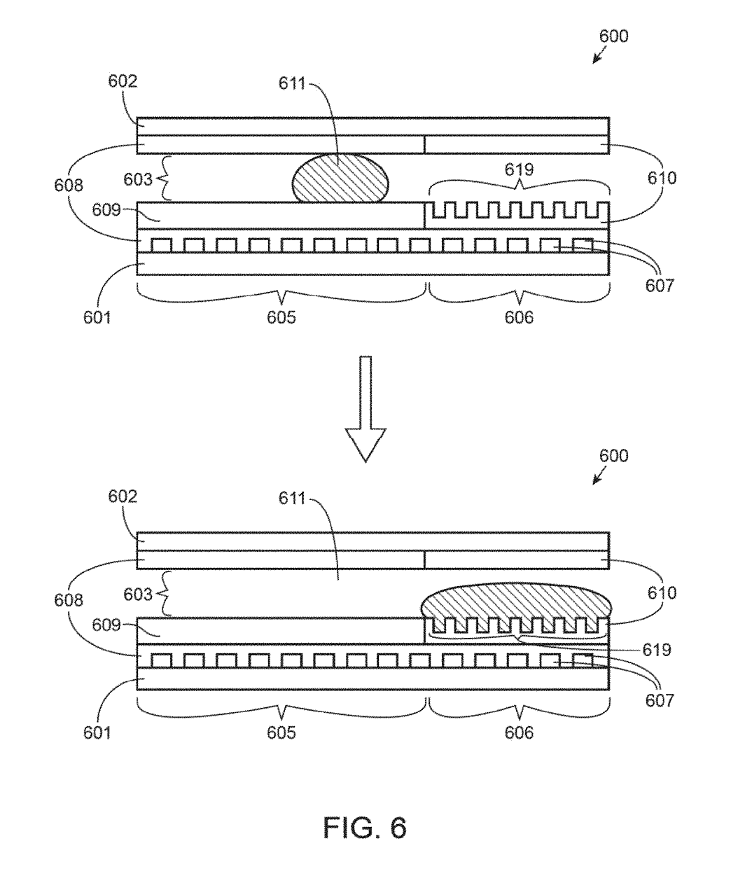

FIG. 6 depicts another embodiment of an integrated digital microfluidics and analyte detection device. The device 600 includes a bottom layer 601 over which an array of electrodes 607 is formed. The array of electrodes is covered by a dielectric layer 608. A hydrophobic layer 609 is disposed only in the first portion 605 of the bottom substrate. A hydrophilic layer 610 is disposed on the second portion 606 of the bottom substrate 601. An array of wells is located in the second portion in the hydrophilic layer 610. A top substrate 602 separated from the bottom substrate by a gap/space 603 is also depicted. The top substrate 602 includes a dielectric layer 608 disposed on a bottom surface of the top substrate over the first portion of the bottom substrate. The top substrate includes a hydrophilic layer 610 disposed on a bottom surface of the top substrate across from the second portion of the bottom substrate. An aqueous droplet 611 does not wet the hydrophobic layer and upon reaching the hydrophilic second portion the droplet 611 spreads over the array of wells 619, thereby facilitating movement of the aqueous phase via passive capillary forces. In a similar manner, the above concept may be reversed to facilitate wetting and spreading of an organic-based immiscible fluid over the wells. In this case, the top and bottom substrate on the second portion can be coated with a hydrophobic material/coating, thereby allowing an organic-based immiscible fluid to flow over the wells via passive capillary forces.

As used herein, digital microfluidics refers to use of a a series of electrodes to manipulate droplets in a microfluidics device, e.g., move droplets, split droplets, merge droplets, etc. in a small space. As used herein, the terms “droplet(s)” and “fluidic droplet(s)” are used interchangeably to refer to a discrete volume of liquid that is roughly spherical in shape and is bounded on at least one side by a wall or substrate of a microfluidics device. Roughly spherical in the context of the droplet refers to shapes such as spherical, partially flattened sphere, e.g., disc shaped, slug shaped, truncated sphere, ellipsoid, hemispherical, or ovoid. The volume of the droplet in the devices disclosed herein may range from about 10 μl to about 5 pL, such as, 10 μl-1 pL, 7.5 μl-10 pL, 5 μl-1 nL, 2.5 μl-10 nL, or 1 μl-100 nL, e.g., 10 μl, 5 μl, 1 μl, 800 nL, 500 nL, or lesser.

In some examples, the array of wells includes a plurality of individual wells. The array of wells may include a plurality of wells that may range from 10 to 10⁹ in number per 1 mm². In certain cases, an array of about 100,000 to 500,000 wells (e.g., femtoliter wells) covering an area approximately 12 mm² may be fabricated. Each well may measure about 4.2 μm wide×3.2 μm deep (volume approximately 50 femtoliters), and may be capable of holding a single bead/particle (about 3 μm diameter). At this density, the femtoliter wells are spaced at a distance of approximately 7.4 μm from each other. In some examples, the well array may be fabricated to have individual wells with a diameter of 10 nm to 10,000 nm.

The placement of single beads/particles/analyte molecules in the wells allows for either a digital readout or analog readout. For example, for a low number of positive wells (<˜70% positive) Poisson statistics can be used to quantitate the analyte concentration in a digital format; for high numbers of positive wells (>˜70%) the relative intensities of signal-bearing wells are compared to the signal intensity generated from a single bead/particle/analyte molecule, respectively, and used to generate an analog signal. A digital signal may be used for lower analyte concentrations, whereas an analog signal may be used for higher analyte concentrations. A combination of digital and analog quantitation may be used, which may expand the linear dynamic range. As used herein, a “positive well” refers to a well that has a signal related to presence of a bead/particle/analyte molecule, which signal is above a threshold value. As used herein, a “negative well” refers to a well that may not have a signal related to presence of a bead/particle/analyte molecule. In certain embodiments, the signal from a negative well may be at a background level, i.e., below a threshold value.

The wells may be any of a variety of shapes, such as, cylindrical with a flat bottom surface, cylindrical with a rounded bottom surface, cubical, cuboidal, frustoconical, inverted frustoconical, or conical. In certain cases, the wells may include a sidewall that may be oriented to facilitate the receiving and retaining of a microbead or microparticle present liquid droplets that have been moved over the well array. In some examples, the wells may include a first sidewall and a second sidewall, where the first sidewall may be opposite the second side wall. In some examples, the first sidewall is oriented at an obtuse angle with reference to the bottom of the wells and the second sidewall is oriented at an acute angle with reference to the bottom of the wells. The movement of the droplets may be in a direction parallel to the bottom of the wells and from the first sidewall to the second sidewall.

In some examples, the array of wells can be fabricated through one or more of molding, pressure, heat, or laser, or a combination thereof. In some examples, the array of wells may be fabricated using nanoimprint/nanosphere lithography. Other fabrication methods well known in the art may can also be used.

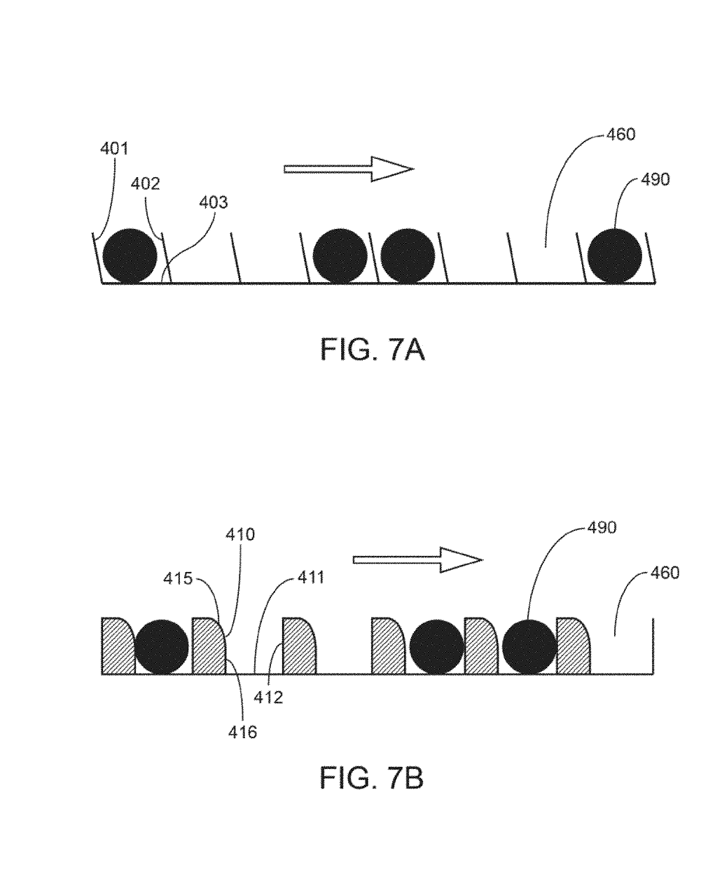

FIGS. 7A-7B illustrate several exemplary sidewall orientations of the wells. As illustrated in FIGS. 7A-B, the wells comprise a first sidewall opposite to a second sidewall. FIG. 7A illustrates a vertical cross-section showing individual wells 460 in the array of wells. FIG. 7A illustrates a first sidewall 401 and a second sidewall 402. The first side wall is at an obtuse angle with reference to a bottom surface 143 of the well and the second side wall is at an acute angle with reference to a bottom surface 143 of the well. The arrow illustrates the direction in which a liquid droplet moves across the array. This orientation of the sidewalls of the wells facilitates receiving and retaining beads/particles/analyte molecules 490.

In FIG. 7B, a top portion 415 of the first sidewall 410 is oriented at an obtuse angle with reference to a bottom 412 of the wells and a bottom portion 416 of the first sidewall 410 is oriented perpendicular to the bottom 412 of the wells, and the second sidewall 411 is oriented perpendicular to the bottom 412 of the wells, where movement of liquid droplets is in a direction parallel to the bottom of the wells and from the first sidewall to the second sidewall, where the top portion of the first sidewall is at an opening of the wells.

The integrated devices described herein may be fabricated by a number of methods. In certain cases, the methods may involve a combination of laser ablation, spray coating, roll to roll, flexographic printing, and nanoimprint lithography (NIL) to construct the first substrate, series or plurality of electrode, dielectric layer and hydrophobic layer.

In some examples, a plurality of rollers may unwind a first roll to drive the first substrate to a first position. A conductive material may then be applied to the first substrate. The conductive material may be patterned into a series or plurality of electrodes. In some examples, the printer device comprising one or more coating rollers to apply the at least one of the hydrophobic or the dielectric material to the at least one electrode pattern on the first substrate. In some examples, the coating rollers are to apply an anti-fouling material to the first substrate.

In some examples, the system further comprises a merger to align the first substrate with the second substrate. In some examples, the merger comprises two rollers. Also, some of the disclosed examples include a curing station to cure the hydrophobic material or the dielectric material. Some of the disclosed examples also include a bonding station to bond at least a first portion of the first substrate with at least a first portion of the second substrate. The bonded portions include the electrode pattern. The method also includes associating the first substrate and the second substrate at a spaced apart distance. The space between the first and second substrates may be about 0.01 mm to 1 mm in height, e.g., 0.1 μm, 0.5 μm, 1 μm, 5 μm, 10 μm, 20 μm, 50 μm, 100 μm, 140 μm, 200 μm, 300 μm, 400 μm, 500 μm, 1 μm-500 μm, 100 μm-200 μm, etc.

In some examples, the method includes embossing the first substrate to create one or more projections on the first substrate. In such examples, the projections are to separate the first substrate and the second substrate at the spaced apart distance.

The devices of the present disclosure may be operated manually or automatically or semiautomatically. In certain cases, the devices may be operated by a processor that runs a program for carrying out the steps required for generating an analyte related signal and detecting the signal. As used hereon, the phrase “analyte related signal” or “analyte associated signal” refers to a signal that is indicative of presence of an analyte and is proportional to the amount of the analyte in a sample. The signal may be fluorescence, chemiluminescence, colorimetric, turbidimetric, etc. In certain cases, the read out may be digital, for example, the number of positive counts (e.g., wells) is compared to the number of negative counts (e.g., wells) to obtain a digital count.

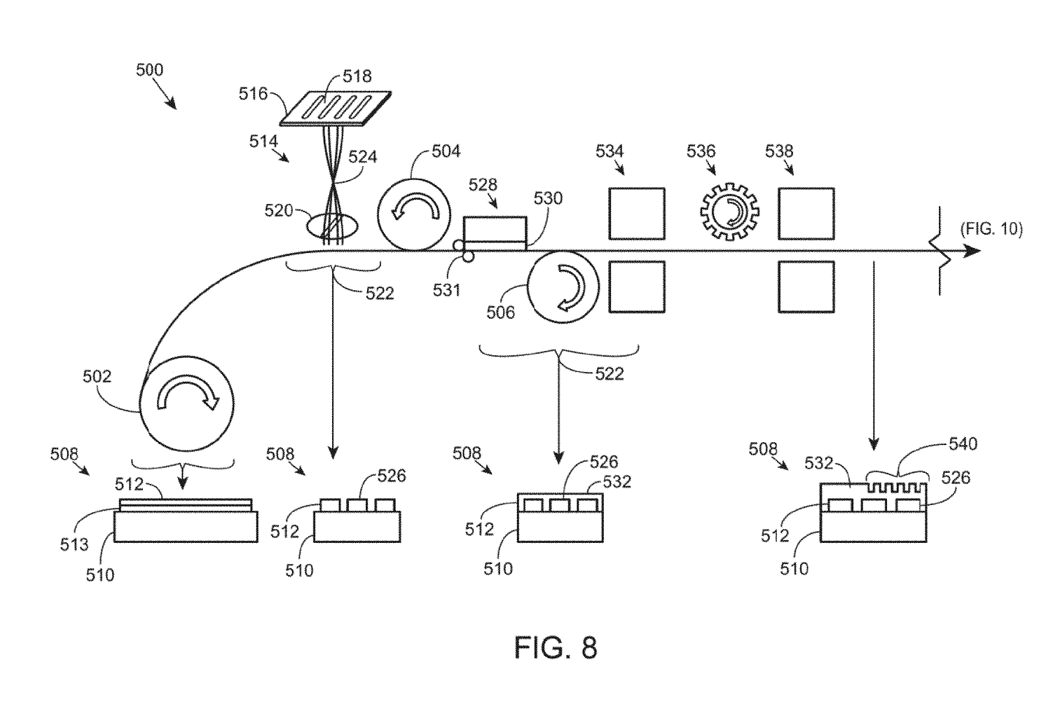

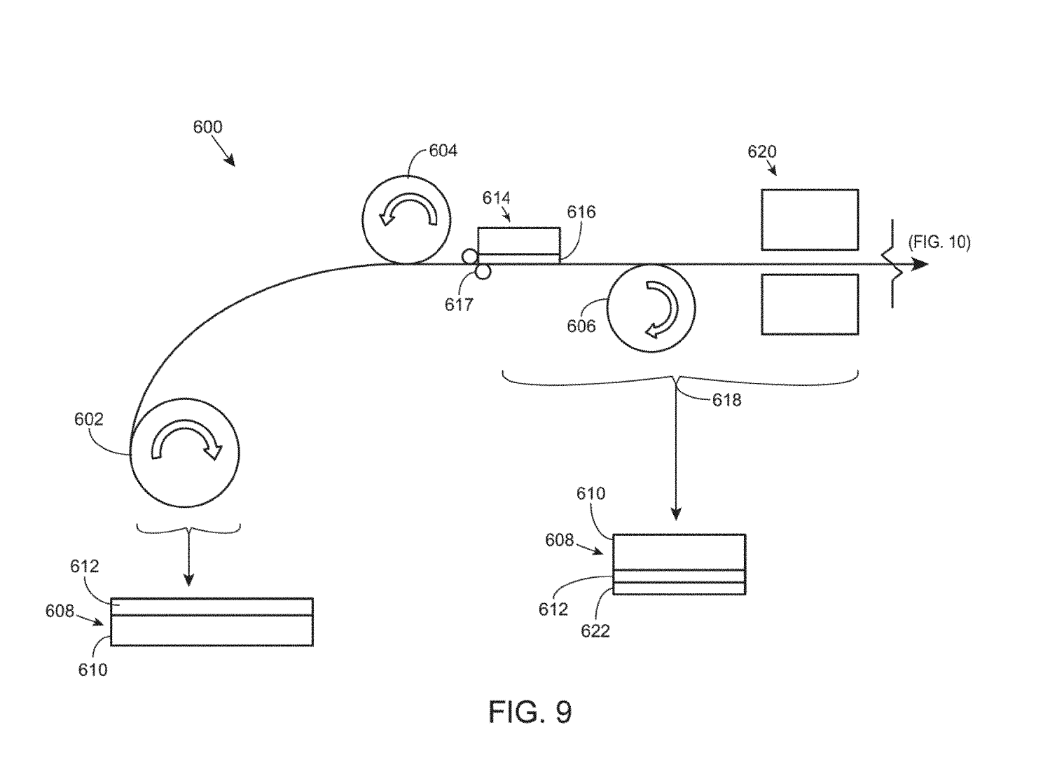

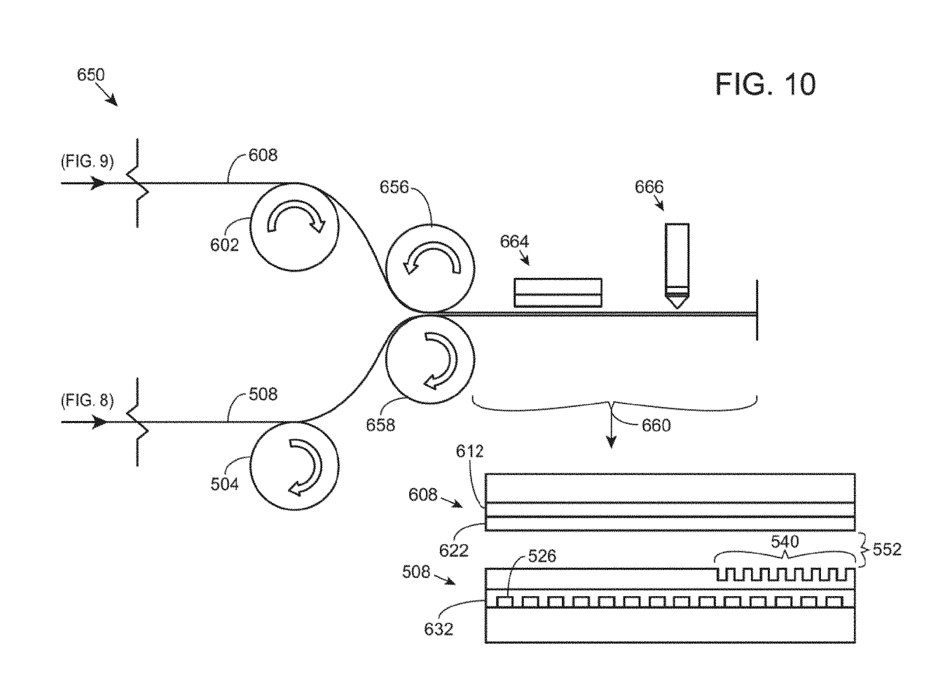

FIG. 8 is a diagram of a first exemplary system or assembly 500 for creating a base substrate of an integrated digital microfluidics and analyte detection device. The first example assembly 500 includes a series or a plurality of rollers, including a first roller 502, a second roller 504, and a third roller 506, which operate in synchronized rotation to drive a base substrate 508 through the first example assembly 500. The first example assembly 500 can include rollers in addition to the first through third rollers 502, 504, 506 to move the base substrate 508 through the assembly using roll-to-roll techniques. Other examples may use conveyors, pulleys and/or any other suitable transport mechanism(s).

In the first example assembly 500, the first roller 502 rotates to unwind the base substrate 508, which, in some examples, is a single sheet in a rolled configuration. The base substrate 508 includes a first layer 510 and a second layer 512. In this example, the first layer 510 comprises a non-conductive flexible substrate or web, such as for example a plastic, and the second layer 512 includes a conductive material. The conductive material of the second layer 512 can be, for example, a metal such as gold, silver, or copper, or a non-metallic conductor, such as a conductive polymer. In other examples different metal(s) or combination(s) of metal(s) and/or conductive polymer(s) may be used. In some examples, the base substrate 508 includes an optional adhesive layer 513 disposed between the non-conductive first layer 510 and the conductive second layer 512. As an example, the adhesive layer 513 can comprise chrome, with a layer of gold disposed on top of the chrome adhesive layer 513 to form the conductive second layer 512. Thus, in the base substrate 508 of FIG. 8, the non-conductive first layer 510 and the conductive second layer 512 are pre-adhered to form the base substrate 508 prior to being unwound by the first roller 502.

In the example base substrate 508 of FIG. 8, the non-conductive first layer 510 has a thickness of less than about 500 nm. As will be described below, such a thickness allows for the base substrate 508 to move through the example first assembly 500 via the plurality of rollers. Also, in some examples, the thickness of the nonconductive first layer 510 is greater than a thickness of the conductive second layer 512. As an example, the thickness of the conductive second layer 512 can be approximately 30 nm. In other examples, the thickness of the conductive second layer 512 is less than about 500 nm. In some examples, the thickness of the non-conductive first layer 510 and/or the conductive second layer 512 is selected based on, for example, the materials of the first and/or second layers 510, 512 and/or an operational purpose for which the droplet actuator formed from the base substrate 508 is to be used.

The first roller 502 drives the base substrate 508 to a laser ablation station 514. The laser ablation station 514 includes a mask 516 containing a master pattern 518 that is to be projected onto the conductive second layer 512 of the base substrate 508. The master pattern 518 associated with the mask 516 may be predefined based on characteristics such as resolution (e.g., number of electrodes per an area of the base substrate 508 to be ablated), electrode size, configuration of lines defining the electrode pattern, inter-digitation of the electrodes, gaps or spacing between the electrodes, and/or electrical traces for connecting the electrodes to an instrument, such as, a power source. In some examples, the characteristics of the master pattern 518 are selected based on one or more operational uses of the droplet actuator with which the base substrate 508 is to be associated (e.g., for use with biological and/or chemical assays). Also, in some examples, the master pattern 518 is configurable or reconfigurable to enable the laser ablation station 514 to form different patterns on the base substrate 508. Additionally or alternatively, in some examples the mask 516 is replaceable with one or more alternative masks.

The laser ablation station 514 includes a lens 520. As the base substrate 508 encounters the laser ablation station 514 as result of the rotation of the rollers (e.g., the first roller 502), a portion 522 of the base substrate 508 passes under or past the lens 520. The portion 522 may be, for example, a rectangular or square section of the base substrate 508 having an area less than the area of the base substrate 508 and including the conductive second layer 512. The lens 520 images or projects at least a portion of the master pattern 518 onto the conductive second layer 512 associated with the portion 522. A laser beam 524 is directed onto the portion 522 via the mask 516 and the lens 520 such that the laser beam 524 selectively penetrates the conductive second layer 512 based on the projected master pattern 518. In some examples, the non-conductive first layer 500 or a portion (e.g., a fraction of the thickness of the non-conductive first layer 510) may also be penetrated by the laser beam 524 based on the projected master pattern 518. The solid portions of the mask 516 block the laser beam 524, and the open portions of the mask 516 allow the laser beam 524 to pass through the mask 516 and into contact with the base substrate 508. The laser beam 524 can be associated with, for example, an excimer laser.

As a result of exposure to the laser beam 524, the irradiated nonconductive first layer 510 of the portion 522 absorbs energy associated with the laser beam 524. The irradiated non-conductive first layer 510 undergoes photochemical dissociation, resulting in a selective breaking up of the structural bonds of nonconductive first layer 510 and ejection of fragments of the non-conductive first layer 510 and portions of the conductive second layer 512 overlaying the irradiated non-conductive first layer 510 in accordance with the master pattern 518. In some examples, a depth (e.g., a radiation intensity) to which the laser beam 524 penetrates the base substrate 508 is predefined based on a depth (e.g., a thickness) of the non-conductive first layer 510 and/or the conductive second layer 512. In some examples, the laser beam 524 penetration depth is adjustable to change the depth at which the laser beam 524 ablates the conductive second layer 512 as a result of the fragmentation of the underlying nonconductive first layer 510. In some examples, this adjustment is dynamic as the example system 500 operates. Also, in some examples, the base substrate 508 undergoes cleaning after exposure to the laser beam 524 to remove particles and/or surface contaminants.

As illustrated in FIG. 8, after exposure to the laser ablation station 514, the portion 522 of the base substrate 508 includes an electrode array 526. The electrode array 526 is made up of a plurality of electrodes formed into the conductive second layer 512. As a result of the exposure to the laser beam 524 and fragmentation of the non-conductive first layer 510, portions of the conductive second layer 512 are removed from the base substrate 508. The removed portions associated with the electrode array 526 are based on the master pattern 518. In some examples, the removed portions match the open portions of the mask 516.

Returning to FIG. 8, after the portion 522 undergoes laser ablation at the laser ablation station 514 to form the electrode array 526, the portion 522 is moved, via rotation of the first through third rollers 502, 504, 506, to a printer 528. In the first example assembly 500, the printer 528 includes an apparatus or an instrument capable of applying at least one layer of material 530 having a hydrophobic and/or a dielectric property to the electrode array 526. In the first example assembly 500, the printer 528 can deposit the hydrophobic and/or dielectric material 530 via deposition techniques including, but not limited to, web-based coating (e.g., via rollers associated with the printer 528), slot-die coating, spin coating, chemical vapor deposition, physical vapor deposition, and/or atomic layer deposition. The printer 528 can also apply other materials in addition to the hydrophobic and/or dielectric material 530 (e.g., anti-fouling coatings, anti-coagulants). Also, the printer 528 can apply one or more layers of the material(s) with different thicknesses and/or covering different portions of the base substrate 508.

As described above, in the first example assembly 500, at least one of the first through third rollers 502, 504, 506 advance the base substrate 508 to the printer 528 for application of the hydrophobic and/or dielectric material 530 to the electrode array 526. In some examples, the printer 528 includes a plurality of registration rollers 531 to facilitate accuracy in feeding and registration of the base substrate 508 as part of operation of the printer 528 in applying the hydrophobic and/or dielectric material 530, for example, via roller coating methods.

In the first example assembly 500, the hydrophobic and/or dielectric material 530 is applied to the electrode array 526 to completely or substantially completely insulate the electrode array 526.

In some examples, the hydrophobic and/or dielectric material 530 is deposited via the printer 528 in substantially liquid form. To create a structural or treated layer 532 on the base substrate 508 to support a droplet, the portion 522 is moved via the rollers (e.g., the first through third rollers 502, 504, 506) through a curing station 534. At the curing station 534, the hydrophobic and/or dielectric material is treated and/or modified to form the first treated layer 532. Treating and/or modifying the hydrophobic and/or dielectric material can include curing the material. For example, at the curing station 534, heat is applied to facilitate the hardening of the hydrophobic and/or dielectric material 530. In some examples, the portion 522 is exposed to an ultraviolet light to cure the hydrophobic and/or dielectric material 530 and form the treated layer 532 to insulate the electrode array 526. In other examples, the curing and/or modification of the hydrophobic and/or dielectric material is accomplished without heat and/or a photon source. In some examples, the treated layer 532 supports a droplet as an electric field is applied (e.g., in connection with electrode array 526) to manipulate the droplet. For example, during an electrowetting process, a contact angle of the droplet with respect to the treated layer 532 changes as a result of an applied voltage, which affects the surface tension of the droplet on the treated surface 532. Electrowetting is merely exemplary, the droplet may be moved using other forces as well.

After passing through the curing station 534, the portion 522 is prepared to serve as a bottom substrate of a droplet actuator and/or as a digital microfluidic chip. Because the base substrate 508 includes the non-conductive first layer 510 bonded with the conductive second layer 512, as disclosed above, additional adhesion of, for example the electrode array 526 to the non-conductive first layer 510 is not required. Such a configuration increases the efficiency of the preparation of the base substrate 508 for the droplet actuator by reducing processing steps. Also, as described above, when the portion 522 is at any one of the laser ablation station 514, the printer 528, or the curing station 534, other portions of the base substrate 508 are concurrently moving through the others of the respective stations 514, 528, 534 of the first example assembly 500. For example, when the portion 522 is at the curing station 534, the first through third rollers 502, 504, 506 are continuously, periodically, or aperidiocally advancing one or more other portions of the base substrate 508 through, for example, the laser ablation station 514 and/or the printer 528. In such a manner, preparation of the base substrate 508 for the droplet actuator is achieved via a substantially continuous, high-speed, automated process.

After the curing step, a pattern roller is rolled over a second portion of the base substrate to create an array of wells 540. The array wells 540 may subsequently be coated with a hydrophilic material (not shown).

Although the base substrate 508 may be considered as including successive portions, during some example operations of the first example assembly 500, the base substrate 508 remains as a single sheet as the successive portions undergo processing to create the electrode arrays 526 (e.g., via the electrode pattern) and receive the coating of hydrophobic and/or dielectric material 530. Thus, to create one or more droplet actuators using the processed base substrate 508, the base substrate 508, in some examples, is cut (e.g., diced) to form individual units comprising the electrode arrays 526, as will be further disclosed below. In some examples, prior to dicing, the base substrate 508, including the portion 522, is rewound in a rolled configuration similar to the initial rolled configuration of the base substrate 508 prior to being unwound by the first roller 502. Such rewinding may be accomplished via one or more rollers as part of the roll-to-roll processing. In such examples, the base substrate 508 may be diced or otherwise separated at a later time. In other examples, the rollers (e.g., the second and third rollers 504, 506), advance the base substrate 508 for merging with a top substrate.

FIG. 9 illustrates a second example assembly 600 for creating an example top substrate of a droplet actuator having a single electrode. The second example assembly 600 includes a series or a plurality of rollers, including a first roller 602, a second roller 604, and a third roller 606, which operate in synchronized rotation to drive a top substrate 608 through the second example assembly 600. The second example assembly 600 can include rollers in addition to the first through third rollers 602, 604, 606 to move the top substrate 608 through the assembly 600.

In the second example assembly 600, the first roller 602 rotates to unwind the top substrate 608, which, in some examples, is a sheet in a rolled configuration. The example top substrate 608 of FIG. 9 includes a first layer 610 and a second layer 612. As with the example base substrate 508, in this example, the example first layer 610 of the top substrate 608 comprises a non-conductive material such as, for example, a plastic, and the example second layer 612 includes a conductive material, such as a metal including, for example, one or more of gold, chrome, silver, indium tin oxide, or copper and/or any other suitable metal(s), conductive polymer(s), or combination(s) of metal(s) and/or conductive polymer(s). In some examples, the conductive second layer 612 is adhered to the nonconductive first layer via an adhesive layer (e.g., chrome).

In the second example assembly 600, the first through third rollers 602, 604, 606 rotate to advance the top substrate 612 to a printer 614. The printer 614 coats the conductive second layer 612 with a hydrophobic and/or dielectric material 616 (e.g. Teflon® or parylene C, or a dielectric such as a ceramic). The printer 614 is substantially similar to the printer 528 of the first example assembly 500 of FIG. 8. For example, the printer 614 can apply the hydrophobic and/or dielectric material 616 to the top substrate 608 via web-based coating, slot-die coating, spin coating, chemical vapor deposition, physical vapor deposition, atomic layer deposition, and/or other deposition techniques. The printer 614 can include registration rollers 617 to facilitate alignment of the top substrate 608 with respect to the printer 614 during application of the hydrophobic and/or dielectric material 616 and/or other coating materials.

After receiving the coating of the hydrophobic and/or dielectric material 616, the second roller 504 and the third roller 506 advance the portion 618 to a curing station 620. As disclosed in connection with the curing station 534 of FIG. 8, the curing station 620 of the second example assembly 600 facilitates the modification (e.g., curing) of the hydrophobic material via heat to form a treated layer 622. The treated layer 622 insulates the conductive second layer 612, which serves as the single electrode of the top substrate 608, by completely or substantially completely covering the conductive second layer 612. Thus, in coating the second layer 612 of the portion 618, electrical potential conducting portion of the top substrate 608 is insulated from a droplet that may be applied to a droplet actuator that includes the portion 618.

After passing through the curing station 620, the portion 618 is prepared to serve as a top substrate of a droplet actuator. Because the top substrate 608 includes the non-conductive first layer 610 pre-adhered to the conductive second layer 612, additional adhesion of, for example, an electrode to the non-conductive first layer 610 is not required, thereby increasing the efficiency of the preparation of the top substrate 608 for the droplet actuator.

In the second example assembly 600, the first through third rollers 602, 604, 606 rotate to advance the top substrate 608 such that portions of the top substrate pass through one of the printer 614 or the curing station 620 in substantially continuous, periodic and/or aperiodic succession as part of the roll-to-roll operation of the second example assembly 60. Thus, although the second example assembly 600 is described in association with the portion 618, it is to be understood that successive portions of the top substrate 608 are prepared in substantially the manner as the portion 618 as a result of rotation of the first through third rollers 602, 604, 606. In such as manner, the top substrate 308 is provided with a treated layer 622 along the length of the top substrate 608.

In the example top substrate 608, the conductive second layer 612 serves an electrode. However, in some examples, the conductive second layer 612 undergoes laser ablation to form one or more electrode arrays. In such examples, the second example assembly 600 includes a laser ablation station. Thus, prior to receiving the hydrophobic material 616, the top substrate 608 is exposed to a laser beam, which creates an electrode pattern in the irradiated conductive second layer 612. Also, in some examples, the electrode array is not formed on/in the base substrate but only on/in the top substrate 608.

During operation of the second example assembly 600, the top substrate remains single sheet as successive portions of the top substrate 608 are coated with the hydrophobic material 616. As part of the fabrication of one or more droplet actuators, the top substrate 608 is aligned with the base substrate. In some examples, after passing through the curing station 620, the top substrate is rewound into a rolled configuration via one or more rollers. In such examples, the finished roll may be diced or otherwise cut and/or separated into individual units that are aligned at a spaced apart distance and bonded with individual diced units of the base substrate to create a droplet actuator.

In other examples, after passing through the curing station 620, the rollers (e.g., the first through third rollers 602, 604, 606) continue to advance the top substrate 608 to merge the top substrate 608 with the base substrate via automated roll-to-roll processing. In such examples, to prepare the top substrate 608 for alignment with the base substrate 508, the first through third rollers 602, 604, 606 rotate so as to reverse the orientation of the top substrate relative to the base substrate such that the treated layer of the base substrate faces the treated layer 622 of the top substrate 608 when the base substrate 508 and the top substrate 608 are aligned in parallel configuration.

As show in FIG. 10, the third example assembly 650 includes a third roller 656 and a fourth roller 608 that form a pair of merging rollers to which the base substrate 508 and the top substrate 608 are fed via the respective first roller 652 and the second roller 654 of the third example assembly 650. As each of the merging rollers 656, 658 rotates, the base substrate 658 and the top substrate 658 are aligned in a parallel configuration at a predetermined spaced apart distance, or gap.

The example third assembly 650 includes a bonding station 664. The bonding station 664 joins, or bonds, the base substrate 508 and the top substrate 608 as part of fabricating the droplet actuator. For example, at the bonding station 664, one or more adhesives may be selectively applied to a predefined portion of the base substrate 508 and/or the top substrate 608 (e.g., a portion of the base substrate 508 and/or the top substrate 608 defining a perimeter of the resulting droplet actuator) to create a bond between the base substrate 508 and the top substrate 608 while preserving the gap 662. In some examples, bonding the substrates 508, 608 at the bonding station 664 including forming the gap 662 (e.g., in advance of applying the adhesive).

Examples of adhesive(s) that may be used at the bonding station 664 include epoxies, foils, tapes, and/or ultraviolet curable adhesives. In some examples, layers of polymers such as SU-8 and/or polydimethylsiloxane (PDMS) are applied to the base substrate 508 and/or the top substrate 608 to bond the substrates. Also, in some examples, the bonding station 664 provides for curing of the adhesive(s) via, for example, ultraviolet light. The bonding station 664 may apply one more methods involving, for example, heat (e.g. thermal bonding), pressure, curing, etc. to bond the base substrate 658 and the top substrate 608.

In the example third assembly 650, the merged portion 660 can be selectively cut, diced or otherwise separated to form one or more droplet actuators, as substantially represented in FIG. 10 by the merged portion 660. The example third assembly 650 includes a dicing station 666. The dicing station 666 can be, for example, a cutting device, a splitter, or more generally, an instrument to divide the continuous merged portion 660 into discrete units corresponding to individual droplet actuators. The merged portion 660 may be cut into individual droplet actuators based on, for example, the electrode pattern such that each droplet actuator includes a footprint of the electrode array and the other electrodes that are formed via the electrode pattern.

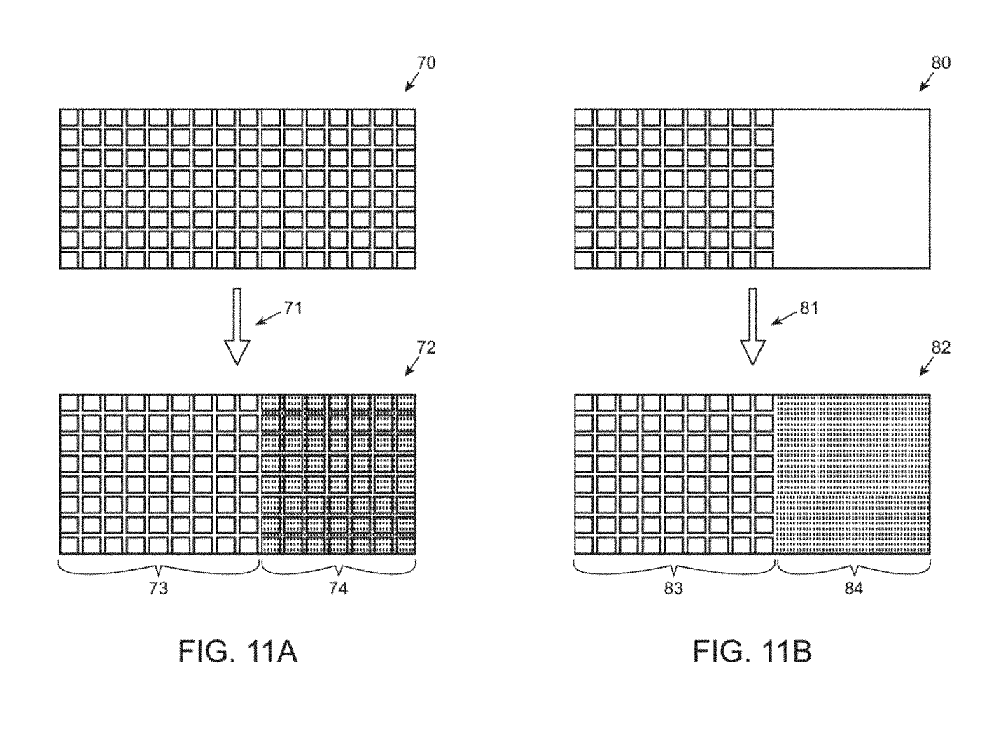

FIG. 11A depicts a top view of the bottom substrate 70 on which an array of electrodes is present in the first portion 73 and second portion 74. The bottom substrate 72, after step 71 of fabrication of an array of wells on the second portion, is shown. FIG. 11B depicts a top view of a bottom substrate 80 with an array of electrodes disposed only in the first portion 83. The bottom substrate 82 is depicted after the step 81 in which an array of wells is formed in the second portion 84.

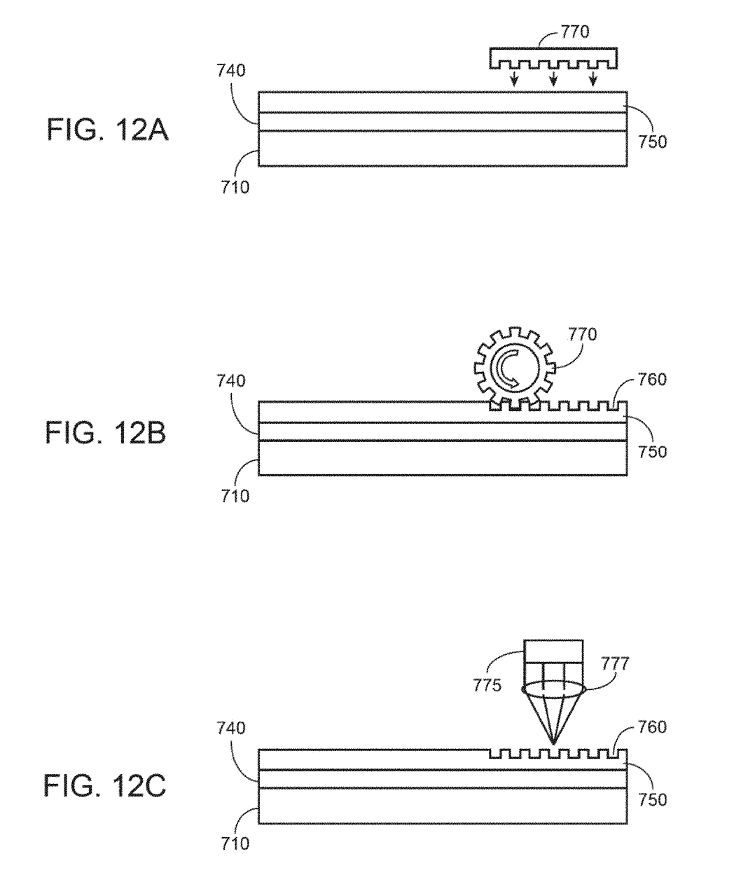

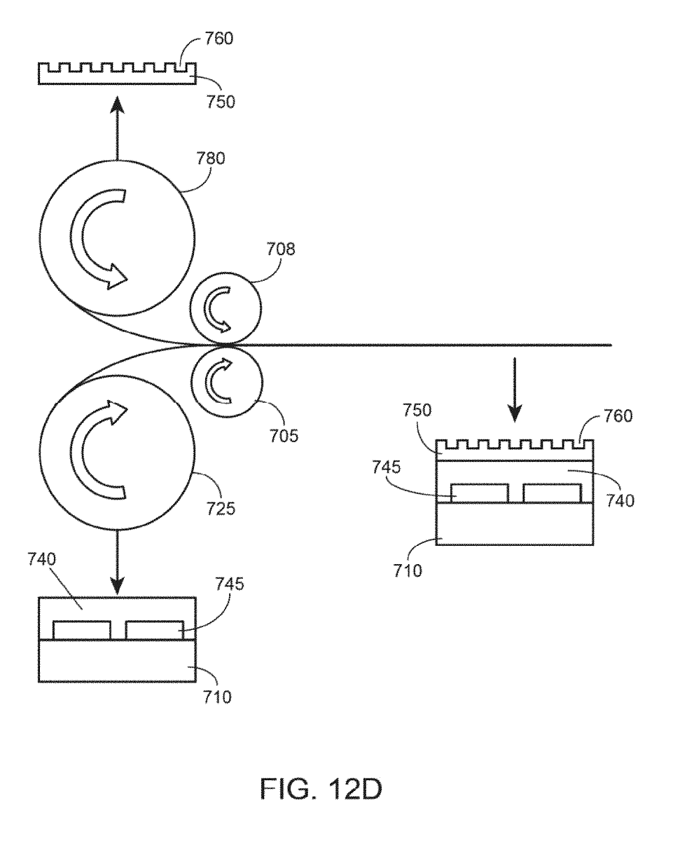

The well array may be fabricated onto the dielectric/hydrophobic layer, hydrophobic layer (if present), or hydrophilic layer (if present). One exemplary method for fabricating a well array onto the hydrophobic layer of the first substrate uses thermal or ultraviolet nanoimprint lithography. FIG. 12A illustrates one exemplary method for fabricating a well array by utilizing a flat nanoimprint mold 770 to apply sufficient pressure to the hydrophobic layer 750 at the second portion of the first substrate 710 in order to form the well array 760 pattern. In this example, the nanoimprint stamper may be a flat stamping element whose stamping contours correspond to the upper surface of the second layer.

FIG. 12B illustrates another exemplary method in which a nanoimprint roller 775 may be utilized to apply the pattern of well arrays to the hydrophobic layer of the second portion of the first substrate. The nanoimprint roller may imprint the pattern onto the hydrophobic layer 750 of the first substrate 710 by advancing the roller 775 in one direction. As the roller advances in the one direction, the roller leaves behind an imprint of a pattern of the well array 760 that corresponds to the imprint pattern on the roller. In one example, the roller 775 rolls in a counter clock-wise direction as the roller 775 imprints pattern onto the hydrophobic layer 750 of the first substrate 710. It is understood that the roller or stamper may be changed to form wells of suitable volume, for example, a femtolitre roller or stamper may be used for forming femtoliter wells.

FIG. 12C illustrates another exemplary method of forming a pattern of well arrays to the hydrophobic layer of the second portion of the first substrate. In this example, a laser may be applied to ablate the upper surface of the hydrophobic layer 750. The laser ablation step can produce a well array 760 pattern on the second layer. Some examples of suitable lasers for ablating the second layer include parameters with femtosecond and picosecond lasers. In some examples, the laser ablation step includes use of a special mask to define the well array pattern required. In some examples, the laser 775 utilizes a focusing element 777 (e.g., lens) to accurately target and ablate the pattern. In some examples, following the laser ablation step, the well array may be coated with a dielectric and/or hydrophobic layer.

FIG. 12D illustrates yet another example of forming a pattern of well arrays 760 onto the dielectric layer 740 of the second portion of the first substrate 710. As illustrated in FIG. 12D, the method utilizes roll-to-roll fabrication to separately fabricate microfluidic component and the well array. In one example, a first roll 725 contains a microfluidic component, which includes the first substrate 710, where the first substrate comprises a series or plurality of electrodes 745, and a dielectric layer 740 disposed over the upper surface of the first substrate and covering the series or plurality of electrodes 745. A second roll 780 contains a substrate 750 with the pattern of well array 760 already included on the substrate. In some examples, the pattern of well array 760 previously included on the substrate 750 can be applied through thermal or UV nanoimprint lithography. In other examples, the pattern of well array can be previously included on the substrate through laser ablation. As illustrated in FIG. 12D, the imprinted second roll 780 may also include a hydrophobic coating imprinted onto the substrate of the well array. The separate rolls are unwound via rollers 705 and 708, and then subject to a lamination process where the two films may be laminated together by overlying the well substrate over the microfluidic component substrate to form a stacked configuration of the well array and microfluidic components.

As described herein, “roll-to-roll” may include the equivalent term “reel-to-reel” and operates by moving a substrate through various components at high speeds, including, for example, rates of meters per second. Roll-to-roll assemblies facilitate the unwinding of a rolled substrate, the advancement of the substrate through the components, and the rewinding of the processed substrate into a roll.

As previously noted, the detection module formed by the second portions of the first and second substrates is used for detecting an analyte related signal. In some examples, detection of the analyte or biological sample of interest may occur through optical signal detection. For example, shining an excitation light (e.g., laser) in order to measure the signal intensity result. In other examples, the analyte desired may be detected by measuring an optical signal emanating from each well chamber and quantified by quantifying the result. For example, the number of positive counts (e.g., wells) is compared to the number of negative counts (e.g., wells) via digital analysis. A variety of signals from the wells of the device may be detected. Exemplary signals include fluorescence, chemiluminescence, colorimetric, turbidimetric, etc.

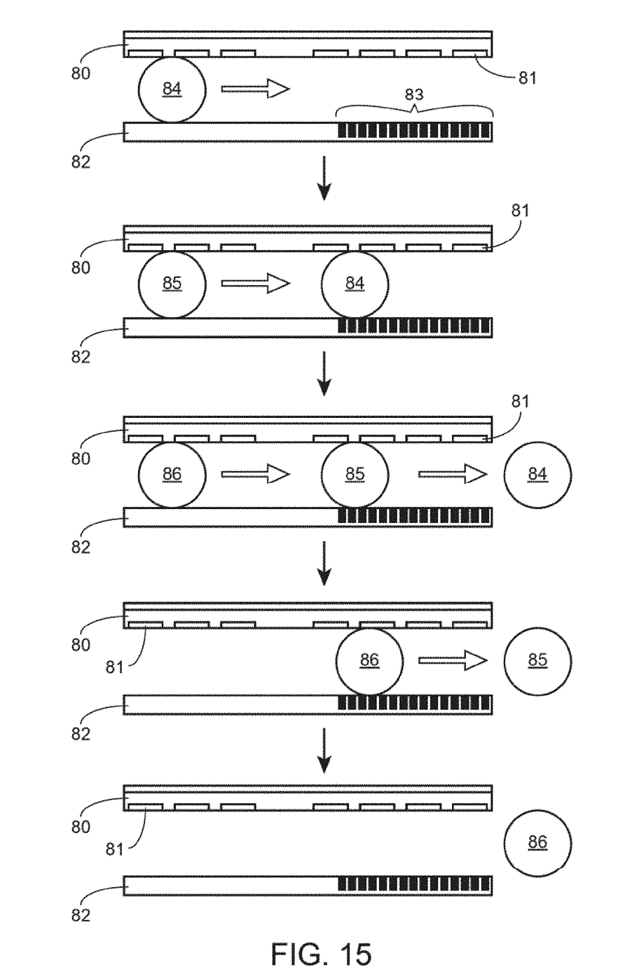

The devices described herein may be used to generate an analyte related signal and quantitate the signal. Exemplary method is depicted in FIG. 15. The device in FIG. 15 includes a top substrate 80 with an array of electrodes 81. The top substrate is positioned in a spaced apart manner from the bottom substrate 82 which includes an array of wells 83 in a second portion of the device. A droplet 84 containing particles or beads or analyte molecules (not shown) may be moved to the array of wells 83 using the electrodes 81. After a sufficient period of time to allow the particles or beads or analyte molecules to move into the wells, the droplet 84 may be moved to a waste chamber/absorption pad and the like. A droplet of buffer 85 may then be moved to the array of wells to remove any particles or beads not deposited into the wells. In some cases, the buffer droplet may push the droplet 84 over to the waste chamber. A droplet of immiscible fluid 86 may be moved over the array of wells and seal the wells. Any excess droplet 86 may be removed prior to optically interrogating the wells.

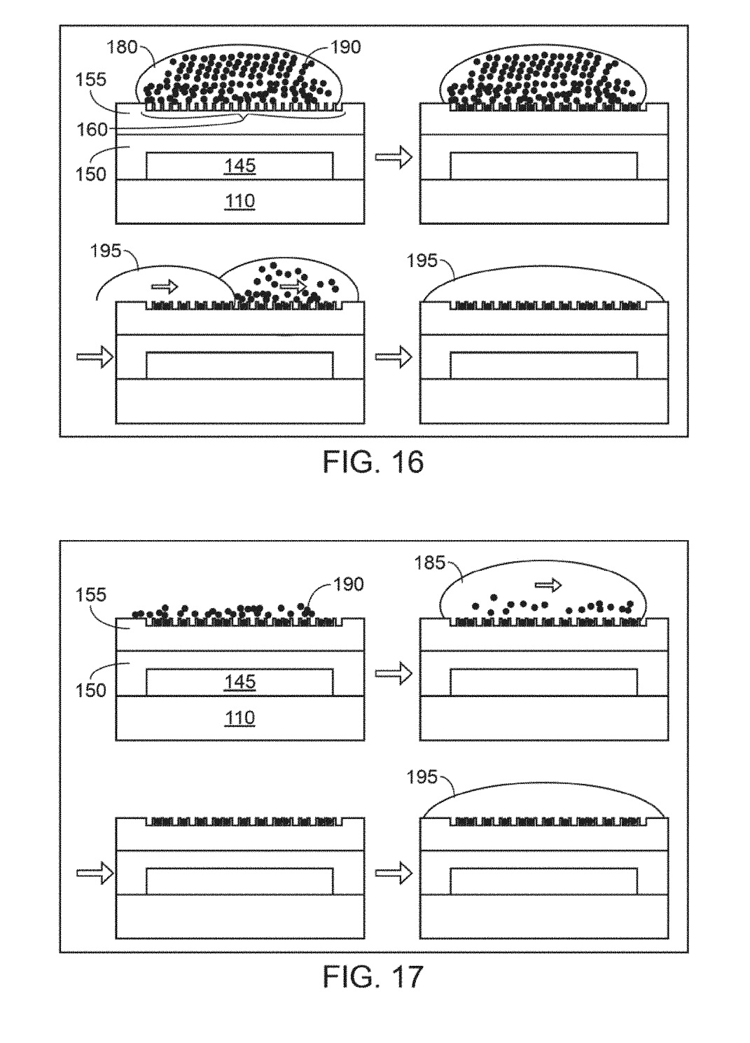

FIG. 16 depicts a method in which the digital microfluidics electrodes (e.g. electrode 145) position the droplet 180 containing particles/beads or analyte molecules 190 over the array of wells 160. After a period of time sufficient for deposition of particles/beads/analyte molecules into the wells, the droplet is displaced by a droplet of immiscible liquid 195 (or an immiscible liquid as explained herein). The droplet of immiscible liquid functions to move droplet 180 with any bead/particles/analyte molecules not deposited into the wells away from the wells and to cover the wells.

FIG. 17 depicts another method for removing any beads not deposited into wells. In FIG. 17, many beads 190 are remaining over the wells after removal of the droplet containing the beads. These beads are washed away using an aqueous droplet. 185 After removal of the aqueous droplet, the array of wells contains the deposited beads. An immiscible fluid 195 is then moved over the array of wells to seal the wells.

A number of forces may be utilized to facilitate the movement of particles/beads from a droplet positioned over the array of wells into the wells. Such forces include gravity, electrical force, magnetic force, etc. Permanent magnets or electromagnets may be used as source of magnetic force. In certain embodiments, the magnets are not located on the integrated microfluidic and detection chip. Analyte molecules may be deposited into the wells via diffusion.

According to other aspects of the disclosed subject matter, the present disclosure describes a microfluidics device used in conjunction with a nanopore device and an integrated microfluidics nanopore device. The disclosed microfluidics device used in conjunction with a nanopore device and an integrated microfluidics nanopore device may be used in the method of analyte analysis, as described above. However, in certain cases, the devices described herein may be used for other applications. Likewise, in certain cases, the methods described herein may be used with other devices.

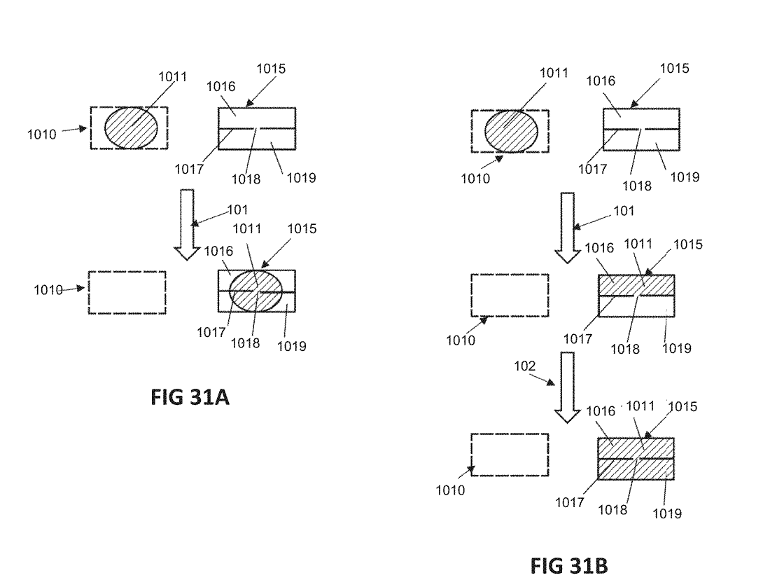

A microfluidics device used in conjunction with a nanopore device is depicted in FIGS. 31A and 31B. The microfluidics device 1010 is depicted with a fluid droplet 1011 which is to be analyzed in the nanopore device 1015. The fluid droplet may include a tag (e.g., a cleaved tag or an aptamer) that is to be counted using the nanopore device. The nanopore device 1015 includes a first chamber 1016, a layer 1017 with a nanopore 1018, and a second chamber 1019. FIGS. 31A and 31B depict a liquid transfer step 101 in which the fluid droplet 1011 is removed from the microfluidics device 1010 and placed into the nanopore device 1015. As depicted in FIG. 31A, the fluid droplet 1011 is deposited over the layer 1017 in a manner that results in the droplet being split apart across the layer 1017 and positioned at the nanopore 1018. The fluid droplet may be introduced into the nanopore device 1015 via an entry port (not shown). The entry port may be positioned over a section of the layer 1017. For example, the entry port may be located in an opening in a wall of a chamber in which the layer containing nanopore is positioned. In FIG. 31B, the liquid droplet 1011 is deposited in the first chamber 1016. A buffer addition step 102, introduces a buffer in the second chamber 1019. In other embodiments, buffer may be added to the second chamber 1019 prior to the introduction of the liquid droplet 1011 into the first chamber 1016. In yet other embodiments, the liquid droplet 1011 may be deposited in the second chamber 1019 before or after buffer is added to the first chamber 1016. In FIG. 31A, a step of addition of a buffer to either chamber is not needed.

In another embodiment, the device may be an integrated device. The integrated device may include a microfluidics module and a nanopore module that may be built separately and then combined to form the integrated device or the microfluidics module and the nanopore module may be built-in together in a single device.

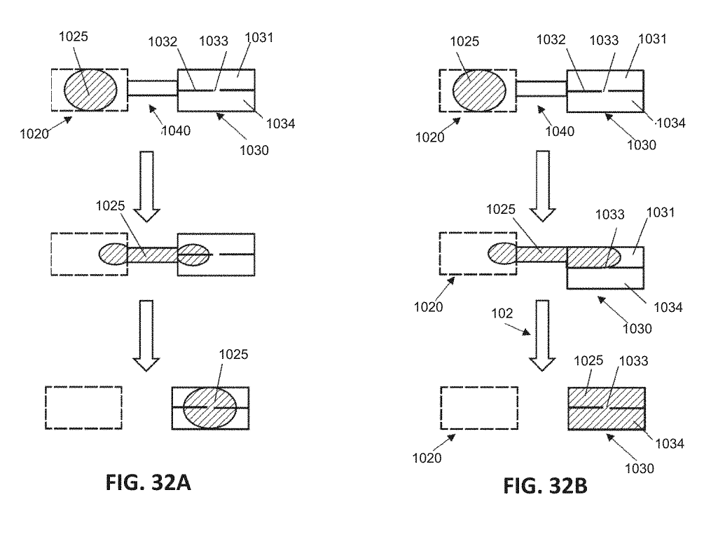

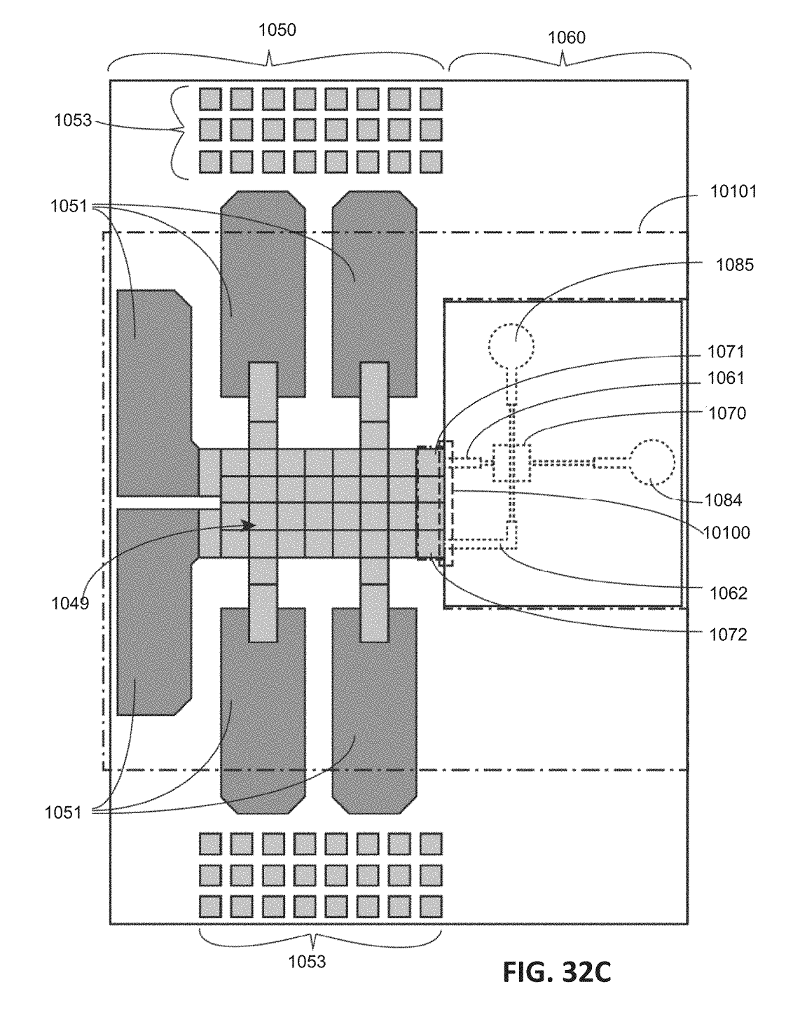

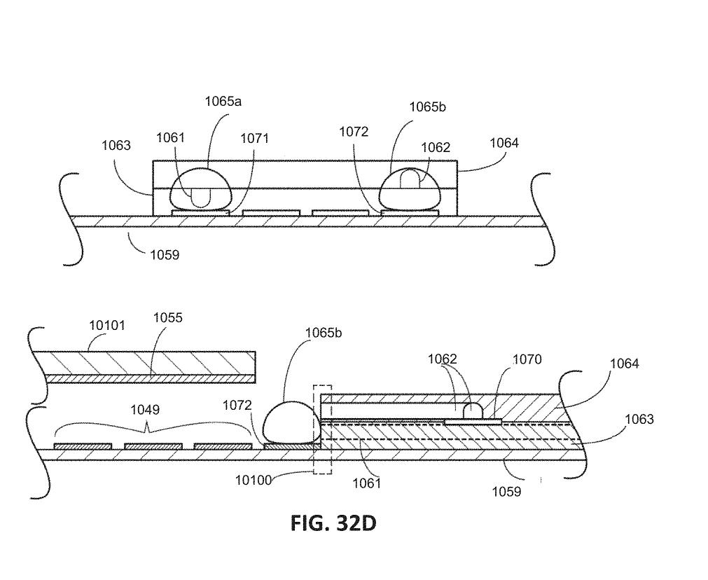

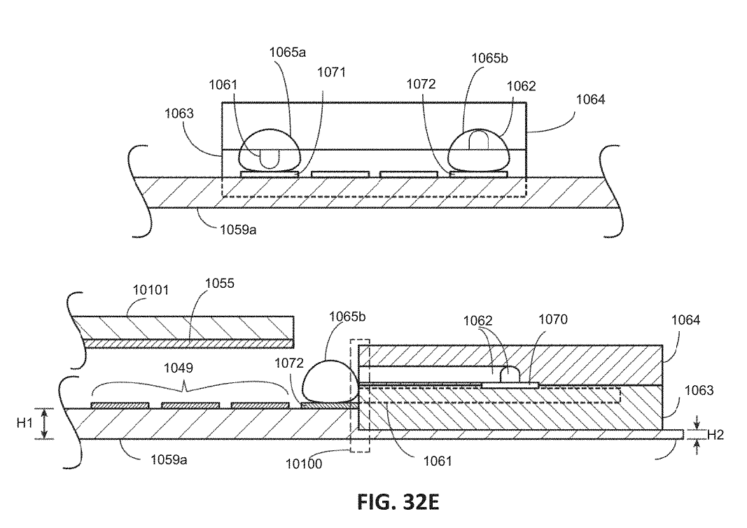

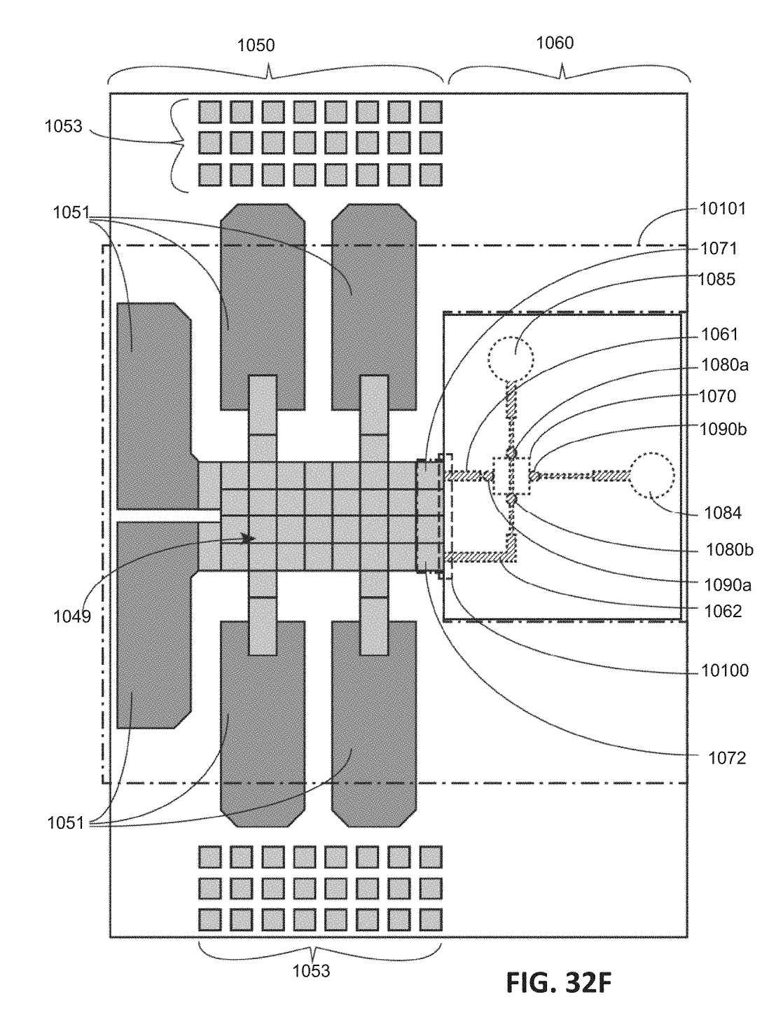

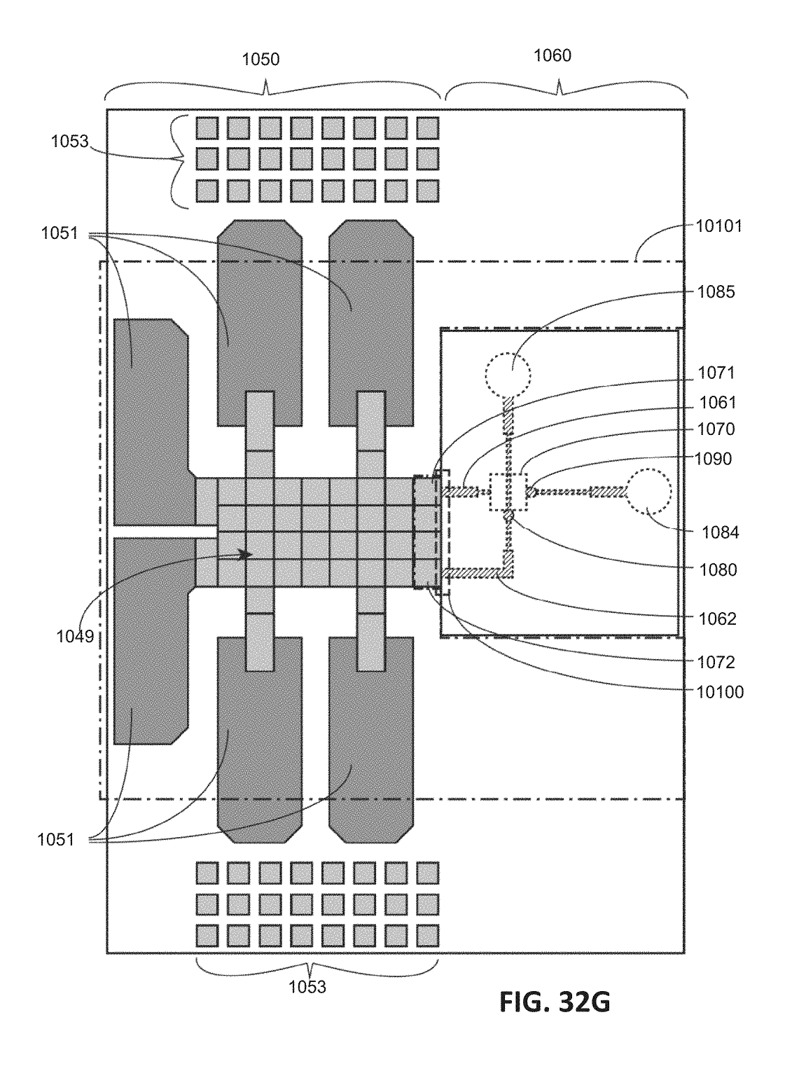

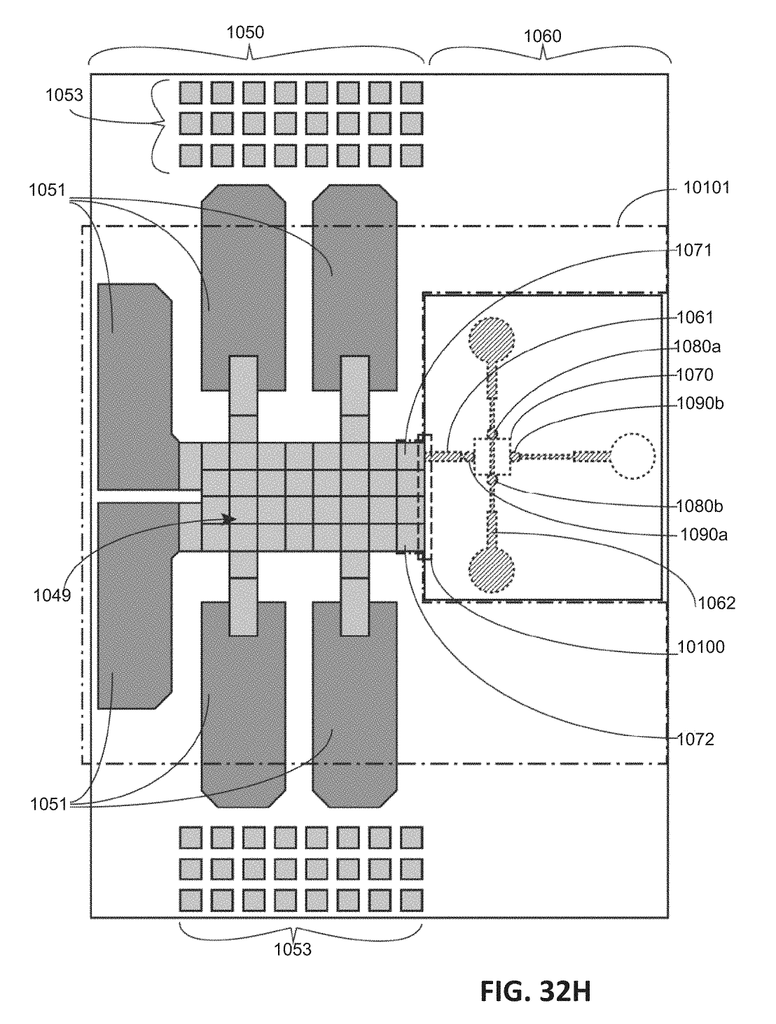

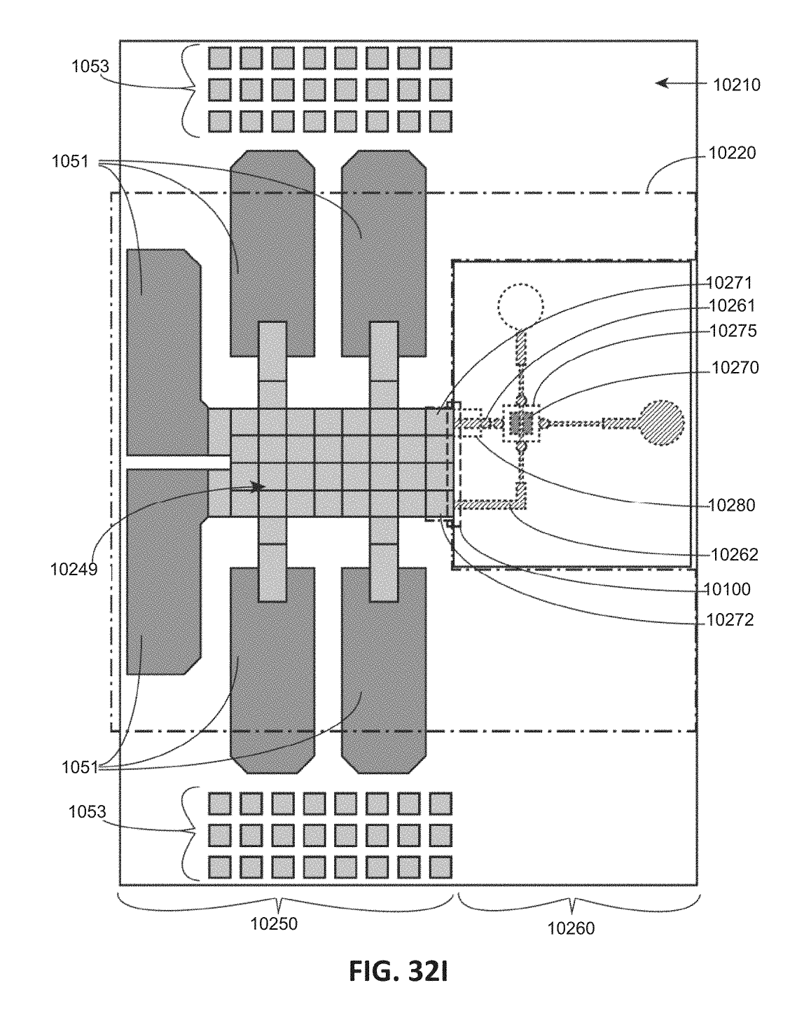

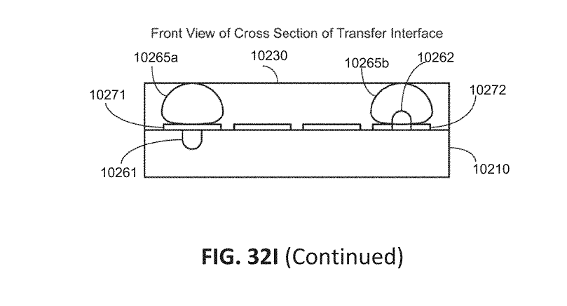

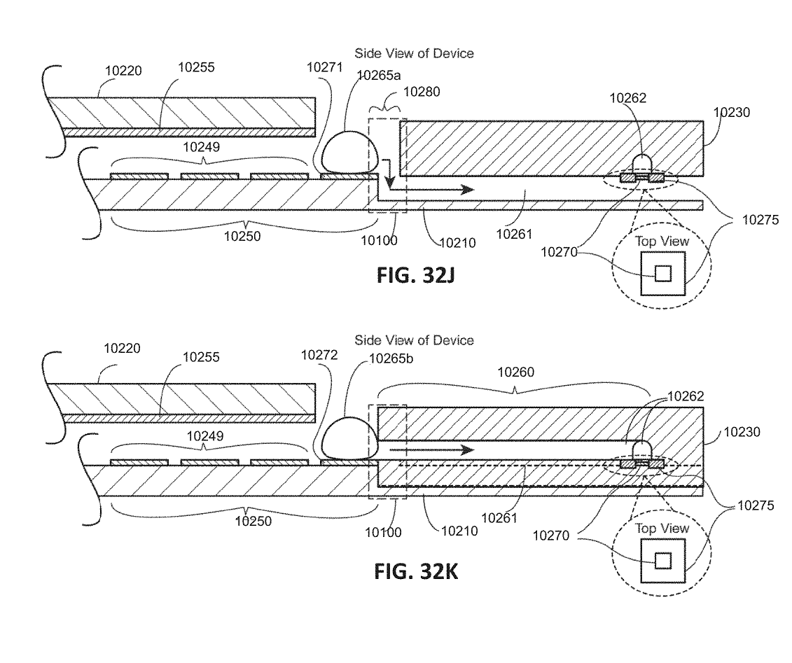

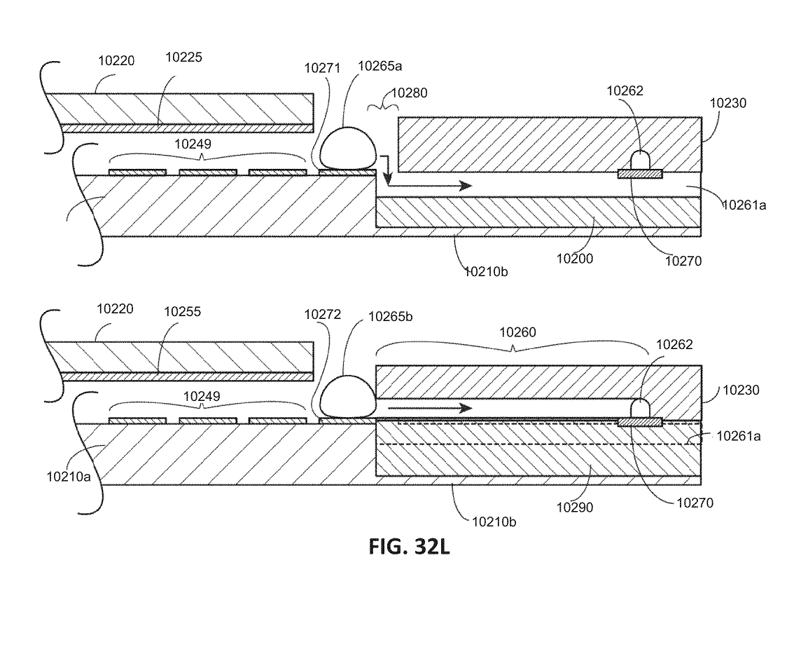

FIGS. 32A and 32B depict a schematic of an integrated device that has a microfluidics module combined with a nanopore module and the two modules are integrated by connecting them using a channel. Although FIGS. 32A and 32B depict a device that includes individual modules that are combined to generate an integrated device, it is understood that the device of FIGS. 32A and 32B can also be manufactured as a unitary device in which the two modules are connected.

In FIGS. 32A and 32B, top panel, a microfluidics module 1020 is depicted with a fluid droplet 1025 which is to be analyzed in the nanopore device 1030. The nanopore module 1030 includes a first chamber 1031, a layer 1032 with a nanopore 1033, and a second chamber 1034. The microfluidics module 1020 is integrated with the nanopore module 1030 via a channel 1040. The channel fluidically connects the two modules and facilitates the movement of the droplet 1025 from the microfluidics module 1020 to the nanopore module 1030. The middle panel illustrates the movement of the droplet 1025 from the microfluidics module 1020 to the nanopore module 1030 via the channel 1040. As shown in FIG. 32A, the channel may connect the microfluidics module 1020 to an entry port in the nanopore module 1030. The entry port (not shown) may be positioned such that the fluid droplet 1025 is deposited over the layer 1032 in a manner that results in the droplet being split apart across the layer 1032 and positioned at the nanopore 1033. At the end of the transfer process, the fluid droplet is positioned across the nanopore 1033 (FIG. 32A, bottom panel). In other embodiments, the channel 1040 may connect the microfluidics module 1020 to an entry port in a first or second chamber of the nanopore module 1030. Such an embodiment is shown in FIG. 32B, where the channel 1040 connects the microfluidics module 1020 to an entry port in a first chamber 1031 of the nanopore module 1030. Following or prior to the transfer of the liquid droplet 1025 into the first chamber 1031, a buffer may be added to the second chamber. In step 102 of FIG. 32B, buffer is added to the second chamber 1034 following the transfer of the droplet 1025 to the first chamber 1031. Optionally, after the transfer is completed, the channel 1040 may be removed and the two modules separated. The microfluidics and nanopore devices and modules shown in FIGS. 31A, 31B, 32A and 32B, respectively, are each individually functional.

FIGS. 32C-32H depicts an embodiment of an integrated device which includes a digital microfluidics module 1050 and a nanopore module 1060. The digital microfluidics module is depicted with an array of electrodes 1049 that are operatively connected to a plurality of reagent reservoirs 1051 used for generation of droplets to be transported to the nanopore module. One or more of the reservoirs 1051 may contain a reagent or a sample. Different reagents may be present in different reservoirs. Also depicted in the microfluidics module 1050 are contact pads 1053 that connect the array of electrodes 1049 to a power source (not shown). Trace lines connecting the array of electrodes 1049 to the contact pads are not depicted. The array of electrodes 1049 transport one or more droplets (such as buffer droplet or a droplet containing buffer and/or tag (e.g., cleaved tag or dissociated aptamer)) to one or both of the transfer electrodes 1071 and 1072 located at the interface 10100 between the digital microfluidics module 1050 and a nanopore module 1060. The digital microfluidics module 1050 and the nanopore module 1060 are operatively connected at the interface 10100. The nanopore module 1060 includes at least two microfluidic capillary channels 1061 and 1062 that intersect with each other at the location at which a nanopore layer 1070 is disposed. The two microfluidic capillary channels 1061 and 1062 are located in two different substrates in the nanopore module (depicted in FIG. 32D). Thus, the nanopore module includes a first substrate 1063 (e.g., bottom substrate) that includes a microfluidic capillary channel 1061 in a top surface of the first substrate 1063 and further includes a second substrate 1064 (e.g., top substrate) with a microfluidic capillary channel 1062 in the first surface of the second substrate. The second substrate 1064 overlays the microfluidic capillary channel 1061 and the first substrate 1063 underlays the microfluidic capillary channel 1062. The capillary channel 1062 overlays capillary channel 1061 at the point of intersection of the two channels at the location of the nanopore layer 1070 (see also FIG. 32D, bottom panel). The two capillary channels are physically separated at the intersection by the nanopore layer 1070 placed at the intersection. The nanopore layer 1070 includes at least one nanopore (not shown) that is positioned at the intersection of the capillary channels and allows transport of molecules from one capillary channel to the other through the nanopore. The capillary channels 1061 and 1062 open at the interface 10100 at a first ends of the capillary channels and open to a reservoir/vent (1084 and 1085, as seen in FIG. 32C) at the second ends of the capillary channels. Also depicted in FIG. 32C is a cover substrate 10101 that is positioned over the array of electrodes 1049. The cover substrate 10101 defines a gap in the microfluidics module in which droplets are manipulated. The cover substrate 10101 may optionally include an electrode 1055 (e.g., a reference electrode) disposed on a bottom surface of the cover substrate 10101 providing a bi-planar electrode configuration for manipulating droplets in the microfluidics module 1050. In absence of a bi-planar electrode configuration, droplets may be manipulated in the microfluidics module 1050 by using coplanar electrode actuation, for example using the array of electrode 1049 or another coplanar electrode configuration. For example, the coplanar electrodes described in U.S. Pat. No. 6,911,132 may be used for manipulating droplets in the microfluidics module 1050.

FIG. 32D, top panel, shows a schematic of a front view of a cross-section of the interface 10100 at which the digital microfluidics module 1050 and a nanopore module 1060 are operatively connected. A schematic of a side view of a cross-section of the device at the transfer electrode 1072 is depicted in the bottom panel of FIG. 32D. FIG. 32D, top panel shows two droplets (1065a and 1065b) positioned on two transfer electrodes 1071 and 1072 that are located at the interface 10100 between microfluidics module 1050 and a nanopore module 1060. As illustrated in FIG. 32D, top panel, the droplet 1065a positioned at electrode 1071 is aligned with the opening in the capillary channel 1061 while the droplet 1065b positioned at electrode 1072 is aligned with the opening in capillary channel 1062. FIG. 32D, bottom panel illustrates a side view of a cross-section of the integrated device showing placement of droplet 1065b on transfer electrode 1072. The droplet 1065b is positioned to move into the capillary channel 1062. Capillary channel 1061 is also shown; however, the capillary channel is at a distance from the transfer electrode 1072 and is aligned with transfer electrode 1071 (not shown). The cover substrate 10101 with an electrode 1055 disposed on the bottom surface of the cover substrate 10101 is also depicted. In the embodiments of the integrated devices depicted in FIG. 32D-32H, the nanopore module is disposed on the same substrate as the electrode array of the microfluidics module.

The vertical distance between the top surface of the transfer electrodes and the entrance to the capillary channels may be determined by the thickness of the substrates forming the lower part of the microfluidics module and the nanopore module. The vertical distance may be set based on the volume of the droplets to be transferred to the nanopore module. The vertical distance may be adjusted by varying the thickness of the substrates. For example, the substrates (e.g., substrate 1063) of the nanopore module may kept relatively thin or the thickness of the substrate on which the transfer electrodes are disposed can be increased (for example by using a thicker substrate) to ensure that the droplet is aligned with the entrance of the capillary channel. An exemplary device in which the droplets are brought into alignment with the entrance to the capillary channels by using a microfluidics module having a thicker bottom substrate is depicted in FIG. 32E. The device shown in FIG. 32E has the same configuration as described for FIGS. 32C-32D. However, the thickness of the substrate 1059a on which the electrode array is positioned is increased relative to the thickness of the part of the substrate on which the nanopore module is disposed. FIG. 32E, top panel depicts a front view of a cross section at the interface 10100 between the microfluidics module and the nanopore module. FIG. 32E, bottom panel depicts a side view of a cross section at the position of the transfer electrode 1072 and capillary channel 1062. As illustrated in FIG. 32E, the substrate 1059a on which the electrode array 1049 and the transfer electrodes 1071 and 1072 are disposed is thicker than the substrate 1059b on which the nanopore module is disposed. As shown in FIG. 32E, bottom panel, substrate 1059a has a first height H1 while substrate 1059b has a second height H2, where H1 is greater than H2. The difference in height between the substrates 1059a and 1059b results in alignment of the capillary channels 1061 and 1062 in the nanopore module with the droplets positioned on electrodes 1071 and 1072, respectively. Also depicted in the bottom panel of FIG. 32E is the channel 1061. As evident from FIG. 32C, capillary channel 1062 is perpendicular to the capillary channel 1061 at the location of the nanopore layer 1070. Channel 1061 is aligned with the transfer electrode 1071 and is configured to receive droplet 1065a positioned on transfer electrode 1071. While the two capillary channels are depicted to be perpendicular to each other at the point of intersection, other configurations are also envisioned where the two channels intersect at an angle other than 90 degrees.

Upon contact with the capillary channel, the droplets move into the capillary channel via any suitable means, such as, capillary action. The movement of a droplet into the capillary channel may be facilitated by additional methods/materials. For example, the droplets may move into the capillary channel via diffusion, Brownian motion, convection, pumping, applied pressure, gravity-driven flow, density gradients, temperature gradients, chemical gradients, pressure gradients (positive or negative), pneumatic pressure, gas-producing chemical reactions, centrifugal flow, capillary pressure, wicking, electric field-mediated, electrode-mediated, electrophoresis, dielectrophoresis, magnetophoresis, magnetic fields, magnetically driven flow, optical force, chemotaxis, phototaxis, surface tension gradient driven flow, Marangoni stresses, thermo-capillary convection, surface energy gradients, acoustophoresis, surface acoustic waves, electroosmotic flow, thermophoresis, electrowetting, opto-electrowetting, or combinations thereof. In addition or alternatively, movement of a droplet into the capillary channel may be facilitated by using for example, an actuation force, such as those disclosed herein; using hydrophilic coating in the capillary; varying size (e.g. width and/or height and/or diameter and/or length) of the capillary channel).

In the embodiments depicted in FIGS. 32C-32H, the flow of a fluid across capillaries channels 1061 and 1062 is controlled at least in part by changing the cross-section of the capillaries—the fluid initially moves relatively quickly till it enters a narrower portion of the capillaries. One or both droplets may be droplets containing analyte to be detected or counted (or cleaved tag or dissociated aptamer) or conductive solution (e.g., buffer not containing an analyte) for analysis via the nanopore. In certain cases, one droplet 1065a may be a droplet containing an analyte/tag/aptamer while the other droplet 1065b may be a buffer droplet. While a single droplet is depicted for each channel, in practice, multiple droplets may be transported to the nanopore module. For example, the multiple droplets may be transported to the nanopore module in a sequential manner. In some cases, multiple droplets may be gathered at one or both transfer electrodes to generate a larger droplet which is transported to the nanopore module.

FIG. 32F illustrates an exemplary configuration of the various electrodes used in the integrated device. As noted above, a single continuous electrode 1055 (not shown in FIG. 32F) is positioned in a spaced apart manner from the array of electrodes 1049 in the microfluidics module 1060. The array of electrodes includes a series of individually controllable electrodes. The electrode 1055 is disposed on a lower surface of the cover substrate 10101. Electrode 1055 and the array of electrodes move the droplets over to the transfer electrodes. While it is depicted that electrode 1055 does not cover the transfer electrodes 1071 and 1072, in certain exemplary devices, the cover substrate 10101 and the electrode 1055 may extend over the transfer electrodes. In embodiments where the electrode 1055 does not cover the transfer electrodes, co-planar electrodes may be used to move droplets to the transfer electrode (e.g., coplanar actuation as described in U.S. Pat. No. 6,911,132). As described herein, the single electrode 1055 may serve as a reference or a grounding electrode, while the array of electrodes 1049 may be individually controllable (for example, the array of electrodes may be actuation electrodes that can be actuated independently). Electrode pairs: pair 1080a and 1080b and pair 1090a and 1090b are positioned in the nanopore module. Electrode pairs 1080a, 1080b and 1090a, 1090b are used to establish opposite polarity across the nanopore layer 1070 for driving charged molecules through the nanopore(s) in the nanopore layer 1070. In some embodiments, the electrode pair 1080a and 1080b may be positive electrodes and the electrode pair 1090a and 1090b may be negative electrodes. FIG. 32G illustrates an alternative electrode configuration for the nanopore module where two electrodes 1080 and 1090 (instead of four) are used for establishing a polarity difference across the nanopore layer 1070. These examples demonstrate the use of either symmetrical (four electrodes) or asymmetrical (two electrodes) electrode configurations that generate an electric potential gradient across the nanopore layer for translocating charged molecules through the nanopore.

FIG. 32H illustrates an alternative configuration of the capillary channels where only one channel 1061 is connected to the microfluidics module at the interface 10100. The other channel 1062 is connected to two reservoirs that may be filled with a conductive liquid to facilitate transfer of charged molecules across the nanopore.

In certain cases, the integrated devices provided herein may be fabricated by forming reservoirs and array of electrodes for the digital microfluidics module portion on a first area of a top surface of a first substrate. A second substrate may be prepared by disposing a single electrode (e.g., electrode 1055) on the bottom surface of the second substrate and positioned over the array of individually controllable electrodes in a spaced apart manner to provide facing orientation between the single electrode and the array of electrodes for bi-planar droplet actuation. As used herein, “droplet actuation” refers to manipulation of droplets using a microfluidics device as disclosed herein or using a droplet actuator as disclosed in U.S. Pat. No. 6,911,132, U.S. Pat. No. 6,773,566, or U.S. Pat. No. 6,565,727, the disclosures of which are incorporated herein by reference. Thus, the configuration of the bi-planar electrodes or the array of electrodes of the devices disclosed herein may be similar to those disclosed in U.S. Pat. No. 6,911,132, U.S. Pat. No. 6,773,566, or U.S. Pat. No. 6,565,727. The electrode 1055 on the second substrate may also be referred to as a reference electrode. The electrodes in the microfluidics module may optionally be coated with a dielectric material. A hydrophobic coating may also be provided on the dielectric.

In certain embodiments, a microchannel may be formed on a third substrate which may be disposed on a second area of the first substrate on which the array of electrodes 1049 is disposed. For example, a third substrate may be bonded onto a second area on the first substrate in which the microfluidics electrode array is disposed in the first area. The substrate may have a pre-formed microchannel or a microchannel may be formed after the bonding step. A fourth substrate with a second microchannel may be disposed on top of the substrate containing the microchannel to provide an integrated device as depicted in FIG. 32C-32H. The nanopore layer may be disposed on either microchannel at the location of the intersection of the two microchannels. Thus, the substrates forming the nanopore module may include microchannels that are open at either ends and on one side. The placement of the fourth substrate over the third substrates closes the microchannels thereby forming capillary channels (e.g., 1061 and 1062).

In certain embodiments, a microchannel may be formed on a separate substrate which may be disposed on to the first substrate on which the microfluidics array of electrodes is disposed. For example, another substrate may be bonded onto the second area on the first substrate in which the microfluidics electrode array is disposed in the first area. The substrate may have a pre-formed microchannel or a microchannel may be formed after the bonding step. Another substrate with a second microchannel may be disposed on top of the substrate containing the microchannel to provide an integrated device as depicted in FIG. 32C-32H. The nanopore layer may be disposed on either microchannel at the location of intersection of the two microchannels 1061 and 1062.

In some embodiments, a microchannel may be introduced in the second area adjacent the first area on the first substrate on which the microfluidics array of electrodes is disposed. For example, the microchannel may be etched on the top surface in the second area. A nanopore layer may be placed at a location on the microchannel. The nanopore layer may include preformed nanopore(s). In alternative embodiments, nanopore(s) may be formed after positioning the layer at a location on the microchannel. A third substrate may be prepared by introducing a microchannel on a bottom surface of third substrate. The third substrate may be positioned over the second area on the first substrate such that the top surface of the second area of the first substrate is in contact across its top surface with the bottom surface of the third substrate thereby creating closed capillary channels 1061 and 1062.

FIGS. 32I-32K depict devices in which the digital microfluidics module 10250 and nanopore module 10260 share a common bottom (first) substrate 10210 on which the array of electrodes 10249 (a series of individually controllable electrodes) for the microfluidics module is disposed on a first area and a microfluidic channel 10261 is formed in a second area. The microfluidic channel 10261 in the first substrate is aligned with the transfer electrode 10271. A second substrate 10220 having a single continuous electrode 10255 (e.g., a reference electrode) is disposed in a spaced apart manner from the array of electrodes 10249 in the digital microfluidics module 10250. A third substrate 10230 comprising a microfluidic channel 10262 formed in a lower surface of the third substrate is placed over the second area of the first surface 10210 thereby covering the top surface of the first substrate in which the microfluidic channel 10261 is formed. The first substrate and the third substrate in the nanopore module enclose the microfluidic channels 10261 and 10262 thereby providing capillary channels 10261 and 10262. It is understood that “microfluidic channel(s)” and “microchannel(s)” are used herein interchangeably to refer to a passage or a cut out in a surface of a substrate. Upon placement of a substrate over the passage, the passage is enclosed forming a capillary channel. Similar to the FIG. 32C, the capillary channels may be fluidically connected to the microfluidics module at one end at the interface 10100 between the microfluidics module 10250 and the nanopore module 10260 and with a reservoir or vent on the other end. In other embodiments, the second capillary channel 10262 may be configured similarly to the capillary channel 1062 in FIG. 32H, i.e., the second capillary channel 10262 may not be connected to the microfluidics module at either end and may be connected to a reservoir/vent at both ends. A top view of the device is depicted in FIG. 32I and a front view of a cross-section of the device at the interface between the modules is depicted in FIG. 32I (continued). As is evident from the front view, the droplet 10265a is on a plane higher than the entrance to the capillary channel 10261. In order to allow the droplet 10265a to flow into the capillary channel 10261, a notch 10280 is created in a side edge of the third substrate 10230 to provide space for movement of the droplet down into the microchannel 10261. Thus, the fluidic connection between the microfluidics module and the nanopore module is provided by a vertical port formed by the notch 10280 providing an opening in a top part of the first capillary channel 10261 at one end of the first capillary channel 10261 at the interface 10100. It is understood that the notch 10280 is FIG. 32I is not drawn to size and may be of any suitable size that allows for fluid communication between the transfer electrode 10271 and the first capillary channel 10261 at the interface 10100. Further, the notch may be varied in size. For example, the notch may be a cut-out that extends along a length of the side edge of the third substrate 10230 at interface 10100 and may be proportioned to match the width of the transfer electrode 10271 or the width of the capillary channel 10261 or a length in between. The cut-out may be extended nominally along the width of third substrate 10230 such that a relatively minor region of the capillary channel 10261 is uncovered. In other embodiments, the cut-out may extend over a substantial length of the capillary channel 10261. A layer 10270 containing a nanopore is positioned across the first capillary channel 10261 at the position at which the two capillary channels intersect. The layer 10270 is positioned in a support substrate 10275. In certain cases, the first substrate 10210 may be a glass substrate and the support substrate 10275 may be a PDMS gasket.

A side view of a cross-section of the device shown in FIG. 32I is depicted in FIG. 32J. The cross-section is at the region of the device where the first capillary channel 10261 is aligned with the first transfer electrode 10271. Also depicted is a portion of the microfluidics module 10250 with the array of electrodes 10249, the second substrate 10220 with a single electrode 10255 (e.g., reference electrode) positioned in a spaced apart manner from the array of electrodes 10249. As shown in FIG. 32J, the single electrode 10255 does not cover the transfer electrodes. While not illustrated in these Figures, the second substrate 10220 and the single electrode 10255 (which may be a reference electrode) may cover the transfer electrodes 10271 and 10272, providing a bi-planar electrode configuration. In this embodiment, droplets can be moved to the transfer electrodes 10271 and 10272 using the bi-planar electrodes. The first capillary 10261 is located in the first substrate 10210 and is located in a plane lower than the plane on which the droplet 10265a is present. The third substrate 10230 which includes the second microchannel (which is enclosed by the top surface of first substrate 10210 to provide the capillary channel 10262) is disposed over the first substrate. The third substrate 10230 includes the notch 10280 (or cut out) at the side edge adjacent to the microfluidics module at the interface 10100. The notch 10280 opens the capillary 10261 on a top portion at the end of the capillary channel 10261 providing a vertical port for entrance to the capillary channel 10261. As shown by the direction of the arrow, the droplet travels down to the capillary 10261 and then proceeds to flow towards the intersection of the first and the second capillary channels. The second capillary channel 10262 intersects with the first capillary 10261 at the location of the nanopore layer 10270. A support substrate 10275 positioned over the first capillary channel 10261 (and under the second capillary channel 10262) is depicted. The support substrate 10275 includes the nanopore layer 10270. As shown in a top view of the nanopore layer is shown in the inset, the support substrate 10275 surrounds the nanopore layer. In some embodiments, the support substrate may be a first layer with a cut out in the center and a second layer with a cut out in the center. The nanopore layer may be disposed at the cut out in between the first and the second layers. A nanopore layer in a support substrate may be used in devices where the bottom substrate 10210 is made of glass.

FIG. 32K shows an additional side view of a cross-section of the device shown in FIG. 32I. In FIG. 32J, the cross section is at the location of the first transfer electrode 10271. In FIG. 32K, the cross section is at the location of the second transfer electrode 10272. As shown in FIG. 32K, the entrance to the second capillary channel 10262 is aligned with the position of the droplet 10265b present on the second transfer electrode 10272. Also depicted in FIG. 32K is the first capillary channel 10261 which intersects with the second capillary channel 10262 at the location of the nanopore layer 10270.

In another embodiment, as shown in FIG. 32L, the first substrate 10210 may be include a first portion 10210a on which the array of electrodes 10249 and transfer electrodes 10271 and 10272 are disposed and a second portion 10210b on which a substrate 10290 containing capillary channel 10261a is disposed. Similar to the device shown in FIG. 32I-32K, the capillary channel 10261a is below the plane on which the transfer electrodes are located. Capillary channel 10262 is located in substrate 10230 where the entrance to the capillary channel 10262 is at the same plane as the transfer electrodes in the microfluidics module 10250. Further, similar to FIGS. 32I-32K, entrance to the capillary channel 10262 is aligned with the transfer electrode 10272. Thus, a droplet positioned on electrode 10272 can travel substantially horizontally to the capillary channel 10262. Similar to the device shown in FIGS. 32I-32K, the substrate 10230 includes a notch 10280 in a side edge of substrate 10230 to provide space for a droplet positioned on transfer electrode 10271 to travel down to capillary 10261a which is located in substrate 10290. Also depicted in FIG. 32L is the nanopore layer 10270. In this embodiment, the nanopore layer is directly disposed on the substrate 10290 in absence of the support layer 10275. For example, in embodiments where both substrates containing the channels are formed from PDMS, the nanopore layer may be directly disposed in between the substrates in absence of a support substrate. FIG. 32L, top panel depicts a side view of a cross section through the device at the location at which the transfer electrode 10271 and capillary channel 10261a are located. FIG. 32L, bottom panel depicts a side view of a cross section through the device at the location at which the transfer electrode 10272 and the capillary channel 10262 are located. From the top, the device looks same as the device shown in FIG. 32I. Thus, the transfer electrodes 10271 and 10272 are spaced apart same as the transfer electrodes 1071 and 1072 in the device shown in FIG. 32I.

The electrodes in the nanopore module for the transport of molecules across the nanopore layer via nanopore(s) may be fabricated after positioning of the nanopore layer in the device. For example, the electrodes may be disposed in openings introduced into the substrates and positioned in the capillary channels such that they are exposed in the capillary channels and will be in contact with the fluid present in the capillary channels. The distance of the electrodes from the nanopore may be determined empirically based on resistance, width, diameter, and/or length of the capillary channel(s).

The nanopore layer may be disposed on either channel. The nanopore layer may be adhered to the surface of the substrate containing the microchannel by plasma bonding or via a compressible element, such as a gasket. In certain cases, the substrate containing the first channel may be a glass substrate. In this embodiment, a support substrate, such as, a PDMS layer may be used for positioning the nanopore layer. For example, the nanopore layer may be provided with a PDMS gasket.

Any suitable method may be employed to form the channels on the substrate. In certain cases, lithography or embossing may be used to create the channels for the nanopore module. In other embodiments, the channels may be etched into the substrates. In certain embodiments, a combination of suitable methods may be used to form channels in the substrates. For example, a channel may be formed in a glass substrate using an etching process and another channel may be formed in a PDMS substrate using an appropriate method, such as, soft lithography, nanoimprint lithography, laser ablation or embossing (e.g., soft embossing). The height/width/diameter of the microchannels may be determined empirically. The height/width/diameter of the microchannels may be in the range of 0.5 μm to about 50 μm, e.g., 0.5 μm-40 μm, 1 μm-30 μm, 2 μm-20 μm, 3 μm-10 μm, 5 μm-10 μm, such as, 0.5 μm, 1 μm, 2 μm, 3 μm, 4 μm, 5 μm, 6 μm, 7 μm, 8 μm, 9 μm, 10 μm, 15 μm, 20 μm, 30 μm, 40 μm, or 50 μm. As noted herein, the height/width/diameter of the channels may vary along the length of the channels.

In certain embodiments, the nanopore layer (e.g., 1070 or 10270) may include a coating of insulating material on one or both sides of the nanopore layer. The insulating material may reduce contact capacitance and decrease noise associated with detection of translocation of a molecule through the nanopore(s) in the nanopore layer. In another embodiment, the surface area of the nanopore layer exposed to a fluid in the capillary channels in fluid contact with the nanopore layer (e.g., capillary channels 1061 and 1062 or 10261 and 10262) may be reduced. Reducing the surface area of the nanopore layer that is in contact with a fluid containing the molecules to be detected or counted by the nanopore(s) may minimize contact capacitance and reduce background noise. The surface area of the nanopore layer in contact with a fluid in the capillary channels may be reduced by reducing the size of the capillary channel at the location of the nanopore layer. For example, the height or width or both (e.g., diameter) of the capillary channels at the location of the nanopore layer may be reduced. In another embodiment, the surface area of the nanopore layer may be reduced. In certain devices, a combination of these embodiments for reducing contact capacitance may be included. For example, in certain embodiments, an integrated device as disclosed herein may include capillary channels that have a decreased dimension at the location of the nanopore layer and/or may include a nanopore layer that is coated with an insulating material (e.g., PDMS) on one or both sides of the nanopore layer and/or may include a nanopore layer having a minimal surface area.

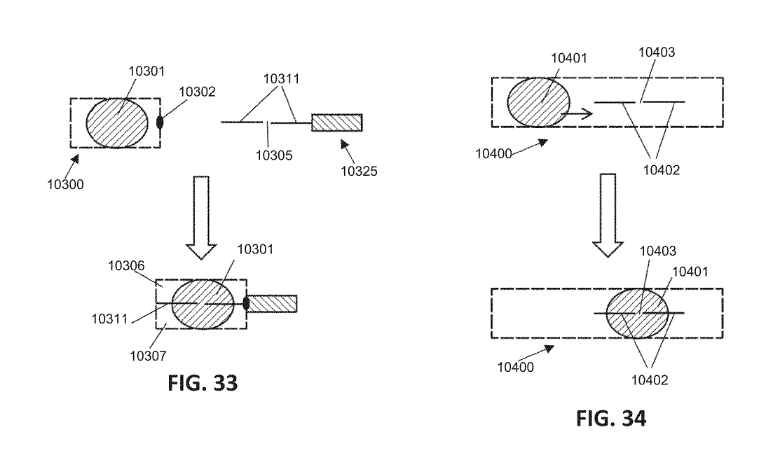

FIG. 33 illustrates another exemplary integrated device which includes a microfluidics module 10300 and a nanopore module 10325. In contrast to the nanopore module in FIGS. 31A, 31B, 32A and 32B, the nanopore module 10325 is not functional as a standalone device but functions as a nanopore once integrated with the microfluidics module 10300. The microfluidics module 10300 includes an opening 10302 sized to allow insertion of the nanopore module 10325. As depicted in FIG. 33, the microfluidics module includes a fluidic droplet 10301 that is to be analyzed using the nanopore module 10325, which contains a layer 10311 with a nanopore 10305. Upon insertion of the nanopore module 10325 into the microfluidics module 10300, a first chamber 10306 and a second chamber 10307 separated by the layer 10311 are created. The layer 10311 also splits the fluid droplet 10301 across the nanopore 10305.

FIG. 34 provides an integrated device 10400 in which the digital microfluidics modules includes a built-in nanopore module. In FIG. 34, the nanopore module is positioned downstream from the area in the microfluidics module where a fluidic droplet 10401 is generated. The microfluidics module moves the droplet 10401 to the nanopore module such that the droplet 10401 is split across the layer 10402 and is positioned at the nanopore 10403. FIG. 34 shows a top view of the device. The top substrate has not been shown for clarity. The nanopore 10403 in the nanopore layer 10402 has been depicted, although from the top view, nanopore 10403 will not be visible. The nanopore layer 10402 can be attached to either the bottom substrate or the top substrate.

In FIGS. 31A, 31B, 32A, 32B, 33 and 34, although a single nanopore is shown, it is understood that the layer may include one or more nanopores. In addition, more than one droplet may be positioned in the nanopore module or device. The droplet(s) may be analyzed by applying a voltage across the nanopore(s). Applying the voltage may result in movement of charged molecules across the nanopore(s). When a tag translocates through the nanopore(s), a decrease in electrical current across the nanopore provides an indication of the translocation. In certain embodiments, the chambers of the nanopore module may not be filled with a conductive solution (e.g., buffer)—the conductive solution may be provided by the fluid droplet once it is positioned across the nanopore layer. In certain cases, the first and second chambers, across which voltage is applied for measuring translocation of a tag/aptamer present in the fluid droplet, may be defined by the walls of the nanopore device and the nanopore layer (e.g., see FIGS. 31B and 32B). The first and second chambers may be empty prior to the introduction of the fluid droplet or may contain a conductive fluid. In other cases, the first and second chambers may be defined walls of the microfluidics module and the nanopore layer (e.g., see FIG. 33). In other cases, the first and second chambers may be defined by the fluidic droplet split across the nanopore layer (e.g., see FIGS. 31A, 32A, and 34). In certain cases, the voltage for conducting charged molecules across the nanopore(s) may be applied to the fluid droplet, for example, in embodiments where a conductive solution is not present in the chambers. Voltage may be applied to the fluid droplet via electrodes that are in direct or indirect contact with the fluid droplet. It is understood that the dimension of the nanopore layer is larger than that of the droplet such that droplet is split across the layer and connected only via the nanopore(s).

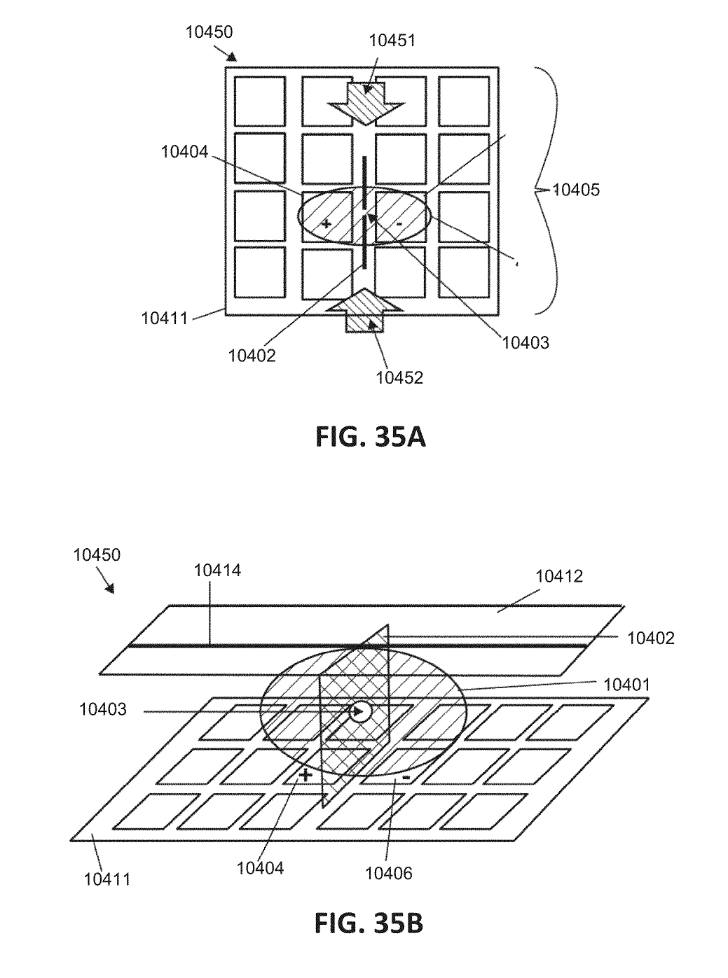

FIGS. 35A, 35B, 36 and 37 illustrate movement of droplets in devices that have a digital microfluidics module and a nanopore layer. In FIG. 35A, components of an integrated digital microfluidics/nanopore device 10450 are depicted. A top view shows that a droplet 10401 that is to be analyzed using the nanopore 10403 in the nanopore layer 10402 is positioned across the nanopore layer 10402. The nanopore 10403 is shown here for illustration purposes, although from a top view, the nanopore is not visible. The device 10450 includes a substrate 10411 on which an array of electrodes 10405 is disposed. The array of electrodes is used to position a droplet 10401 by splitting the droplet across nanopore layer 10402. Arrows 10451 and 10452 depict the direction in which the droplet 10401 may be moved across the array of electrodes to the nanopore layer 10402. Upon positioning of the droplet 10401 across the nanopore layer 10402, the electrodes 10404 and 10406 positioned below the droplet 10401 may be activated to provide a differential voltage across the nanopore layer 10402, thereby facilitating movement of molecules (e.g., cleaved tag or aptamer) in the droplet 10401 across the nanopore 10403. The electrodes 10404 and 10406 are dual function electrodes, they serve to move the droplet to the nanopore layer and to drive the tag/aptamer across the nanopore 10403.

FIG. 35B depicts a side view of the device 10450, a top substrate 10412 omitted in the top view shown in FIG. 35A is depicted here. The top substrate 10412 is shown to include an electrode 10414. Electrode 10414 may be a single electrode or an electrode array. The nanopore layer extends from the top substrate to the bottom substrate. The droplet 10401 is split across the nanopore layer 10402. Although bi-planar electrodes are depicted in FIG. 35B, the device may not include electrodes in both substrates; rather the top or the bottom substrate may include co-planar electrodes. The electrodes 10404 and 10406 in the vicinity of the droplet 10401 have opposite polarity and drive the tag/aptamer across the nanopore 10403.

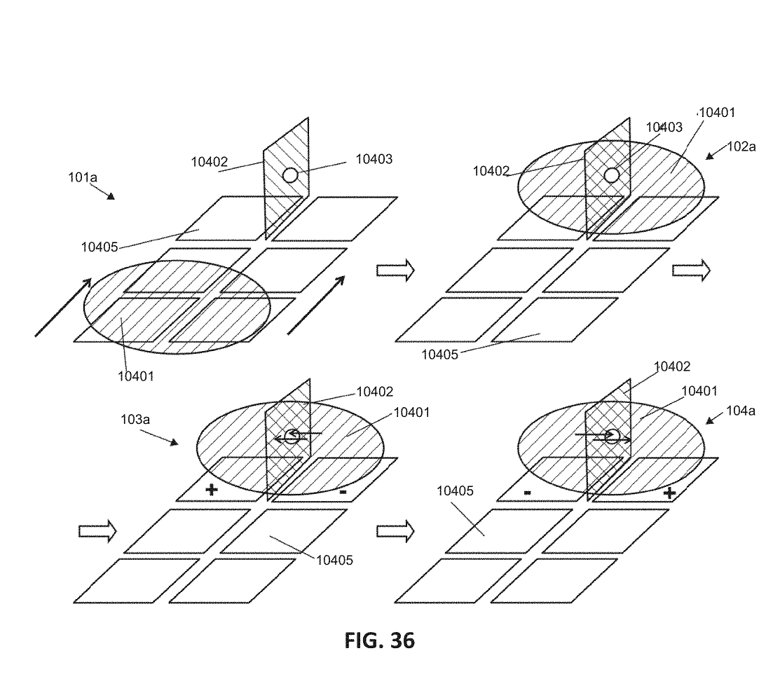

FIG. 36 shows the splitting of droplet 10401 across nanopore layer 10402 having a nanopore 10403. 101a depicts the droplet being moved by the electrodes 10405 in the direction indicated by the arrows towards the nanopore layer 10402. In 102a, the droplet 10401 has been split by the nanopore layer 10402 and positioned such that the droplet is connected via the nanopore 10403. In 103a, the electrodes positioned across the nanopore layer 10402 below the droplet 10401, are activated to provide an anode (−) and cathode (+). The activated electrodes drive the negatively charged molecules (including the tags/aptamers being counted) present in the droplet 10401 through the nanopore 10403. As the tags/aptamers translocate through the nanopore 10403, the number of tags/aptamers may be counted as explained herein. Step 103a serves to collect all the tags/aptamers that were divided across the nanopore layer, when the droplet was split, in one side of the droplet.

Once substantially all the tags/aptamers have been translocated to one side of the nanopore membrane, the polarity of the electrodes may be reversed, as shown in 104a, and the tags/aptamers translocated to the other side of the nanopore layer 10402 and counted. The number of tags counted in step 103a should be approximately half of the count obtained in step 104a. The steps of reversing polarity of electrodes and counting the tags/aptamers may be repeated any number of times to obtain multiple readings of the number of tags/aptamers in the droplet.

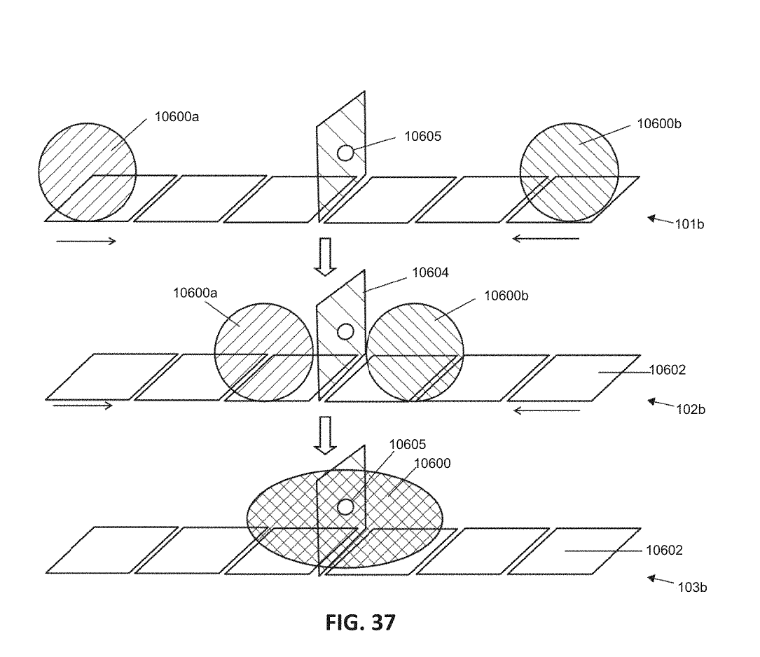

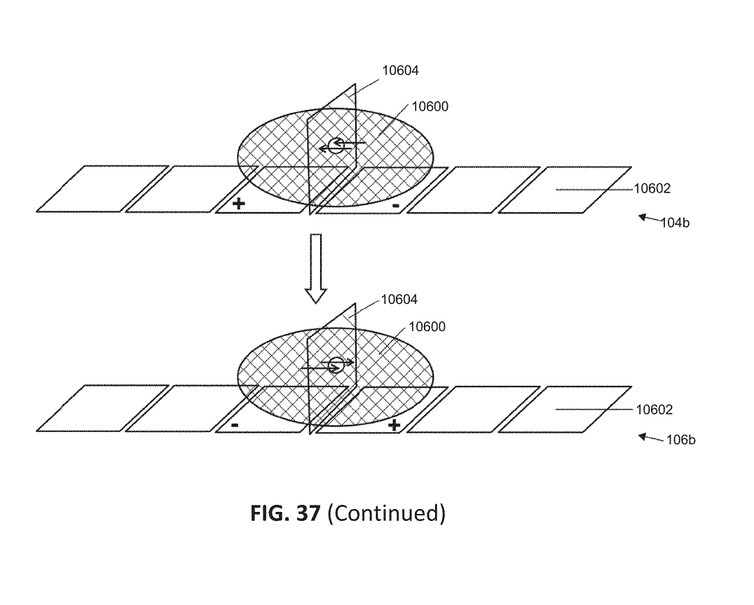

FIG. 37, 101b and 102b show two droplets 10600a and 10600b being moved to the nanopore layer 10604 in the directions indicated by the arrows. Once at the nanopore layer 10604, the droplets wet the nanopore layer and are fluidically connected via the nanopore 10605 (103b). In step 104b, an electrode positioned below the droplet 10600a is activated to serve as a cathode and an electrode positioned below the droplet 10600b is activated to serve as an anode and the negatively charged cleaved tags/dissociated aptamers are driven to the droplet 10600a and counted. In step 105b, the polarity of the electrodes is reversed and the negatively charged cleaved tags/dissociated aptamers present in droplet 10600a are driven to droplet 10600b and counted. The steps of reversing polarity of electrodes and counting the cleaved tags/dissociated aptamers may be repeated any number of times to obtain multiple readings of the number of cleaved tags/dissociated aptamers in the droplet. The two droplets 10600a and 10600b may both be sample droplets (e.g., droplets containing molecules to be counted) or buffer droplets (e.g., for wetting the nanolayer, prior to positioning a sample droplet(s) at the nanopore. In some embodiments, one of the droplets may be a buffer droplet while the other droplet may be the sample droplet. The tags/aptamers may be counted once or multiple times.

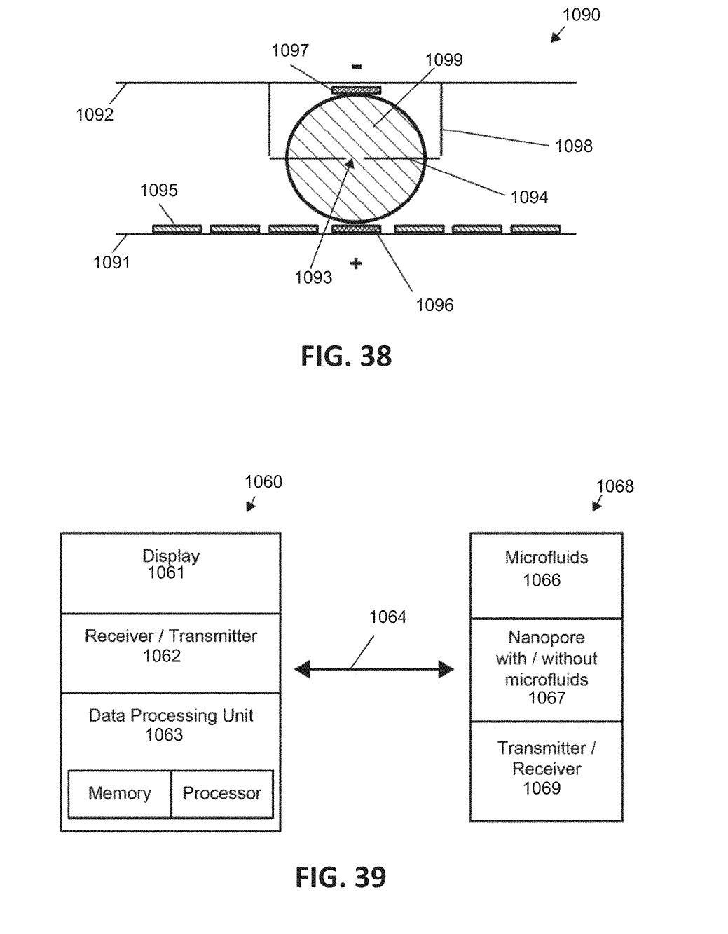

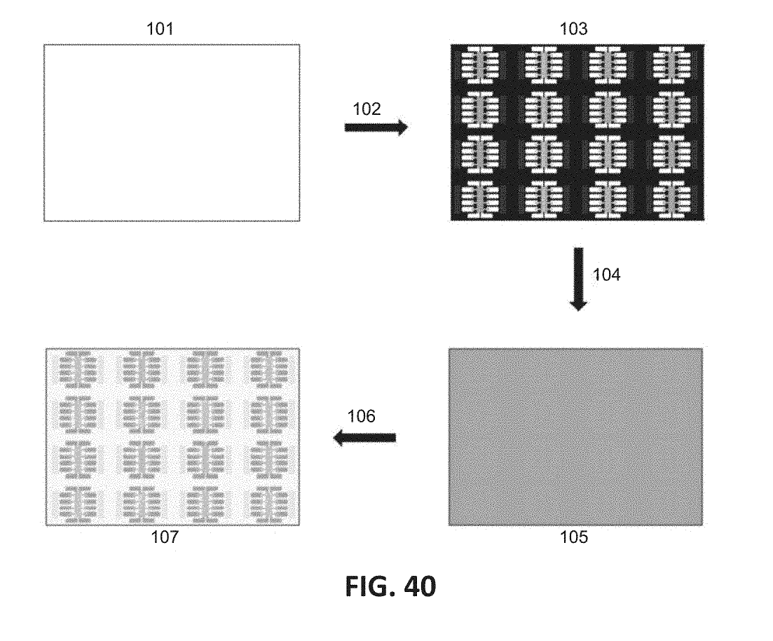

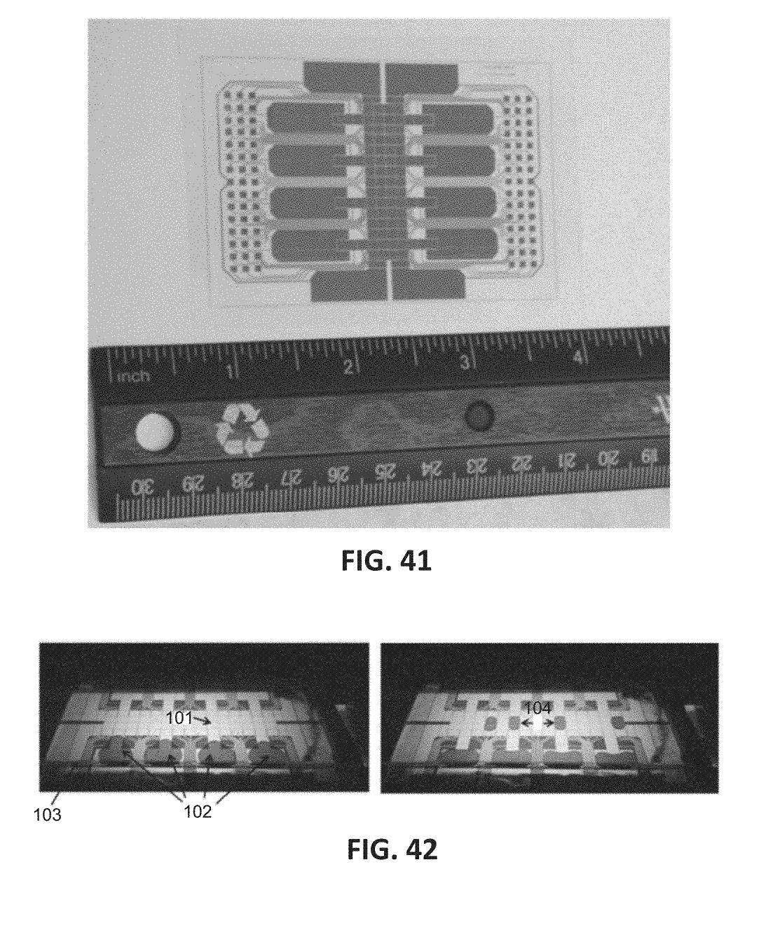

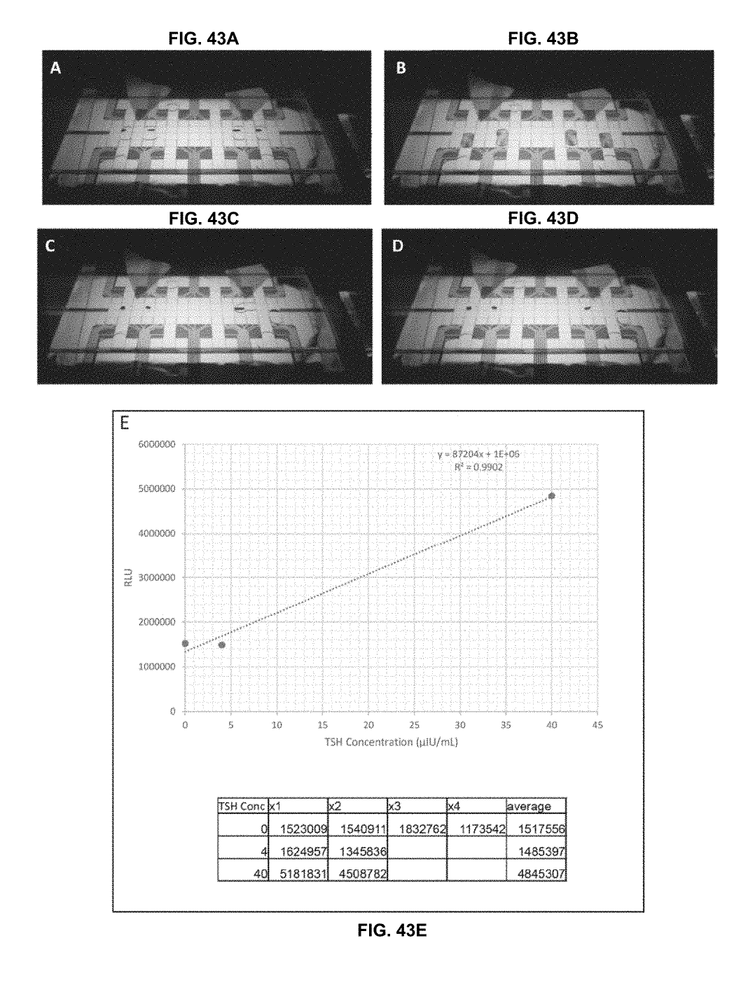

FIG. 38 illustrates an integrated digital microfluidics and nanopore device from a side view. Substrates 1091 and 1092 are positioned in a spaced apart manner. Substrate 1092 includes an electrode 1097 and substrate 1091 includes an electrode array 1095. Support structure 1098 attaches the nanopore layer 1094 to substrate 1092. In other embodiments, support structure 1098 may be attached to the bottom substrate 1091. Electrode array 1095 is used for moving the droplet 1099 to the nanopore layer 1094, where the nanopore layer splits the droplet and fluidically connects the two sides of the droplet via the nanopore 1093. Electrodes 1096 and 1097 serve to drive tags/aptamers in the droplet 1099 through the nanopore 1093. As noted above, the polarity of electrodes 1096 and 1097 may be reversed to translocate the tags/aptamers through the nanopore a number of times.

Although the figures depict a single nanopore, it is understood that more than one nanopore may be present in the nanopore layer. The electrodes that flank the nanopore layer and are used to provide a voltage difference across the nanopore layer may or may not be in direct contact with a droplet positioned at the nanopore layer.

The movement of fluidic droplet in the microfluidics and nanopore devices, modules, and the integrated devices may be carried out via any suitable means. The means for moving a fluidic droplet in different devices/modules and channels, if applicable, may be same or different. For example, fluidic droplets may be moved in the microfluidics device or module using fluidic manipulation force, such as, electrowetting, dielectrophoresis, opto-electrowetting, electrode-mediated, electric-field mediated, electrostatic actuation, and the like or a combination thereof. Movement of a fluidic droplet from a microfluidics module to a nanopore module through a fluidic connection, such as, a channel, may be via diffusion, Brownian motion, convection, pumping, applied pressure, gravity-driven flow, density gradients, temperature gradients, chemical gradients, pressure gradients (positive or negative), pneumatic pressure, gas-producing chemical reactions, centrifugal flow, capillary pressure, wicking, electric field-mediated, electrode-mediated, electrophoresis, dielectrophoresis, magnetophoresis, magnetic fields, magnetically driven flow, optical force, chemotaxis, phototaxis, surface tension gradient driven flow, Marangoni stresses, thermo-capillary convection, surface energy gradients, acoustophoresis, surface acoustic waves, electroosmotic flow, thermophoresis, electrowetting, opto-electrowetting, or combinations thereof. A fluidic droplet may be moved in the nanopore module and positioned across the nanopore layer via fluidic manipulation force, such as, electrowetting, dielectrophoresis, opto-electrowetting, electrode-mediated, electric-field mediated, electrostatic actuation, and the like or a combination thereof. The tag/aptamer in the droplet may be translocated through the nanopore(s) using electric potential, electrostatic potential, electrokinetic flow, electro-osmotic flow, pressure-induced flow, electrophoresis, electrophoretic transport, electro-osmotic transport, diffusion transport, electric-field mediated flow, dielectrophoretic mediated transport of the tag/aptamer, and other methods known to skill in the art or combinations thereof.

Exemplary embodiments of the present disclosure include counting the number of tags present in the droplet positioned across the nanopore layer by first translocating substantially all tags to the same side of the nanopore layer to collect all the tags in a cis or trans chamber, followed by translocating the tags to the other side of the nanopore layer and counting the number of tags traversing through the nanopore(s) in the nanopore layer. As used herein, “cis” and “trans” in the context of a nanopore layer refers to the opposite sides of the nanopore layer. These terms are used to in context of a side of the nanopore layer and also in the context of a chamber on a side of the nanopore layer. As is understood from the description of the devices, the cis and trans chambers may be defined by physical structures defined by walls, substrates, etc. In some cases, the cis and trans chambers may be defined by a droplet placed across a nanopore layer. The droplet may be in contact with a wall or substrate on one or more sides of the droplet. In certain cases, cis and trans chambers may be defined by the droplet, the cis chamber may extend from the cis side of the nanopore layer to the periphery of the portion of the droplet on the cis side and the trans chamber may extend from the trans side of the nanopore layer to the periphery of the portion of the droplet on the trans side. A portion of the droplet on each of cis and trans side may be in contact with a substrate. Thus, the cis and trans chamber may be defined by a combination of the periphery of the droplet, a portion of the substrates and the nanopore layer.

In certain cases, the microfluidics device and/or the microfluidics module may include an inert fluid that is immiscible with the sample droplet and the reagent droplets. For example, the inert fluid may be a heavy fluid that is denser than water, such as oil that is immiscible with the fluidic droplets being generated and processed in the microfluidics module. The inert fluid may facilitate formation of the fluidic droplets as well as increase stability of the shape of the fluid droplets and may further be useful for keeping the different droplets spatially separated from one another. Exemplary inert fluids include polar liquids, silicone oil, fluorosilicone oil, hydrocarbons, alkanes, mineral oil, and paraffin oil. In certain cases, the microfluidics device or module and the inert fluid may be as disclosed in US20070242105, which is herein incorporated by reference in its entirety. In other embodiments, an immiscible fluid is not included in the device. In these embodiments, the ambient air fills the spaces in the device.

As used herein, “droplet(s)” and “fluidic droplet(s)” are used interchangeably to refer to a discreet volume of liquid that is roughly spherical in shape and is bounded on at least two sides by a wall or substrate of the microfluidics device, the nanopore device, microfluidics module, or the nanopore module. Roughly spherical in the context of the droplet refers to shapes such as spherical, partially flattened sphere, e.g., disc shaped, slug shaped, truncated sphere, ellipsoid, hemispherical, or ovoid. The volume of the droplet in the microfluidics and nanopore modules and devices disclosed herein may range from about 10 μL to about 5 μL, such as, 10 μL-1 μL, 7.5 μL-10 μL, 5 μL-1 nL, 2.5 μL-10 nL, or 1 μL-100 nL, e.g., 10 μL, 1 μL, 800 nL, 400 nL, 100 nL, 10 nL, or lesser.

In certain embodiments, the integrated device may include a microfluidics module with a built-in nanopore module. The integrated device may include a first substrate and a second substrate with a gap separating the first and second substrates, the gap (which may be filled with air or an immiscible liquid) providing the space in which a sample droplet is contacted with the first binding member (either immobilized on a magnetic bead or on one of the two substrates); optionally a washing step is performed; followed by contacting the analyte bound to the first binding member with the second binding member; optional mixing and wash step may be performed; and the tag attached to the second binding member is cleaved to generate a droplet containing the cleaved tag. The droplet containing the cleaved tag may then be positioned across a nanopore layer located in the gap between the first and second substrates.

As noted herein, the droplets may be moved in the integrated device via numerous ways, such as, using a programmable fluidic manipulation force (e.g., electrowetting, dielectrophoresis, electrostatic actuation, electric field-mediated, electrode-mediated force, SAW, etc.). In certain cases, the microfluidics device and module may move droplets of sample and reagents for conducting analyte analysis by using electrodes. The electrodes may be co-planar, i.e., present on the same substrate or in a facing orientation (bi-planar), i.e., present in the first and second substrates. In certain cases, the microfluidics device or module may have the electrode configurations as described in U.S. Pat. No. 6,911,132, which is herein incorporated by reference in its entirety. In certain cases, the device may include a first substrate separated from a second substrate by a gap; the first substrate may include a series of electrodes positioned on an upper surface; a dielectric layer may be disposed on the upper surface of the first substrate and covering the series of electrodes to provide a substantially planar surface for movement of the droplets. Optionally, a layer of hydrophobic material may be placed on the upper surface of the dielectric layer to provide a substantially planar surface. In certain cases, the first substrate may include co-planar electrodes—e.g., drive/control and reference electrodes present on a single substrate. In other cases, the second substrate that is positioned over the first substrate may include an electrode on lower surface of the second substrate, where the lower surface of the second substrate is facing the upper surface of the first substrate. The electrode on the second substrates may be covered with an insulating material. The series of electrodes may be arranged in a longitudinal direction along a length of the microfluidics module or in a lateral direction along a width of the microfluidics module or both (e.g., a two-dimensional array or grid). In certain cases, the array of electrodes may be activated (e.g., turned on and off) by a processor of a computer operably coupled to the device for moving the droplets in a programmable manner. Devices and methods for actuating droplets in a microfluidics device are known. In exemplary cases, the microfluidics module may be similar to a droplet actuator known in the field. For example, the first (bottom) substrate may contain a patterned array of individually controllable electrodes, and the second (top) substrate may include a continuous grounding electrode. A dielectric insulator coated with a hydrophobic may be coated over the electrodes to decrease the wettability of the surface and to add capacitance between the droplet and the control electrodes (the patterned array of electrodes). In order to move a droplet, a control voltage may be applied to an electrode (in the array of electrodes) adjacent to the droplet, and at the same time, the electrode just under the droplet is deactivated. By varying the electric potential along a linear array of electrodes, electrowetting can be used to move droplets along this line of electrodes.

The first and second substrates may be made from any suitable material. Suitable materials without limitation include paper, thin film polymer, silica, silicon, processed silicon, glass (rigid or flexible), polymers (rigid, flexible, opaque, or transparent) (e.g., polymethylmethacrylate (PMMA) and cyclic olefin copolymer (COC), polystyrene (PS), polycarbonate (PC), printed circuit board, and polydimethylsiloxane (PDMS). In certain cases, at least the first or the second substrate may be substantially transparent. Substantially transparent substrate may be used in devices where photocleavage of tag attached to a second binding member is performed. In embodiments, where co-planar electrodes are present in one of the substrates, the electrodes may or may not be transparent. In other embodiments, such as, where electrodes are in facing orientation, (present in both substrates) the electrodes on at least one of the substrates may be substantially transparent, for example, the electrodes may be made from indium tin oxide. The electrodes may be made of any suitable material. The electrodes may be made of any conductive material such as pure metals or alloys, or other conductive materials. Examples include aluminum, carbon (such as graphite), chromium, cobalt, copper, gallium, gold, indium, iridium, iron, lead, magnesium, mercury (as an amalgam), nickel, niobium, osmium, palladium, platinum, rhenium, rhodium, selenium, silicon (such as highly doped polycrystalline silicon), silver, tantalum, tin, titanium, tungsten, vanadium, zinc, zirconium, mixtures thereof, and alloys or metallic compounds of these elements. In certain embodiments, the conductive material includes carbon, gold, platinum, palladium, iridium, or alloys of these metals, since such noble metals and their alloys are unreactive in aqueous environment.

In certain cases, the first substrate or the second substrate may have a first binding member immobilized thereon in the gap. For example, a surface of the first substrate that is in facing relationship to a surface of the second substrate may include an area on which a first binding member is disposed. As noted herein, the first binding member (e.g., a polypeptide, for example, a receptor, an antibody or a functional fragment thereof) may be immobilized on the surface of a solid substrate using any conventional method. In certain cases, a first position on the surface of the first or the second substrate in the gap may only include one type of binding member (e.g., a single type of antibody). In other embodiments, a first position on the surface of the first or the second substrate in the gap may only include a plurality of different binding members, for analysis of multiple analytes. Alternatively, the device may include a plurality of locations on the surface of the first or second substrates where each location may include a different first binding member immobilized thereupon.

In embodiments where a surface of the first substrate or the second substrate in the gap has a plurality of locations at which different first binding members are immobilized, the locations may be arranged linearly along a length of the device. A sample droplet may be moved linearly to sequentially contact each of the plurality of the locations. In another embodiment, a sample may be split into multiple droplets and each of the droplets may independently contact the each of the plurality of the locations. As noted herein, the first binding member may not be attached to the first or the second substrate and may be attached to a bead that may be introduced in the microfluidics device as, e.g., a droplet.

As noted herein, a sample and any reagents for assaying the sample may be manipulated as discrete volumes of fluid that may be moved in between the first and second substrates using a programmable fluidic manipulation force (e.g., electrowetting, dielectrophoresis, electrostatic actuation, electric field-mediated, electrode-mediated force, etc.). For example, at least one of the first and second substrates may include an array of electrodes for manipulating discrete volumes of fluid, e.g., moving droplets from one location to another in between the first and second substrates, mixing, merging splitting, diluting, etc. In another example, surface acoustic waves may be used to move droplets for the analyte analysis method.

In another embodiment, the microfluidics module may move droplets of sample and reagents for conducting analyte analysis by using surface acoustics waves. In these embodiments the first substrate may a thin planar material conducive to propagation of surface acoustic waves. The first substrate may be a piezoelectric crystal layer, such a lithium niobate (LiNbO₃), quartz, LiTaO₃ wafer. In certain cases, the piezoelectric wafer may be removably coupled to a supersubstrate, where surface acoustic waves (SAWs) generated from a transducer is transmitted to the supersubstrate via a coupling medium disposed between the piezoelectric crystal layer and the supersubstrate. The upper surface of the supersubstrate may be overlayed by a second substrate and a droplet may be moved in a space between the second substrate and upper surface of the supersubstrate via SAWs generated by an interdigitated transducer connected to the piezoelectric crystal layer. In certain cases, the microfluidics module may be a SAW microfluidics device described in WO2011/023949, which is herein incorporated by reference.

In an alternate embodiment, the microfluidics module may include a first surface separated from a second surface with a space between the first surface and the second surface, where sample and reagent droplets are manipulated for performing the sample analysis disclosed herein. The microfluidics device may further include a layer of surface acoustic wave (SAW) generation material coupled to the first surface; and a transducer electrode structure arranged at the SAW generation material layer to provide surface acoustic waves (SAWs) at the first surface for transmission to droplets on the first surface, where the first surface has at least one SAW scattering element for affecting the transmission, distribution and/or behavior of SAWs at the first surface, and where the SAW generation material is selected from the group consisting of: polycrystalline material, textured polycrystalline material, biaxially textured polycrystalline material, microcrystalline material, nanocrystalline material, amorphous material and composite material. In certain cases, the SAW generation material may be ferroelectric material, pyroelectric material, piezoelectric material or magnetostrictive material. The arrangement of the SAW scattering elements may provide, in effect, a phononic crystal structure that interacts with or affects the acoustic field at the first surface to affect movement of droplet on the first surface. In certain cases, the microfluidics module may be a SAW microfluidics device described in US20130330247, which is herein incorporated by reference. The SAW microfluidics device may be used in conjunction with a nanopore device or may have a nanopore module integrated therewith.

The devices described herein may be used in conjunction with another device or devices, such as, a power source, an acoustic wave generator, and the like.

The device that may be used for carrying out the method steps described herein may also include means for supplying reagent and collecting waste materials. Such means may include chambers, absorption pads, reservoirs, etc. These means may be fluidically connected to the device.

The microfluidics module may be fluidically connected to reservoirs for supplying sample analysis reagents, such as, first binding member, second binding member, wash buffer, cleavage inducing reagent and the like. The nanopore module may be fluidically connected to a reservoir for collecting waste materials, reservoirs for supplying conductive solution to the cis and trans chambers and the like.

The integrated device may be automatic or semi-automatic and may be removably coupled to a housing comprising a source of electricity for supplying voltage to the electrodes and a random access memory for storing instructions for contacting the sample with a first binding member, wherein the first binding member is immobilized on a solid support and wherein the first binding member specifically binds to the analyte; contacting the analyte with a second binding member, wherein the second binding member specifically binds to the analyte and wherein the second binding member comprises a cleavable tag attached thereto; removing second binding member not bound to the analyte bound to the first binding member; cleaving the tag attached to the second binding member bound to the analyte bound to the first binding member; translocating the tag through or across nanopores in a layer; determining the number of tags translocating through the layer; measuring the analyte in the sample based on the numbers of tags translocating through the layer or the time to translocate a known number of tags for a fixed interval of time. As noted herein, the analyte analysis method may be executed using a processor that controls the device. For example, the device may be programmed to perform analyte analysis as disclosed herein, including any optional mixing, incubating, and washing steps as disclosed herein. The housing may further include a processor for executing the instructions stored in the memory. The devices described herein may include a data acquisition module (DAQ) for processing electrical signals from the nanopore device or module. In certain cases, a patch-clamp amplifier for processing electrical signals and achieving optimal signal to noise ratio may also be included.

In certain cases, the devices described herein may be associated with a system for automatically performing at least some steps of the analyte analysis methods. An example of such a system is shown in FIG. 39. The Exemplary system includes a processing component 1060 including a data processing unit 1063 having a processor and memory, operatively coupled to display 1061 and a transmitter/receiver unit 1062 that is in communication 1064 with a receiver/transmitter unit 1069 of a device 1068 of the present disclosure. The device 1068 is controlled by the processing component 1060 that executes instructions (steps of a program) to perform at least some steps of the analyte analysis methods disclosed herein. In certain cases, the processing component 1060 may be a computer, a meter with an opening for insertion of the integrated device (the opening may be a slot sized and shaped to accommodate the device and operably connect to the device), or a combination thereof. The communication 1064 between the processing component 1060 and the device 1068 may be wired or wireless. The device 1068 may be any device described herein with microfluidics 1066 and nanopore 1067 functionality. In certain cases, the movement of a droplet in the devices disclosed herein may be programmed as disclosed in U.S. Pat. No. 6,294,063, which is herein incorporated by reference in its entirety.

The various illustrative processes described in connection with the embodiments herein may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The processor can be part of a computing system that also has a user interface port that communicates with a user interface, and which receives commands entered by a user, has at least one memory (e.g., hard drive or other comparable storage, and random access memory) that stores electronic information including a program that operates under control of the processor and with communication via the user interface port, and a video output that produces its output via any kind of video output format, e.g., VGA, DVI, HDMI, DisplayPort, or any other form.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein. The camera may be a camera based on phototubes, photodiodes, active pixel sensors (CMOS), CCD, photoresistors, photovoltaic cells or other digital image capture technology.

The steps of a method or algorithm described in connection with the embodiments disclosed herein may be embodied directly in hardware, in a software module executed by a processor, or in a combination of the two. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, a cloud, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more example embodiments, the functions described may be implemented in hardware, software, firmware, or any combination thereof. If implemented in software, the functions may be stored on, transmitted over or resulting analysis/calculation data output as one or more instructions, code or other information on a computer-readable medium. Computer-readable media includes both computer storage media and communication media including any medium that facilitates transfer of a computer program from one place to another. A storage media may be any available non-transitory media that can be accessed by a computer. By way of example, such computer-readable media can include RAM, ROM, EEPROM, CD-ROM or other optical disk storage, magnetic disk storage or other magnetic storage devices, or any other medium that can be used to carry or store desired program code in the form of instructions or data structures and that can be accessed by a computer. The memory storage can also be rotating magnetic hard disk drives, optical disk drives, or flash memory based storage drives or other such solid state, magnetic, or optical storage devices. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

In certain cases, the device may be a microfluidic device, such as a lab-on-chip device, continuous-flow microfluidic device, or droplet-based microfluidic device, where analyte analysis may be carried out in a droplet of the sample containing or suspected of containing an analyte. Exemplary microfluidic devices that may be used in the present methods include those described in WO2007136386, U.S. Pat. No. 8,287,808, WO2009111431, WO2010040227, WO2011137533, WO2013066441, WO2014062551, or WO2014066704. In certain cases, the device may be digital microfluidics device (DMF), a surface acoustic wave based microfluidic device (SAW), a fully integrated DMF and nanopore device, or a fully integrated SAW and nanopore device. In some embodiments, the DMF element and a nanopore element are operatively coupled in the fully integrated DMF and nanopore device, or a SAW element and a nanopore element are operatively coupled in the fully integrated SAW and nanopore device. In some embodiments, the DMF device or the SAW device is fabricated by roll to roll based printed electronics method. In some embodiments, the DMF element or the SAW element is fabricated by roll to roll based printed electronic methods. In some embodiments, the fully integrated DMF and nanopore device or the fully integrated SAW and nanopore device comprise a microfluidic conduit. In some embodiments, the microfluidic conduit couples the DMF element to the nanopore element, and the microfluidic conduit comprises a fluidic flow that is induced by passive forces or active forces.

Exemplary electrowetting techniques can be found in U.S. Pat. No. 8,637,242. Electrophoresis on a microscale such as that described in WO2011057197 may be also utilized. An exemplary dielectrophoresis technique is described in U.S. Pat. No. 6,294,063.

The devices of the present disclosure are generally free of external pumps and valves and are thus economical to manufacture and use. The devices and associated systems disclosed herein, as well as all the methods disclosed herein, are useful for applications in the field, such as, for analysis of a sample at the source of the sample, such as, at point-of-care (e.g., in the clinics, hospitals, physician's office, core laboratory facility, in home, and the like). In some cases, a device or system of the present disclosure (e.g., also as used in the methods disclosed herein) includes a heat source or a light source configured to induce, when the heat source or light source is activated, cleavage of a thermally cleavable or a photocleavable linker linking the tag to the analyte, as described herein.

The present disclosure also describes a microfluidics device used in conjunction with a nanopore-enabled device and an integrated microfluidics nanopore-enabled device. A nanopore-enabled device refers to a device which includes a layer or membrane in which a nanopore can be created. A nanopore-enabled device of the present disclosure includes two chambers separated by the layer or membrane, where the two chambers include an ionic liquid, (e.g., a salt solution, with or without an analyte of interest) for conducting current. A nanopore may be created in the layer of the nanopore-enable device by applying a voltage across the layer using the ionic liquid (e.g., salt solution, with or without an analyte of interest) in the chambers. As will be understood any of the nanopore devices (used in conjunction with a microfluidics device or integrated with a microfluidics module) described herein may initially be provided as a nanopore-enabled device that includes a layer in which a nanopore can be formed but is devoid of a nanopore. A nanopore may be created in the nanopore-enabled device during use, such as, prior to using the nanopore for detecting translocation of a tag. In certain embodiments, an ionic liquid, e.g., salt solution, containing the tag to be detected by the nanopore may be used for both creating the nanopore and for translocating a tag across the created nanopore.

In some embodiments, a quality of the nanopore that is created by applying voltage across the layer, as described above, is assessed by the level of noise in a current measured when a baseline voltage is applied across the nanopore layer or membrane.

In some cases, the nanopore created by applying voltage across the layer, as described above, may be conditioned to physically alter the nanopore and to obtain a desired electroosmotic property, e.g., increase the pore size and/or to reduce noise in the measured current across the nanopore when a voltage is applied across the nanopore layer or membrane. Thus, in some embodiments, a method of generating a nanopore in an integrated digital microfluidics nanopore-enabled device may include conditioning the nanopore. Conditioning may include: alternately applying a first voltage having a first polarity and a second voltage having a second polarity opposite the first polarity across the nanopore layer or membrane, wherein the first and second voltages are each applied at least once; and measuring an electroosmotic property related to a size of the nanopore. In some cases, the electroosmotic property related to a size of the nanopore is measured before the conditioning, to obtain an initial estimate of the size of the nanopore.

The electroosmotic property may be any suitable property that provides an estimate for the size of the nanopore. In some cases, the electroosmotic property is represented by a current-voltage curve obtained over a range of voltages (a range of −1 V to 1 V, e.g., −500 mV to 500 mV, −250 mV to 250 mV, −200 mV to 200 mV, 10 mV to 500 mV, 10 mV to 250 mV, 10 mV to 200 mV, including 15 mV to 200 mV). In some cases, the electroosmotic property is a conductance or resistance measured across the nanopore layer or membrane.

The first and second voltage may have any suitable magnitude for modifying the nanopore and to obtain the desired electroosmotic properties. In some cases, the first and second voltages have a magnitude or 100 mV or more, e.g., 200 mV or more, 500 mV or more, 750 mV or more, 1.0 V or more, 2.0 V of more, 3.0 V or more, including 4.0 V or more, and in some cases has a magnitude of 10 V or less, e.g., 9.0 V or less, 8.0 V or less, 6.0 V or less, including 4.0 V or less. In some embodiments, the first and second voltages have a magnitude in the range of 100 mV to 10 V, e.g., 200 mV to 9.0 V, 250 mV to 9.0 V, 500 mV to 9.0 V, 1.0 V to 8.0 V, including 2.0 V to 6.0 V.

The first and second voltages may each be applied for any suitable length of time for modifying the nanopore and to obtain the desired electroosmotic properties. In some cases, the first and second voltages are each applied for 10 milliseconds (ms) or more, e.g., 100 ms or more, 200 ms or more, 500 ms or more, 1 second (s) or more, 2 s or more, including 3 s or more, and in some cases, is applied for 10 s or less, e.g, 5 s or less, 4 s or less, 3 s or less, 2 s or less, 1 s or less, 500 ms or less, 200 ms or less, including 100 ms or less. In some cases, the first and second voltages are each applied for a duration in the range of 10 ms to 100 ms, 100 ms to 200 ms, 200 ms to 500 ms, 500 ms to 1 s, 1 s to 2 s, 2 s to 3 s, 3 s to 4 s, 3 s to 5 s, or 3 s to 10 s.

The first and second voltages may each be applied any suitable number of times for modifying the nanopore and to obtain the desired electroosmotic properties. In some cases, the first and second voltages are each applied twice or more, three times or more, 4 times or more, 5 times or more, 7 times or more 10 times or more, 20 times or more, 30 times or more, 50 times or more, 100 times or more, 200 times or more, including 500 times or more, and in some embodiments, is applied for 10,000 time or less, e.g., 5,000 times or less, 1,000 times or less, 500 times or less, 400 times or less, 200 times or less, 100 times or less, including 50 times or less. In some embodiments, the first and second voltages are each applied from two to 50 times, 10 to 50 times, 30 to 50 times, 50 to 100 times, 100 to 200 times, 100 to 500 times, 500 to 1,000 times, 500 to 1,000 times, or 500 to 10,000 times.

### 4. Integration of a Nanopore Module on One Side of a DMF Module

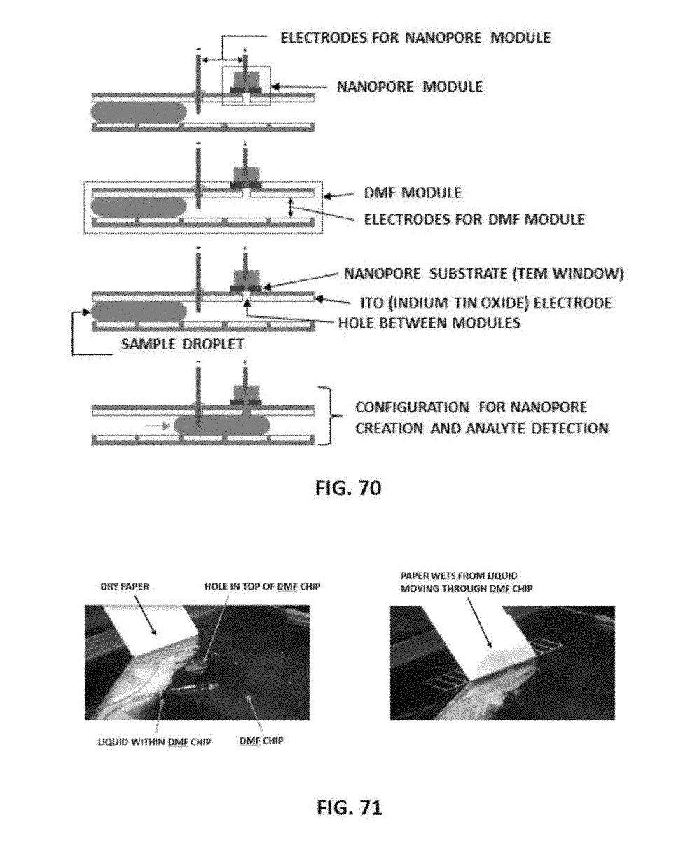

An aspect of the present disclosure includes an integrated device that includes a digital microfluidics (DMF) module and a nanopore layer positioned on one exterior side of the DMF module (FIG. 70). The nanopore of the nanopore layer may be accessed by a droplet in an internal space of the DMF module through a hole (also referred to as an “opening”) that is present in the first (e.g., top) or second (e.g., bottom) substrate of the DMF module or through a side of the DMF module between the first and second substrate. As described above, the nanopore layer may include a nanopore membrane or substrate, which in some cases may be a commercially available silicon nitride (SiNx) membrane in a transmission electron microscope (TEM) window. The nanopore layer forms a seal over the hole such that, in the absence of a nanopore (i.e. prior to fabrication of a nanopore, as described herein), a volume of liquid in the DMF module is physically isolated from any volume of liquid on or around the outside of the nanopore layer. In some cases, the nanopore layer is part of a nanopore module, where the nanopore layer separates a compartment within the nanopore module from a volume of liquid in the DMF module (e.g., a liquid droplet in the hole of the substrate, as described above). The nanopore layer or module is sealed to the outer surface of the substrate such that a volume of liquid (e.g., a liquid droplet in the hole of the substrate) is physically isolated from the outside environment.

The hole in the substrate through which a liquid droplet in the DMF has access to the nanopore layer may be dimensioned to be suitable for a liquid droplet to move through the hole by capillary action. Thus, the hole in the substrate may be a capillary channel. The hole may have any suitable cross-sectional shape and dimensions to support movement of a liquid droplet through the hole passively, e.g., by capillary action. In some cases, the diameter of the hole is wider on the side of the DMF than the diameter of the hole on the external side (i.e., the side facing the nanopore layer). In some cases, the angle between the bottom surface of the substrate and the wall of the hole is right angle or obtuse (e.g., 90° or greater, e.g., 95° or greater, including 100° or greater).

The integrated DMF-nanopore module device may include a pair of electrodes, which may find use in fabricating the nanopore in the nanopore layer and/or for detecting an analyte of interest that has been processed by the DMF module, as described elsewhere herein. The pair of electrodes may be made of any suitable material, including, but not limited to, indium tin oxide (ITO). The pair electrodes may be configured in any suitable manner. In some embodiments, one electrode is positioned in a compartment in the nanopore module, and a second electrode is positioned in the DMF module, by physically penetrating the substrate to access the volume of liquid on the other side of the nanopore layer (FIG. 70).

In some embodiments, the first electrode may be the same electrode as the single continuous electrode (e.g., the reference electrode) used in the DMF module, and the second electrode may be disposed on the top surface (i.e., outer surface) of the substrate opposite the bottom surface on which the first electrode is positioned (FIG. 73). In such cases, the top surface may be treated in a similar manner as the bottom surface (e.g., coating with an electrode material, such as indium tin oxide, and a polymer, such as polytetrafluoroethylene (including Teflon®). Thus, in some cases, where the second electrode is an electrode on the top surface of the substrate to which the nanopore layer/module is attached, the volume of liquid on the outside surface of the nanopore layer relative to the DMF module is in electrical contact with the second electrode. The electrical path for the nanopore fabrication may be represented as: second electrode->liquid (external)->nanopore membrane (without a nanopore)->liquid (internal to DMF module)->first electrode (same as the single continuous electrode of the DMF). The second electrode may also be absent from the area where the nanopore layer/module is attached so as to force current into the liquid on the outside of the nanopore membrane, which in some cases may be contained within the nanopore module.

In some embodiments, as shown in FIG. 74, the first electrode is the same electrode as the single continuous electrode (e.g., the reference electrode) used in a first DMF module (e.g., “bottom DMF chip” in FIG. 74), and the second electrode may be provided by a second DMF module (e.g., “top DMF chip” in FIG. 74) having a hole in a corresponding top substrate associated with the single continuous electrode of the second DMF module, and the nanopore layer is interposed between the two DMF modules between the holes in the respective substrates. Thus, the first and second DMF modules may be reversed in orientation relative to each other such that the top substrate associated with the single continuous electrode of the first DMF module is proximal to and faces the top substrate associated with the single continuous electrode of the second DMF module. The two DMF modules may be positioned relative to each other such that, when there is a nanopore in the nanopore layer, the two DMF modules are fluidically and electrically coupled together through the nanopore membrane. Prior to formation of the nanopore, the two volumes of fluid in the two DMF modules may be isolated from each other. In some cases, a structural layer is interposed between the two DMF modules to provide structural support and reduce bending.

Also provided herein is a method of making a nanopore in a nanopore-enabled layer, in an integrated DMF-nanopore module device, as described above. An implementation of the method may include positioning an ionic liquid, e.g., a salt solution (e.g., LiCl, KCl, etc.) to the hole in the DMF module using any suitable method, as described herein, and allowing capillary action to move the liquid through the hole (see, e.g., FIG. 70). An ionic liquid, e.g., a salt solution, may be positioned on the other side of the nanopore-enabled layer (i.e., the nanopore membrane before making a nanopore) The nanopore module is sealed from the DMF module, using any suitable method, such as, but not limited to PDMS, pressure, wax, adhesive, etc., such that the liquid volume in the hole is isolated from a liquid volume on the other side of the nanopore membrane. Application of an electric field, such as a voltage across the nanopore-enabled layer leads to the eventual formation of a nanopore, which can be readily detected, e.g., as a dielectric breakdown in a current trace.

After creation of a nanopore in the nanopore layer, in some cases, a conditioning process may be carried out to physically modify the nanopore and clean the signal. In some cases, the conditioning includes varying the voltage applied across the nanopore over time.

After nanopore fabrication, the DMF module may be re-activated to complete any liquid pre-processing steps for translocation (e.g. replace solution in the DMF, such as replacing KCl with LiCl). After pre-processing, the DMF liquid volume, e.g., a liquid sample containing an analyte of interest, may be positioned in the hole. The DMF system may then be de-activated and the nanopore module may be enabled to allow and detect translocation events.

### 5. Variations on Methods and on Use of the Device

The disclosed methods of determining the presence or amount of analyte of interest present in a sample, and the use of the microfluidics device, may be as described above. The methods and use of the disclosed microfluidics device may also be adapted in view of other methods for analyzing analytes. Examples of well-known variations include, but are not limited to, immunoassay, such as sandwich immunoassay (e.g., monoclonal-polyclonal sandwich immunoassays, including enzyme detection (enzyme immunoassay (EIA) or enzyme-linked immunosorbent assay (ELISA), competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, etc. In some instances, the descriptions below may overlap the method described above; in others, the descriptions below may provide alternates.

i. Immunoassay

The analyte of interest, and/or peptides or fragments thereof, may be analyzed using an immunoassay. The presence or amount of analyte of interest can be determined using the herein-described antibodies and detecting specific binding to analyte of interest. Any immunoassay may be utilized. The immunoassay may be an enzyme-linked immunoassay (ELISA), a competitive inhibition assay, such as forward or reverse competitive inhibition assays, or a competitive binding assay, for example. In some embodiments, a detectable label (e.g., such as one or more fluorescent labels one or more tags attached by a cleavable linker (which can be cleaved chemically or by photocleavage)) is attached to the capture antibody and/or the detection antibody. Alternately, a microparticle or nanoparticle employed for capture, also can function for detection (e.g., where it is attached or associated by some means to a cleavable linker).

A heterogeneous format may be used. For example, after the test sample is obtained from a subject, a first mixture is prepared. The mixture contains the test sample being assessed for analyte of interest and a first specific binding partner, wherein the first specific binding partner and any analyte of interest contained in the test sample form a first specific binding partner-analyte of interest complex. Preferably, the first specific binding partner is an anti-analyte of interest antibody or a fragment thereof. The order in which the test sample and the first specific binding partner are added to form the mixture is not critical. Preferably, the first specific binding partner is immobilized on a solid phase. The solid phase used in the immunoassay (for the first specific binding partner and, optionally, the second specific binding partner) can be any solid phase known in the art, such as, but not limited to, a magnetic particle, a bead a nanobead, a microbead, a nanoparticle, a microparticle, a membrane, a scaffolding molecule, a film, a filter paper, a disc, or a chip (e.g., a microfluidic chip).

After the mixture containing the first specific binding partner-analyte of interest complex is formed, any unbound analyte of interest is removed from the complex using any technique known in the art. For example, the unbound analyte of interest can be removed by washing. Desirably, however, the first specific binding partner is present in excess of any analyte of interest present in the test sample, such that all analyte of interest that is present in the test sample is bound by the first specific binding partner.

After any unbound analyte of interest is removed, a second specific binding partner is added to the mixture to form a first specific binding partner-analyte of interest-second specific binding partner complex. The second specific binding partner is preferably an anti-analyte of interest (such as an antibody) that binds to an epitope on analyte of interest that differs from the epitope on analyte of interest bound by the first specific binding partner. Moreover, also preferably, the second specific binding partner is labeled with or contains a detectable label (e.g., which can be a tag attached by a cleavable linker, as described herein).

The use of immobilized antibodies or fragments thereof may be incorporated into the immunoassay. The antibodies may be immobilized onto a variety of supports, such as magnetic or chromatographic matrix particles, latex particles or modified surface latex particles, polymer or polymer film, plastic or plastic film, planar substrate, a microfluidic surface, pieces of a solid substrate material, and the like.

ii. Sandwich Immunoassay

A sandwich immunoassay measures the amount of antigen between two layers of antibodies (i.e., a capture antibody (i.e., at least one capture antibody) and a detection antibody (i.e. at least one detection antibody)). The capture antibody and the detection antibody bind to different epitopes on the antigen, e.g., analyte of interest. Desirably, binding of the capture antibody to an epitope does not interfere with binding of the detection antibody to an epitope. Either monoclonal or polyclonal antibodies may be used as the capture and detection antibodies in the sandwich immunoassay.

Generally, at least two antibodies are employed to separate and quantify analyte of interest in a test sample. More specifically, the at least two antibodies bind to certain epitopes of analyte of interest or an analyte of interest fragment forming an immune complex which is referred to as a “sandwich”. One or more antibodies can be used to capture the analyte of interest in the test sample (these antibodies are frequently referred to as a “capture” antibody or “capture” antibodies), and one or more antibodies with a detectable label (e.g., a fluorescent label, a tag attached by a cleavable linker, etc.) that also bind the analyte of interest (these antibodies are frequently referred to as the “detection” antibody or “detection” antibodies) can be used to complete the sandwich. In some embodiments, an aptamer may be used as the second binding member and may serve as the detectable tag. In a sandwich assay, the binding of an antibody to its epitope desirably is not diminished by the binding of any other antibody in the assay to its respective epitope. In other words, antibodies are selected so that the one or more first antibodies brought into contact with a test sample suspected of containing analyte of interest do not bind to all or part of an epitope recognized by the second or subsequent antibodies, thereby interfering with the ability of the one or more second detection antibodies to bind to the analyte of interest.

In one embodiment, a test sample suspected of containing analyte of interest can be contacted with at least one capture antibody (or antibodies) and at least one detection antibodies either simultaneously or sequentially. In the sandwich assay format, a test sample suspected of containing analyte of interest (such as a membrane-associated analyte of interest, a soluble analyte of interest, fragments of membrane-associated analyte of interest, fragments of soluble analyte of interest, variants of analyte of interest (membrane-associated or soluble analyte of interest) or any combinations thereof) is first brought into contact with the at least one capture antibody that specifically binds to a particular epitope under conditions which allow the formation of an antibody-analyte of interest complex. If more than one capture antibody is used, a multiple capture antibody-analyte of interest complex is formed. In a sandwich assay, the antibodies, preferably, the at least one capture antibody, are used in molar excess amounts of the maximum amount of analyte of interest or the analyte of interest fragment expected in the test sample.

Optionally, prior to contacting the test sample with the at least one first capture antibody, the at least one capture antibody can be bound to a solid support which facilitates the separation the antibody-analyte of interest complex from the test sample. Any solid support known in the art can be used, including but not limited to, solid supports made out of polymeric materials in the form of planar substrates or beads, and the like. The antibody (or antibodies) can be bound to the solid support by adsorption, by covalent bonding using a chemical coupling agent or by other means known in the art, provided that such binding does not interfere with the ability of the antibody to bind analyte of interest or analyte of interest fragment. Moreover, if necessary, the solid support can be derivatized to allow reactivity with various functional groups on the antibody. Such derivatization requires the use of certain coupling agents such as, but not limited to, maleic anhydride, N-hydroxysuccinimide, azido, alkynyl, and 1-ethyl-3-(3-dimethylaminopropyl)carbodiimide.

After the test sample suspected of containing analyte of interest is brought into contact with the at least one capture antibody, the test sample is incubated in order to allow for the formation of a capture antibody (or capture antibodies)-analyte of interest complex. The incubation can be carried out at a pH of from about 4.5 to about 10.0, at a temperature of from about 2° C. to about 45° C., and for a period from at least about one (1) minute to about eighteen (18) hours, from about 2-6 minutes, or from about 3-4 minutes.

After formation of the capture antibody (antibodies)-analyte of interest complex, the complex is then contacted with at least one detection antibody (under conditions which allow for the formation of a capture antibody (antibodies)-analyte of interest-detection antibody (antibodies) complex). If the capture antibody-analyte of interest complex is contacted with more than one detection antibody, then a capture antibody (antibodies)-analyte of interest-detection antibody (antibodies) detection complex is formed. As with the capture antibody, when the at least one detection (and subsequent) antibody is brought into contact with the capture antibody-analyte of interest complex, a period of incubation under conditions similar to those described above is required for the formation of the capture antibody (antibodies)-analyte of interest-detection antibody (antibodies) complex. Preferably, at least one detection antibody contains a detectable label (e.g., a fluorescent label, a tag attached by a cleavable linker, etc.). The detectable label can be bound to the at least one detection antibody prior to, simultaneously with or after the formation of the capture antibody (antibodies)-analyte of interest-detection antibody (antibodies) complex. Any detectable label known in the art can be used, e.g., a fluorescent label, a cleavable linker as discussed herein, and others known in the art.

The order in which the test sample and the specific binding partner(s) are added to form the mixture for assay is not critical. If the first specific binding partner is detectably labeled (e.g., a fluorescent label, a tag attached with a cleavable linker, etc.), then detectably-labeled first specific binding partner-analyte of interest complexes form. Alternatively, if a second specific binding partner is used and the second specific binding partner is detectably labeled (e.g., a fluorescent label, a tag attached with a cleavable linker, etc.), then detectably-labeled complexes of first specific binding partner-analyte of interest-second specific binding partner form. Any unbound specific binding partner, whether labeled or unlabeled, can be removed from the mixture using any technique known in the art, such as washing.

Next, signal, indicative of the presence of analyte of interest or a fragment thereof is generated. Based on the parameters of the signal generated, the amount of analyte of interest in the sample can be quantified. Optionally, a standard curve can be generated using serial dilutions or solutions of known concentrations of analyte of interest by mass spectroscopy, gravimetric methods, and other techniques known in the art.

Provided herein are methods for measuring or detecting an analyte of interest present in a biological sample. In such methods, a sample droplet containing the target analyte of interest may be merged with a droplet containing beads (such as magnetic beads) on which a first specific binding partner that specifically binds to the target analyte of interest present in the sample is attached. Merging creates a single droplet which may be incubated for a time sufficient to allow binding of the first specific binding partner to an analyte of interest present in the sample droplet. Optionally, the single droplet may be agitated to facilitate mixing of the sample with the first specific binding partner. Mixing may be achieved by moving the single droplet back and forth, moving the single droplet around over a plurality of electrodes, splitting a droplet and then merging the droplets, or using SAWs, and the like. Next, the single droplet may be subjected to a magnetic force to retain the beads at a location in the device while the droplet may be moved away to a waste chamber or pad and replaced with a droplet containing a second binding member. The second specific binding partner may be detectably labeled. The label may be any label that can be optically detected. For example, the label may be a fluorescent label. An optional wash step may be performed, prior to adding the second binding member, by moving a droplet of wash buffer to the location at which the beads are retained using the force, e.g., magnetic. The beads may or may not be resuspended in the wash buffer. If magnetic beads are used, a a magnetic force can be applied to the magnetic beads and the wash buffer is transported to a waste location. After a period of time sufficient for the second specific binding partner to bind the analyte of interest bound to the first binding member, the droplet containing the second specific binding partner may be moved away while the beads are retained at the location. The beads may be washed using a droplet of wash buffer. Following the wash step, a droplet containing the labeled beads which has a complex of the first binding member, analyte of interest and the second binding partner may be moved over to the detection module (such as by removal of the magnetic force if magnetic beads are used). As explained herein, the immunoassay may be carried out in the sample preparation module. The labeled beads may be allowed to settle into the array of wells in the detection module. The beads may settle using gravitational force or by applying electric or magnetic force. Following a wash step to remove any beads not located inside the wells, the wells may be sealed by using a hydrophobic liquid.

iii. Forward Competitive Inhibition

In a forward competitive format, an aliquot of labeled analyte of interest (e.g., analyte having a fluorescent label, a tag attached with a cleavable linker, etc.) of a known concentration is used to compete with analyte of interest in a test sample for binding to analyte of interest antibody.

In a forward competition assay, an immobilized specific binding partner (such as an antibody) can either be sequentially or simultaneously contacted with the test sample and a labeled analyte of interest, analyte of interest fragment or analyte of interest variant thereof. The analyte of interest peptide, analyte of interest fragment or analyte of interest variant can be labeled with any detectable label, including a detectable label comprised of tag attached with a cleavable linker. In this assay, the antibody can be immobilized on to a solid support. Alternatively, the antibody can be coupled to an antibody, such as an antispecies antibody, that has been immobilized on a solid support, such as a microparticle or planar substrate.

Provided herein are methods for measuring or detecting an analyte of interest present in a biological sample. In such methods, a sample droplet containing the target analyte of interest may be merged with a droplet containing magnetic beads on which a first specific binding partner that specifically binds to the target analyte of interest present in the sample is attached and analyte labeled with a detectable label (such as a fluorescent label). Optionally, the single droplet may be agitated to facilitate mixing of the sample with the first specific binding partner and the labeled analyte. Mixing may be achieved by moving the single droplet back and forth, moving the single droplet around over a plurality of electrodes, splitting a droplet and then merging the droplets, or using SAWs, and the like. Next, the single droplet may be subjected to a force (such as a magnetic force) to retain the beads at a location in the device while the droplet may be moved away to a waste chamber or pad and replaced with a droplet containing a second binding member. An optional wash step may be performed by moving a droplet of wash buffer to the location at which the beads are retained using the magnetic force. The beads may or may not be resuspended in the wash buffer; a force is applied to the beads (such as a magnetic force if magnetic beads are used) and the wash buffer is transported to a waste location. After a period of time sufficient for the first specific binding partner to bind to the analyte of interest, the droplet may be moved away while the beads are retained at the location. Following the optional wash step, a droplet containing the labeled beads which has a complex of the first binding member and analyte of interest may be moved over to the detection module (such as by removing a magnetic force if magnetic beads are used). As explained herein, the immunoassay may be carried out in the sample preparation module. The labeled beads may be allowed to settle into the array of wells in the detection module. The beads may settle using gravitational force or by applying a force, e.g., electric or magnetic. Following a wash step to remove any beads not located inside the wells, the wells may be sealed by using a hydrophobic liquid.

Additionally or alternatively, the method includes contacting the sample with a binding member, wherein the binding member is immobilized on a solid support and wherein the binding member specifically binds to the analyte; contacting the sample, which may contain analyte bound to the binding member, with a labeled analyte, wherein the labeled analyte is labeled with a cleavable tag; removing labeled analyte not bound to the binding member; cleaving the tag attached to the labeled analyte bound to the binding member; translocating the cleaved tag through or across one or more nanopores in a layer; and assessing the tag translocating through the layer, wherein measuring the number of tags translocating through the layer measures the amount of analyte present in the sample, or detecting tags translocating through the layer detects that the analyte is present in the sample. In some embodiments, measuring the tags translocating through the layer is assessed, wherein the number of tags translocating through the layer measures the amount of analyte present in the sample. In some embodiments, detecting the tags translocating through the layer is assessed, wherein detecting tags translocating through the layer detects that the analyte is present in the sample.

Provided herein are methods for measuring or detecting an analyte present in a biological sample. The method includes contacting the sample with a binding member, wherein binding member is immobilized on a solid support and wherein binding member specifically binds to the analyte; contacting the sample, which may contain analyte bound to the binding member, with a labeled analyte, wherein the labeled analyte comprises an aptamer; removing labeled analyte not bound to the binding member; dissociating the aptamer bound to the labeled analyte bound to the binding member and translocating the dissociated aptamer through or across one or more nanopores in a layer; and assessing the aptamer translocating through the layer, wherein measuring the number of aptamers translocating through the layer measures the amount of analyte present in the sample, or detecting aptamers translocating through the layer detects that the analyte is present in the sample. In some embodiments, measuring the aptamers translocating through the layer is assessed, wherein the number of aptamers translocating through the layer measures the amount of analyte present in the sample. In some embodiments, detecting the aptamers translocating through the layer is assessed, wherein detecting tags translocating through the layer detects that the analyte is present in the sample.

The labeled analyte of interest, the test sample and the antibody are incubated under conditions similar to those described above in connection with the sandwich assay format. Two different species of antibody-analyte of interest complexes may then be generated. Specifically, one of the antibody-analyte of interest complexes generated contains a detectable label (e.g., a fluorescent label, a tag, etc.) while the other antibody-analyte of interest complex does not contain a detectable label. The antibody-analyte of interest complex can be, but does not have to be, separated from the remainder of the test sample prior to quantification of the detectable label. Regardless of whether the antibody-analyte of interest complex is separated from the remainder of the test sample, the amount of detectable label in the antibody-analyte of interest complex is then quantified. The concentration of analyte of interest (such as membrane-associated analyte of interest, soluble analyte of interest, fragments of soluble analyte of interest, variants of analyte of interest (membrane-associated or soluble analyte of interest) or any combinations thereof) in the test sample can then be determined, e.g., as described above. If helpful, determination can be done by comparing the quantity of detectable label in the antibody-analyte of interest complex to a standard curve. The standard curve can be generated using serial dilutions of analyte of interest (such as membrane-associated analyte of interest, soluble analyte of interest, fragments of soluble analyte of interest, variants of analyte of interest (membrane-associated or soluble analyte of interest) or any combinations thereof) of known concentration, where concentration is determined by mass spectroscopy, gravimetrically and by other techniques known in the art.

Optionally, the antibody-analyte of interest complex can be separated from the test sample by binding the antibody to a solid support, such as the solid supports discussed above in connection with the sandwich assay format, and then removing the remainder of the test sample from contact with the solid support.

iv. Reverse Competition Assay

In a reverse competition assay, an immobilized analyte of interest can either be sequentially or simultaneously contacted with a test sample and at least one labeled antibody. Provided herein are methods for measuring or detecting an analyte of interest present in a biological sample. In such methods, a sample droplet containing the target analyte of interest may be merged with a droplet containing a first specific binding partner that specifically binds to the target analyte of interest present in the sample and is labeled with a detectable label (such as a fluorescent label, enzymatic label, etc.) and magnetic beads to which the analyte of interest is attached. Merging creates a single droplet which may be incubated for a time sufficient to allow binding of the first specific binding partner to an analyte of interest present in the sample droplet. Optionally, the single droplet may be agitated to facilitate mixing of the sample with the first specific binding partner. Mixing may be achieved by moving the single droplet back and forth, moving the single droplet around over a plurality of electrodes, splitting a droplet and then merging the droplets, or using SAWs, and the like. Next, the single droplet may be subjected to a magnetic force to retain the beads at a location in the device while the droplet may be moved away to a waste chamber or pad and replaced with a droplet containing a second binding member. An optional wash step may be performed by moving a droplet of wash buffer to the location at which the beads are retained using the magnetic force. The beads may or may not be resuspended in the wash buffer; a magnetic force is applied to the magnetic beads and the wash buffer is transported to a waste location. After a period of time sufficient for the first specific binding partner to bind the analyte of interest bound, the magnetic force may be removed and a droplet containing the labeled beads which has a complex of the first binding member, analyte of interest may be moved over to the detection module. As explained herein, the immunoassay may be carried out in the sample preparation module. The labeled beads may be allowed to settle into the array of wells in the detection module. The beads may settle using gravitational force or by applying electric or magnetic force. Following a wash step to remove any beads not located inside the wells, the wells may be sealed by using a hydrophobic liquid.

Additionally or alternatively, the method includes contacting the sample with a binding member, wherein the binding member specifically binds to the analyte, and the binding member is labeled with a cleavable tag; contacting the sample, which may contain analyte bound to the binding member, with a immobilized analyte, wherein the immobilized analyte is immobilized on a solid support; removing binding member not bound to the immobilized analyte; cleaving the tag attached to the binding member bound to the immobilized analyte; translocating the cleaved tag through or across one or more nanopores in a layer; and assessing the tag translocating through the layer, wherein measuring the number of tags translocating through the layer measures the amount of analyte present in the sample, or detecting tags translocating through the layer detects that the analyte is present in the sample. In some embodiments, measuring the tags translocating through the layer is assessed, wherein the number of tags translocating through the layer measures the amount of analyte present in the sample. In some embodiments, detecting the tags translocating through the layer is assessed, wherein detecting tags translocating through the layer detects that the analyte is present in the sample.

Provided herein are methods for measuring or detecting an analyte present in a biological sample. The method includes contacting the sample with a binding member, wherein the binding member specifically binds to the analyte, and the binding member comprises an aptamer; contacting the sample, which may contain analyte bound to the binding member, with a immobilized analyte, wherein the immobilized analyte is immobilized on a solid support; removing binding member not bound to the immobilized analyte; dissociating the aptamer bound to the binding member that is bound to the immobilized analyte and translocating the dissociated aptamer through or across one or more nanopores in a layer; and assessing the aptamer translocating through the layer, wherein measuring the number of aptamers translocating through the layer measures the amount of analyte present in the sample, or detecting aptamers translocating through the layer detects that the analyte is present in the sample. In some embodiments, measuring the aptamers translocating through the layer is assessed, wherein the number of aptamers translocating through the layer measures the amount of analyte present in the sample. In some embodiments, detecting the aptamers translocating through the layer is assessed, wherein detecting tags translocating through the layer detects that the analyte is present in the sample.

The analyte of interest can be bound to a solid support, such as the solid supports discussed above in connection with the sandwich assay format.

The immobilized analyte of interest, test sample and at least one labeled antibody are incubated under conditions similar to those described above in connection with the sandwich assay format. Two different species analyte of interest-antibody complexes are then generated. Specifically, one of the analyte of interest-antibody complexes generated is immobilized and contains a detectable label (e.g., a fluorescent label, a tag attached with a cleavable linker, etc.) while the other analyte of interest-antibody complex is not immobilized and contains a detectable label. The non-immobilized analyte of interest-antibody complex and the remainder of the test sample are removed from the presence of the immobilized analyte of interest-antibody complex through techniques known in the art, such as washing. Once the non-immobilized analyte of interest antibody complex is removed, the amount of detectable label in the immobilized analyte of interest-antibody complex is then quantified following cleavage of the tag. The concentration of analyte of interest in the test sample can then be determined by comparing the quantity of detectable label as described above. If helpful, this can be done with use of a standard curve. The standard curve can be generated using serial dilutions of analyte of interest or analyte of interest fragment of known concentration, where concentration is determined by mass spectroscopy, gravimetrically and by other techniques known in the art.

v. One-Step Immunoassay or Capture on the Fly Assay

In a one-step immunoassay or capture on the fly assay, a solid substrate is pre-coated with an immobilization agent. The capture agent, the analyte and the detection agent are added to the solid substrate together, followed by a wash step prior to detection. The capture agent can bind the analyte and comprises a ligand for an immobilization agent. The capture agent and the detection agents may be antibodies or any other moiety capable of capture or detection as described herein or known in the art. The ligand may comprise a peptide tag and an immobilization agent may comprise an anti-peptide tag antibody. Alternately, the ligand and the immobilization agent may be any pair of agents capable of binding together so as to be employed for a capture on the fly assay (e.g., specific binding pair, and others such as are known in the art). More than one analyte may be measured. In some embodiments, the solid substrate may be coated with an antigen and the analyte to be analyzed is an antibody.

In some embodiments, a solid support (such as a microparticle) pre-coated with an immobilization agent (such as biotin, streptavidin, etc.) and at least a first specific binding member and a second specific binding member (which function as capture and detection reagents, respectively) are used. The first specific binding member comprises a ligand for the immobilization agent (for example, if the immobilization agent on the solid support is streptavidin, the ligand on the first specific binding member may be biotin) and also binds to the analyte of interest. The second specific binding member comprises a detectable label and binds to an analyte of interest. The solid support and the first and second specific binding members may be added to a test sample (either sequentially or simultaneously). The ligand on the first specific binding member binds to the immobilization agent on the solid support to form a solid support/first specific binding member complex. Any analyte of interest present in the sample binds to the solid support/first specific binding member complex to form a solid support/first specific binding member/analyte complex. The second specific binding member binds to the solid support/first specific binding member/analyte complex and the detectable label is detected. An optional wash step may be employed before the detection. In certain embodiments, in a one-step assay more than one analyte may be measured. In certain other embodiments, more than two specific binding members can be employed. In certain other embodiments, multiple detectable labels can be added. In certain other embodiments, multiple analytes of interest can be detected.

The use of a one step immunoassay or capture on the fly assay can be done in a variety of formats as described herein, and known in the art. For example the format can be a sandwich assay such as described above, but alternately can be a competition assay, can employ a single specific binding member, or use other variations such as are known.

vi. Combination Assays (Co-Coating of Microparticles with Ag/Ab)

In a combination assay, a solid substrate, such as a microparticle is co-coated with an antigen and an antibody to capture an antibody and an antigen from a sample, respectively. The solid support may be co-coated with two or more different antigens to capture two or more different antibodies from a sample. The solid support may be co-coated with two or more different antibodies to capture two or more different antigens from a sample.

Additionally, the methods described herein may use blocking agents to prevent either specific or non-specific binding reactions (e.g., HAMA concern) among assay compounds. Once the agent (and optionally, any controls) is immobilized on the support, the remaining binding sites of the agent may be blocked on the support. Any suitable blocking reagent known to those of ordinary skill in the art may be used. For example, bovine serum albumin (“BSA”), phosphate buffered saline (“PBS”) solutions of casein in PBS, Tween 20™ (Sigma Chemical Company, St. Louis, Mo.), or other suitable surfactant, as well as other blocking reagents, may be employed.

As is apparent from the present disclosure, the methods and devices disclosed herein, including variations, may be used for diagnosing a disease, disorder or condition in a subject suspected of having the disease, disorder, or condition. For example, the sample analysis may be useful for detecting a disease marker, such as, a cancer marker, a marker for a cardiac condition, a toxin, a pathogen, such as, a virus, a bacteria, or a portion thereof. The methods and devices also may be used for measuring analyte present in a biological sample. The methods and devices also may be used in blood screening assays to detect a target analyte. The blood screening assays may be used to screen a blood supply.

### 6. Surface Acoustic Wave Device, System, and Methods

Systems, device, and methods related to an integrated surface acoustic wave (SAW) sample preparation and analyte detection device are provided by the subject disclosure.

In one example, the device includes a sample preparation component, e.g., a substrate with a surface that allows for liquid or fluids to propagate across the surface thereof via manipulation by acoustic forces. In the same example, the device includes an analyte detection component configured to receive the propagated liquid and perform analyte detection on the received liquid.

“Surface acoustic waves (SAW)” and grammatical equivalents thereof as used herein refer generally to propagating acoustic waves in a direction along a surface. “Traveling surface acoustic waves” (TSAWs) enable coupling of surface acoustic waves into a liquid. In some examples, the coupling may be in the form of penetration or leaking of the surface acoustic waves into the liquid. In some examples, the surface acoustic waves are Raleigh waves. Propagation of the surface acoustic waves can be performed by streaming the surface acoustic waves through a liquid. Propagation of surface acoustic waves may be conducted in a variety of different ways and by using different materials, including generating an electrical potential by a transducer, such as a series or plurality of electrodes.

The electrodes may be patterned onto a planar substrate. In some examples, the planar substrate may be a piezoelectric layer. In some examples, the electrodes may be fabricated onto the piezoelectric layer using standard lithography and lift off/wet etching processes. The structure of the electrodes, spacing between electrodes, the number of electrodes (i.e., resolution) on the substrate may vary. In some examples, interdigitated (IDT) transducers or electrodes are used. In some examples, the sample preparation component may include a liquid. In some examples, there may be multiple layers. The different layers may have different arrangement or configuration of scattering structures for scattering surface acoustic waves. As a result, liquid droplet movement across the different layers may differ due to the varied scattering structures present.

In some examples, SAW are propagated when a single transducer or electrode is activated. In other examples, a plurality (e.g., pair) of electrodes fabricated on the substrate surface may generate two traveling SAWs propagating towards each other. In some examples, SAW displacement is activated when a radio frequency (RF) range is applied to the electrodes. Upon being activated, the electrodes or transducers emit an electric potential across the surface of the substrate, where the substrate is subjected to mechanical stress. Examples of mechanical stress are continuous contraction and expansion of the surface of the substrate. As a result of this continuous deformation of the substrate, surface acoustic waves are propagated across the surface.

Surface acoustic waves can be measured according to amplitude and frequency. Therefore, the frequency and amplitude of the electric potential generated by the electrodes is responsible for the amplitude and frequency of SAW.

Propagation of SAW may be in a linear direction. In some examples, SAW may propagate across the longitudinal length of the substrate surface. In other examples, SAW may propagate across the width of the substrate surface. In other examples, propagation of SAW may be in a non-linear direction and motion. Because fluid is a dissipative system, the response to harmonic forcing via SAW may not necessarily be harmonic.

When a TSAW contacts liquid, the liquid absorbs part of the SAW's energy and may refract it in the form of longitudinal waves. Absorption of the refracted acoustic energy induces fluid flow or propagation across the surface of the substrate. When a surface acoustic wave is propagated along the surface of the sample preparation component, the SAW may come into contact with the liquid. As a result of the liquid interacting with SAW, results in the SAW being transferred into the liquid. SAWs manipulate fluid by means of “contact free manipulation”, which is meant the liquids are propagated to the detection component by the acoustic waves leaking or penetrating into the fluid. As a result, there is a minimization of outside contamination of the biological sample or analyte.

In some examples, exemplary driving fluid actions includes pumping, mixing, jetting, etc. As a result, the liquid is propagated along the surface of the sample preparation component.

In some examples, the liquid can be dispensed as a droplet to be actuated onto the surface of the sample preparation component prior to the activation of the SAW electrodes. Droplet actuation can be used for positioning droplets and dispensing droplets onto the sample preparation component.

In other examples, instead of liquid droplet-based microfluidics, a SAW driven pump may be used to pump liquid onto the open surface. In some examples, fluid may be pumped through enclosed channels.

The liquid may be any test sample containing or suspected of containing any analyte of interest. As used herein, “analyte”, “target analyte”, “analyte of interest” refer to the analyte being measured in the methods and devices disclosed herein. The liquid droplets may also refer to particles or beads in an aqueous solution. Samples may include biological fluid samples such as, for example, blood, plasma, serum, saliva, sweat, etc.

In some examples, the liquid can be disposed as a single particle. In other examples, the liquid can be disposed as a group of particles (e.g., thousands of particles). The liquid droplets may vary according to a wide range of length scales, size (nm to mm), as well as shape.

The propagation of surface acoustic waves may also be affected by the presence of phononic structures patterned onto the surface of the sample preparation platform. These phononic structures may control the propagation of the sound acoustic waves. For example, the phononic structures may control the direction, movement, velocity of the SAW; thus, providing enhanced functionality. The phononic structures may be fabricated onto the substrate using standard lithography, lift off/wet etching processes, embossing/nanoimprint lithography, and micromachining, pressure, heat, and laser modification of the substrate to form these phononic structures. These phononic structures may assume a variety of shapes and sizes as well. In some examples, the phononic structures may be pillars, cones, or holes that form a lattice within the substrate.

### 7. Surface Acoustic Waves Sample Preparation Component

“Sample preparation component” and grammatical equivalents thereof as used herein refer to a generally planar surface on which the liquid droplets are initially dispersed upon and where steps of immunoassay as described herein may be carried out. In some examples, the substrate may be made of materials with high acoustic reflection.

In some examples, the sample preparation component includes a superstrate coupled to a substrate. In some examples, the superstrate is removably coupled to the substrate. In other examples, the superstrate is permanently coupled to the substrate. Some examples include making the substrate from a polymer-based or paper-material. The polymer-based substrate may be treated with a hydrophobic coating or fabrication may add a hydrophobic layer over the polymer-based substrate or with another substrate such that the substrate is impermeable to aqueous fluid.

In some examples, the sample preparation component may also include an assay reagent included on the superstrate. The sample preparation component further includes a superstrate coupled to a substrate.

In yet another example, the sample preparation component may include a series of scattering structures included on the superstrate. Examples of the scattering structures may include phononic structures, which are described in greater detail below.

In some examples, the substrate may be a piezoelectric material. The piezoelectric layer may be made from a composite layer, such as single crystal lithium niobate (LiNbO3). The superstrate may further include a series or plurality of electrodes or transducer. In some examples, surface acoustic waves generated by the electrodes or IDT may also be coupled into the superstrate.

In some examples, the superstrate may be made from a variety of materials, such as plastics (e.g., PET, PC, etc.).

In some examples, the superstrate may be fabricated of a material with a relatively high electromechanical coupling coefficient. In some examples, electrodes may be fabricated onto piezoelectric materials. In one example, LiNbO3 may be used as a substrate to pattern electrodes in SAW microfluidic applications. In another example, silicon may be used as a substrate material to pattern electrodes. Other examples of material applicable for fabricating a SAW-generating substrate include polycrystalline material, microcrystalline material, nanocrystalline material, amorphous material or a composite material. Other examples of material applicable for fabricating a SAW-generating substrate include ferroelectrical material, pyroelectric material, piezoelectric material or magnetostrictive material.

As described herein, the substrate is a material capable of generating surface acoustic waves and propagating acoustic waves.

In addition to the analyte or biological sample to be analyzed, the sample preparation component may also include buffer or wash fluids. In some examples, these buffer or wash fluids may facilitate the propagation of liquids across the sample preparation component and onto the detection component. In other instances, these fluids may be used to wash away any remaining liquid or biological samples once they have being positioned into the well array. Examples of such fluids include air, inert gases, hydrophobic liquids, hydrophilic liquids, oils, organic-based solvents, and high-density aqueous solutions. In certain cases, the device may be filled with a filler fluid which may be air, inert gases, hydrophobic liquids, hydrophilic liquids, oils, organic-based solvents, and high-density aqueous solutions.

In some examples, SAW induced fluidic movement can be visualized by introducing small dyes or particles into the liquid droplet.

The sample preparation surface has a surface on which the liquid may be propagated along the surface. The surface of the sample preparation surface may be any convenient surface in planar or non-planar conformation. The surface may be coated with a hydrophobic material to facilitate movement of the liquid along the surface. In some examples, the hydrophobic material may include octadecyltrichlorosilane (OTS). In other examples, the surface may be patterned to facilitate liquid movement.

In some examples, the substrate of the sample preparation surface may be elastic or flexible. The substrate on which the surface is formed upon may be elastic so that the surface is able to deform so as to facilitate the propagation of surface acoustic waves across the surface.

In certain embodiments, the surface of the substrate may include microfluidic channels to facilitate propagating fluid. In other embodiments, a microfluidic channel is included internal of the substrate to transmit fluid into the substrate.

In some examples, a cover seal may be provided over the upper surface of the substrate of the sample preparation component. In certain instances, the cover seal may prevent contamination of the liquid contents of the surface. In other instances, the cover seal may be a liquid impermeable layer. In other instances, the cover seal may be made from a flexible material such as plastics, silicon, or other type of rubber. In other instances, the cover seal may be made from a non-flexible material such as a glass or other non-flexible material. In some examples, the cover seal may be impenetrable to heat, ultraviolet light, or other electromagnetic radiation to prevent deformation of either the surface or liquid contents present on the surface.

In some examples, a suitable spacer may be positioned between the substrate and the cover seal. By “suitable spacer” as used herein, refers to an element positioned between the substrate of the sample preparation component and the cover seal. In some examples, the suitable spacer may facilitate liquid droplets to move between the surface and the cover seal. In other examples, the suitable spacer may reduce coupling between the traveling surface acoustic waves and the surface.

In the first example sample preparation component, the first substrate incorporates a material with a relatively high electromechanical coupling coefficient and having a flexible and deformable surface. For example, the first substrate may be a piezoelectric material or silicon.

In some examples, electrodes are arranged on the surface or embedded within the piezoelectric layer. The term “electrodes”, as used in this context, refers to electric circuit including a electrode, a series or plurality of electrodes (e.g., more than one), a transducer. The electrode may also be patterned into the piezoelectric layer. In some examples, the electrodes may be fabricated onto the substrate using standard lithography and lift off/wet etching processes. The structure of the electrodes, spacing between electrodes, the number of electrodes (i.e., resolution) on the substrate may vary. In some examples, interdigitated (IDT) transducers or electrodes are used. IDT is defined as a combination of a series or plurality of electrodes and a piezoelectric layer on which the series or plurality of electrodes are included on. In some examples the transducer electrode structures are formed onto the piezoelectric layer. In other examples, the transducer electrode structures are embedded within the piezoelectric layer.

In some examples, surface acoustic waves are propagated when a single transducer or electrode is activated. In other examples, a plurality (e.g., pair) of electrodes fabricated on the substrate surface may generate two traveling surface acoustic waves propagating towards each other. In some examples, surface acoustic waves displacement is activated when a radio frequency (RF) range is applied to the electrodes. Upon being activated, the electrodes or transducers emit an electric potential across the surface of the substrate, where the material is subjected to mechanical stress. Examples of mechanical stress are continuous contraction and expansion of the surface of the substrate. As a result of this continuous deformation of the substrate, surface acoustic waves are propagated across the surface.

In some examples, wavelength of surface acoustic waves is dependent upon the pitch of the transducer (IDT) or series or plurality of electrodes.

In one example, the sample preparation component may include a series of phononic structure that are included on the surface of the superstrate. The phononic structures may control the propagation of the acoustic waves. For example, the phononic structures may control the direction, movement, velocity of the surface acoustic waves. The phononic structures may assume a variety of shapes and sizes as well. In some examples, the phononic structures may be pillars, cones, or holes that form a lattice within the substrate. The pattern of phononic structures on the surface of the superstrate may be predefined based on characteristics such as resolution (e.g., number of electrodes per area on the surface), electrode size, inter-digitation of the electrodes, and/or gaps or spacing between the electrodes. In some examples, characteristics of the pattern are selected based on one or more operational uses of the droplet actuator with which the SAW sample prep component is to be associated (e.g., for use with biological and/or chemical assays). In other configurations, the pattern of electrodes may be reconfigurable to enable different patterns to suit different applications. In some examples, an increase in the size or dimensions of the series or plurality of electrodes or each individual electrode may also reduce the amount of hydrophobic material applied between adjacent electrodes. Thus, the features of the electrode pattern may maximize the surface area of the SAW platform. Furthermore, increased inter-digitation of the series or plurality of electrodes/transducers facilitates the ease with which liquid is propagated across the surface via manipulation of their electrical potentials.

In the first example sample preparation component, hydrophobic material may be applied to the series or plurality of electrodes and surface of the substrate to make the superstrate impermeable to aqueous solutions. As a result of the hydrophobic material, a liquid actuated through a droplet or fluid pump is in a beaded configuration forming a contact angle with the hydrophobic layer of the surface of the substrate. In operation, SAW acoustic waves propagate across the surface coupling to the liquid, for example by penetrating or leaking into the liquid. The amplitude or frequency of the SAW acoustic wave may control the resulting frequency and motion of the moving liquid.

In certain embodiments, the surface acoustic waves propagate along the surface of the substrate and are then coupled into the superstrate. Thereafter, the surface acoustic waves continue to propagate and are guided by phononic structures that may be formed in the superstrate.

In some examples, where SAW acoustic wave are generated by two or more electrodes, it may result in controlling the direction of the liquid that is coupled to the resulting surface acoustic waves. The direction of the propagating liquid may be in a linear direction or non-linear direction. In some examples, the propagation of the liquid droplet may be in a rolling motion. In other examples, propagation of the liquid droplet may be in a sliding motion across the surface. In some examples, where there is a lack of phononic structures on the surface, propagation of the SAW and propagation of the resulting liquid droplet are in the same direction. In other examples, where there is a presence of phononic structures on the surface, propagation of SAW and propagation of resulting liquid droplets are in opposing directions or different directions.

In some examples, the hydrophobic material is a polytetrafluoroethylene material (e.g., Teflon®) or a fluorosurfactant (e.g., FluoroPel™) applied to the surface of the superstrate.

### 8. Analyte Detection Component

In some embodiments, the analyte detection component may include an array of wells in which molecules, particles, beads, or cells may be isolated for analyte or biological sample detection purpose. TSAWs (traveling surface acoustic waves) generate acoustic streaming over the surface are across the fluid channels to push fluid (either droplets or cells) towards the well array.

The shape and geometry of the wells may vary according to the type of procedure or application required. In some examples, the wells may vary between being deep chambers to shallow chambers. The wells may be any of a variety of shapes, such as, cylindrical with a flat bottom surface, cylindrical with a rounded bottom surface, cubical, cuboidal, frustoconical, inverted frustoconical, or conical. In certain cases, the wells may include a sidewall that may be oriented to facilitate the receiving and retaining of a microbead or microparticle present in liquid droplets that have been moved over the well array. In some examples, the wells may include a first sidewall and a second sidewall, where the first sidewall may be opposite the second side wall. In some examples, the first sidewall is oriented at an obtuse angle with reference to the bottom of the wells and the second sidewall is oriented at an acute angle with reference to the bottom of the wells. The movement of the droplets may be in a direction parallel to the bottom of the wells and from the first sidewall to the second sidewall. The array of wells may have sub-femtoliter volume, femtoliter volume, sub-nanolitre volume, nanolitre volume, sub-microliter volume, or microliter volume. For example the array of wells may be array of femtoliter wells, array of nanoliter wells, or array of microliter wells. In certain embodiments, the wells in an array may all have substantially the same volume. The array of wells may have a volume up to 100 μl, e.g., about 0.1 femtoliter, 1 femtoliter, 10 femtoliter, 25 femtoliter, 50 femtoliter, 100 femtoliter, 0.5 pL, 1 pL, 10 pL, 25 pL, 50 pL, 100 pL, 0.1 nL, 1 nL, 10 nL, 25 nL, 50 nL, 500 nL, 0.1 microliter, 1 microliter, 10 microliter, 25 microliter, 50 microliter, or 100 microliter.

In certain cases, the sample preparation component and the analyte detection component may be fabricated from a single planar surface using, for example, a continuous web-fed manufacturing process. In such an example, the sample preparation component and the digital analyte detection component may be positioned adjacent to each other.

In some examples, the sample preparation component may include a sample inlet. By “sample inlet” as used herein, refers to a tubular member, channel, or pipe for introducing liquid to the sample preparation component. For example, the sample inlet may introduce a biological sample onto the surface of the substrate. In other example, the sample inlet may introduce a biological sample internally within the substrate.

In other examples, the sample preparation component and the digital analyte detection component may be positioned over one another in a stacked configuration, separated by a space for droplet manipulation. In the example of the sample preparation component being positioned over the analyte detection component in a stacked configuration or vice versa (the analyte detection component being positioned over the sample preparation component), an inlet or channel may be positioned between the two components. The inlet or channel may direct a sample or analyte between the two components.

Phononic structures may be fabricated or included on the superstrate of the sample preparation component. In certain cases, the phononic structures are imprinted or embossed onto the superstrate. In such examples, the embossing or imprinting of the phononic structures is in a single step. In other examples, it may be multiple steps. Imprinting or embossing of phononic structures may be through the combination of an application of pressure, heat, or ultraviolet light in the presence of a mold, mask, or pattern. In one example, pressure elicited from a mold onto the superstrate may induce deformation of the a surface of the superstrate.

After the phononic structures are included on the superstrate, it may be cured for a sufficient period of time to allow for hardening or deformation of the phononic structures. In addition, the phononic structures may be subject to reagents that modify the physical properties of the phononic structures.

In some examples, the reagents for analyte detection may be printed during fabrication of the integrated sample preparation and analyte detection device in a dehydrated form. Rehydration of the reagents occurs through use of a sample or buffer.

In some examples, the array of wells includes individual well chambers, with each well chamber having a first end and a second end. In one example, the first end of the well may be open, while the second end of the well is closed. In other examples, both the first end of the wells and the second end of the well chambers are closed. Closure of the first end of the well chambers may be through both a permanent closure mechanism and a temporary closure mechanism. By “permanent” as used herein is meant that the closure mechanism is intended to remain a fixture of the chamber of the well. By “temporary” as used herein is meant that the closure mechanism can be removed without affecting the structure, integrity, or rigidity of the closure mechanism. In some aspects, the closure of the well chamber first end may be through a combination of a permanent and a temporary closure mechanism. In one example, the temporary closure mechanism may be a liquid, such as an oil fluid, that can fill the first end of the well chamber. In certain examples, the oil drop may fill the first well end after an analyte, biological sample, or analyte related detectable label has been previously deposited into the well. In other examples, the oil drops may be closure of the first end of the well regardless of the presence of an analyte or biological sample within the well.

The array of wells has a pattern of well chambers (e.g., the formation of wells in the array) suitable for receiving a plurality of labels, beads, labeled beads, tags, and the like. The pattern of the array of the wells may vary according to resolution and spacing between well chambers.

In some examples, the pattern of the well array can be fabricated using nanoimprint lithography. In other examples, the pattern of the well array can be fabricated through a combination of any one of molding, pressure, heat, or laser.

The size of the well array may vary. In some examples, the well array may be fabricated to have individual well chambers with a diameter of 100 nm and with a periodicity of 500 nm.

In some examples the well array may be substantially as described in the section related to digital microfluidics and detection module.

In some examples, detection of the analyte or biological sample of interest may occur through optical signal detection. For example, shining an excitation light (e.g., laser) in order to measure the signal intensity result. In other examples, the analyte desired may be detected by measuring an optical signal emanating from each well chamber and quantified by quantifying the result. For example, the number of positive counts (e.g., wells) is compared to the number of negative counts (e.g., wells) to obtain a digital count. Alternately or in addition, a signal correlated to analyte concentration may be measured (analog quantitation). A variety of signals from the wells of the device may be detected. Exemplary signals include fluorescence, chemiluminescence, colorimetric, turbidimetric, etc.

### 9. Adjacent Configuration of Sample Preparation and Analyte Detection Device

In some embodiments, the array of wells is positioned on the same superstrate as the sample preparation component. In some examples, the superstrate and the array of wells may be positioned on a first substrate. The first substrate may be divided into a first portion at which droplets to be analyzed are initially disposed and a second portion towards which the droplets are moved for analyte detection. The superstrate may be present on the first portion of the first substrate and the array of wells may be positioned on a second portion of the first substrate. As such the superstrate which forms the sample preparation component and the array of wells which form the analyte detection component may be directly adjacent. As used herein, the term “directly adjacent” refers to there being a lack of object separating or dividing the sample prep component and the array of wells. In examples, where the sample prep component and array of wells are directly adjacent to each other, the propagation of the liquid droplets across the surface of the sample prep component is seamlessly transitioned onto the surface of the array of wells. In other examples, the array of wells is positioned indirectly adjacent to the sample prep component. As used herein, the term “indirectly adjacent” refers to there being an object or element separating or dividing the sample prep component.

In some examples, to facilitate liquid movement and improve position accuracy of the droplets into the individual well chambers, the substrate surface of the sample preparation component may be patterned or coated with a hydrophilic material. In other examples, reagents such as oils and emulsions may be used to seal the well arrays.

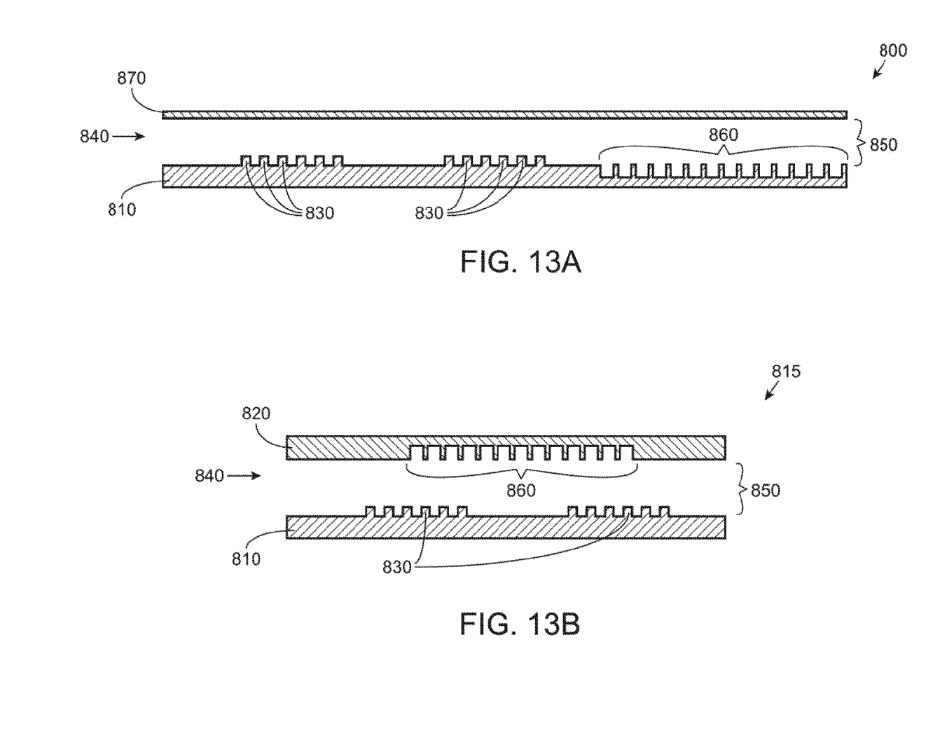

FIG. 13A illustrates a side view of a sample preparation component positioned adjacent to an analyte detection component. As shown in FIG. 13A, the sample preparation component includes a superstrate 810. The superstrate 810 includes a series of phononic structures 830. The size, shape, and dimensions of the phononic structures may vary. As shown in FIG. 13A, the sample preparation component is positioned to be directly adjacent to the analyte detection component comprising an array of wells 860. Where these components are positioned adjacent to each other, liquid propagated across the surface of the superstrate 810 can be collected into individual well chambers on the well array 860. In this particular example, a sample inlet channel 840 is positioned between the superstrate 810 and the cover 870. The superstrate 810 and the cover 870 are separated by space/gap 850 defining a space where liquid droplets are manipulated (e.g., merged, split, agitated, etc.). However, in other examples, a sample inlet channel is not included. The size, dimensions, and variations of the sample inlet channel may vary. For example, the sample inlet channel may introduce a fluid onto the surface of the superstrate 810. In other examples, the sample inlet channel may introduce a fluid internally within the superstrate 810.

In some examples, a cover seal may be provided over the surface of the sample preparation component. In certain instances, the cover seal may prevent contamination of the liquid contents of the surface. In other instances, the cover seal is a liquid impermeable layer. In other instances, the cover seal is made from a flexible material such as plastics, silicon, or other type of rubber. In other instances, the cover seal is made from a non-flexible material such as a glass or other non-flexible material. In some examples, the cover seal may be impenetrable to heat, ultraviolet light, or other electromagnetic radiation to prevent deformation of either the surface or liquid contents present on the surface of the sample preparation component.

In some examples, a heat sink may be provided in order to dissipate the heat generated by generation of surface acoustic waves across the surface of the substrate.

### 10. Stacked Configuration of Sample Preparation and Analyte Detection Device

In some embodiments, the array of wells (detection component) is positioned over the sample preparation component separated by a space where the droplets are manipulated. In some examples, an inlet or channel may be positioned between the two components. The inlet or channel may direct a sample or analyte between the two components.

In some examples, the well array may be imprinted or embossed onto a first substrate and the phononic structure may be present on a superstrate positioned in a spaced apart manner from the first substrate. The superstrate may be supported by a second substrate.

In some examples, the step of coupling the first substrate that includes the array of wells with the superstrate may be facilitated with the use of a bonding agent, adhesive agent, tapes, glues, soldering, or other affixing agent capable of coupling the array of wells to the superstrate. In other examples, the step of coupling the array of wells onto the phononic structures of the sample prep component may be achieved through use of mechanical fasteners, fixers, bolts, and other mechanical components such as latches. In other examples, the step of coupling the array of wells onto the phononic structures of the sample prep component may occur through setting and positioning the array of wells over the phononic structures of the sample prep component. In some examples, the phononic structures of the substrate may be in parallel orientation to the well array component.

The spacing between the phononic structures of the superstrate and the well array may vary according to the type of application to be performed, the size of the liquid droplet being actuated onto the surface of the substrate, the size, shape and arrangement of phononic structures, the size of the sample channel/inlet, and the amplitude of the surface acoustic waves propagating across the surface.

FIG. 13B illustrates a side view of a stacked configuration of a superstrate and well array component. As shown in FIG. 13B, the superstrate 810 includes a series of phononic structures 830. The phononic structures 830 are arranged in an array of repeating structural elements. The size, shape, and dimensions of the phononic structures may vary. In this example, an array of wells 860 is also present. In this example, the array of wells 860 is positioned directly over the superstrate. As illustrated in FIG. 13B, the opening of the wells may be directly opposite the phononic structures. In this particular example, a sample inlet channel 840 is positioned between the well array and the superstrate. However, in other examples, a sample inlet channel is not included. The size, dimensions, and variations of the sample inlet channel may vary. For example, the sample inlet channel may introduce a fluid onto the surface of the superstrate 810. In other examples, the sample inlet channel may introduce a fluid internally within the superstrate 810. The substrate 820 that includes the array of wells 860 is positioned in a spaced apart manner from the superstrate 810 and is separated from the superstrate 810 by a gap/space 850.

The array of wells as shown in FIGS. 13A-B can vary in size and/or shape. For example, the well array can be substantially shallow or deep. The resolution of the well array is affected by the spacing between each well chamber. For example, minimal spacing between the well chambers allows for a greater number of wells to collect a greater number of analytes or biological samples. In some examples, well array may be formed via ablating the substrate. The pattern of the well array may be formed by using a special pattern or special mask, and subjecting the mask to laser ablation.

### 11. Fabricating Surface Acoustic Wave Sample Preparation and Detection Device

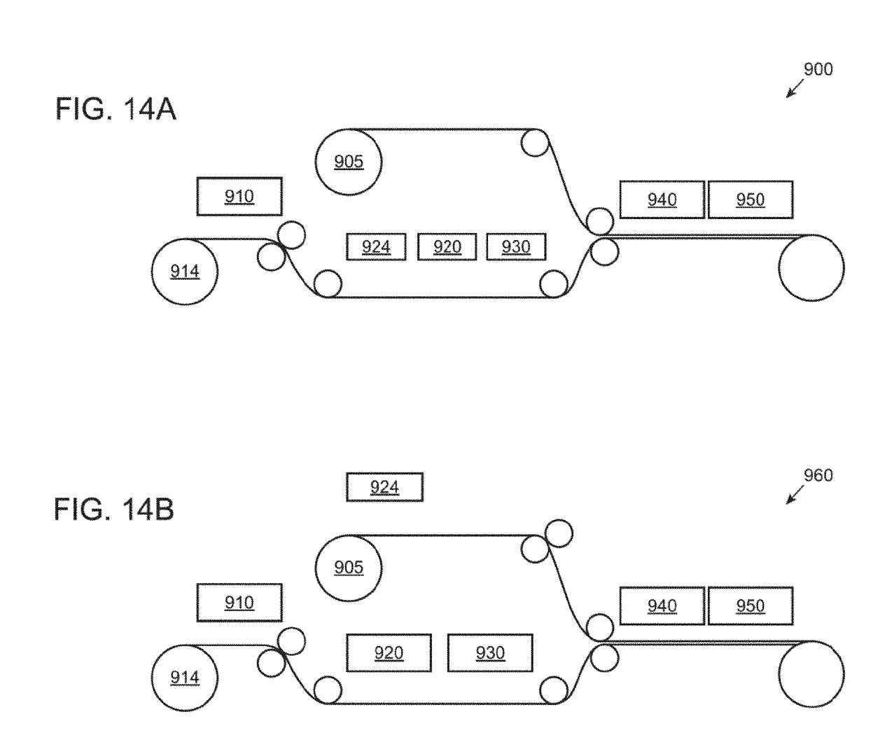

FIGS. 14A-14B illustrate exemplary methods for separately fabricating the SAW devices disclosed in the foregoing sections. FIG. 14A illustrates that the sample preparation component and well array component are positioned adjacent to each other by fabricating the phononic structures and the array of wells on a single base substrate. A superstrate (e.g., see FIG. 13A, superstrate 810) is placed on an assembly line 900. Propagation of the superstrate along the assembly line 900 is facilitated by a conveyer belt-like mechanism utilizing a series of rollers. A roll 914 of the superstrate is unspooled and is subjected to an embossing unit 910, which subjects the material to intense heat, pressure, or ultraviolet light in order to form phononic structures on the superstrate or embedded within the superstrate using a mold. The array of wells is created using laser ablation 924. Thereafter, the superstrate passes through a plurality of rollers to a surface treatment component 920, which modifies properties of the superstrate. Thereafter, the superstrate passes through an inkjet printer 930 that deposits assay reagents on the superstrate. In some examples, the resulting structures may be subject to a curing step. In other examples, the resulting structures may be subjected to surface treatment to modify their physical properties, for example, incorporating functionalized reagents required for assay protocols. A cover (e.g., FIG. 13A, cover 870) is then laminated 940 onto the superstrate. The cover may be provided as a roll 905 which is unspooled and moved using rollers. Prior to placing the cover on the superstrate, a suitable spacer is placed between the superstrate and the cover to enable liquid droplets to move between the two surfaces. The assembled structure may be diced 950 to generate individual devices.

FIG. 14B illustrates an exemplary method for fabricating the device depicted in FIG. 13B. A roll 914 of superstrate (e.g., see FIG. 13B, superstrate 810) is subjected to a fabrication process using an embossing unit 910, which subjects the superstrate to intense heat, pressure, or ultraviolet light in order to form repeating structural elements of phononic structures in the presence of a mold. Thereafter, the superstrate passes through a surface treatment component 920 to modify properties of the superstrate surface. Thereafter, the superstrate passes through an inkjet printer 930, to deposit assay reagents in situ. To form the detection module comprising an array of wells, a roll 906 of a first substrate (e.g., see FIG. 13B, substrate 820) is subjected to laser ablation 924. At the lamination unit 940, both the superstrate and the first substrate containing well array are combined together and subsequently bonded in a spaced apart configuration. As a result, the superstrate and the substrate are aligned vertically within a stack configuration. Thereafter, the stacked substrates are subject to a dicing component 950, for example, to generate individual devices.

The devices and systems and method described herein that propagate droplet actuation may also include a variety of other forces that affect droplet actuation. For example, movement of the droplets across the surfaces may include electric field-mediated forces, electrostatic actuation (such as electrical actuation), electrowetting, dielectrophoresis, electric field gradients or electrode-mediated forces. In embodiments where a combination of surface acoustic waves and digital microarray electrodes are used for droplet manipulation the SAW devices described herein may include a series or plurality of electrodes.

The integrated devices disclosed herein may be used to prepare a variety of samples, such as biological sample, for detection of an analyte of interest. In certain cases, the device may be used for carrying out digital immunoassay and detect presence or absence of particles/beads that are correlated to the presence or absence of an analyte.

### 12. Counting and Data Analysis

The number of translocation events can be determined qualitatively or quantitatively using any routine techniques known in the art. In some embodiments, the number of translocation events can be determined by first calculating the anticipated current change found in a double stranded DNA translocation event under experimental test conditions using the equation:

$$Δ G = \frac{σπ {d}_{DNA}^{2}}{4 L} , \qquad ( S1 )$$

as referenced in Kwok et al., “Nanopore Fabrication by controlled Dielectric Breakdown” Supplementary Information Section 8 and Kwok, H.; Briggs, K.; and Tabard-Cossa, V.; “Nanopore Fabrication by Controlled Dielectric Breakdown”—PLoS ONE 9(3): e92880 (2014). Using this anticipated current blockage value, the binary file data of the experimental nanopore output can be visually or manually scanned for acceptable anticipated current blockage events. Using these events, the Threshold and Hysteresis parameters required for the CUSUM nanopore software can be applied and executed. The output from this software can be further analyzed using the cusumtools readevents.py software and filtering blockage events greater than 1000 pA (as determined from the first calculation). The flux events, time between events and other calculations can be determined from the readevents.py analysis tool. Additional calculations can be made on the CUSUM generated data using JMP software (SAS Institute, Cary, N.C.). Other methods of threshold settings for data analysis known in the art can be used.

### 13. Qualitative Analysis

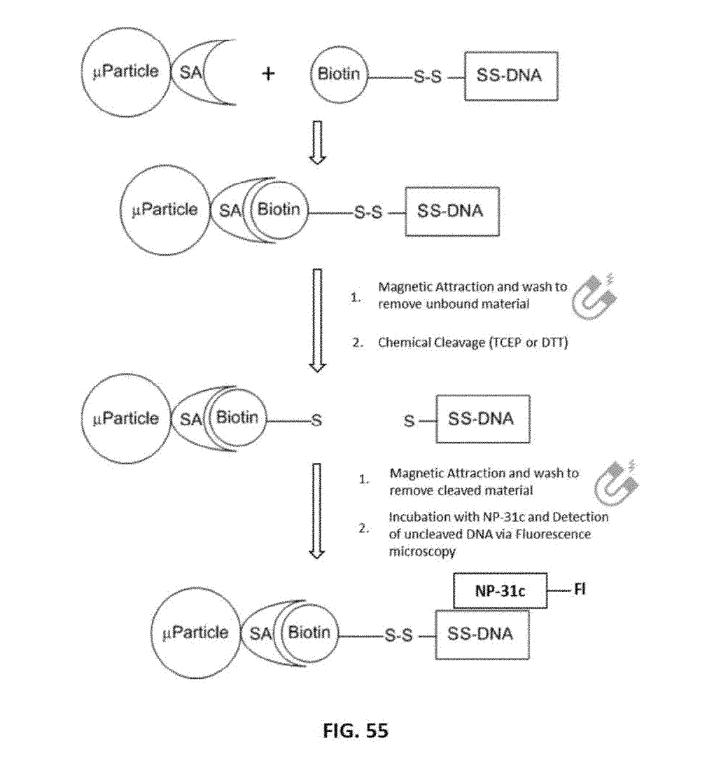

A qualitative assay can be conducted using the methods and process of steps as described herein. A direct assay can be conducted using the cleavable linker conjugate, as described in Example 15, with a thiol based cleavage step, as shown in FIG. 55. It is understood that other cleavable linker approaches to conducting such an assay may also include, but are not limited to, various other methods of cleavage of a linker so as to allow for the counting of various tags, as described herein. Additionally, aptamers can be employed. For example, such other alternative cleavage methods and/or reagents in addition to the method described in Example 15 can include those described in Example 14, Example 16, Example 17, Example 18 and Example 19, in addition to other cleavage methods described herein and known to those skilled in the art. It is also understood that while the assay format demonstrated in this Example (Example 28) represents a direct assay, other formats such as sandwich immunoassay formats and/or various competitive assay formats, and including capture on the fly formats, such as are known to those skilled in the art, can be implemented as well to conduct an assay using the described methods.

For example, the sandwich immunoassay format for the detection of TSH (thyroid stimulating hormone), as described in Example 3, demonstrated the ability to conduct such an assay on a low cost DMF chip. Additionally, a number of various bioconjugation reagents useful for the generation of immunoconjugate or other active specific binding members having cleavable linkers can be synthesized using various heterobifunctional cleavable linkers such as those described in Example 8, Example 9, Example 10, Example 11, Example 12 and Example 13, in addition to other cleavable linkers that are otherwise known to those skilled in the art. Immunoconjugates useful for the practice of the present invention can be synthesized by methods such as those described in Example 10, Example 11, Example 12 and Example 13 as well as by other methods known to those skilled in the art. Additionally, Example 2 shows the functionality of various fluidic droplet manipulations on a low cost chip that can facilitate various steps needed to carry out various assay formats including sandwich and competitive assay formats, and including capture on the fly formats, as well as other variations thereof known to those skilled in the art. Example 21 shows the fabrication of a nanopore that can be used to count cleavable label in an assay but it is understood that other methods for nanopore fabrication known to those skilled in the art can also be used for this purpose. Example 14 also represents another construct useful for the conduct of an assay where a cleavage is effected, thus leading to a countable label being released so as to be countable using the nanopore counting method, as described within this example. This construct and others that would be apparent to those skilled in the art can be used in an assay as described herein. Example 26 shows generally how counting can be done so as to be able to measure translocation events relating to the presence of a variety of labels traversing the nanopore. FIG. 59 shows the concept of thresholding of the signal so as to be able to manipulate the quality of data in a counting assay. FIG. 58 shows qualitative assay data that is representative of the type of data that can be used to determine the presence of an analyte using such assay methods as described within this example. It is also understood that while dsDNA was used as a label in this particular example, other labels, such as the label described in Example 12 and/or Example 26 can also be utilized, including, but not limited to nanobeads, dendrimers and the like. Moreover, other known labels also can be employed. Such constructs as needed to generate appropriate reagents can be synthesized through various examples described herein in this application, or otherwise via methods known to those skilled in the art.

### 14. Quantitative Analysis

A quantitative assay can be conducted using the methods and process of steps as described herein. A direct assay can be conducted using the cleavable linker conjugate, as described in Example 15, with a thiol based cleavage step, and as shown in FIG. 55. It is understood that other cleavable linker approaches to conducting such an assay may also include, but are not limited to, various other methods of cleavage of a linker so as to allow for counting of various tags using a nanopore, as described herein. Additionally, aptamers can be employed. For example, such other cleavage methods in addition to the method described in Example 15 can include, but is not limited to, those described in Example 16, Example 17, Example 18 and Example 19, in addition to other methods described herein and known to those skilled in the art. It is also understood that while the assay format demonstrated in this Example (Example 29) represents a direct assay, other formats such as sandwich immunoassay formats and/or various competitive assay formats, and including capture on the fly formats, such as are known to those skilled in the art, can be implemented as well to conduct an assay.

For example, the sandwich immunoassay format for the detection of TSH (thyroid stimulating hormone), as described in Example 3, demonstrated the ability to conduct such an assay on a low cost DMF chip. Additionally, a number of various bioconjugation reagents useful for the generation of immunoconjugate or other active specific binding members having cleavable linkers can be synthesized by those skilled in the art using various heterobifunctional cleavable linkers and conjugates synthesized by methods such as those described in Example 8, Example 9, Example 10, Example 11, Example 12 and Example 13, in addition to other cleavable linkers or conjugates that could be synthesized by methods that are known to those skilled in the art. Additionally, Example 2 shows the functionality of various fluidic droplet manipulations on a low cost chip that can facilitate various steps needed to carry out various assay formats including sandwich and competitive assay formats, and including capture on the fly formats, as well as other variations thereof known to those skilled in the art. Example 14 also represents another construct useful for the conduct of an assay where a cleavage is effected, thus leading to a countable label being released so as to be countable using the nanopore counting method as described within this example. This construct as well as other that would be apparent to those skilled in the art can be used in an assay as described herein.

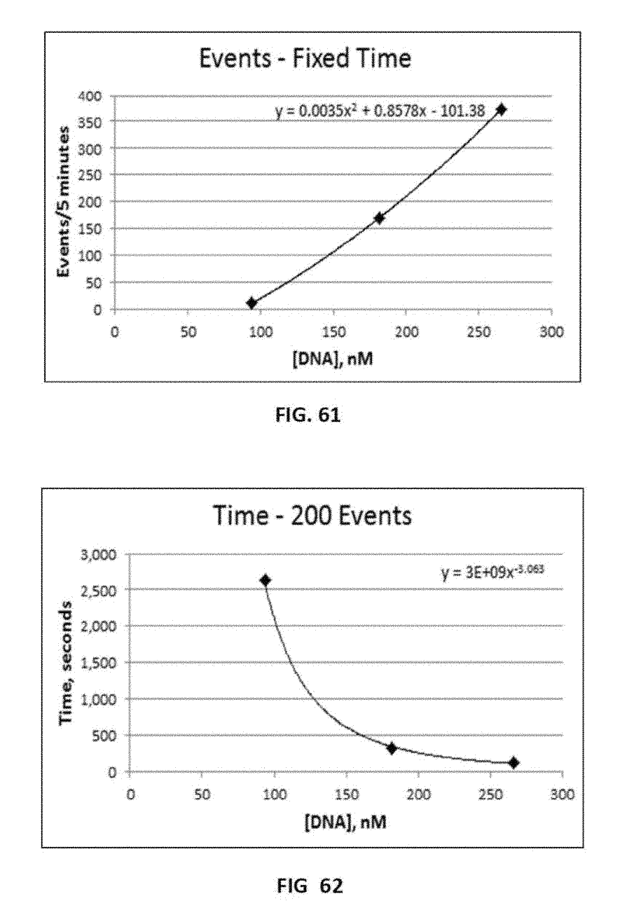

Example 26 shows generally how counting can be performed so as to be able to measure translocation events relating to the presence of a label traversing the nanopore. FIG. 59 shows the concept of thresholding of the signal so as to be able to manipulate the quality of data in a counting assay. FIGS. 61, 62 and 63 show quantitative assay data output that is representative of the type of data that can be used to determine the amount of an analyte using such assay methods as described within this example. FIG. 64 shows a standard curve generated from a construct that has been cleaved using a chemical method. It is also understood that while dsDNA was used as a label in this particular example, other labels, such as the label described in Example 12, can also be utilized, including, but not limited to, nanobeads, dendrimers and the like. Moreover, other known labels also can be employed. Such constructs as needed to generate appropriate reagents can be synthesized as described herein, or via methods known to those skilled in the art.

### 15. Kits and Cartridges

Also provided herein is a kit for use in performing the above-described methods with or without the disclosed device. The kit may include instructions for analyzing the analyte with the disclosed device. Instructions included in the kit may be affixed to packaging material or may be included as a package insert. The instructions may be written or printed materials, but are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, “instructions” may include the address of an internet site that provides the instructions.

The kit may include a cartridge that includes a microfluidics module with a built-in analyte detection, as described herein. In some embodiments, the microfluidics and analyte detection may be separate components for reversible integration together or may be fully or irreversibly integrated in a cartridge. The cartridge may be disposable. The cartridge may include one or more reagents useful for practicing the methods disclosed above. The cartridge may include one or more containers holding the reagents, as one or more separate compositions, or, optionally, as admixture where the compatibility of the reagents will allow. The cartridge may also include other material(s) that may be desirable from a user standpoint, such as buffer(s), a diluent(s), a standard(s) (e.g., calibrators and controls), and/or any other material useful in sample processing, washing, or conducting any other step of the assay. The cartridge may include one or more of the specific binding members described above.

Alternatively or additionally, the kit may comprise a calibrator or control, e.g., purified, and optionally lyophilized analyte of interest or in liquid, gel or other forms on the cartridge or separately, and/or at least one container (e.g., tube, microtiter plates or strips) for use with the device and methods described above, and/or a buffer, such as an assay buffer or a wash buffer, either one of which can be provided as a concentrated solution. In some embodiments, the kit comprises all components, i.e., reagents, standards, buffers, diluents, etc., which are necessary to perform the assay. The instructions also can include instructions for generating a standard curve.

The kit may further comprise reference standards for quantifying the analyte of interest. The reference standards may be employed to establish standard curves for interpolation and/or extrapolation of the analyte of interest concentrations. The kit may include reference standards that vary in terms of concentration level. For example, the kit may include one or more reference standards with either a high concentration level, a medium concentration level, or a low concentration level. In terms of ranges of concentrations for the reference standard, this can be optimized per the assay. Exemplary concentration ranges for the reference standards include but are not limited to, for example: about 10 fg/mL, about 20 fg/mL, about 50 fg/mL, about 75 fg/mL, about 100 fg/mL, about 150 fg/mL, about 200 fg/mL, about 250 fg/mL, about 500 fg/mL, about 750 fg/mL, about 1000 fg/mL, about 10 pg/mL, about 20 pg/mL, about 50 pg/mL, about 75 pg/mL, about 100 pg/mL, about 150 pg/mL, about 200 pg/mL, about 250 pg/mL, about 500 pg/mL, about 750 pg/mL, about 1 ng/mL, about 5 ng/mL, about 10 ng/mL, about 12.5 ng/mL, about 15 ng/mL, about 20 ng/mL, about 25 ng/mL, about 40 ng/mL, about 45 ng/mL, about 50 ng/mL, about 55 ng/mL, about 60 ng/mL, about 75 ng/mL, about 80 ng/mL, about 85 ng/mL, about 90 ng/mL, about 95 ng/mL, about 100 ng/mL, about 125 ng/mL, about 150 ng/mL, about 165 ng/mL, about 175 ng/mL, about 200 ng/mL, about 225 ng/mL, about 250 ng/mL, about 275 ng/mL, about 300 ng/mL, about 400 ng/mL, about 425 ng/mL, about 450 ng/mL, about 465 ng/mL, about 475 ng/mL, about 500 ng/mL, about 525 ng/mL, about 550 ng/mL, about 575 ng/mL, about 600 ng/mL, about 700 ng/mL, about 725 ng/mL, about 750 ng/mL, about 765 ng/mL, about 775 ng/mL, about 800 ng/mL, about 825 ng/mL, about 850 ng/mL, about 875 ng/mL, about 900 ng/mL, about 925 ng/mL, about 950 ng/mL, about 975 ng/mL, about 1000 ng/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 200 μg/mL, about 300 μg/mL, about 400 μg/mL, about 500 μg/mL, about 600 μg/mL, about 700 μg/mL, about 800 μg/mL, about 900 μg/mL, about 1000 μg/mL, about 2000 μg/mL, about 3000 μg/mL, about 4000 μg/mL, about 5000 μg/mL, about 6000 μg/mL, about 7000 μg/mL, about 8000 μg/mL, about 9000 μg/mL, or about 10000 μg/mL.

Any specific binding members, which are provided in the kit may incorporate a tag or label, such as a fluorophore, enzyme, aptamer, dendrimer, bead, nanoparticle, nanobead, microparticle, microbead, polymer, protein, biotin/avidin label, or the like, or the kit can include reagents for labeling the specific binding members or reagents for detecting the specific binding members and/or for labeling the analytes or reagents for detecting the analyte. If desired, the kit can contain one or more different tags or labels. The kit may also include components to elicit cleavage, such as a cleavage mediated reagent. For example, a cleavage mediate reagent may include a reducing agent, such as dithiothreitol (DTT) or tris(2-carboxyethyl)phosphine) TCEP. The specific binding members, calibrators, and/or controls can be provided in separate containers or pre-dispensed into an appropriate assay format or cartridge. The tag may be detected using the disclosed device.

The kit may include one or more specific binding members, for example, to detect one or more target analytes in the sample in a multiplexing assay. The number of different types of specific binding members in the kit may range widely depending on the intended use of the kit. The number of specific binding members in the kit may range from 1 to about 10, or higher. For example, the kit may include 1 to 10 specific binding members, 1 to 9 specific binding members, 1 to 8 specific binding members, 1 to 7 specific binding members, 1 to 6 specific binding members, 1 to 5 specific binding members, 1 to 4 specific binding members, 1 to 3 specific binding members, 1 to 2 specific binding members, 2 to 10 specific binding members, 2 to 9 specific binding members, 2 to 8 specific binding members, 2 to 7 specific binding members, 2 to 6 specific binding members, 2 to 5 specific binding members, 2 to 4 specific binding members, 3 to 10 specific binding members, 3 to 9 specific binding members, 3 to 8 specific binding members, 3 to 7 specific binding members, 3 to 6 specific binding members, 3 to 5 specific binding members, 3 to 4 specific binding members, 4 to 10 specific binding members, 4 to 9 specific binding members, 4 to 8 specific binding members, 4 to 7 specific binding members, 4 to 6 specific binding members, 5 to 10 specific binding members, 5 to 9 specific binding members, 5 to 8 specific binding members, 5 to 7 specific binding members, 5 to 6 specific binding members, 6 to 10 specific binding members, 6 to 9 specific binding members, 6 to 8 specific binding members, 6 to 7 specific binding members, 7 to 10 specific binding members, 7 to 9 specific binding members, 7 to 8 specific binding members, 8 to 10 specific binding members, 8 to 9 specific binding members, or 9 to 10 specific binding members. Each of the one or more specific binding members may bind to a different target analyte and each specific binding member may be labeled with a different detectable label, tag and/or aptamer. For example, the kit may include a first specific binding member that binds to a first target analyte, a second specific binding member that binds to a second target analyte, a third specific binding member that binds to a third target analyte, etc. and the first specific binding member is labeled with a first detectable label, tag and/or aptamer, the second specific binding member is labeled with a second detectable label, tag and/or aptamer, the third specific binding member is labeled with a third detectable label, tag and/or aptamer, etc. In addition to the one or more specific binding members, the kits may further comprise one or more additional assay components, such as suitable buffer media, and the like. The kits may also include a device for detecting and measuring the tag and/or an aptamer, such as those described herein. Finally, the kits may comprise instructions for using the specific binding members in methods of analyte detection according to the subject invention, where these instructions for use may be present on the kit packaging and/or on a package insert.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components. One or more of the components may be in liquid form.

The various components of the kit optionally are provided in suitable containers as necessary. The kit further can include containers for holding or storing a sample (e.g., a container or cartridge for a urine, saliva, plasma, cerebrospinal fluid, or serum sample, or appropriate container for storing, transporting or processing tissue so as to create a tissue aspirate). Where appropriate, the kit optionally also can contain reaction vessels, mixing vessels, and other components that facilitate the preparation of reagents or the test sample. The kit can also include one or more sample collection/acquisition instruments for assisting with obtaining a test sample, such as various blood collection/transfer devices such as microsampling devices, micro-needles, or other minimally invasive pain-free blood collection methods; blood collection tube(s); lancets; capillary blood collection tubes; other single fingertip-prick blood collection methods; buccal swabs, nasal/throat swabs; 16-gauge or other size needle, circular blade for punch biopsy (e.g., 1-8 mm, or other appropriate size), surgical knife or laser (e.g., particularly hand-held), syringes, sterile container, or canula, for obtaining, storing or aspirating tissue samples; or the like. The kit can include one or more instruments for assisting with joint aspiration, cone biopsies, punch biopsies, fine-needle aspiration biopsies, image-guided percutaneous needle aspiration biopsy, bronchoaveolar lavage, endoscopic biopsies, and laparoscopic biopsies.

If the tag or detectable label is or includes at least one acridinium compound, the kit can comprise at least one acridinium-9-carboxamide, at least one acridinium-9-carboxylate aryl ester, or any combination thereof. If the tag or detectable label is or includes at least one acridinium compound, the kit also can comprise a source of hydrogen peroxide, such as a buffer, solution, and/or at least one basic solution. If desired, the kit can contain a solid phase, such as a magnetic particle, bead, membrane, scaffolding molecule, film, filter paper, disc, or chip.

If desired, the kit can further comprise one or more components, alone or in further combination with instructions, for assaying the test sample for another analyte, which can be a biomarker, such as a biomarker of a disease state or disorder, such as infectious disease, cardiac disease, metabolic disease, thyroid disease, etc.

The present invention has multiple aspects, illustrated by the following non-limiting examples.

### EXAMPLES

### Example 1

### Fabrication of Low-Cost DMF Chip

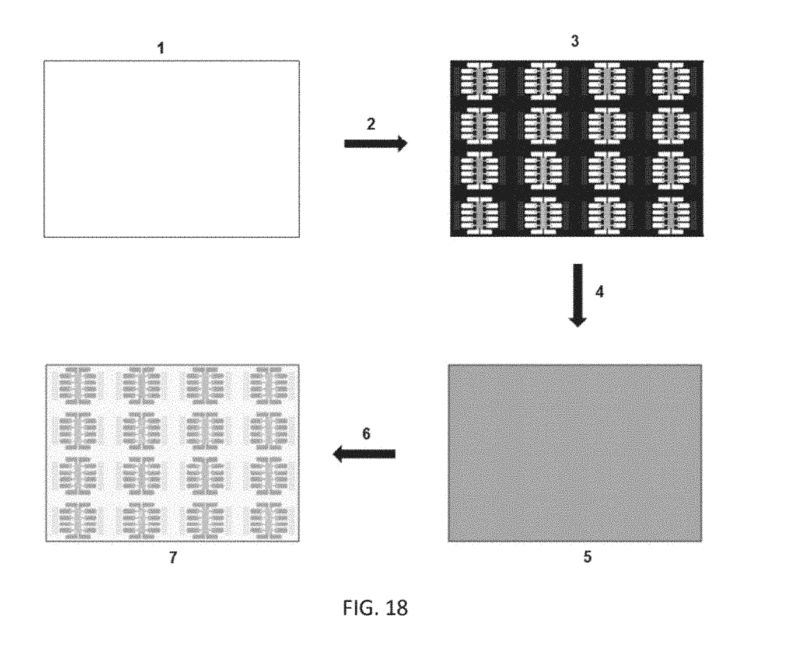

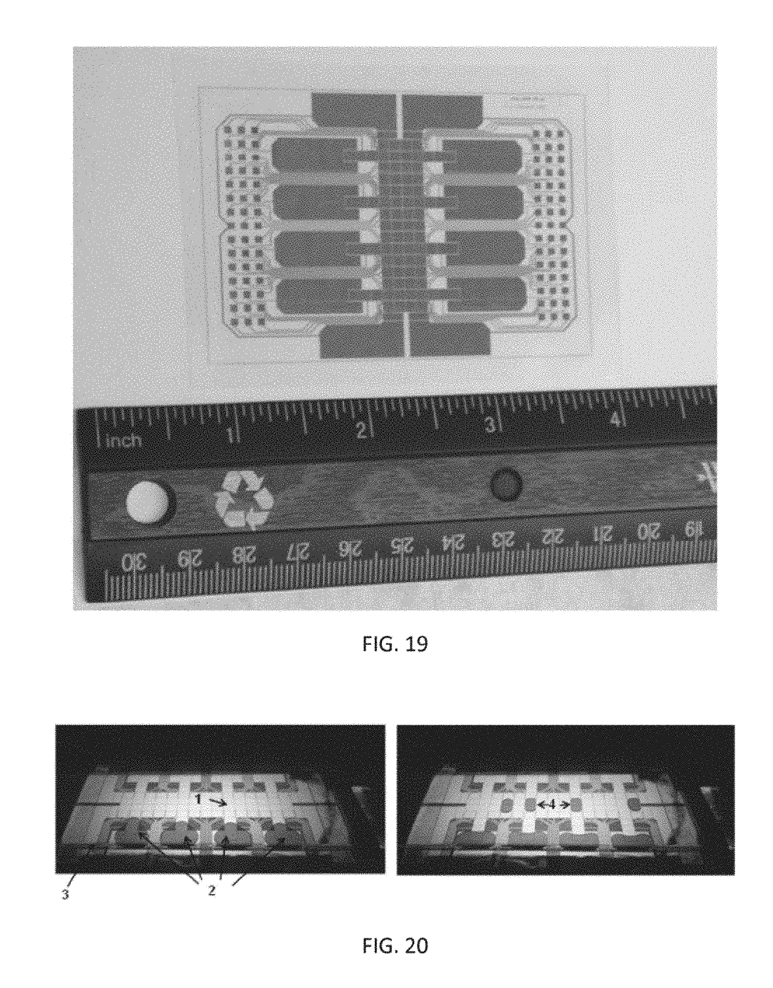

Low-cost flexible DMF chips were fabricated using roll-to-roll (R2R) flexographic printing combined with a wet lift-off process for electrode patterning. A schematic of the fabrication process is depicted in FIG. 18. A roll of Melinex ST506 polyethylene terephthalate (PET) 5.0 mil substrate (1) was used as the starting material for DMF electrode printing. A layer of yellow ink (Sun Chemical) was flexo-printed (2) on the PET substrate using a 1.14 mm thick printing plate (Flint MCO3) at a rate of 10 m/minute using an ink transfer volume of 3.8 m1/m2 on an Anilox roller assembly. A negative image of the DMF electrode pattern results from the flexo printing step (3). Prior to metal deposition, the ink was dried two times in a hot air oven (2×100° C.). An EVA R2R Metal Evaporator was used to deposit a layer of silver metal onto the printed PET substrate to form a uniform coating of silver at a thickness of 80 nm (4). The metalized ink-film substrate (5) was subjected to a wet lift-off process using a combination of acetone plus ultrasound in a sonication bath at a speed of 1 m/minute (6). This chemical/physical treatment allows the silver-ink layer to dissolve, while keeping the silver-only layer intact. Removal of the ink-silver layer resulted in a DMF printed electrode pattern consisting of 80 actuation electrodes (2.25×2.25 mm) with either 50 or 140 μm electrode gap spacing (7). As a QC check, a total of 80-90 random chips from a single roll were visually inspected for electrode gap spacing and connector lead width variation. Typical yields of chips, determined to have acceptable gap specifications, were close to 100%. A single fabricated flexible chip is depicted in FIG. 19. The fabricated flexible chip measures 3″×2″ and includes electrodes, reservoirs, contact pads and leads.

A dielectric coating was applied to the electrodes and reservoirs by using either rotary screen printing or Gravure printing. For rotary screen printing, Henkel EDAC PF-455B was used as a dielectric coating by printing with a Gallus NF (400 L) screen at a printing speed of 2 m/minute and a UV curing rate of 50%. Typical dielectric thickness was 10-15 μm. For Gravure printing, cylinders were designed to print a high-viscosity dielectric ink, such as IPD-350 (Inkron), at a speed of 2 m/minute using an ink volume of 50 m1/m2. Typical dielectric thickness for Gravure printing was 7-8 μm. A final hydrophobic layer was printed using either Millidyne Avalon 87 or Cytonix Fluoropel PFC 804 UC coating with Gravure cylinders (140-180 L) and a printing speed of 8 m/minute, followed by four successive oven drying steps (4×140° C.). Typical hydrophobic thickness was 40-100 nm.

Alternatively, for small batches of individual chips, the dielectric and hydrophobic coatings may be applied using chemical vapor deposition (CVD) and spin coating, respectively.

### Example 2

### Functional Testing of Low-Cost DMF Chip

A 3″×2″ PET-based DMF bottom chip manufactured as outlined in Example 1 above was tested for actuation capability. FIG. 20 depicts a 3″×2″ PET-based DMF chip (1) over which a 0.7 mm thick glass substrate (3) is positioned. The glass substrate (3) includes a transparent indium tin oxide (ITO) electrode on a lower surface of the glass substrate and a Teflon coating over the ITO electrode. The DMF chip includes 80 silver actuation electrodes with a straight edge electrode design and a 50 μm gap between electrodes, along with 8 buffer reservoirs (see Example 1 above).

The bottom electrodes were coated with a layer of dielectric Parylene-C (6-7 μm thick) and a final coating of Teflon (50 nm thick) by CVD and spin-coating, respectively. Approximately 50 μl of PBS buffer with 0.1% surfactant (2) was pipetted into four adjacent reservoirs on the bottom DMF chip. Droplet sizes ranged from 700-1,500 nl (one or two droplets) and were checked for both vertical and horizontal lateral movement (4), in addition to circular sweep patterns necessary for mixing. Droplet actuation was achieved using a voltage of 90 Vrms. Approximately 90% of the actuation electrodes on the chip were tested and found to be fully functional.

### Example 3

### TSH Immunoassay on Low-Cost DMF Chip

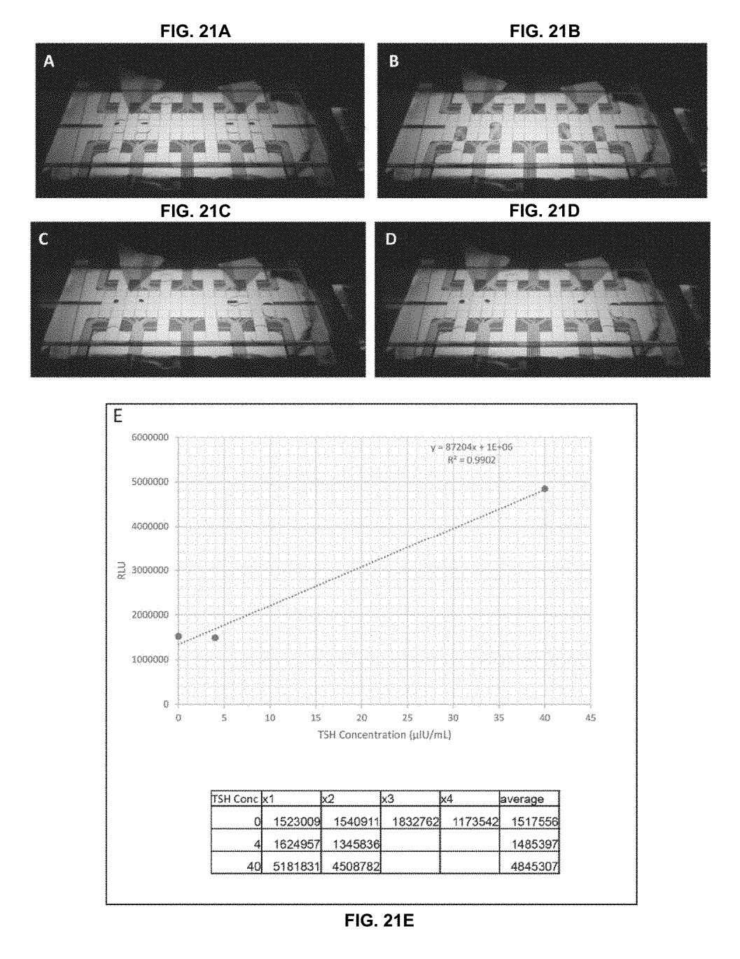

The 3′×2″ PET-based DMF chip overlayed with the glass substrate as described in Example 2 above, was tested for its ability to carry out a thyroid stimulating hormone (TSH) immunoassay, using chemiluminescence detection. Mock samples included TSH calibrator material spiked into tris buffered saline (TBS) buffer containing a blocking agent and a surfactant. Three samples were tested—0, 4, 40 μIU/ml. 2 μl of anti-beta TSH capture antibody, coated on 5 μm magnetic microparticles (3×108 particles/ml), was dispensed from the microparticle reservoir into the middle of the DMF electrode array. The magnetic microparticles were separated from the buffer by engaging a neodymium magnet bar under the DMF chip (FIG. 21A) (3 in.×½ in.×¼ in. thick, relative permeability μr=1.05, remnant field strength Br=1.32 T) under the DMF chip (FIG. 21A). 5 μl of sample was moved to the microparticle slug, followed by mixing the microparticle suspension (FIG. 21B) over a four-electrode square configuration for 5 minutes. The microparticles were separated from the sample by the magnet, and the supernatant was moved to a waste reservoir (FIGS. 21C and 21D). 2 μl of 1 μg/ml anti-TSH detection antibody conjugated to horseradish peroxidase (HRP) was moved to the microparticle slug and mixed for 2 minutes. The microparticles were separated by the magnet, and the supernatant was moved to the waste reservoir. The microparticles containing the immunoassay sandwich complex were washed a total of four times with 4×2 μl of PBS wash buffer containing 0.1% surfactant. Wash buffer from each wash step was moved to waste after the step was completed. Chemiluminescent substrate consisted of 1 μl of SuperSignal H2O2 and 1 μl luminol (ThermoFisher Scientific), which was moved to the microparticle slug, followed by mixing for 6 minutes. Chemiluminescent signal was measured at 427 nm emission (347 nm excitation) using an integrated Hamamatsu H10682-110 PMT with a 5 V DC source. A dose-response curve was plotted against relative luminescence (see FIG. 21E).

### Example 4

### Fabrication and Design of DMF Top Electrode Chips and Well Array

Top Electrode Design:

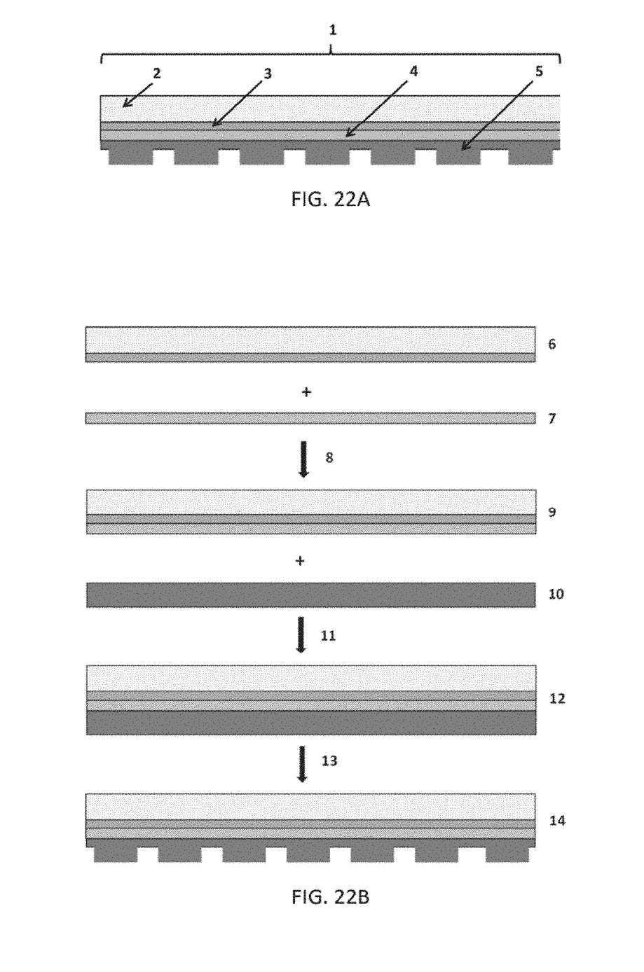

The low-cost flexible DMF top electrode chips containing the well array were fabricated using roll-to-roll (R2R) gravure printing and UV imprinting. With reference to FIG. 22, the basic design (1) incorporated two sets of well arrays printed on a flexible substrate of polyethylene terephthalate (PET) that was used as the top electrode for the DMF chip. The design consisted of 100 nm thick layer of indium-tin oxide (ITO) (3) printed on Melinex ST504 PET substrate (2) (Solutia, OC50 ST504). A coating of PEDOT:PSS primer (4) was used to improve adhesion of the UV embossing resist (5), which contained the final well arrays.

Roll-To-Roll Fabrication of Top Electrode:

FIG. 22B shows a schematic of a fabrication process for the DMF top electrode. Gravure printing (8) was used to coat a 20 nm thick primer layer of PEDOT:PSS (7) (Clevios VP AI4083), diluted with isopropanol to reduce the viscosity, on 5-mil Melinex ST504 PET substrate (6). The roll size was 250 m×200 mm×125 μm; printing speed was 10 m/minute. The resulting primer-coated ITO electrode (9) was transferred to a second R2R printing line where a layer of UV resist (10) was applied with gravure coating (11) to form the precursor material for UV embossing (13). Contact with the nanoarray mold, followed by UV curing, produced the final R2R top electrode film (14), ready to be cut.

Well Design:

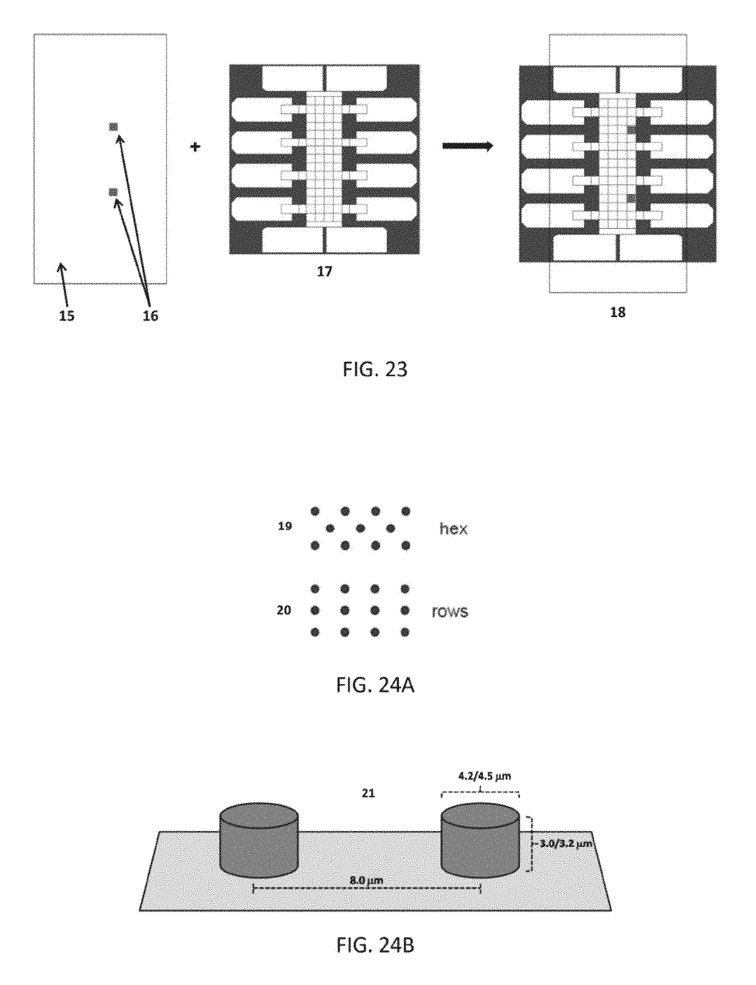

A well design is shown in FIG. 23. The top chip containing the ITO common electrode was designed to be cut into 3″×1.4″ strips (15), each containing two nano-dimensioned well arrays (16) positioned to align with two exterior actuation electrodes on the bottom chip (17). Placement of the top electrode over the bottom chip gave the final DMF chip assembly (18). Each array contained approximately 60,000 wells (245×245).

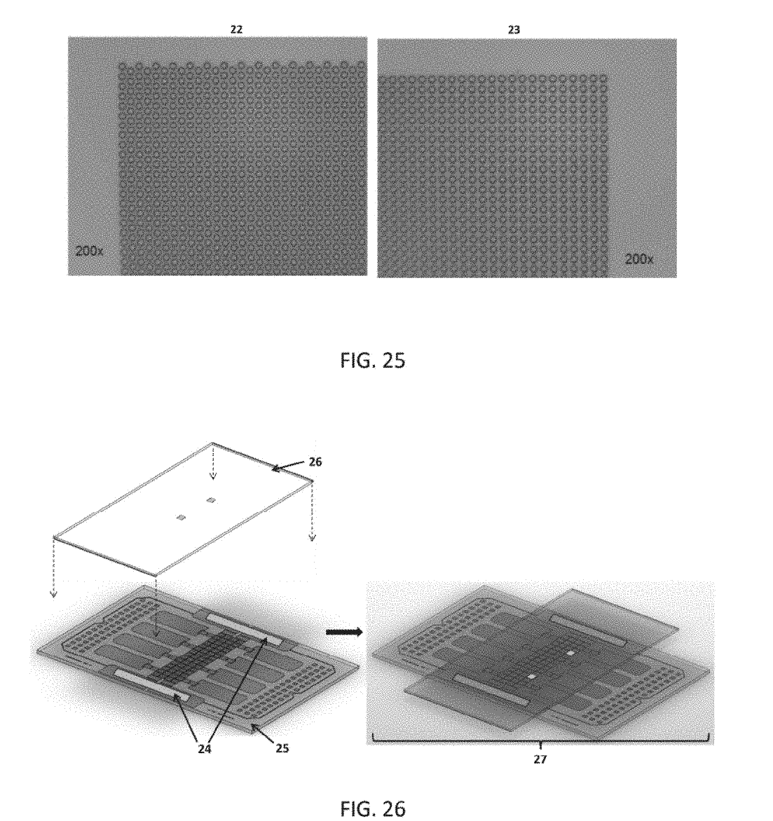

With reference to FIGS. 24A and 24B, two different well spacing formats were used in the initial design—hexagonal (19) and straight rows (20). In addition, two shim designs were used to print two different well sizes (21) 4.2 μm wide×3.0 μm deep with a pitch of 8.0 μm; 4.5 μm×3.2 μm with a pitch of 8.0 μm. Varying the well size and geometric spacing was done in order to optimize for subsequent microparticle loading of 2.7 μm diameter beads. Post-fab QC was conducted on various R2R runs to check for proper well spacing, size and integrity. If deformed wells were observed or the well spacing and/or size was incorrect, a new shim was manufactured and the array was re-printed. FIG. 25 shows optical images at 200× magnification for hexagonal (22) and straight row (23) arrays.

### Example 5

### Assembly of DMF Top Electrode Chips Containing a Well Array

DMF Plastic Chip Assembly:

FIG. 26 schematically describes assembly of the integrated DMF-well device from DMF top electrode chip and a nano-dimensioned well array, as described in Example 4 above. The DMF chip is assembled by placing 2×2 pieces of 90 μm double-sided tape (24) (3M) on opposite sides of the bottom DMF chip (25). The top electrode, containing the embedded arrays (26), is centered on top of the bottom chip so that the position of the two arrays aligns with the two underlying actuation electrodes. The final assembled chip (27) has a gap height of 180 μm (2×90 μm tape).

### Example 6

### TSH Immunoassay on Low-Cost DMF-Well Integrated Chip

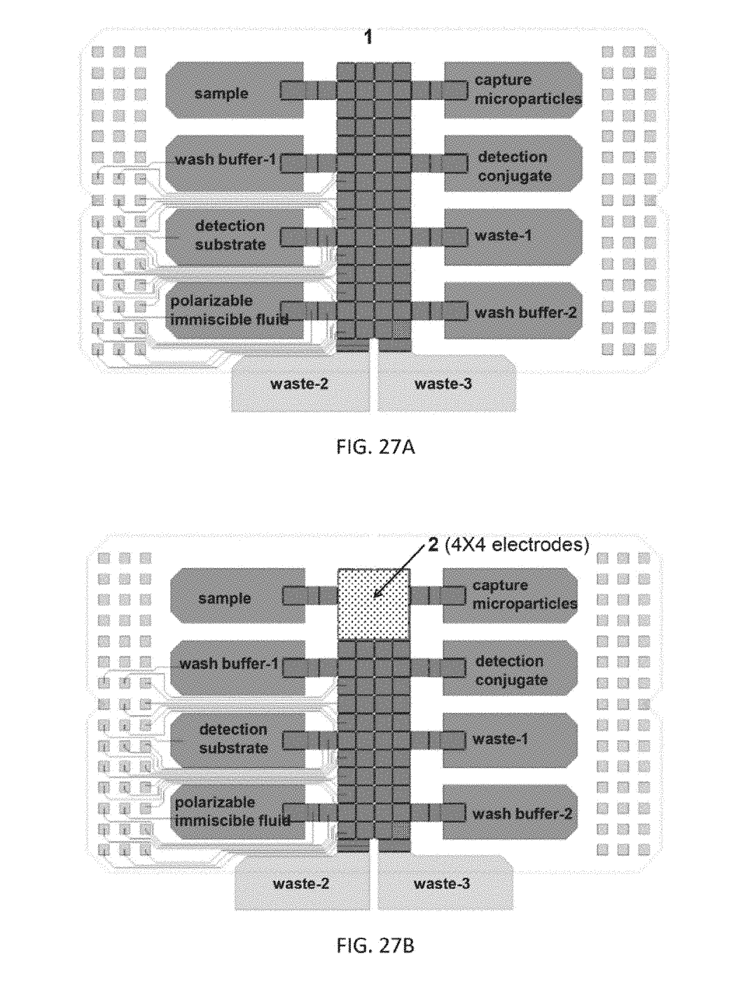

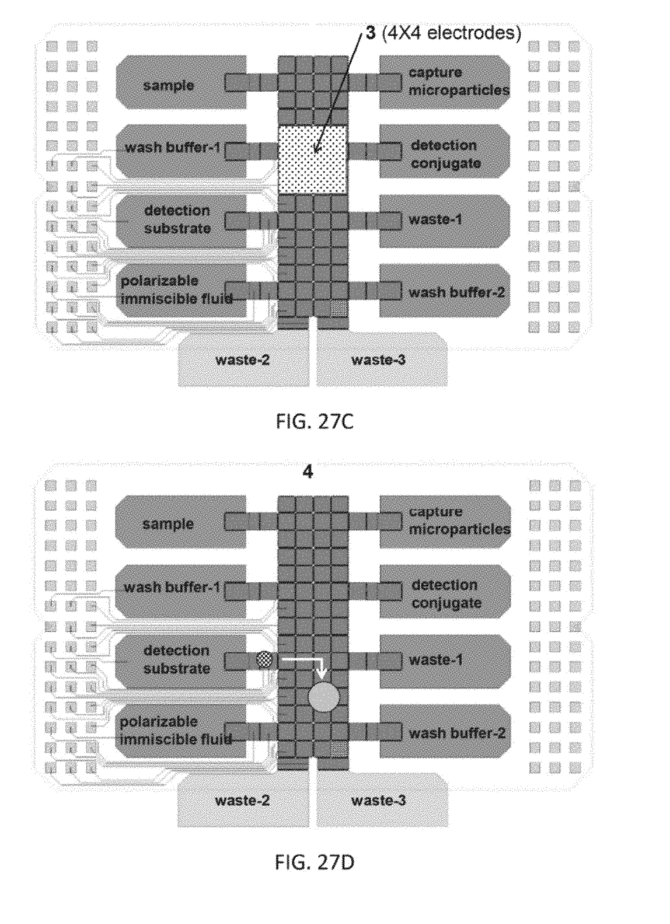

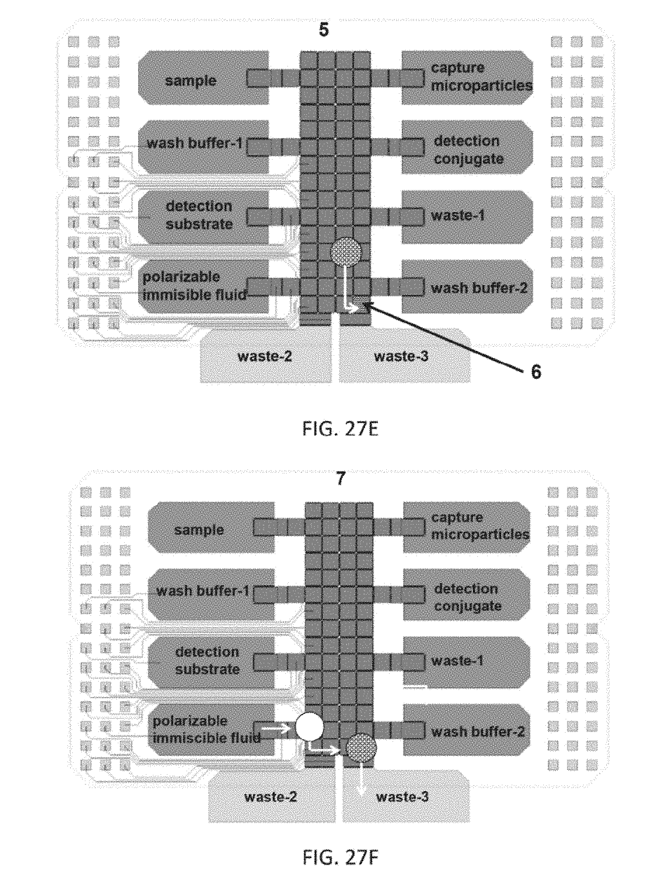

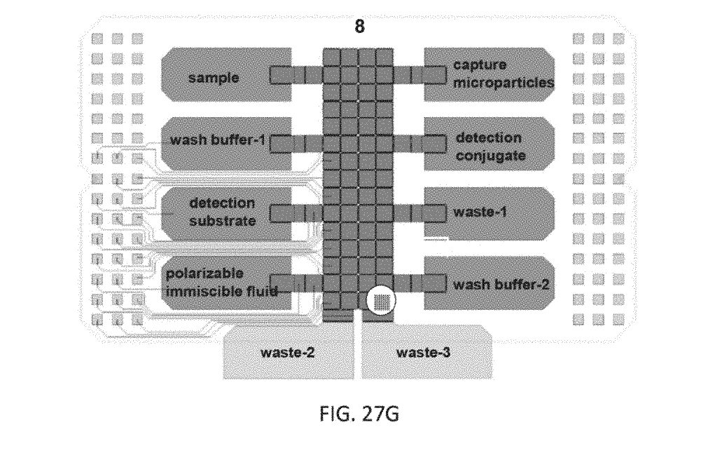

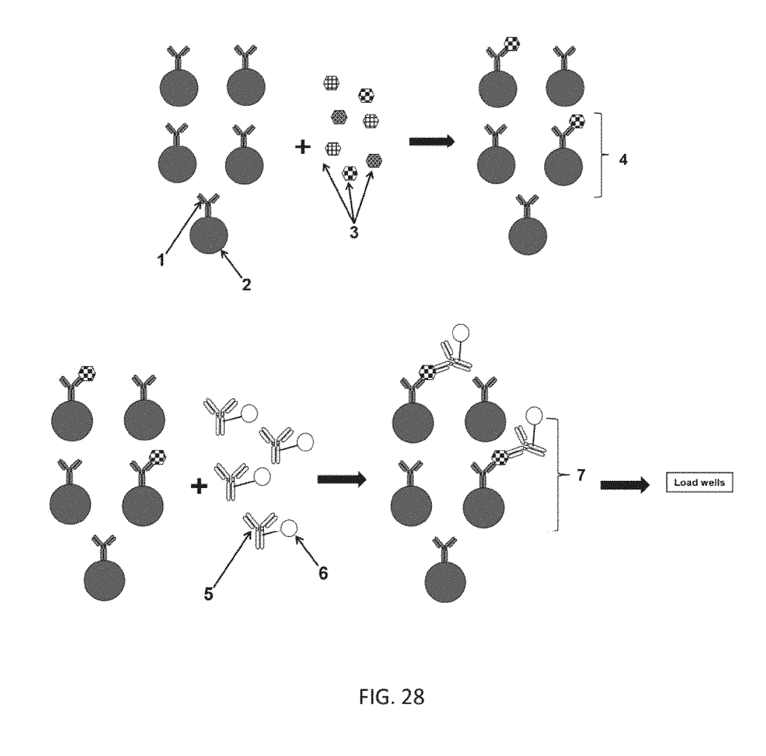

An immunoassay for TSH is described using a combination of DMF, micro-dimensioned well array and digital detection of a fluorescent substrate (FIGS. 27A-27G). A DMF chip (top and bottom electrodes) is pre-loaded with immunoassay (IA) reagents, as shown in (1) (FIG. 27A). The assay is carried out on the DMF chip using air as the filler fluid. The sample may be a biological sample such as whole blood, serum, plasma, urine, sputum, interstitial fluid, or similar matrix. The capture microparticles consists of a suspension of solid-phase magnetic microparticles coated with anti-beta TSH antibodies at a density of 2×107-2×108 particles/ml. Approximately 1-2 μl of sample is moved onto the DMF chip and combined with 1-2 μl of microparticles, followed by mixing within Zone 1 (2) of the DMF chip (FIG. 27B). Zone 1 consists of 16 DMF electrodes reserved for combining, mixing and washing of the sample to form a capture complex of TSH on the magnetic microparticles. Incubation time can range from 1-10 minutes, followed by 1-3 washes of 1-2 μl wash buffer (PBS, 0.1% surfactant) from wash buffer 1 reservoir. Supernatant is removed from the IA complex by engaging a magnet below the DMF chip and moving the supernatant to waste reservoir 1.

The microparticle slug containing the captured TSH antigen on solid phase is resuspended in 1-2 μl of anti-beta TSH detection conjugate antibody labeled with biotin (streptavidin-β-galactosidase complex, at a concentration of 40 pM in PBS). The mixture is allowed to incubate, with mixing over 4×4 electrodes, in Zone 2 on the DMF electrode array (3) for 1-10 minutes (FIG. 27C). The magnet is engaged to capture the magnetic microparticles, and the supernatant is removed to reservoir waste 1. The slug is washed 1-3 times with 1-2 μl wash buffer (PBS, 0.1% surfactant) from wash buffer 2 reservoir. Supernatant is removed from the IA complex by engaging a magnet below the DMF chip and moving the supernatant to waste reservoir 2. The microparticle slug, containing the immunoassay sandwich complex, is resuspended by moving 1-2 μl of 100 μM detection substrate (4) (resorufin-β-D-galactopyranoside, RGP) (FIG. 27D). The mixture is incubated for 1-3 minutes to allow for the enzymatic turnover of RGP—a fluorescent product produced from the enzymatic turnover of RGP by β-galactosidase.

The mixture is moved (5), as shown in FIG. 27E, to a spot on the DMF chip that contains an array of femtoliter wells (6), either on the bottom or top substrate. The size of the femtoliter well size is slightly larger than the size of the microparticle being used in the assay. The number of wells may range from 1,000-2,000,000. The microparticles are deposited in the wells by using either gravity (passive loading) or a magnet (active loading). Excess supernatant is moved to waste-3 reservoir.

The femtoliter wells are sealed by moving 1-5 μl of a polarizable immiscible fluid (i.e. organic solvent, oil, etc.) to the array position (7, 8), as shown in FIGS. 27F and 27G, using electrowetting on dielectric (EWOD), dielectrophoresis (DEP) force(s), or surface acoustic waves (SAW) thereby sealing the wells. Some examples of suitable polarizable immiscible fluids include silicone oil, fluorosilicone oil, mineral oil, 1-hexanol, THF, m-dichlorobenzene, chloroform, and the like (S. Fan, et al., Lab On Chip, 9, 1236, 2009; D. Chatterjee, et al., Lab On Chip, 6, 199, 2006). The filler fluid for the entire assay is air.

The number of total particles in the wells is determined by white light illumination with a wide field microscope/CCD camera, followed by imaging at 574 nm/615 nm excitation/emission (exposure time=3-10 seconds) for determining the number of beads containing a detection label. The final TSH concentration is determined from a standard curve run with TSH calibrators. Digital quantitation is determined by using the Poisson equation and the ratio of positive to negative beads.

### Example 7

### Nanodimensioned Well Top Loading with Polarizable Fluid

General Immunoassay Format:

The 3′×2″ PET-based DMF chip can be used to run an ELISA-based sandwich immunoassay, coupled with digital fluorescence detection in the well array. With reference to FIG. 28, a sample (3), containing a specific antigen to be analyzed, is mixed with magnetic microparticles (2) coated with a capture antibody (1) and mixed to allow for immunocapture of the desired antigen. After washing, the captured antigen (4) is mixed with a second detection antibody (5) labeled with a detection moiety (6). The bead mixture, containing the sandwich immunoassay complex (7), is washed again to remove unbound detection antibody. The microparticles are loaded in the top substrate of the well array by moving the aqueous droplet to the array and applying a magnet to pull the beads into the wells. The wells are sealed by moving a droplet of polarizable immiscible fluid over the wells using DMF forces. A CCD camera images the array to determine the number of positive and negative microparticles. The sample is quantitated using Poisson statistics. All immunoassay processing steps are carried out on a DMF chip using air as the filler fluid.

TSH Immunoassay—DMF

One-two μl of anti-TSH capture antibody, coated on 2.7 μm magnetic microparticles (3×108 particles/ml), is dispensed from the microparticle reservoir on the DMF chip into the middle of the DMF electrode array. The magnetic microparticles are separated from the buffer by engaging a magnet, located under the DMF chip, and moving the supernatant to the waste reservoir. One μl of an aqueous sample is pulled from a DMF sample reservoir and moved to the microparticle slug, followed by a mixing step where the droplet is moved over several electrodes for 1-5 minutes. The microparticles are separated from the sample by applying the bottom magnet, followed by removal of the supernatant to a waste reservoir. One-two μl of anti-TSH detection antibody (0.5 μg/ml) conjugated to β-galactosidase (β-gal) is moved to the microparticle slug and mixed for 2-5 minutes. The microparticles are separated using the bottom magnet and the supernatant is moved to the waste reservoir. The microparticles containing the immunoassay sandwich complex are washed a total of four times with 4×2 μl of PBS wash buffer containing 0.1% surfactant. Wash buffer from each wash step is moved to waste after the step is completed. One μl of 100 μM resorufin-β-D-galactopyranoside (RGP) is taken from the RGP reservoir and moved to the microparticle slug, followed by mixing for 15-30 seconds. The beads are now ready for deposition into the well array.

TSH Immunoassay—Digital Array Detection:

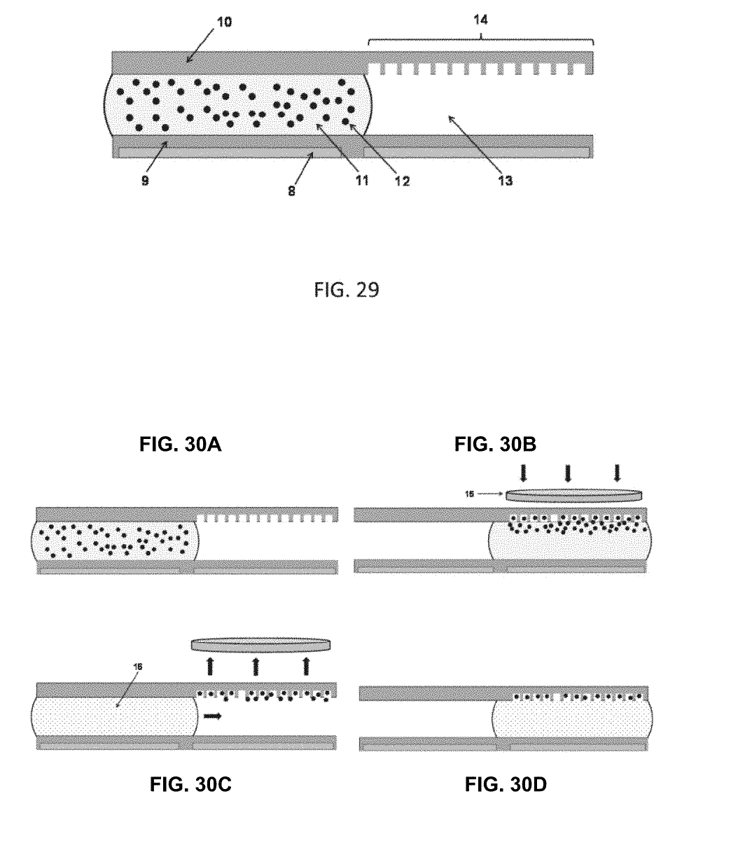

As shown in FIG. 29, the basic components for DMF-directed top loading of the microparticles into the array includes the bottom PET-based electrode chip with 80-nm thick silver electrodes (8) (electrod gap <100 μm, 2.25 mm×2.25 mm), 5-10 μm thick dielectric/hydrophobic layer (9), the top PET-based ITO electrode (10) chip containing the array of wells (14) (configured to hold no more than one microparticle) and an aqueous droplet (11) containing 2.7 μm magnetic microparticles (12). The filler fluid is air (13). The gap height between the top and bottom electrodes is approximately 180 μm (from 2 pieces of 90 μm double-sided tape).

Transport and sealing is accomplished by using a combination of DMF forces (EWOD, DEP, and/or electromediated force) to move aqueous and immiscible fluids, such as silicone oil. It has been previously shown that different driving voltages are required to move both aqueous and oil droplets on the same DMF chip (S-K Fan, et al., Lab on Chip, 9, 1236, 2009).

After addition of the fluorescent substrate RGP to the aqueous droplet containing the immunoassay complex (FIG. 30A), the droplet is moved to an electrode positioned below a well array containing approx. 60,000 wells (245×245 array; 4.2 μm diameter; 3.0 μm depth; 8.0 μm pitch) using a voltage of 20-50 Vrms (1 KHz). A top magnet (15) is engaged to promote efficient loading of the microparticles into the wells (FIG. 30B); total deposition time is 30-60 seconds. The aqueous droplet is moved away as the top magnet is dis-engaged, leaving behind a thin layer of deposited and surface-bound beads (FIG. 30C). A droplet of silicone oil is moved from a reservoir using a voltage of 200-300 V (DC) and moved to the electrode positioned under the array (FIG. 30D), thereby washing away any surface-bound microparticles, while sealing microparticles contained in the wells. The fluorescence generated from enzymatic turnover of RGP to resorufin is monitored by a CCD camera at 574/615 nm (excitation/emission). The ratio of “on” microparticles to “off” microparticles is determined. The TSH concentration in the sample is determined by interpolation from a TSH calibration curve.

### Example 8

### Synthesis of Photocleavable 2-Nitrobenzyl Succinimidyl/Maleimidyl Bifunctional Linker

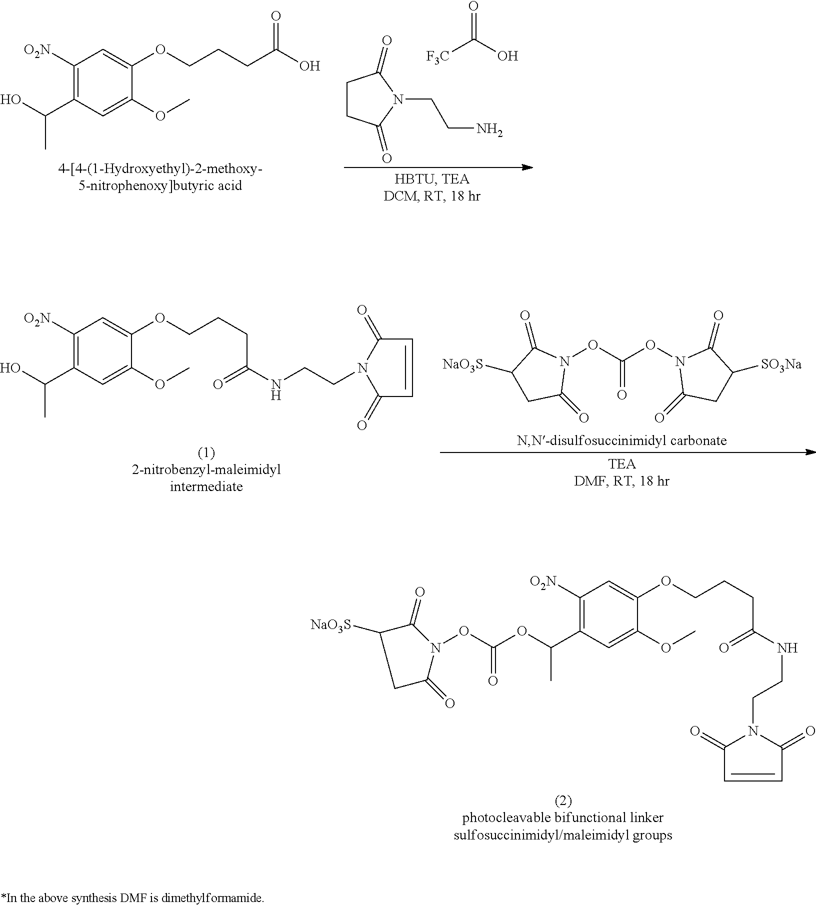

Synthesis of Compound 2.

Synthesis of the photocleavable sulfosuccinimidyl/maleimidyl linker is derived from Agasti, et al., J. Am. Chem. Soc., 134(45), 18499-18502, 2012. Briefly, starting material 4-[4-(1-hydroxyethyl)-2-methoxy-5-nitrophenoxy]butyric acid (0.334 mmol) is dissolved in dry dichloromethane (DCM) under argon atmosphere. The flask is cooled to 0° C. by placing it in an ice bath. Compound 2-(1H-benzotriazole-1-yl)-1,1,3,3-tetramethyluronium hexafluorophosphate (HBTU) (0.368 mmol) and trimethylamine (TEA) (0.835 mmol) are added to the solution. The reaction mixture is stirred at 0° C. for 5 min and subsequently N-(2-aminoethyl)maleimide trifluoroacetate salt (0.368 mmol) is added. After stirring at 0° C. for 15 min, the reaction mixture is allowed to rise to room temperature (RT) and further stirred for 18 h. After dilution of the reaction mixture with DCM (45 ml), the organic phase is washed with water (2×), saturated NaCl solution (1×) and dried over sodium sulfate. The organic layer is concentrated under reduced pressure and purified by flash chromatography using a SiO₂ column (eluent: 100% DCM to 3% methanol in DCM, v/v). Compound 1 (0.024 mmol) is dissolved in anhydrous dimethylformamide (1 ml). N,N′-disulfosuccinimidyl carbonate (DSC) (0.071 mmol) and TEA (0.096 mmol) are successively added to the solution. The reaction mixture is stirred at RT for 18 h. The reaction mixture is purified by directly loading onto a C18 reverse phase column (eluent: 5% acetonitrile in water to 95% acetonitrile in water, v/v). Starting material and other chemicals used for the synthesis may be purchased from Sigma-Aldrich.

### Example 9

### Synthesis of Photocleavable Sulfosuccinimidyl/DBCO 2-Nitrobenzyl Bifunctional Linker

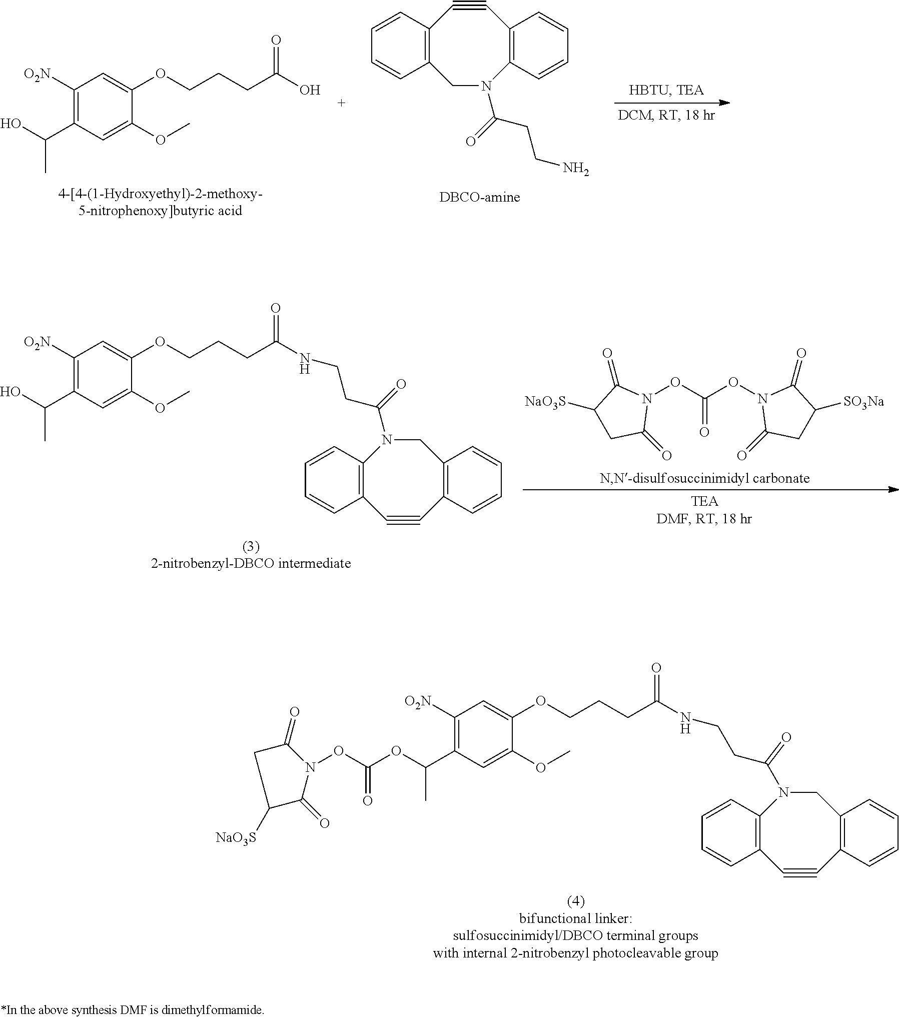

Synthesis of Compound 4.

Synthesis of the photocleavable sulfosuccinimidyl/dibenzocyclooctyl (DBCO) alkynyl linker is derived from a similar procedure described in Agasti, et al., J. Am. Chem. Soc., 134(45), 18499-18502, 2012. Briefly, starting material 4-[4-(1-hydroxyethyl)-2-methoxy-5-nitrophenoxy]butyric acid (0.334 mmol) is dissolved in dry dichloromethane (DCM) under argon atmosphere. The flask is cooled to 0° C. by placing it in an ice bath. Compound 2-(1H-benzotriazole-1-yl)-1,1,3,3-tetramethyluronium hexafluorophosphate (HBTU) (0.368 mmol) and trimethylamine (TEA) (0.835 mmol) are added to the solution. The reaction mixture is stirred at 0° C. for 5 min and subsequently DBCO-amine (0.368 mmol) is added. After stirring at 0° C. for 15 min, the reaction mixture is allowed to rise to RT and further stirred for 18 h. After dilution of the reaction mixture with DCM (45 ml), the organic phase is washed with water (2×), saturated NaCl solution (1×) and dried over sodium sulfate. The organic layer is concentrated under reduced pressure and purified by flash chromatography using a SiO₂ column (eluent: 100% DCM to 3% methanol in DCM, v/v). Compound 3 (0.024 mmol) is dissolved in anhydrous dimethylformamide (1 ml). N,N′-disulfosuccinimidyl carbonate (DSC) (0.071 mmol) and TEA (0.096 mmol) are successively added to the solution. The reaction mixture is stirred at RT for 18 h. The reaction mixture is purified by directly loading onto a C18 reverse phase column (eluent: 5% acetonitrile in water to 95% acetonitrile in water, v/v). Starting material and other chemicals used for the synthesis may be purchased from Sigma-Aldrich.

### Example 10

### Coupling and Photochemical Cleavage of Antibody-DNA Conjugate Using Sulfosuccinimidyl/Maleimidyl 2-Nitrobenzyl Bifunctional Linker

Bioconjugation and Cleavage of Antibody and DNA.

DNA molecules may be conjugated to antibodies using the following scheme. DNA may be thiolated at the 5′ terminus by replicating a DNA sequence in a PCR reaction using two PCR primers where one or both primers are labeled with a 5′-thiol group. Labeled DNA (100 μM final concentration) is dissolved in 50 mM HEPES (pH=7.0) with stirring. Compound 2 (2 mM) is added and the reaction is allowed to proceed at RT for 2 hours. After coupling, excess unreacted maleimide groups are quenched with excess dithiothreitol (DTT). The conjugate is purified on a gel filtration column (Sephadex G-25) or by extensive dialysis at 4° C. in an appropriate conjugate storage buffer. Purified DNA-succinimidyl linker (50 μM final concentration) is dissolved in 100 mM PBS (pH=7.5) with stirring. Native antibody (50 μM final concentration) is added and the reaction is allowed to proceed at RT for 2 hours. The Ab-DNA conjugate is purified using a Sephadex column (Sephadex G25) operated with 100 mM PBS, pH 7.5, or BioGel P-30 gel filtration media.

The conjugate may be cleaved prior to nanopore detection by illuminating with a UV lamp at 365 nm. This example may also be used on DNA dendrimers using the same bioconjugation chemistry.

### Example 11

### Coupling and Photochemical Cleavage of Antibody-DNA Conjugate Using Sulfosuccinimidyl/DBCO 2-Nitrobenzyl Bifunctional Linker

Bioconjugation and Cleavage of Antibody and DNA.

DNA molecules may be conjugated to antibodies using the following scheme. DNA may be aminated at the 5′ terminus by replicating a DNA sequence in a PCR reaction using two PCR primers where one or both primers are labeled with a 5′-amine group. Labeled DNA (100 μM final concentration) is dissolved in 100 mM PBS (pH=7.5) with stirring. Compound 4 (2 mM final concentration) is added and the reaction is allowed to proceed at RT for 2 hours. The DNA-DBCO linker is purified on a gel filtration column (Sephadex G-25) or by extensive dialysis at 4° C. in an appropriate conjugate storage buffer. Purified DNA-DBCO linker (50 μM final concentration) is dissolved in 50 mM Tris (pH=7.0) with stirring. Copper-free Click chemistry is used to couple the DNA-DBCO linker to the antibody. Azido-labeled antibody (Kazane et al., Proc. Natl. Acad. Sci., 109(10), 3731-3736, 2012) (25 μM final concentration) is added and the reaction is allowed to proceed at RT for 6-12 hours. The Ab-DNA conjugate is purified using a Sephadex column (Sephadex G25) operated with 100 mM PBS, pH 7.5, or BioGel P-30 gel filtration media.

The conjugate may be cleaved prior to nanopore detection by illuminating with a UV lamp at 365 nm. This example may also be used on DNA dendrimers using the same bioconjugation chemistry.

### Example 12

### Nanoparticle-Antibody Conjugates for Digital Immunoassays (Nanopore Counting)

This example describes covalent conjugation of an antibody to 26 nm carboxylated polystyrene nanoparticles (NP, PC02N), such as those which can be obtained from Bangs Labs (Fishers, Ind., USA). The 26 nm NPs have a surface charge of 528.7 μeq/g and a parking area of 68.4 sq. Å/group (per manufacturer information).

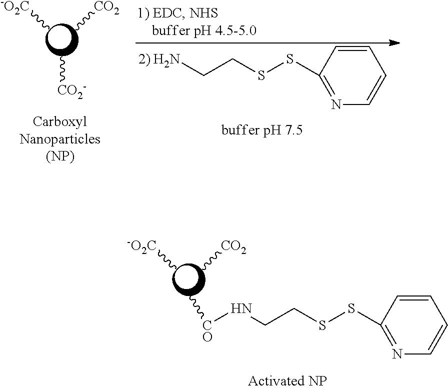

Activation of Carboxyl-Polystyrene Nanoparticles:

1.0 mL (100 mg/mL) of 26 nm carboxylated-NP is washed with 10 mL of 0.1M MES (2-[N-morpholino]ethane sulfonic acid, pH 4.5-5.0. After the wash, the pellets are resuspended in 100 mL of 0.1M MES pH 4.5-5.0 for a 1.0 mg/mL NP concentration (0.1% solids). 10.0 mL nanoparticle suspension (10 mg NP, 5.28 μeq carboxyl) is transferred to a vial and reacted with 10 pL (5.28 μmoles, 1.0 equiv/CO₂H eq) of a freshly prepared 10 mg/mL EDC solution in water (1-Ethyl-3-[3-dimethylaminopropyl]carbodiimide hydrochloride) and 17 μL (7.93 μmoles, 1.5 equiv/1 equiv EDC) of a 10 mg/mL solution of sulfo-NHS solution in water (N-hydroxysulfosuccinimide, Sigma, Cat#56485) at room temperature for 15 min with continuous mixing. The reacted suspension is centrifuged at 6,500 g and the solution is discarded. The pellet is washed with 20 mL of 20 mM PBS/5 mM EDTA pH 7.5 and spun down by centrifugation at 6,500 g. The supernatant is removed. The succinimide-activated carboxyl-NP pellet is resuspended in 50 mM PBS pH 7.5 and 9.8 μL (52.8 nmoles, 0.01 equiv/l CO₂H eq) of a 1.0 mg/mL pyridine dithioethylamine solution in water is immediately added and allowed to react with continuous stirring for 2-4 h at room temperature. The pyridyl-derivatized carboxyl-NP is washed with 10 mL of 20 mM PBS/5 mM EDTA pH 7.5 and resuspended in 10.0 mL of the same buffer. The nanoparticle concentration is determined using UV-Vis spectroscopy (600 nm, scatter) using a carboxyl-NP calibration curve. Pyridyl-ligand loading on the NP is determined by reducing a defined amount of NP with 10 mM TCEP or DTT, removing the reducing agent by centrifugation, resuspending the pyridyl-activated NP pellet in PBS/EDTA pH 7.2 and reacting with the Ellman reagent (measure A412 of the supernatant). The activated NP is stored at 4° C. if not used on the same day for antibody conjugation.

A range of EDC/NHS and pyridine dithioethyl amine molar inputs are evaluated to determine the desired stoichiometry for preparing distinct antibody-nanoparticle conjugates. Reaction parameters (pH, temperature, time) are assessed to achieve the desired NP activation outcome.

Antibody Reduction:

1.0 mL of a 10 mg/mL antibody solution (10 mg) is mixed well with 38 μL of a freshly prepared 30 mg/mL 2-MEA solution (10 mM reaction concentration) (2-mercaptoethylamine hydrochloride), then capped and placed at 37° C. for 90 min. The solution is brought to room temperature and the excess 2-MEA is removed with a desalting column, pre-equilibrated in 20 mM PBS/5 mM EDTA pH 7.5. The concentration of the reduced antibody is determine using UV-Vis absorbance at A280 (protein absorbance) and A320 (scatter correction). The number of free thiols is determined using the Ellman test. The conditions are optimized as needed to generate 2 or 4 free thiols (Cys in the antibody hinge region). The reduced antibody is used immediately for coupling to pyridyl-derivatized carboxyl-NP.

Coupling of Reduced Antibody to Activated-Nanoparticle:

Assumptions made: (1) antibody parking area is 45 nm²; (2) 26 nm nanoparticle surface area is 2,120 nm²; (3) 47 antibody molecules theoretically fit on the surface of a 26 nm NP.

Procedure:

To 10 mL (10 mg) of a 0.1% solution of pyridyl-activated carboxy-nanoparticles in 20 mM PBS/5 mM EDTA (pH 7.5), 0.10 mg (0.66 nmoles, 0.10 mL) of the reduced antibody is added at 1.0 mg/mL in the same EDTA containing buffer. The mixture is allowed to react at room temperature with mixing for 2 h, centrifuged to remove unbound molecules, and aspirated. The pellet is washed with 10 mL of PBS pH 7.2, centrifuged, and aspirated. The antibody-NP conjugate is suspended in 10.0 mL of PBS pH 7.2. The conjugate NP concentration (% solids) is determined using UV-Vis spectroscopy (600 nm). The particle conjugate is examined by SEM and the size/charge distribution is determined using the ZetaSizer. Size exclusion chromatography can be used to isolate distinct conjugates from a potential distribution of conjugate population. Antibody-to-NP incorporation ratio can be determined by flow cytometry using fluorescently labelled antigen conjugate or using a Micro BCA (uBCA) assay. A range of antibody-to-NP molar inputs can be evaluated, along with conjugation temperature and pH to generate a homogenous population of distinct conjugates (i.e., NP incorporation ratio of 2 or 4).

Nanopore Counting Immunoassay

The scheme above illustrates the nanopore counting assay utilizing the reduced antibody-activated nanoparticle conjugate whose preparation is described above. The immune complex formed in the course of the immunoassay can be cleaved by reduction of the disulfide bond linker to form the free antibody-analyte-antibody complex and free nanoparticle tag, which permits the nanoparticle tag to be counted upon passage through the nanopore.

### Example 13

### Synthesis of CPSP Conjugates

A. CPSP Antibody Conjugate.

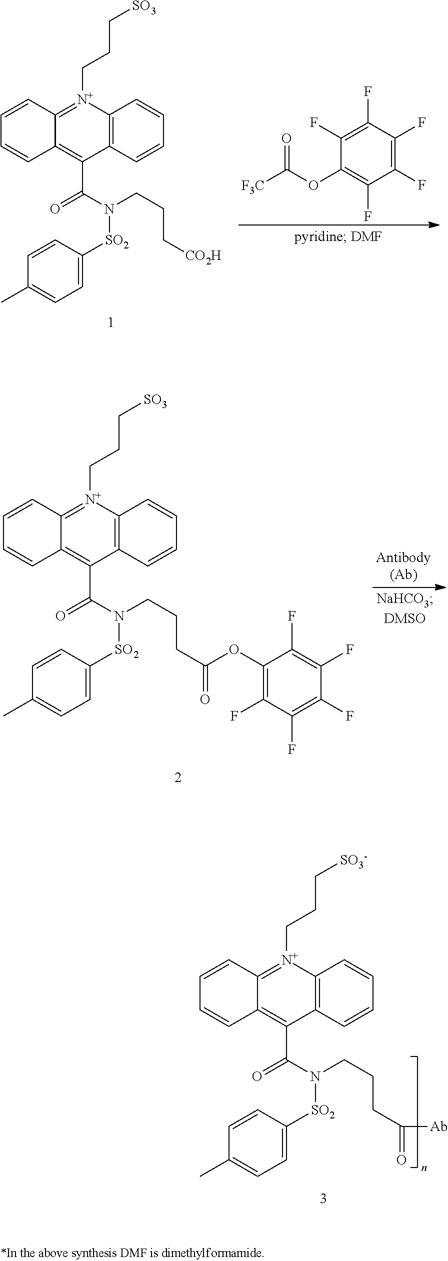

### 3-(9-((4-Oxo-4-(perfluorophenoxy)butyl)tosyl)carbamoyl)acridin-10-ium-10-yl)propane-1-sulfonate (2)

A 25 mL round bottom flask equipped with a magnetic stirrer and a nitrogen inlet was charged with 3-(9-((3-carboxypropyl)(tosyl)carbamoyl)acridin-10-ium-10-yl)propane-1-sulfonate (CPSP) (1) (1 mmol), pyridine (5 mmol) and dimethylformamide (10 mL). The solution was cooled in an ice bath and pentafluorophenyl trifluoroacetate (1.3 mmol) was added dropwise. The ice bath was removed and the reaction was stirred at room temperature for 3 hours. The volatile components were removed from the reaction in vacuo and the residue was taken up in methanol and purified by reverse phase HPLC to give the title compound.

CPSP Antibody Conjugate (3):

A solution of 2 (1 μL of a 10 mM solution in DMSO) was added to an antibody solution (100 μL of a 10 μM solution in water) and aqueous sodium bicarbonate (10 μL of a 1M solution). The resulting mixture was stirred at room temperature for 4 hours. Purification of the product was achieved on a spin column to give the CPSP antibody conjugate 3. The value of “n” varies in an antibody-dependent fashion. The incorporation can be controlled to some extent by raising or lowering the active ester concentration (i.e., compounds 2, 5, 9 and 13) and/or by raising or lowering the pH during the reaction, but always results in a distribution of incorporation values. The average incorporation ration (“I.R.”) is determined experimentally after the reaction. Typically, “n” is any value between 1 and 10.

B. CPSP Antibody Conjugate with Spacer.

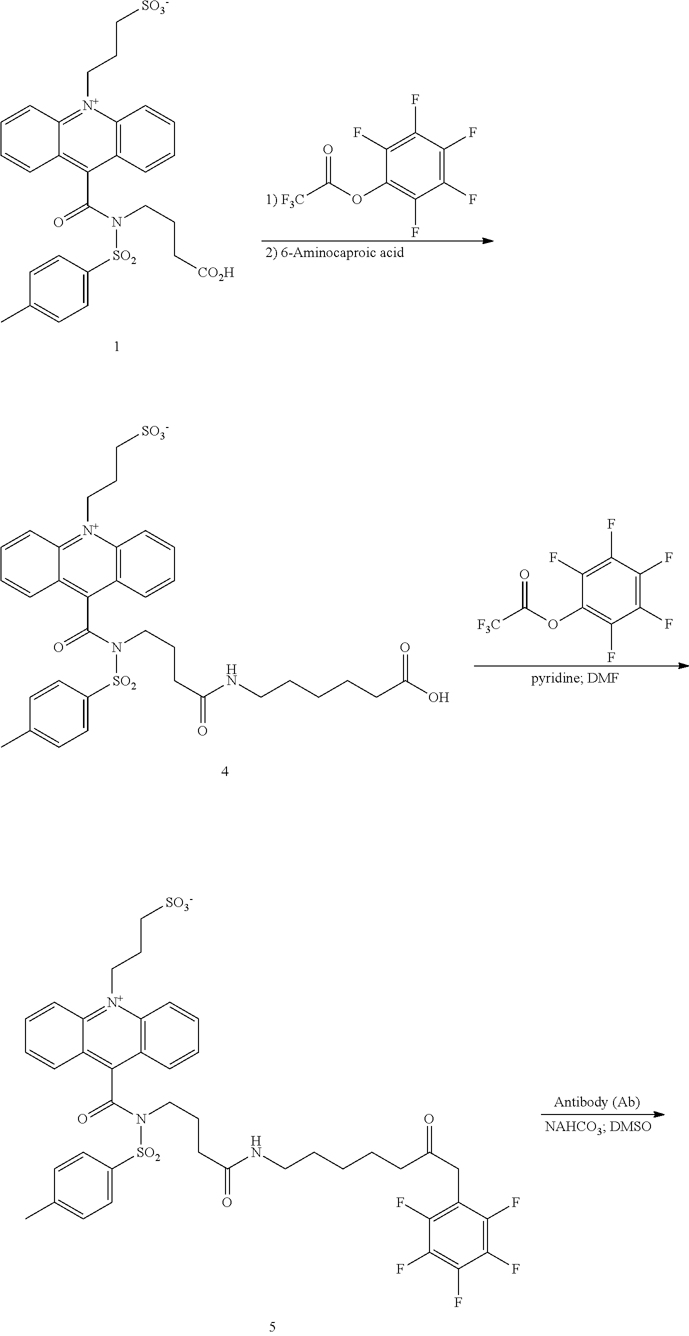

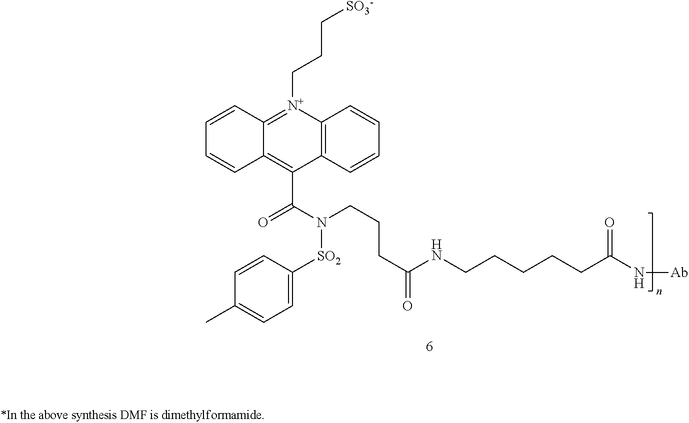

### 3-(9-((4-((5-Carboxypentyl)amino)-4-oxobutyl)(tosyl)carbamoyl)acridin-10-ium-10-yl)propane-1-sulfonate (4)

A 25 mL round bottom flask equipped with a magnetic stirrer and a nitrogen inlet was charged with 3-(9-((3-carboxypropyl)(tosyl)carbamoyl)acridin-10-ium-10-yl)propane-1-sulfonate CPSP (1) (1 mmol), pyridine (5 mmol) and dimethylformamide (10 mL). The solution was cooled in an ice bath and pentafluorophenyl trifluoroacetate (1.3 mmol) was added dropwise. The ice bath was removed and the reaction was stirred at room temperature for 3 hours. 6-Aminocaproic acid (1.3 mmol) was then added to the reaction in small portions followed by N,N-diisopropylethylamine (5 mmol), and the reaction was stirred for 1 hour at room temperature. After this time, the volatile components were removed from the reaction in vacuo and the residue was purified by reverse phase HPLC to give the title compound.

### 3-(9-((4-Oxo-4-((6-oxo-6-(perfluorophenoxy)hexyl)amino)butyl)(tosyl)carbamoyl) acridin-10-ium-10-yl)propane-1-sulfonate (5)

A 25 mL round bottom flask equipped with a magnetic stirrer and a nitrogen inlet was charged with 4 (1 mmol), pyridine (5 mmol) and dimethylformamide (10 mL). The solution was cooled in an ice bath and pentafluorophenyl trifluoroacetate (1.3 mmol) was added dropwise. The ice bath was removed and the reaction was stirred at room temperature for 3 hours. After this time, the volatile components were removed from the reaction under a stream of nitrogen and the residue was purified by reverse phase HPLC to give the title compound.

CPSP Antibody Conjugate with Spacer (6):

A solution of 5 (1 μL of a 10 mM solution in DMSO) was added to an antibody solution (100 μL of a 10 μM solution in water) and aqueous sodium bicarbonate (10 μL of a 1M solution). The resulting mixture was stirred at room temperature for 4 hours. Purification of the product was achieved on a spin column to give the CPSP antibody conjugate with spacer 6. Typically, “n” is any value between 1 and 10.

C. CPSP Oligonucleotide-Antibody Conjugate.

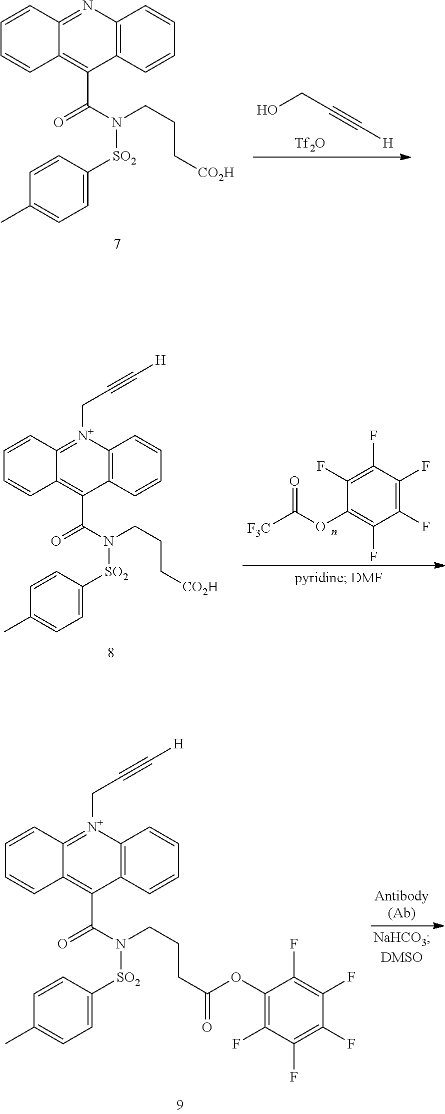

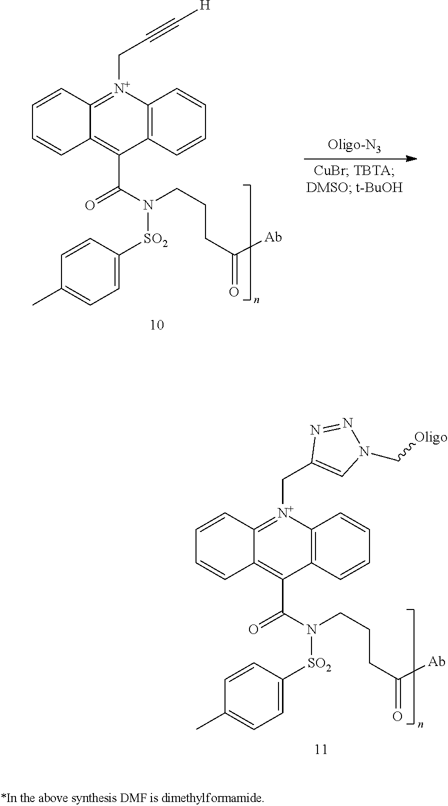

### 9-((3-Carboxypropyl)(tosyl)carbamoyl)-10-(prop-2-yn-1-yl)acridin-10-ium (8)

A 100 mL round bottom flask equipped with a magnetic stirrer and a nitrogen inlet was charged with propargyl alcohol (10 mmol), 2,6-di-tert-butylpyridine (10 mmol) and methylene chloride (50 mL) and cooled to −20° C. Triflic anhydride was then added dropwise to the solution and the reaction was stirred for 2 hours at −20° C. Pentane (25 mL) was added to the reaction and the resulting precipitated salts were separated by filtration. The volatile components were evaporated in vacuo and the residue was redissolved in methylene chloride (25 mL) in a 100 mL round bottom flask. 4-(N-Tosylacridine-9-carboxamido)butanoic acid (CP-acridine) (7) (1 mmol) was added in small portions and the reaction was stirred at room temperature for 18 hours. The volatile components were evaporated in vacuo and the residue was taken up in methanol (5 mL) and purified by reverse phase HPLC to give the title compound.

### 9-((4-oxo-4-(perfluorophenoxy)butyl)(tosyl)carbamoyl)-10-(prop-2-yn-1-yl)acridin-10-ium (9)

A 25 mL round bottom flask equipped with a magnetic stirrer and a nitrogen inlet was charged with 8 (1 mmol), pyridine (5 mmol) and dimethylformamide (10 mL). The solution was cooled in an ice bath and pentafluorophenyl trifluoroacetate (1.3 mmol) was added dropwise. The ice bath was removed and the reaction was stirred at room temperature for 3 hours. The volatile components were removed from the reaction in vacuo and the residue was taken up in methanol and purified by reverse phase HPLC to give the title compound.

CPSP Antibody Conjugate (10):

A solution of 9 (1 μL of a 10 mM solution in DMSO) was added to an antibody solution (100 μL of a 10 μM solution in water) and aqueous sodium bicarbonate (10 μL of a 1M solution). The resulting mixture was stirred at room temperature for 4 hours. Purification of the product was achieved on a spin column to give the CPSP antibody conjugate 10. Typically, “n” is any value between 1 and 10.

CPSP Oligonucleotide-Antibody Conjugate (11):

A mixture of an oligoazide (10 nmol in 5 μL water), CPSP antibody conjugate 10 (10 nmol in 10 μL water) and a freshly prepared 0.1 M “click solution” (3 μL—see below) was shaken at room temperature for 4 hours. The reaction was diluted with 0.3M sodium acetate (100 μL) and the DNA conjugate was precipitated by adding EtOH (1 mL). The supernatant was removed and the residue was washed 2× with cold EtOH (2×1 mL). The residue was taken up in water (20 μL) and the solution of the CPSP oligonucleotide-antibody conjugate 11 was used without further purification. Typically, “n” is any value between 1 and 10.

“Click Solution”:

CuBr (1 mg) was dissolved in 70 μL DMSO/t-BuOH 3:1 to form a 0.1 M solution. (This solution must be freshly prepared and cannot be stored.) Tris(benzyltriazolylmethyl)amine (TBTA) (54 mg) was dissolved in 1 mL DMSO/t-BuOH 3:1 to form a 0.1 M solution. (This solution can be stored at −20° C.) 1 volume of the 0.1 M CuBr solution was added to 2 volumes of the 0.1 M TBTA solution to provide a “click solution.”

D. CPSP Oligonucleotide-Antibody Conjugate with Spacer.

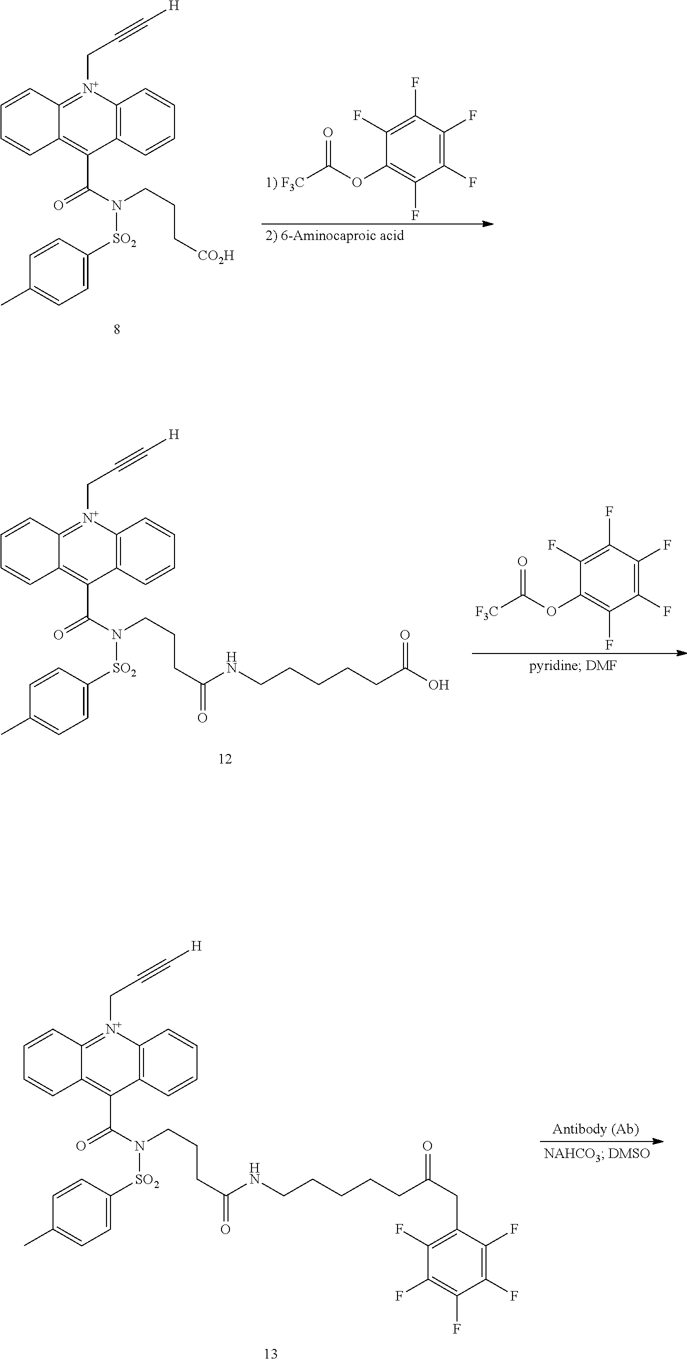 [SMILES: C#CC[n+]1c2ccccc2c(C(=O)N(CCCC(=O)O)S(=O)(=O)c2ccc(C)cc2)c2ccccc21 ; O=C(Oc1c(F)c(F)c(F)c(F)c1F)C(F)(F)F ; C#CC[n+]1c2ccccc2c(C(=O)N(CCCC(=O)NCCCCCC(=O)O)S(=O)(=O)c2ccc(C)cc2)c2ccccc21 ; O=C(Oc1c(F)c(F)c(F)c(F)c1F)C(F)(F)F ; C#CC[n+]1c2ccccc2c(C(=O)N(CCCC(=O)NCCCCCC(=O)Cc2c(F)c(F)c(F)c(F)c2F)S(=O)(=O)c2ccc(C)cc2)c2ccccc21]

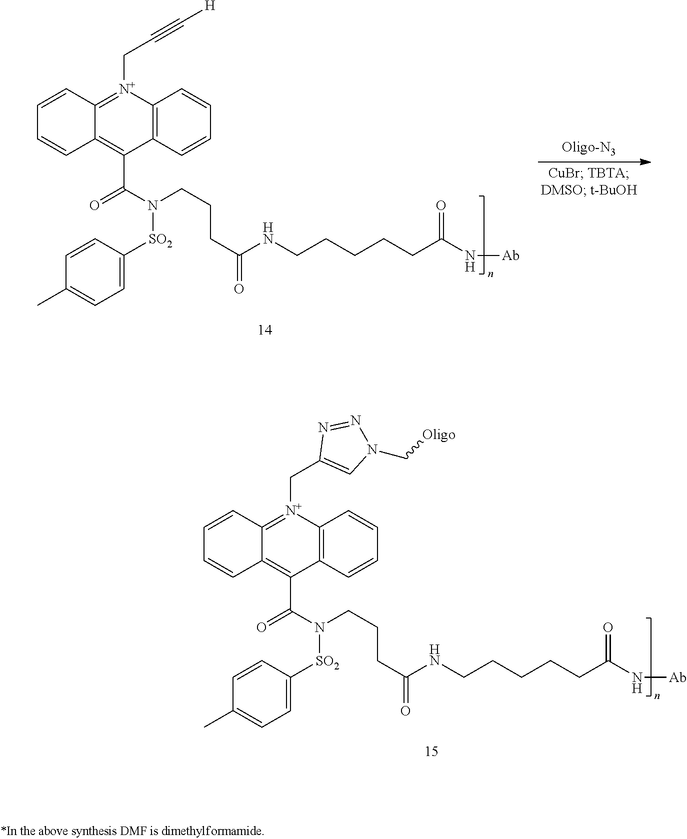

### 9-((4-((5-carboxypentyl)amino)-4-oxobutyl)(tosyl)carbamoyl)-10-(prop-2-yn-1-yl)acridin-10-ium (12)

A 25 mL round bottom flask equipped with a magnetic stirrer and a nitrogen inlet was charged with 8 (1 mmol), pyridine (5 mmol) and dimethylformamide (10 mL). The solution was cooled in an ice bath and pentafluorophenyl trifluoroacetate (1.3 mmol) was added dropwise. The ice bath was removed and the reaction was stirred at room temperature for 3 hours. 6-Aminocaproic acid (1.3 mmol) was then added to the reaction in small portions followed by N,N-diisopropylethylamine (5 mmol), and the reaction was stirred for 1 hour at room temperature. After this time, the volatile components were removed from the reaction in vacuo and the residue was purified by reverse phase HPLC to give the title compound.

### 9-((4-oxo-4-((6-oxo-6-(perfluorophenoxy)hexyl)amino)butyl)(tosyl)carbamoyl)-10-(prop-2-yn-1-yl)acridin-10-ium (13)

A 25 mL round bottom flask equipped with a magnetic stirrer and a nitrogen inlet was charged with 12 (1 mmol), pyridine (5 mmol) and dimethylformamide (10 mL). The solution was cooled in an ice bath and pentafluorophenyl trifluoroacetate (1.3 mmol) was added dropwise. The ice bath was removed and the reaction was stirred at room temperature for 3 hours. After this time, the volatile components were removed from the reaction under a stream of nitrogen and the residue was purified by reverse phase HPLC to give the title compound.

CPSP Antibody Conjugate with Spacer (14):

A solution of 13 (1 μL of a 10 mM solution in DMSO) was added to an antibody solution (100 μL of a 10 μM solution in water) and aqueous sodium bicarbonate (10 μL of a 1M solution). This was stirred at room temperature for 4 hours. Purification of the product was achieved on a spin column to give the CPSP antibody conjugate with spacer 14. Typically, “n” is any value between 1 and 10.

CPSP Oligonucleotide-Antibody Conjugate (15):

A mixture of an oligoazide (e.g., such as is commercially available) (10 nmol in 5 μL water), CPSP antibody conjugate with spacer 14 (10 nmol in 10 μL water) and a freshly prepared 0.1 M “click solution” (3 μL—see Example 13.C) was shaken at room temperature for 4 hours. Typically, “n” is any value between 1 and 10. The reaction was diluted with 0.3M sodium acetate (100 μL) and the DNA conjugate was precipitated by adding EtOH (1 mL). The supernatant was removed and the residue was washed 2× with cold EtOH (2×1 mL). The residue was taken up in water (20 μL) and the solution of the CPSP oligonucleotide-antibody conjugate with spacer 15 was used without further purification.

Cleavage of CPSP Antibody Conjugates with or without Spacer and CPSP Oligonucleotide-Antibody Conjugate with or without Spacer.

The CPSP antibody conjugates with or without spacer and CPSP oligonucleotide-antibody conjugate with or without spacer, as described, are cleaved or “triggered” using a basic hydrogen peroxide solution. In the ARCHITECT® system, the excited state acridone intermediate produces a photon, which is measured. In addition, the cleavage products are an acridone and a sulfonamide. The conjugates of Example 13.A-D are used with the disclosed device by counting the acridone and/or sulfonamide molecules.

E. CPSP Oligonucleotide Conjugate with No Antibody.

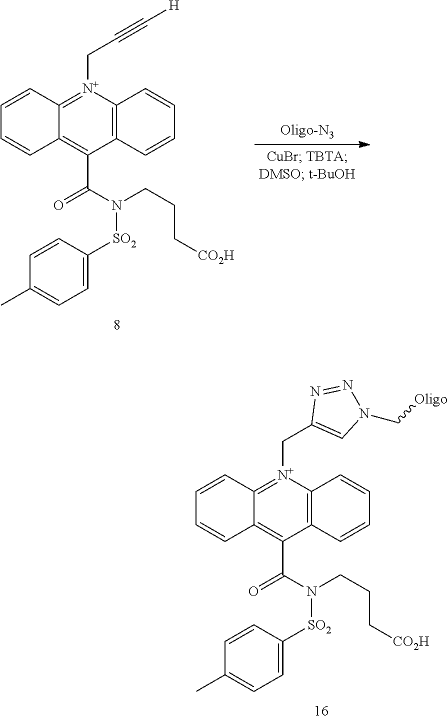

CPSP Oligonucleotide Conjugate with No Antibody (16):

A mixture of an oligoazide (e.g., such as is commercially available) (10 nmol in 5 μL water), propargyl-CPSP 8 (10 nmol in 10 μL water) and a freshly prepared 0.1 M “click solution” (3 μL—see Example 13.C) can be shaken at room temperature for 4 hours. The reaction can be diluted with 0.3M sodium acetate (100 μL) and the DNA conjugate precipitated by adding EtOH (1 mL). The supernatant can be removed and the residue washed 2× with cold EtOH (2×1 mL). The residue can be taken up in water (20 μL) and the solution of the CPSP oligonucleotide-antibody conjugate with spacer 16 can be used without further purification.

### Example 14

### Synthesis of Cleavable DNA-Biotin Construct

Synthesis of Non-Biotinylated Double-Stranded DNA (NP1):

Two single-stranded 50-mers were synthesized using standard phosphoramidite chemistry (Integrated DNA Technologies). Oligo NP1-1S consisted of a 50 nucleotide DNA sequence containing an amino group on the 5′-terminus, separated from the DNA by a C-12 carbon spacer (SEQ ID NO: 1) (1, MW=15,522.3 g/mole, ∈=502,100 M⁻¹ cm⁻¹). Oligo NP1-2AS consisted of a 50 nucleotide DNA sequence complementary to NP1-1S (SEQ ID NO: 2) (2, MW=15,507.1 g/mole, ∈=487,900 M⁻¹ cm⁻¹). Both oligonucleotides were quantitated and lyophilized prior to subsequent manipulation.

1
NP1-1S:
H₂N-AGTCATACGAGTCACAAGTCATCCTAAGATACCATACACATACCAA
GTTC
2
NP1-2AS:
GAACTTGGTATGTGTATGGTATCTTAGGATGACTTGTGACTCGTATGACT
3
Final ds-DNA Design-NP1:
H₂N-AGTCATACGAGTCACAAGTCATCCTAAGATACCATACACATACCAA
GTTCTCAGTATGCTCAGTGTTCAGTAGGATTCTAIGGTATGTGTATGGTT
CAAG

Synthesis of Non-Biotinylated Double-Stranded 50-bp DNA Construct:

NP1-2AS (1.44 mg, 93.4 nmoles) was reconstituted in 0.5 mL distilled water to give a 187 μM solution. NP1-1S (1.32 mg, 85.3 nmoles) was reconstituted in 0.5 mL of 50 mM phosphate, 75 mM sodium chloride buffer pH 7.5 to give a 171 μM solution. The double-stranded construct (3) (SEQ ID NO: 1—forward strand (top); SEQ ID NO: 2—reverse strand (bottom)) was made by annealing 60 μL of NP1-1S solution (10.2 μmoles) with 40 μL of NP1-2AS solution (7.47 μmoles). The mixture was placed in a heating block at 85° C. for 30 min, followed by slow cooling to room temperature over 2 hours. Double-stranded material was purified by injecting the total annealing volume (100 μL) over a TosoH G3000SW column (7.8 mm×300 mm) equilibrated with 10 mM PBS buffer, pH 7.2. The column eluent was monitored at 260 and 280 nm. The double-stranded material (3) eluted at 7.9 minutes (approx. 20 minutes). The DNA was concentrated to 150 μL using a 0.5 mL Amicon filter concentrator (MW cut-off 10,000 Da). The final DNA concentration was calculated to be 40.5 μM, as determined by A₂₆₀ absorbance.

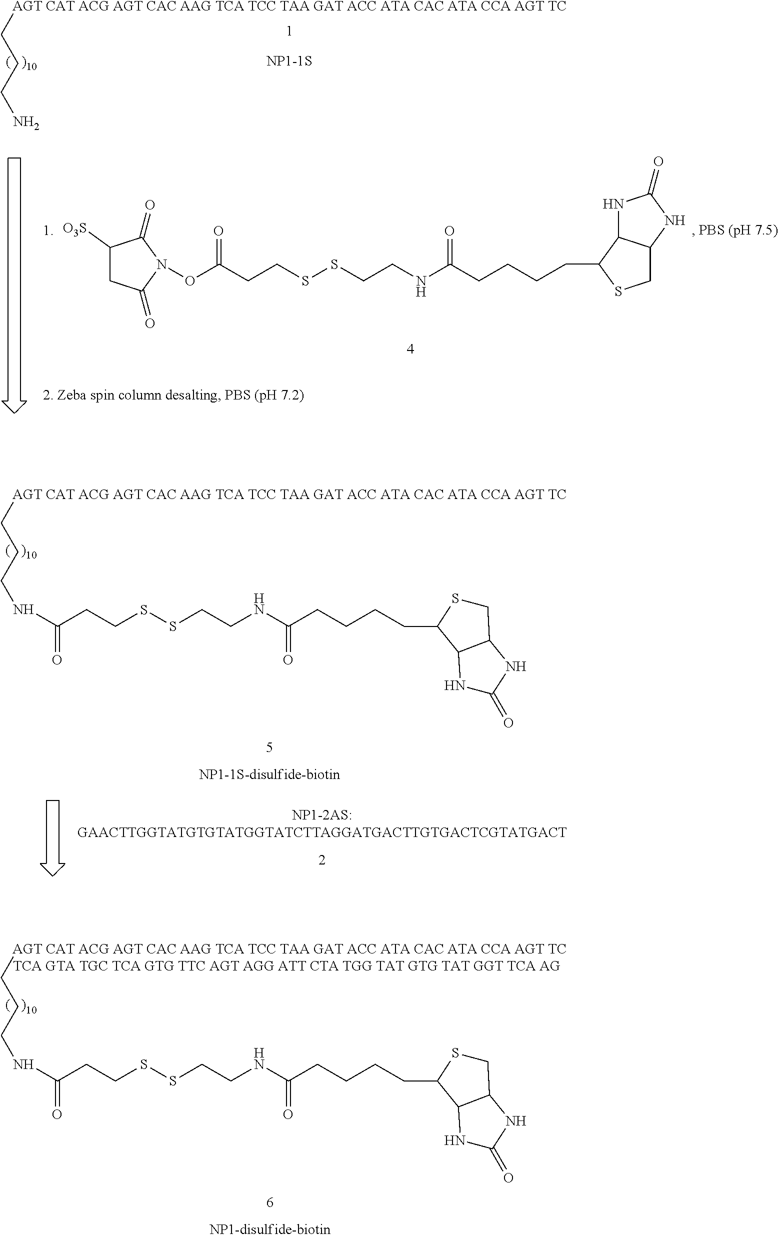

Biotinylation of Single-Stranded 5′-Amino Oligo:

A 100 mM solution of sulfo-NHS-SS-Biotin (4, ThermoFisher Scientific) was made by dissolving 6 mg of powder in 0.099 mL of anhydrous DMSO (Sigma Aldrich). The solution was vortexed and used immediately for biotinylating the 5′-amino-DNA. Approx. 100 μL of ssDNA (1, 171 μM, 17.1 μmoles, 0.265 mg) (SEQ ID NO: 1) solution in 50 mM PBS, pH 7.5 was mixed with 3.4 μL of 0.1 mM biotinylating reagent in DMSO (34.1 μmoles, 20-fold molar excess over the ssDNA). The mixture was mixed and allowed to react at room temperature for 2 hours. Two 0.5 mL Zeba spin desalting columns (MW cut-off 7,000 Da, ThermoFisher Scientific) were equilibrated in 10 mM PBS, pH 7.2. The crude biotinylated ssDNA solution was added to one Zeba column and eluted at 4,600 rpm for 1.3 minutes. The eluent was transferred to a second Zeba column and eluted as described. The concentration of the purified NP1-1S-SS-Biotin (5) (SEQ ID NO:1 was determined by measuring the A₂₆₀ absorbance (2.03 mg/ml, 131 μM).

Formation of Biotinylated Double-Stranded DNA:

Approximately 60 μL of NP1-1S-SS-Biotin solution (5, 7.85 μmoles, 131 μM, 2.03 mg/mL) was mixed with 42 μL of NP1-2AS solution (2, 7.85 μmoles, 187 μmol/L) (SEQ ID NO: 2). The solution was placed in a heating block at 85° C. for 30 minutes, followed by slow cooling to room temperature over 2 hours. The double-stranded product was purified over a TosoH G3000SW column (7.8 mm×300 mm) using 10 mM PBS, pH 7.2 by injecting the entire annealing volume (approx. 100 μL). The double-stranded biotinylated material eluted at 7.9 minutes (20 minutes run time), as monitored by A₂₆₀ absorbance. The eluent volume was reduced to 480 μL using a 0.5 mL Amicon filter concentrator (MW cut-off 10,000 Da). The final NP1-dithio-biotin (6) (SEQ ID NO:1—forward strand (top); SEQ ID NO:2—reverse strand (bottom)) concentration was calculated to be 16.3 μM, as determined by A₂₆₀ absorbance.

### Example 15

### Alternate Synthesis of Cleavable DNA-Biotin Construct

Complementary DNA Sequences (NP-31a and NP-31b):

Two single-stranded 60-mers were synthesized using standard phosphoramidite chemistry (Integrated DNA Technologies). Oligo NP-31a consisted of a 60 nucleotide DNA sequence containing an amino group on the 5′-terminus, separated from the DNA by a C-6 carbon spacer (SEQ ID NO: 3) (1, MW=18,841.2 g/mole, 1.7 μM/OD). Oligo NP-31b consisted of a 60 nucleotide DNA sequence complementary to NP-31a (SEQ ID NO: 4) (2, MW=18292.8 g/mole, 1.8 μM/OD). Both oligonucleotides were quantitated and lyophilized prior to subsequent manipulation.

1
NP-31a:
H₂N-5′GCC CAG TGT CTT TGT AGG AGG AGC AGC GCG TCA
ATG TGG CTG ACG GAC CAT GGC AGA TAG3′
2
NP-31b:
5′CTA TCT GCC ATG GTC CGT CAG CCA CAT TGA CGC GCT
GCT CCT CCT ACAAAG ACA CTG GGC3′
3
ds-DNA Design-NP-31:
H₂N-5′GCC CAG TGT CTT TGT AGG AGG AGC AGC GCG TCA
ATG TGG CTG ACG GAC CAT GGC AGA TAG3′
3′CGG GTC ACA GAA ACA TCC TCC TCG TCG CGC TGA TAC
ACC GAC TGC CTG GTA CCG TCT ATC5′

Biotinylation of Single-Stranded 5′-Amino Oligo NP-31a:

A 10 mM solution of NHS-S-S-dPEG₄-Biotin (4, MW=751.94 g/mole, Quanta BioDesign, Ltd) was prepared by dissolving 15.04 mg of powder in 2.0 mL of dimethylformamide (Sigma Aldrich). The solution was vortexed and used immediately for biotinylating the 5′-amino-DNA. Approx. 100 μL of ssDNA (1, 100 μM, 0.01 μmoles, 0.188 mg) (SEQ ID NO: 3) solution in 10 mM phosphate buffered saline (PBS), pH 7.4 was mixed with 10 μL of 10 mM biotinylating reagent in DMF (0.1 μmoles, 10-fold molar excess versus the ssDNA). The mixture was mixed and allowed to react at room temperature for 2 hours. Two 0.5 mL Zeba spin desalting columns (MW cut-off 7,000 Da, ThermoFisher Scientific) were equilibrated in 10 mM PBS, pH 7.2. The crude biotinylated ssDNA solution was added to one Zeba column and eluted at 4,600 rpm for 2 minutes. The eluent was transferred to a second Zeba column and eluted as described. The concentration of the purified NP-31-SS-Biotin (5) (SEQ ID NO: 3) was determined by measuring the A₂₆₀ absorbance (1.45 mg/mL, 77 μM).

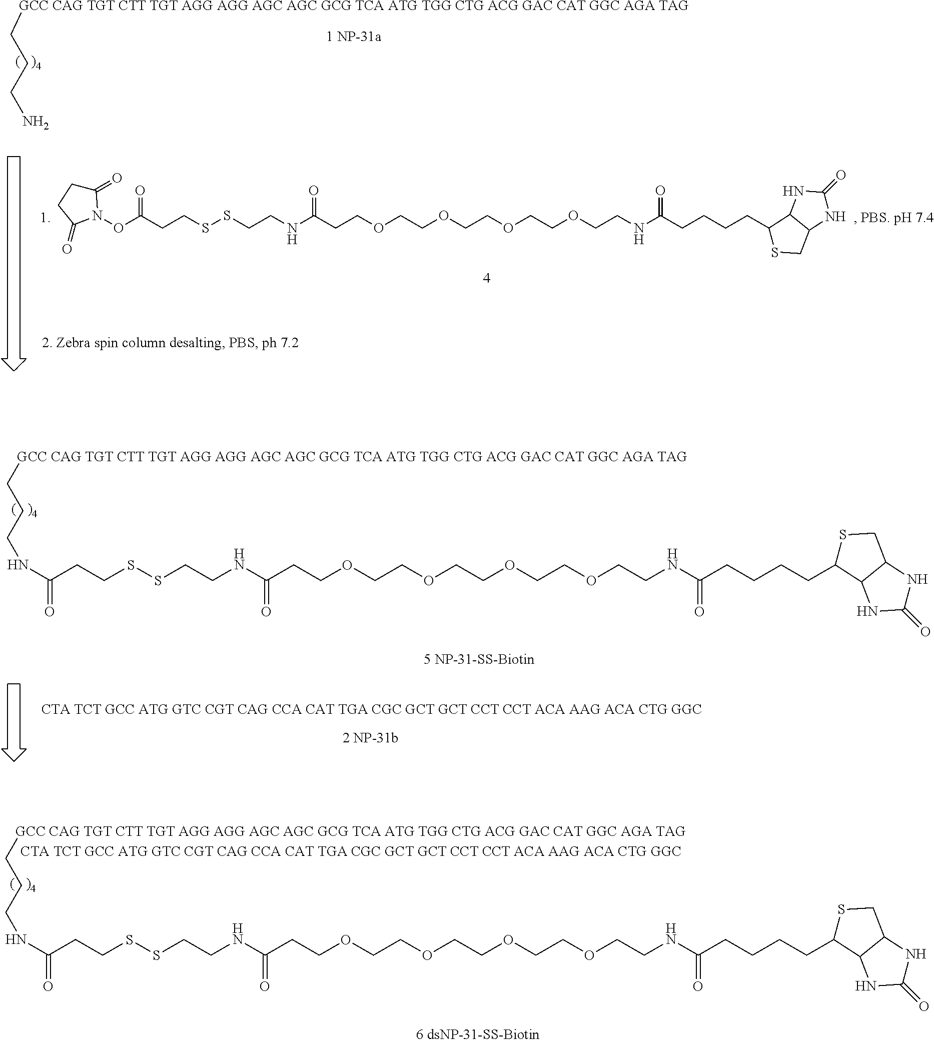

Formation of Biotinylated Double-Stranded DNA:

Approximately 60 μL of NP-31-SS-Biotin solution (5, 77 μM) (SEQ ID NO: 3) was mixed with 50 μL of NP-31b solution (2, 100 μM). The solution was placed in a heating block at 85° C. for 30 minutes, followed by slow cooling to room temperature over 2 hours. The double-stranded product (SEQ ID NO:3—forward strand (top); SEQ ID NO:4—reverse strand (bottom)) was purified over a TosoH G3000SW column (7.8 mm×300 mm) using 10 mM PBS, pH 7.2 by injecting the entire annealing volume (approximately 100 μL). The double-stranded biotinylated material eluted at 7.57 minutes as monitored by A₂₆₀ absorbance. The eluent volume was reduced using a 0.5 mL Amicon filter concentrator (MW cut-off 10,000 Da). The final dsNP-31-SS-Biotin (6) (SEQ ID NO: 3—forward strand (top); SEQ ID NO: 4—reverse strand (bottom)) concentration was calculated to be 11 μM, as determined by A₂₆₀ absorbance.

### Example 16

### Synthesis of Cleavable DNA-Biotin—Thiol-Mediated Cleavage Construct

Binding of ssNP-31-SS-Biotin to Streptavidin Coated Magnetic Microparticles (SA-MP) and Chemical Cleavage (TCEP or DTT):

Chemical cleavage experiments were performed on magnetic microparticles via the following method (see FIG. 55). 100 μL of the modified oligonucleotide ssNP-31-SS-Biotin solution at 77 μM in PBS, pH 7.2 was incubated with 1 μL 0.1% Streptavidin paramagnetic microparticles for 30 minutes at room temperature. Excess oligo was removed by attracting the particles to a magnet and washing 10 times with PBST buffer, pH 7.4. The oligo-bound particles were incubated with varying concentrations of either DTT or TCEP in PBS, pH 7.4 for 15 minutes. Microparticles were washed 10 times with PBST buffer, pH 7.4 to remove any cleaved oligonucleotide. Complementary sequence NP-31c (SEQ ID NO: 5) (7, MW=7494.6 g/mole, 5.2 μM/OD) containing a fluorophore was incubated with the microparticles for 30 minutes in PBS, pH 7.4 to bind with any uncleaved ssNP-31-SS-Biotin remaining intact on the particle. The microparticles were attracted to a magnet and washed 10 times with PBST buffer, pH 7.4 to remove any excess NP-31c segments. Coated microparticles prepared as above that were washed but not subjected to chemical cleavage served as a control. The fluorescent signal on the particles was measured by fluorescence microscopy. Maximum cleavage efficiency was measured at 79% and 93% for DTT and TCEP, respectively as shown in Table 1.

7
NP-31c:
AlexaFluora ® 546-5′CTA TCT GCC ATG GTC CGT CAG3′

TABLE 1

| | Fluorescent Signal on Microparticles | |
|---|---|---|
| | (Relative Light Units) | Cleavage Efficiency |
| DTT (mM) | | |
| 50 | 3579 | 79% |
| 25 | 7417 | 57% |
| 12.5 | 11642 | 32% |
| 0.78125 | 17052 | 0% |
| 0 | 17059 | (Control) |
| TCEP (mM) | | |
| 250 | 460 | 93% |
| 222 | 448 | 94% |
| 187 | 474 | 93% |
| 142 | 512 | 92% |
| 83 | 477 | 93% |
| 45 | 453 | 93% |
| 19 | 452 | 93% |
| 0 | 5023 | (Control) |

### Example 17

### Synthesis of Cleavable DNA-Biotin—Photocleavage Construct

Evaluation of a Photocleavable DNA Sequence and Efficiency of Cleavage on Microparticles:

A photocleavable sequence of single-stranded DNA was synthesized using standard phosphoramidite chemistry (Integrated DNA Technologies). The oligonucleotide consisted of 48 nucleotides composing two Oligo segments (Oligo 8-1 (SEQ ID NO: 6) and Oligo 8-2 (SEQ ID NO: 7)) separated by two photocleavable moieties (8, MW=15,430.1 g/mole, 441800 L mol⁻¹ cm⁻¹). The 5′-terminus contained an amino group separated from the DNA by a C-6 carbon spacer. A complementary strand to Oligo 8-2 was synthesized containing a fluorescent tag (9, MW=7738.8 g/mole, 212700 L mol⁻¹ cm⁻¹) (SEQ ID NO: 8). Both oligonucleotides were quantitated and lyophilized prior to subsequent manipulation.

Oligo 8-1 (SEQ ID NO: 6):
5′AAA AAA GGT CCG CAT CGA CTG CAT TCA3′
Oligo 8-2 (SEQ ID NO: 7):
5′CCC TCG TCC CCA GCT ACG CCT3′
NP-8(8)(Oligo 8-1 (SEQ ID NO: 6) and Oligo 8-2
(SEQ ID NO: 7) joined by two photocleavable
moieties (“PC”)):
H₂N-5′AAAAAAGGTCCGCATCGACTGCATTCA-PC--PC-
CCCTCGTCCCCAGCTACGCCT3′
NP-9 (9)(SEQ ID NO: 8):
AlexaF1uor546-5′AGG CGT AGC TGG GGA CGA GGG3′

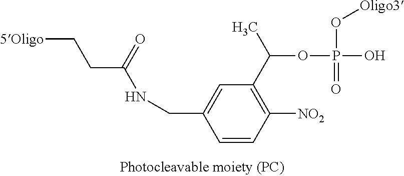

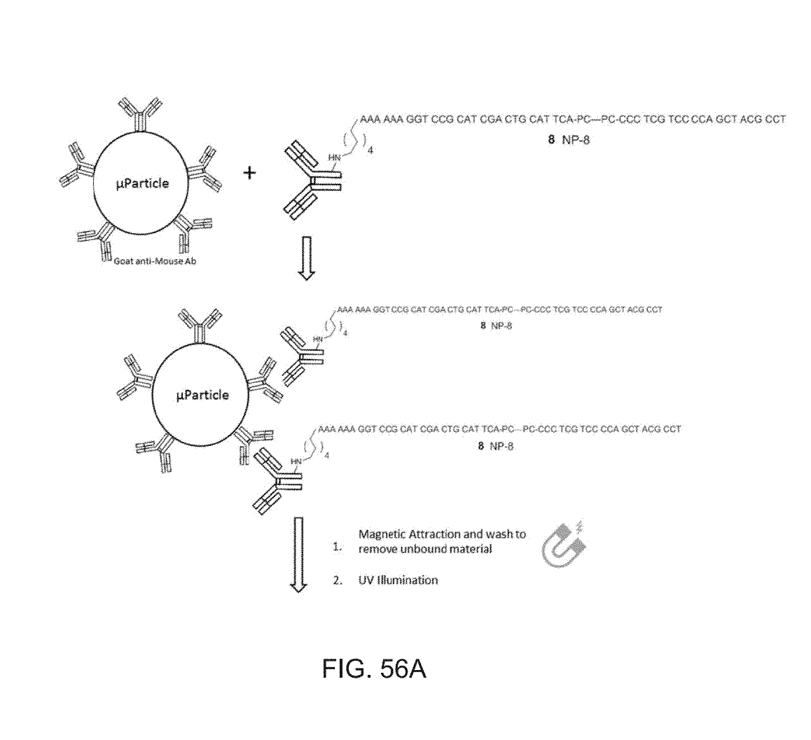

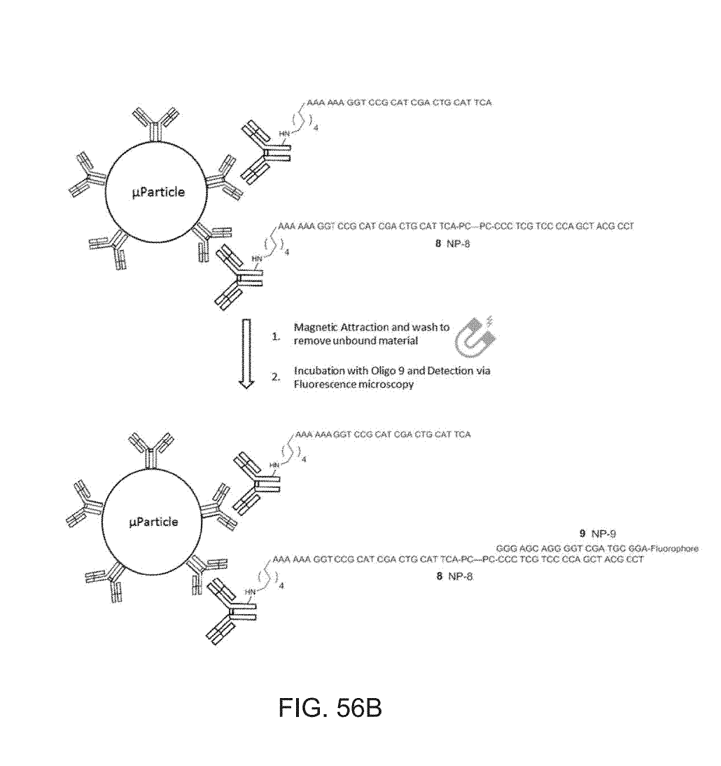

Photocleavage experiments were performed on magnetic microparticles via the following method (see FIGS. 56A and 56B). NP-8 was covalently attached to an antibody to generate an Ab-oligo complex (prepared by Biosynthesis Inc.). 100 μL of 33 nM antibody-oligo complex was incubated with 1 μL 0.1% solids of goat anti-mouse microparticles for 30 minutes at room temperature. Excess antibody-oligo complex was removed by attracting the particles to a magnet and washing 10 times with PBST buffer, pH 7.4. The microparticle complex solution was illuminated under UV light (300-350 nm wavelength) for 5 minutes. The microparticles were attracted to a magnet and washed 10 times with PBST buffer, pH 7.4 to remove any cleaved Oligo segments. Following particle resuspension in PBST buffer, pH 7.4, fluorescently labeled Oligo 9 (SEQ ID NO: 8) was added to the irradiated microparticles and incubated for 30 minutes at room temperature. Coated microparticles prepared as above that were washed but not subjected to UV illumination (uncleaved Oligo 8-2) served as a control. The fluorescent signal (AlexaFluor® 546) on the particles was imaged by a fluorescence microscope. Cleavage efficiency when bound to paramagnetic microparticles was measured at 74% as shown in Table 2.

TABLE 2

| | Fluorescent Signal |
|---|---|
| | on Microparticles |
| | (Relative Light Units) |
| Illumination | 3660 |
| No Illumination (Control) | 13928 |
| Cleavage Efficiency | 74% |

### Example 18

### Thermal Cleavable Linkers

This Example describes thermal cleavable linkers and their cleavage. Such thermal cleavable linkers can be employed, for example, in a DMF chip, droplet-based microfluidic chip, SAW chip, or the like, as described herein.

Thermal cleavable linkers are cleaved by elevating the temperature above a threshold, such as in the thermal separation of double-stranded DNA. Temperature elevation in the DMF chip can be achieved photothermally by transferring energy from light to an absorbing target. In one method, a source of light, such as a laser, having a wavelength of about 980 nm (range about 930 nm to about 1040 nm) can be applied to the DMF chip in the region of the fluid sample. The light can be absorbed by the water molecules in the fluid, resulting in an increase in temperature and cleavage of the linker. The level and duration of heating can be controlled by pulse length, pulse energy, pulse number, and pulse repetition rate. For example, photothermal heating using the absorbance band of water is described, e.g., U.S. Pat. No. 6,027,496.

Photothermal heating also can be achieved by coupling the light source with a dye, or pigment containing target. In this case, a target area of the DMF chip is printed with an absorbing dye or pigment, e.g. carbon black. When the fluid is in contact with the target, the light source, e.g. a commercially available laser diode, is directed at the light-absorbing target, resulting in a localized increase in temperature and cleavage of the linker. The level and duration of heating can be controlled by the absorbance properties of the target, light wavelength, pulse length, pulse energy, pulse number, and pulse repetition rate. For example, photothermal heating using a light source in combination with a light absorbing target is described in U.S. Pat. No. 6,679,841.

In a third method of photothermal heating, an absorbing dye or pigment can be introduced into the fluid in the DMF chip. The light is then transmitted through the DMF chip and the energy transferred to the dissolved or suspended absorbing material, resulting in a localized increase in temperature and cleavage of the linker. The level and duration of heating is controlled by absorbance properties of the target material, light wavelength, pulse length, pulse energy, pulse number, and pulse repetition rate. In one embodiment of this method, the light absorbing target is the magnetic microparticle suspension used in the device. For example, photothermal heating using suspended nanoparticles in a fluid droplet is described in Walsh et al., Analyst, 140(5), 1535-42 (2015). The reference of Walsh et al. also demonstrates some of the control that can be achieved in photothermal applications.

### Example 19

### Thermal Cleavage Accomplished Via Microwave-Induced Particle Hyperthermia

This Example describes the use of microwave-induced particle hyperthermia to facilitate thermal denaturation (such as dsDNA denaturation, retro-Michael reactions, retro-Diels-Alder, and other eliminations) to release a countable moiety via a thermal sensitive cleavable linker, as immunoassay detection can be accelerated with the use of low powered microwave radiation. Such thermal sensitive cleavable linkers can be employed, for example, in a DMF chip, as described herein.

In this example, formation of an orthogonally functionalized short dsDNA segment such as a 15 bp sequence with a double stranded Tₘ in the range of 40-55° C. serves as the thermal release agent. The dsDNA segment can be reacted with antibody via attachment chemistry such as sulfhydryl-maleimide interaction and 26 nm carboxylated polystyrene nanoparticles (NP), such as those which can be obtained from Bangs Labs (Fishers, Ind., USA) via attachment chemistry such as amine-activated carboxylic acid chemistry. The 26 nm NPs have a surface charge of 528.7 μeq/g and a parking area of 68.4 sq. Å/group (per manufacturer information). The antibody and nanoparticle are associated through the dsDNA segment which forms a thermally triggered releasable linker. The thermal linker can be cleaved using a technique such as microwave irradiation to trigger particle hyperthermia and a localized temperature gradient.

DNA Sequence 1 (10) (SEQ ID NO: 9):
H₂N-5′ CAA GCC CGG TCG TAA3′
DNA Sequence 1b (11) (SEQ ID NO: 10):
Maleimide-5′ TTA CGA CCG GGC TTG3′
dsDNA Sequence (12)(SEQ ID NO: 9 - forward strand
(top); SEQ ID NO: 10 - reverse strand (bottom)):
H₂N-5′ CAA GCC CGG TCG TAA3′
3′ GTT CGG GCC AGC ATT5′-Maleimide.

Annealing of the Orthogonally Functionalized Complementary DNA Sequences Complex:

A solution of approximately 100 μM DNA Sequence 1 (SEQ ID NO: 9) in PBS pH 7.5 can be mixed with 1.0 molar equivalents of DNA Sequence 1b (theoretical Tₘ 51.6° C. per Integrated DNA Technologies oligo analyzer tool) (SEQ ID NO: 10) in PBS pH 7.5 and placed in a heating block at 60° C. for 30 minutes, followed by slow cooling to room temperature over 2 hours. The resulting dsDNA product is purified over a TosoH G3000SW column (7.8 mm×300 mm) using 10 mM PBS, pH 7.2 by injecting the entire annealing volume. The eluent volume is reduced using a 0.5 mL Amicon filter concentrator. The final dsDNA concentration is determined by A260 absorbance. The reaction scheme is depicted below (“Mal” is maleimide).

Activation of Carboxyl-Polystyrene Nanoparticles and Addition of Double Stranded DNA:

Carboxy nanoparticles are preactivated as described in Example 12 under section “Activation of carboxyl-polystyrene nanoparticles.” The DNA loading on the NP is determined by thermal denaturation of the bound DNA strands, particle washing, annealing of a fluorescently labelled complementary DNA sequence (such as AlexaFluor546-5′-TTA CGA CCG GGC TTG3′ (SEQ ID NO: 11)) and quantified using a fluorescence microscope.

Antibody Reduction and Conjugation to a NP-dsDNA Complex:

The antibody is reduced as described in Example 12 under section “Antibody reduction.” The reduced antibody can be used immediately for coupling to the NP-dsDNA complex. The resulting conjugate is centrifuged at 6,500 g and the supernatant is removed via decanting. The wash procedure is repeated 5 times with PBS pH 7.5 to remove any free antibody from the nanoparticle. The active antibody to nanoparticle incorporation ratio may be quantified using a fluorescently labeled antigen to the given antibody. The conjugate NP concentration (% solids) is determined using UV-Vis spectroscopy (600 nm). The particle conjugate is examined by SEM and the size/charge distribution is determined using the ZetaSizer.

Microwave-Induced Particle Hyperthermia and Nanopore Counting Immunoassay:

The scheme above illustrates the nanopore counting assay utilizing the thermally denatured antibody-nanoparticle conjugate whose preparation is described above. A sandwich type immunoassay can be prepared using magnetic microparticles coated with an analyte capture agent in which blood analyte is incubated with magnetic microparticles, washed, and incubated with the antibody-nanoparticle conjugate described. Particle hyperthermia can be induced using microwave irradiation to create a local temperature gradient near the particle surface. Particle hyperthermia methods such as those reviewed in Dutz and Hergt (Nanotechnology, 25:452001 (2014)) may be used. The adaptation of these techniques to local thermal denaturation in an immunoassay setting can provide a method to release a counting moiety (such as a nanoparticle). Following removal of the magnetic microparticles, the counting moiety (nanoparticle) is counted upon passage through the nanopore.

### Example 20

### Nanopore Module Fabrication

A nanopore module was fabricated using standard soft lithography fabrication methods coupled with integration of a commercially available silicon nitride (SiNₓ) membrane embedded in a TEM window (Norcada). The module consisted of four separate layers of PDMS—a top and bottom PDMS substrate containing the transfer microchannels, and two optional intermediate PDMS layers to seal the TEM window.

SU8 Master Mold Fabrication:

A clean, dry glass substrate was spincoated with photoresist (SU8-50) to a desired thickness. Areas of the coated substrate were then selectively exposed to near-UV light using a photomask. The mask exposes photoresist to UV light only in regions where the transfer microchannel and reservoir shapes are to remain. Exposure was followed by a bake to cross-link regions of photoresist that were exposed. An SU8 developer was then used to remove remaining, unexposed photoresist from the substrate. The final product is a master mold—a glass substrate with patterned transfer microchannels and reservoirs of hard photoresist.

Intermediate PDMS Layer Fabrication:

For fabricating the intermediate PDMS layers, a solution containing PDMS monomer and its curing agent (Sylgard 184 silicone elastomer) in the ratio of 7:1 PDMS monomer:curing agent was spincoated on a glass slide, followed by heating on a hot plate for 30 minutes at 70° C. The PDMS layers were peeled off the glass substrate and 1.25 mm cut-out was punched through the PDMS layers to provide an opening allowing access to the TEM window. Surface of the PDMS layers was made hydrophilic by plasma treating for 30 seconds using a corona treater at a distance of 8 mm. A second plasma treatment (5 seconds) was used to treat the surface of the PDMS layers and TEM window before bonding the SiNₓ TEM window between the two intermediate PDMS layers.

Top and Bottom PDMS Fabrication:

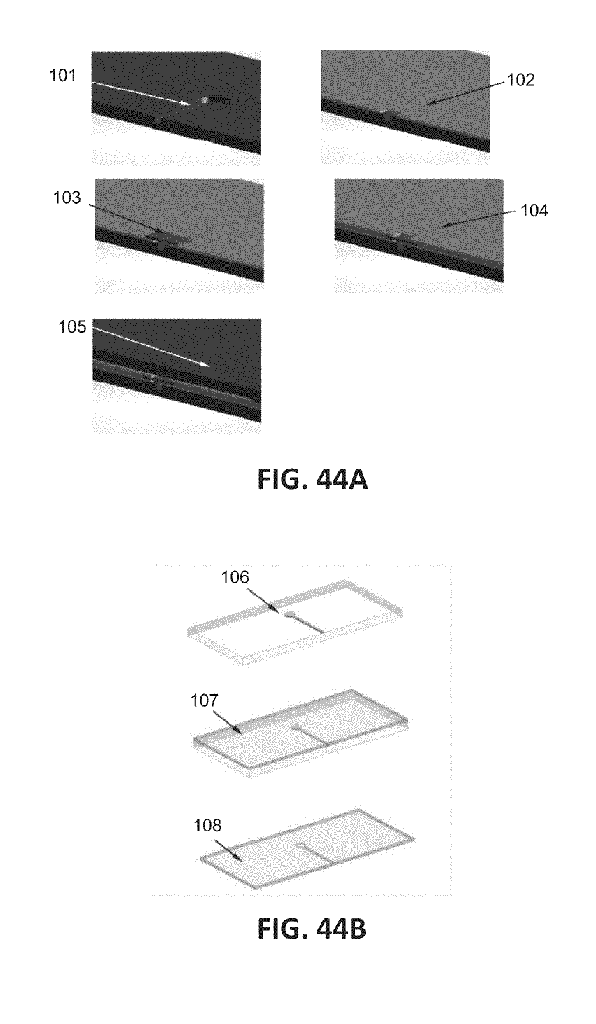

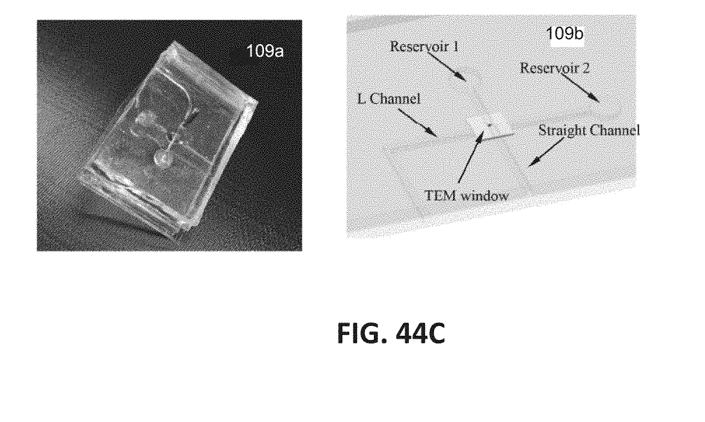

The top and bottom PDMS substrates containing microchannels were fabricated, as shown in FIG. 44B, by mixing PDMS monomer and curing agent in a ratio of 7:1 PDMS monomer:curing agent and pouring over glass containing the SU8 patterned mold (106) patterned with the transfer microchannels and reservoirs (see SU8 Master Mold Fabrication described above). The microchannels measured approximately 110 to 135 μm in width and 50 μm in depth. After degassing for 15 minutes, the SU8 mold was heated on a hot plate for 60 minutes at 70° C. (107). After curing, the PDMS substrates were peeled off the SU8 mold (108) and cut to yield rectangular PDMS substrates having an approximate dimension of 30 mm length×20 mm width×3 mm depth. Access holes (1.25 mm in diameter) were punched through the PDMS substrates to allow subsequent insertion of electrodes into the microchannels. The final assembly is shown in FIG. 44A and includes from bottom to top, bottom PDMS substrate containing one microchannel (101), a first intermediate PDMS layer (102) containing a cut-out positioned over the microchannel, the SiNₓ membrane in TEM window (103), a second intermediate PDMS layer (104) also containing a cut-out, and a top PDMS substrate (105) containing a second microchannel.

Alignment of Top and Bottom PDMS Substrates:

A PDMS bottom substrate (prepared as outlined in “Top and Bottom PDMS Fabrication,” above) was plasma treated for 30 seconds, followed by bonding of a first intermediate PDMS layer (prepared as outlined in “Intermediate PDMS Layer Fabrication,” above) onto the PDMS bottom substrate. Similarly, a PDMS top substrate was plasma treated for 30 seconds, followed by bonding of a second intermediate PDMS layer onto the PDMS top substrate. The cut-outs in the intermediate layers were aligned with the microchannels. Both top and bottom PDMS pieces were oxygen plasma treated for 30 seconds, followed by placement of the SiNx membrane window in between the top and bottom pieces and aligned with the cut-outs in the intermediate PDMS layers. The top piece aligned with the SiNx membrane aligned with the bottom piece were pressed together until all air bubbles were released. The final nanopore PDMS assembly was heated on a hot plate for at 100° C. for 30 minutes and plasma treated for 5 minutes. The final module assembly, shown in FIG. 44C, (109a) contained two channels (one straight and one “L-shaped” channel), each ending in a reservoir for a solution (e.g., a buffer). The TEM window containing the SiNx membrane is positioned at the intersection of the two perpendicular microchannels (FIG. 44C, (109b)).

### Example 21

### Nanopore Fabrication

Nanopore fabrication was accomplished by subjecting a SiNₓ TEM window, housed between two PDMS layers, to a potential bias until dielectric breakdown occurred, thereby opening up a small-diameter hole in the membrane. This allows for in situ formation of a pore within the microfluidic device, prior to detection of analytes. Nanopore formation by dielectric breakdown has been previously shown to be useful for rapid fabrication of small diameter pores in solid-state dielectric membranes (H. Kwok, K. Briggs, V. Tabard-Cossa, PLoS-One, 9(3), 2014).

SiNₓ membrane commercially available as transmission electron microscope (TEM) windows (Norcada) were embedded in the assembled PDMS module as outlined in Example 20 above) and were used to generate the nanopore. The perpendicular microchannel junction exposed a cross sectional area (50 μm×50 μm) of the SiNₓ TEM window to a salt solution (1 M KCl) disposed on opposite sides of the membrane (cis and trans). Ag/AgCl electrodes were placed into each microchannel approximately 3 mm from the center of the SiNₓ TEM window into holes punched through the PDMS substrate. A syringe containing a blunt needle was used to fill both cis and trans microchannels by adding ethanol to the two reservoirs until liquid was observed emerging from the channel openings on the module edge. The resistance was measured to check for proper sealing and to ensure the TEM-SiNₓ membrane was intact. A resistance on the order of MΩ indicated good sealing and a membrane that was intact and undamaged. The ethanol was flushed out of the microchannel with deionized water, and replaced with a 1 M KCl solution by injecting into the two reservoirs. The resistance was measured again to check for proper sealing.

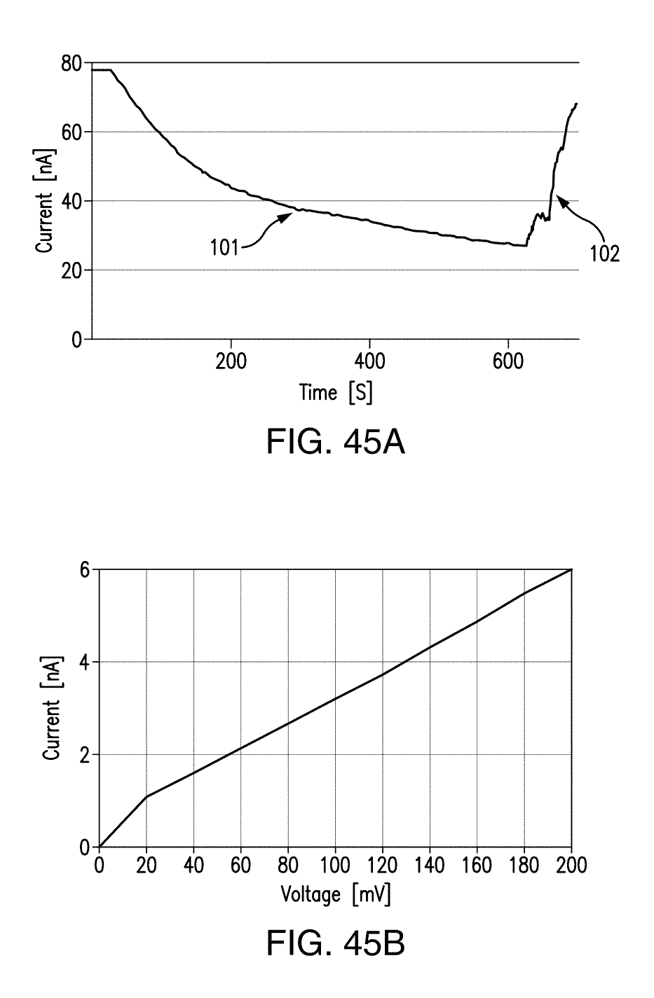

A constant voltage of 4.4 V was applied to the membrane assembly and the leakage current was monitored in real-time. The leakage current measured in real-time is plotted in FIG. 45A. FIG. 45A shows the leakage current (101) prior to nanopore creation. A threshold value of >5 nA was used as the cut-off value, i.e.—to signify pore creation. After approximately 10 minutes, an increase in leakage current was observed (102). The voltage was turned off immediately following the detection of increase in leakage current. The diameter of the created pore was 6.9 nm, as determined by the following relationship:

$$G = {σ ( \frac{4 L}{π {d}^{2}} + \frac{1}{d} )}^{- 1}$$

where G=conductance, σ=bulk conductivity (12.35 S/m measured for KCl), L=thickness of the membrane (10 nm), d=pore diameter (S. Kowalczyk, A. Grosberg, Y. Rabin, C. Dekker, Nanotech., 22, 2011).

After pore creation, a current-voltage (I-V) curve (see FIG. 45B) was used to verify that the nanopore displayed ohmic behavior, indicating the nanopore was symmetrical in shape and the resistance was independent of the applied voltage or current. The same 1 M KCl solution was used for both pore fabrication and I-V curves.

### Example 22

### Dry Microchannel Filling

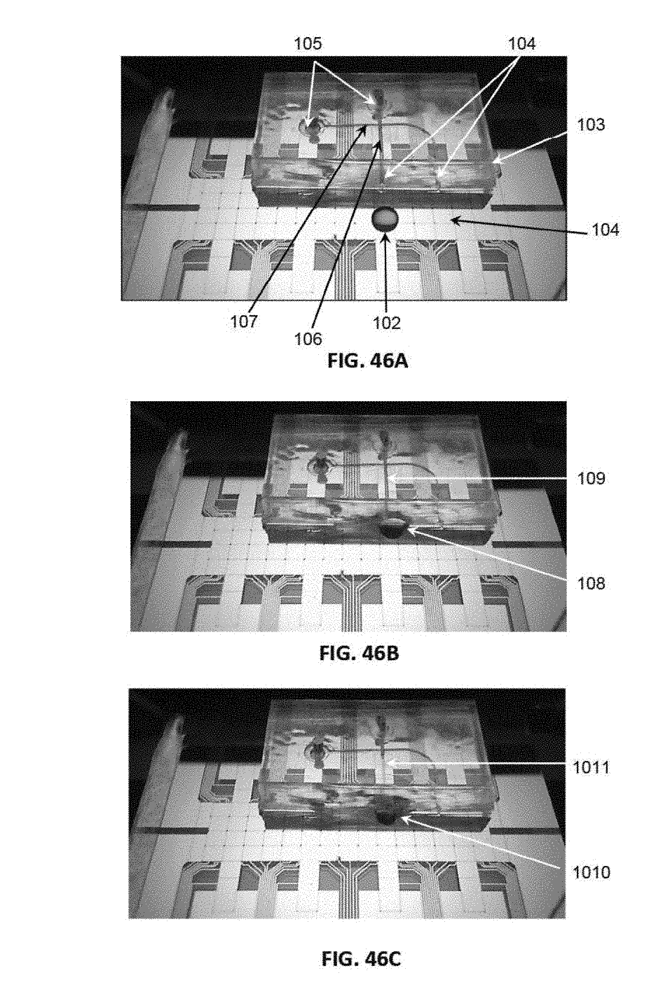

The capillary conduit contained in the assembled PDMS module (i.e., the integrated device including a DMF module and a nanopore module) was tested for its ability to spontaneously fill high-salt solutions from the DMF electrode assembly (FIGS. 46A-46C). Filling was achieved via spontaneous capillary flow (SCF). The nanopore membrane was not included in order to allow for better visualization of the microchannels. With reference to FIG. 46A, a glass DMF chip (3″×2″×0.0276″) containing 80 actuation electrodes (101) (2.25 mm×2.25 mm, Cr-200 nm thickness) was used to move a droplet (102) of 3.6 M LiCl, 0.05% Brij 35 and blue dye (to aid with visualization). The PDMS module (103) contained two openings facing the DMF electrode array (104), two reservoirs (105) and two microchannels—one straight channel (106) and one L-shaped channel (107). The module assembly was placed on the DMF glass surface so that the two channel openings faced the interior of the DMF electrode array. Since a top grounding electrode chip was not used, droplet movement was achieved by using co-planar bottom electrodes to generate the driving potential.

A 10 μL droplet of blue-colored LiCl salt solution was placed on an electrode in the middle of the DMF electrode array. A voltage of 100 Vᵣₘₛ (10 kHz) was used to move the droplet to the transfer electrode adjacent to the straight microchannel opening. As shown in FIG. 46B, after the droplet contacted the PDMS surface (108), the time required to fill the 130 μm diameter straight channel (109) and reach the reservoir was measured. As shown in FIG. 46C, after approximately 30 seconds, the volume of the droplet was visibly smaller (1010) and the channel was half filled (1011). A total time of 53 seconds was required to fill the entire dry microchannel (130 μm diameter).

Wet Microchannel Filling:

A 10 μL droplet of blue-colored LiCl salt solution was placed on an electrode in the middle of the DMF electrode array. A voltage of 100 Vᵣₘₛ (10 kHz) was used to move the droplet to the transfer electrode adjacent to the straight microchannel opening. The channel was pre-filled with ethanol to mimic a pre-wetted channel. After the droplet contacted the PDMS surface, a time of <1 second was required to fill the channel up to the reservoir. This was significantly faster than the dry channel, suggesting pre-wetting with a hydrophilic solution enhances microchannel fill rates.

### Example 23

### DMF Droplet Transfer in Integrated Silicon NP Device

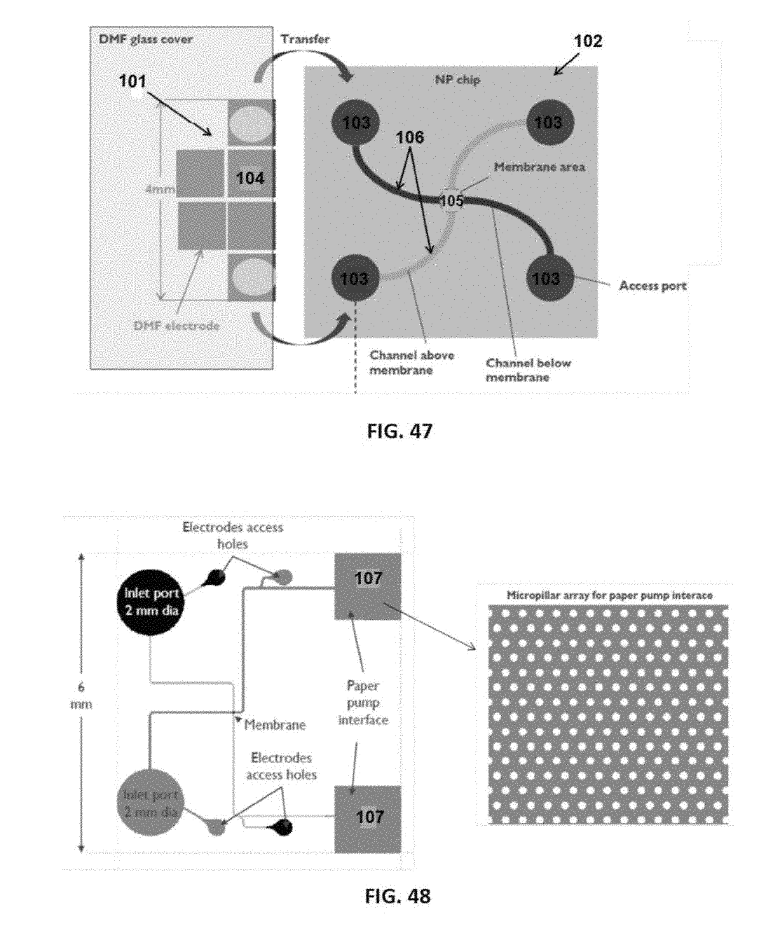

In addition to flexible substrates, such as PDMS, rigid substrates (e.g. silicon) may be used to fabricate the nanopore module. FIG. 47 shows a digital microfluidics (DMF) chip (101), containing actuation electrodes (104), from which droplets are transferred to a silicon microfluidic chip containing a nanopore sensor (102). Droplets are transferred between the two component chips by access ports (103) in the top surface of the microfluidic chips containing the nanopore sensor. Access ports are connected to the nanopore sensor (105) by microfluidic channels (106). Droplets are moved from the access ports, through the microfluidic channels by capillary forces, and movement may be aided by a passive paper pump fabricated from an array of micropillars (107) (FIG. 48). The passive pumps may also remove fluid from the microchannels, enabling different fluidic solutions to be used sequentially without contamination (for example, between solutions for nanopore formation and nanopore sensing).

Fabrication of the silicon nanopore module may include using standard CMOS photolithography and etching processes. FIG. 48 shows an example of a silicon nanopore module design, where the approximate die size is 10 mm×10 mm, with a frontside channel (cis) and a backside channel (trans) for filling the nanopore buffer(s). The frontside channel has a width and depth of 30 μm, and is 11 mm long. The backside channel has a width of 50 μm, a depth of 200 μm, and is 11 mm long. The micropillar dimensions are 30 μm pillar diameter, spacing of 30 μm and depth of 200 μm.

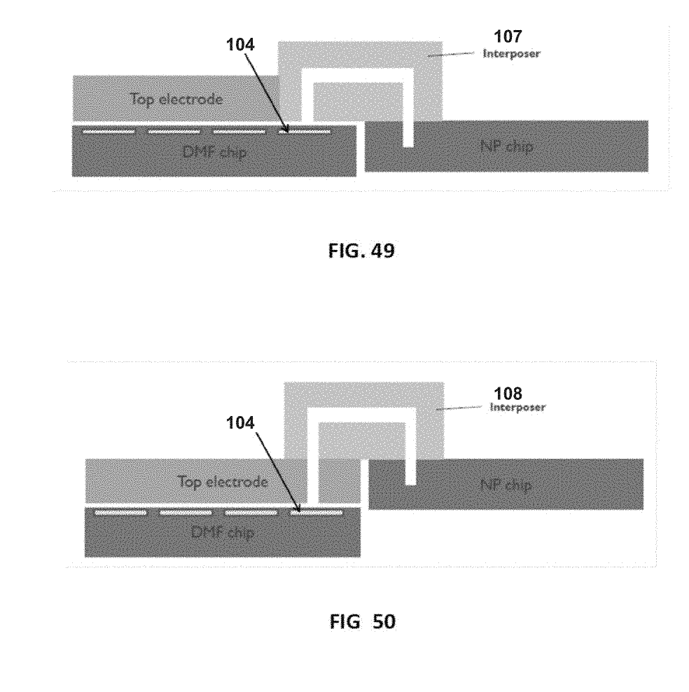

The DMF and nanopore module may be joined using an interface fabricated from molded plastic or by direct bonding (FIGS. 49 and 50). A droplet positioned on an electrode (104) within the DMF chip aligned with an access port is transferred by capillary forces, facilitated by the interposer (107). Alternatively, the top electrode (108) the DMF chip may be modified to further facilitate this process by introducing holes connecting the actuation electrodes (104) with the interposer (108) (FIG. 50).

### Example 24

### Droplet Transfer Between DMF and Nanopore Modules by Capillary Forces

The ability to move high-salt translocation buffer from a DMF chip to a module containing a suitable nanopore membrane was tested in a silicon microfluidic chip. A serpentine microchannel was tested for its ability to passively move a droplet of 1 M KCl (pH=8) using spontaneous capillary flow (SCF) as the sole driving force. The entire microchannel was fabricated in silicon and served as a model for fluidic transfer in a CMOS-based silicon environment. The serpentine microchannel was designed to have two access ports (for fluidic loading). The channel dimensions measured 160 μm in diameter, with an approximate length of 2.5 cm. Droplets of a solution suitable for formation of nanopores by dielectric breakdown were demonstrated to fill the silicon microfluidic structure using passive capillary forces.

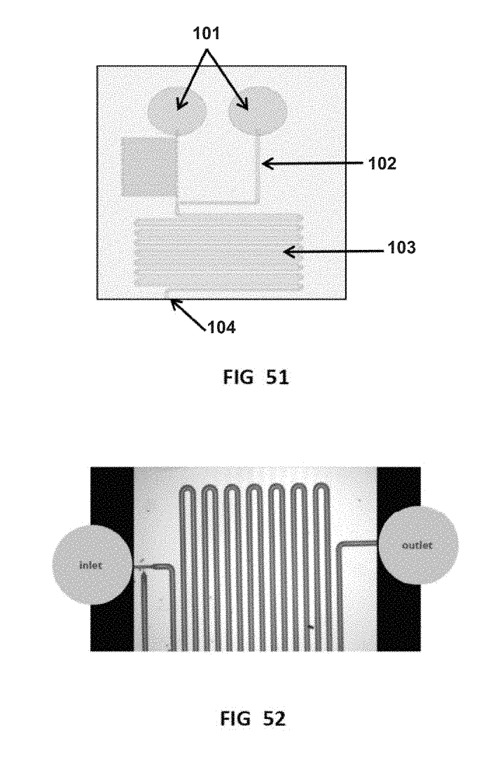

With reference to FIG. 51, individual droplets of 1M KCl solution (pH=8.0) were placed in one of the inlet ports (101) connecting to a transport microfluidic channel (102), leading to a serpentine channel 2.5 cm in length (103). The channel terminated (104) at a port (not shown) exposed to atmospheric pressure. A magnified image of the serpentine channel is shown in FIG. 52. Capillary filling was monitored using a sCMOS camera fitted to an optical microscope. Deposition of the salt solution into the inlet port resulted in spontaneous filling of the microchannel by passive capillary forces at a rate of several mm/second, thereby demonstrating the capability to transfer fluid in the microchannel to a nanopore membrane.

As a further test of transfer rate, the channel was emptied of the KCl solution and dried under a stream of nitrogen. Further droplets of 1M KCl solution (pH=8.0) were placed in the inlet port of the dried microchannel and capillary filling was monitored using and optical microscope. Faster fill rates were observed, compared to the “dry” channel (i.e., compared to the first time the KCl solution was introduced into the channel), thereby showing that pre-filling of the silicon microchannel with a hydrophilic solution enhanced subsequent fluidic filling.

### Example 25

### Fabrication of Integrated Nanopore Sensor with Fluidic Microchannels

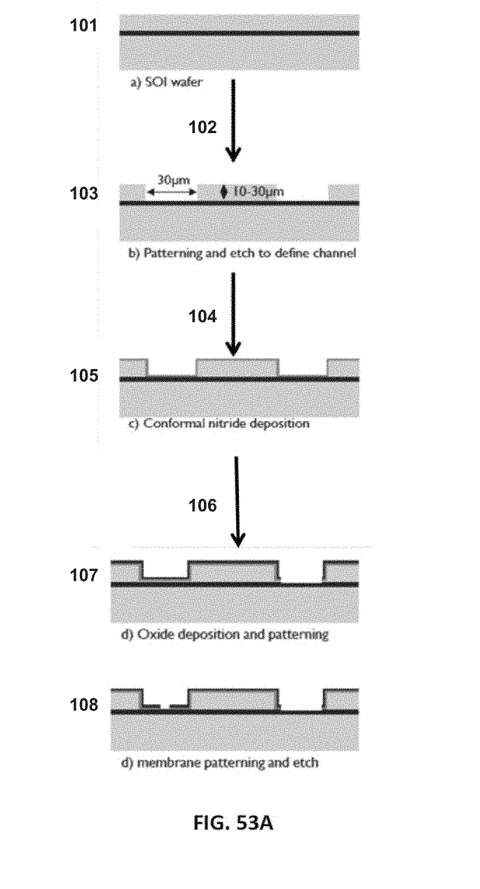

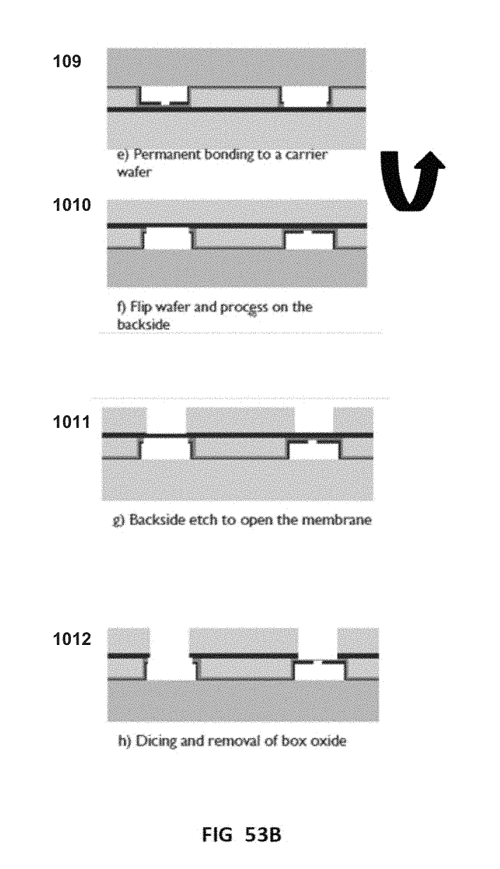

An integrated nanopore sensor within fluidic microchannels is fabricated using photolithography and etching processes to modify a silicon-on-oxide (SOI) wafer (FIGS. 53A-53B).

The SOI wafer (101) is subjected to photolithography and etching (102) to produce a structure suitable for the movement of small fluidic volumes (103) with dimensions of 30 μm width and 10-30 μm channel depth.

A silicon nitride (SiN) material (105) is deposited onto the patterned SOI wafer by evaporation (104).

A layer of oxide material (107) is deposited over the silicon nitride (105) by evaporation (106). The underlying silicon (101) is exposed by selectively removing the overlying oxide and nitride materials covering one of the microstructures using a combination of photolithography and etching (106). This structure will form a microchannel for actuating small volumes of fluid.

The underlying silicon nitride within a second microstructure is selectively exposed by removing the overlying oxide layer only using a combination of photolithography and etching (108).

The exposed microstructures are permanently bonded to a carrier wafer (109) and the structure is inverted for further processing (1010). The oxide material on the inverse side of the SOI wafer is selectively patterned using a combination of photolithography and etching to expose the back side of each microstructure (1011).

### Example 26

### Nanopore Counting Data

This Example describes nanopore counting data for a variety of tags, e.g., ssDNA hybrid molecules with polyethyleneglycols (DNA-STAR), dsDNA, dsDNA labeled with DBCO, and PAMAM succinamic acid dendrimers. Use of these different tags along with different size nanopores was done to provide for nanopore optimization. Different molecular polymer labels were suspended in an appropriate salt buffer and detected using a standard fluidic cell cassette.

Current-voltage (i-V) recordings (voltammetric data) and current-time (i-t) recordings were recorded using in-house instrumentation A computer software program called CUSUM was employed to run through the acquired data and detect events based on the threshold input by the user. Any impact of subjectivity in the assessment was minimized by detection of as many events as possible and filtering afterwards for specific purposes.

Initial experiments were performed with the tags added to the cis side of the membrane. An electric bias of 200 mV was applied to the label solution and current blockades were monitored using the Axopatch 200B amplifier and CUSUM software.

It is known that small molecules can go through nanopores quite fast unless the pore size restricts their passage. The current blockages of fast events can be deformed due to the limited bandwidth of a system. Faster molecules can even be completely undetected by a particular system.

In these studies, only larger polymers and molecules labeled with large group modifiers were detected. Experimental conditions and number of detectable events are shown in Table 3.

TABLE 3

| | | | Electrolyte | | Membrane | Nanopore | Detection | Events |
|---|---|---|---|---|---|---|---|---|
| | Cconc | Background | concentration | | Thickness | Diameter | Voltage | detected by |
| Polymer | (nM) | Electrolyte | (M) | pH | (nm) | (nm) | (mV) | CUSUM |
| 59 bp dsDNA control | 60 | LiCl | 3.6 | 8.0 | 10 | 3.9 | 200 | 414 |
| DBCO backbone | 96 | LiCl | 3.6 | 8.0 | 10 | 3.9 | 200 | 594 |
| dsDNa star | 20 | LiCl | 3.6 | 8.0 | 10 | 3.9 | 200 | 5589 |
| PAMAM (6th gen)- | 100 | KCl | 1.0 | 10 | 10 | 7.8 | 100 | 254 |
| succinamic acid | | | | | | | 150 | 1122 |
| | | | | | | | 200 | 1322 |

These data confirm that DNA dendrimers, polymers, and PAMAM dendrimers can be used as detection labels for solid-state nanopore sensors.

### Example 27

### Nanopore Differentiation of Biomolecules

In this Example, the nanopore was used to differentiate biomolecules (e.g., dsDNA stars, DBCO-modified dsDNA and regular dsDNA). This methodology can be used for multiplexing using different label types.

This Example employed a 50 bp oligonucleotide containing a branch point in the middle (bp #25), where a single-stranded oligonucleotide was covalently linked (DNA-Star); a double-stranded 50 bp oligonucleotide containing a dibenzylcyclooctyne (DBCO) modification in the middle (base #25); and a 5′-thiol modified double-stranded DNA oligonucleotide.

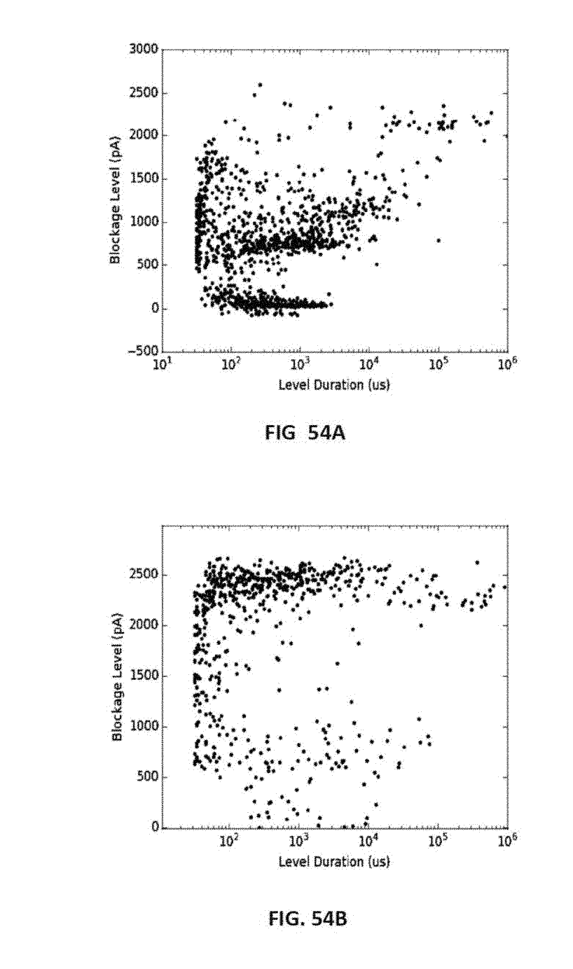

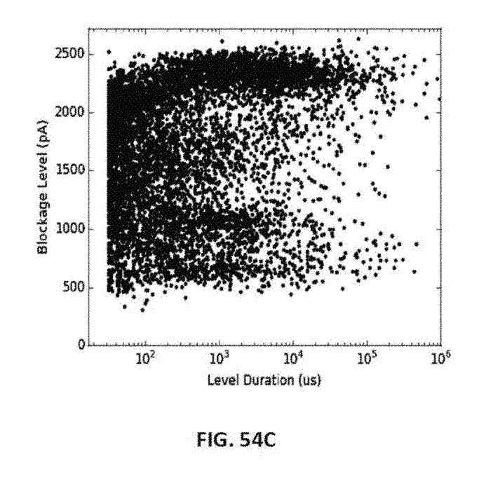

These various modified DNA molecules were analyzed using three different SiNx nanopores in 3.6 M LiCl buffer. DNA-star molecules were analyzed with a 4.0 nm diameter pore; DBCO-modified DNA was analyzed with a 3.7 nm diameter pore; thiol-modified DNA was analyzed with a 4.2 nm diameter pore. Current blockade levels (pA) were plotted against nanopore duration times (μsec), in order to show the ability of the nanopores to differentiate the three different biomolecules. At a population level, the three different labels appear to be distinguishable, as demonstrated by the distinct pattern differences in the scatter plots (FIGS. 54A-54C). Identification of individual events in real-time requires additional levels of blockade level and time information as a way to distinguish signals from noise. The ability to differentiate different nanopore labels demonstrate that nanopores can be employed for multiplexing in various assays.

### Example 28

### Qualitative Analysis

The following example describes a method for conducting a qualitative assay. Basically, in this example, a construct was used to demonstrate the principle of the assay on a DMF chip and the construct was cleaved and the label was released and then counted using a nanopore so as to generate a signal as it translocates the nanopore, thus indicating that the binding of two specific binding member pairs (streptavidin and biotin) wherein this cleavage and subsequent counting of a dsDNA label is correlated to the specific binding having occurred during the assay. Furthermore, appropriate control experiments were conducted to confirm that the signal generated from the label that was counted during the nanopore translocation measurement was due to the specific binding event having occurred during the assay process rather than being correlated to the presence of thiol cleavage reagent being introduced into the assay process flow. The details of the experiments conducted follow.

Thiol-Mediated Cleavage Using DMF:

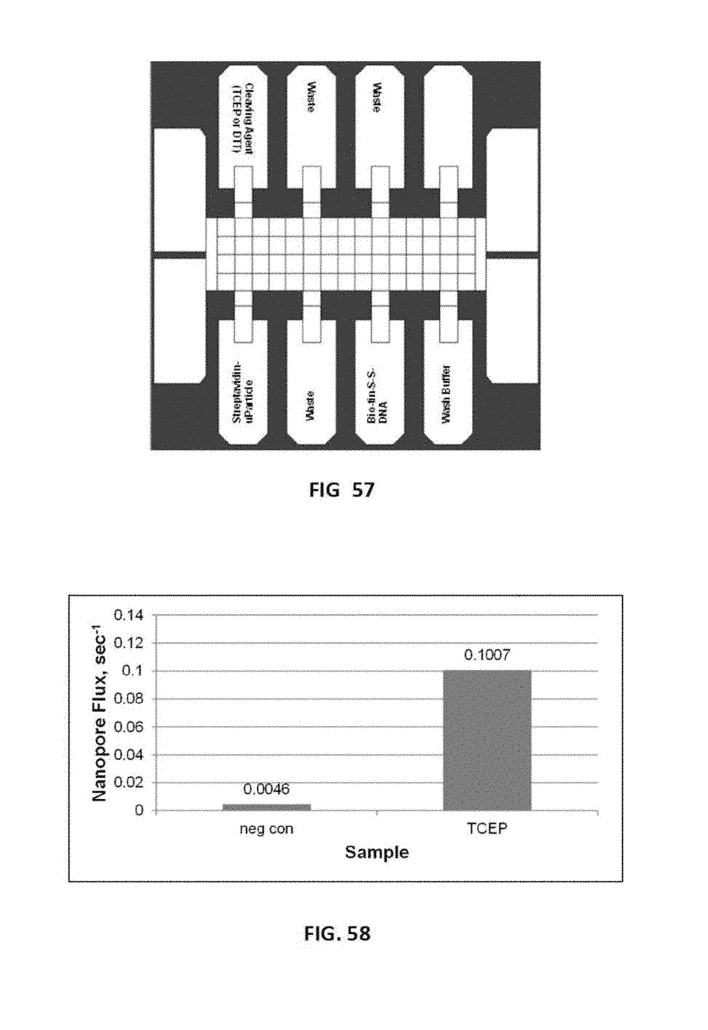

A biotin-labeled double-stranded DNA containing a cleavable disulfide bond ((106) of Example 15) was used as a target for nanopore detection/counting. The binding assay consisted of binding the biotin DNA to streptavidin magnetic microparticles on a DMF chip, followed by a thiol-mediated chemical cleavage step (see FIG. 55). Reagent placement on the DMF chip is shown in FIG. 57. The cleaved DNA target, separated from the species bound to the streptavidin magnet particles, was transferred to a nanopore fluidic cell containing a solid-state silicon nitride (SiNₓ) membrane with a pre-drilled nanopore created by controlled dielectric breakdown (H. Kwok, et al., PLoS, 9(3), 2014). The DNA target material was counted and analyzed using open-source CUSUM software analysis package (NIST).

Appropriate reagents were loaded onto a glass DMF chip (3″×2″×0.0276″) containing 8 reagent reservoirs. Except for waste reservoirs, each reservoir contained approx. 5 μL of each reagent. Concentrations of reagents were as follows: 11 μM Biotin-SS-DNA in PBS (pH=7.2); 10 mg/mL (w/v) M-270 2.7 μm streptavidin-coated magnetic microparticles (Life Technologies); PBS wash buffer (pH=7.2)+0.05% ETKT (Ethylene tetra-KIS (ethoxylate-block-propoxylate) tetro), 50 mM tris-(2-carboxyethyl)phosphine (TCEP). Approximate size of a dispensed DMF droplet was 1.5 μL.

One droplet of M-270 streptavidin-coated microparticles was dispensed and mixed with 1 droplet of dsNP-31-SS-biotin for approx. 40 minutes. Mixing was accomplished by combining the 2 droplets and moved in a circular pattern on the DMF chip over 12 electrodes (3×4). The bottom magnet was engaged to collect the microparticles and the supernatant was moved to a waste reservoir. Next, two droplets of PBS/ETKT buffer were dispensed and moved to the microparticle slug, which was then resuspended in solution. The suspension was mixed for 5 minutes before the magnet was again engaged and the supernatant was removed to the waste reservoir. The particle wash step was repeated a total of 11 times, while gradually increasing the mixing time up to 45 minutes. The last wash supernatant was moved to an empty reservoir. An additional 5 droplets of PBS/ETKT was moved to the same reservoir. The wash and PBS/ETKT in the reservoir was removed using a 34-AWG nonmetallic syringe (Microfil 34-AWG) and transferred to a 1.5 mL Eppendorf tube, in preparation for nanopore analysis. Cleavage was initiated by moving 2 droplets of TCEP reagent to the microparticle slug and mixing for 45 minutes. The bottom magnet was engaged and the supernatant (containing the cleaved DNA) was moved to an empty reservoir. An additional 5 droplets of PBS/ETKT wash buffer was moved to the same reservoir. The final extract was removed from the DMF chip using the 34 gauge nonmetallic syringe and transferred to a 1.5 mL Eppendorf tube, in preparation for nanopore analysis. The cleavage eluent was microfuged for 30 seconds and placed in a magnetic rack for 1 minute, to remove any trace microparticles.

Nanopore Analysis:

Nanopore fabrication was achieved using controlled dielectric breakdown (CBD) of a 10 nm thick SiNₓ membrane embedded in a TEM window (0.05 μm×0.05 μm) (Norcada NT0052, low stress SiNₓ). This method is capable of producing small diameter solid-state pores with high precision and minimal cost. The TEM-SiNₓ membrane was placed in a polytetrafluoroethylene (PTFE) fluidic cell containing two buffer chambers, and sealed using two silicone elastomer gaskets. The fluidic cell contained a 16 μL volume channel in the bottom of the cell, which connected the salt solution in the upper chamber to the nanopore membrane. For nanopore fabrication, the fluidic cell was first filled with degassed ethanol, exchanged with degassed deionized water and then filled with degassed 0.5 M KCl, buffered to pH 10 with sodium bicarbonate in 18 MΩ deionized water. Fabrication was performed using an amplifier using a bias voltage of 8V. The two sides of the fluid cell were connected using silver/silver chloride wires. As described in Kwok et al, while setting a fixed voltage of 8V, the current exhibits a capacitance (reduction of current) in real time. When the current increases, the power is removed from the cell. The sampling rate for the fabrication=25 KHz. An increase of the leakage current indicates formation of a pore, whereby the voltage was turned off. The pore diameter was determined from the following conductance-based equation:

$$G = {σ ( \frac{4 L}{π {d}^{2}} + \frac{1}{d} )}^{- 1}$$

where G=conductance, σ=bulk conductivity (12.35 S/m measured for KCl), L=thickness of the membrane (10 nm), d=pore diameter (S. Kowalczyk, A. Grosberg, Y. Rabin, C. Dekker, Nanotech., 22, 2011). The nanopore was checked for ohmic behavior by generating an I-V curve. The measured diameter of the nanopore was determined to be 4.4 nm, and was subsequently used for detection of the cleaved ds-SS-DNA target.

The fabrication salt buffer was replaced with 3.6 M LiCl, which was used as the sensing buffer for detecting translocation events. A headstage was placed between an Axopatch 200B amplifier and the silver/silver chloride connection to the fluidic cell housing the nanopore membrane.

Approx. 0.2 μL of the TCEP-cleaved ds-DNA target was diluted with 1.8 μL PBS buffer (this represented a 10-fold dilution of the TCEP-cleavage eluent), and the entire volume was loaded and mixed into the nanopore cell chamber, which contained approximately 30 μL of 3.6 M LiCl salt solution. The last DMF wash eluent was used as a negative cleavage control (this was not diluted). The number of DNA translocations was measured for 23 and 65 minutes for the TCEP eluent and negative control, respectively and converted to a flux rate (sec⁻¹). The results depicted in FIG. 58 demonstrate that the ds-SS-DNA target was successfully cleaved from the M-270 streptavidin particles using DMF and detected using a solid-state nanopore as a detector. SNR was determined to be 21.9, as measured from the nanopore flux rate.

Data Analysis:

The number of translocation events were determined by first calculating the anticipated current change found in a double stranded DNA translocation event under experimental test conditions using the equation

$$Δ G = \frac{σπ {d}_{DNA}^{2}}{4 L} , \qquad ( S1 )$$

as referenced in Kwok et al., “Nanopore Fabrication by controlled Dielectric Breakdown” Supplementary Information Section 8 and Kwok, H.; Briggs, K.; and Tabard-Cossa, V.; “Nanopore Fabrication by Controlled Dielectric Breakdown”—PLoS ONE 9(3): e92880 (2014). Using this anticipated current blockage value, the binary file data of the experimental nanopore output was visually manually scanned for acceptable anticipated current blockage events. Using these events, the Threshold and Hysteresis parameters required for the CUSUM nanopore software were applied and executed. The output from this software was further analyzed using the cusumtools readevents.py software and filtering blockage events greater than 1000 pA (as determined from the first calculation). The flux events, time between events and other calculations were determined from the readevents.py analysis tool. Additional calculations were made on the CUSUM generated data using JMP software (SAS Institute, Cary, N.C.). It is understood that this method of threshold setting is one approach to data analysis and setting a threshold and that the present invention is not limited to this method and that other such methods as known to those skilled in the art can also be used.

Summary:

This example describes a qualitative assay by conducting the process of steps as described herein. A direct assay was conducted using the cleavable linker conjugate, as described in Example 15, with a thiol based cleavage step, as shown in FIG. 55. It is understood that other cleavable linker approaches to conducting such an assay may also include, but are not limited to, various other methods of cleavage of a linker so as to allow for the counting of various tags, as described herein. For example, such other alternative cleavage methods and/or reagents in addition to the method described in Example 15 can include those described in Example 14, Example 16, Example 17, Example 18 and Example 19, in addition to other cleavage methods described herein and known to those skilled in the art. It is also understood that while the assay format demonstrated in this Example (Example 28) represents a direct assay, other formats such as sandwich immunoassay formats and/or various competitive assay formats, such as are known to those skilled in the art, can be implemented as well to conduct an assay using the described methods.

For example, the sandwich immunoassay format for the detection of TSH (thyroid stimulating hormone), as described in Example 3, demonstrated the ability to conduct such an assay on a low cost DMF chip. Additionally, a number of various bioconjugation reagents useful for the generation of immunoconjugate or other active specific binding members having cleavable linkers can be synthesized using various heterobifunctional cleavable linkers such as those described in Example 8, Example 9, Example 10, Example 11, Example 12, and Example 13, in addition to other cleavable linkers that are otherwise known to those skilled in the art. Immunoconjugates useful for the practice of the present invention can be synthesized by methods such as those described in Example 10, Example 11, Example 12, and Example 13 as well as by methods known to those skilled in the art. Additionally, Example 2 shows the functionality of various fluidic droplet manipulations on a low cost chip that can facilitate various steps needed to carry out various assay formats including sandwich and competitive assay formats as well as other variations thereof known to those skilled in the art. Example 21 shows the fabrication of a nanopore that can be used to count cleavable label in an assay but it is understood that other methods for nanopore fabrication known to those skilled in the art can also be used for this purpose. Example 14 also represents another construct useful for the conduct of an assay where a cleavage is effected, thus leading to a countable label being released so as to be countable using the nanopore counting method, as described within this example.

Example 26 shows generally how counting can be effected so as to be able to measure translocation events relating to the presence of a variety of labels traversing the nanopore. FIG. 59 shows the concept of thresholding of the signal so as to be able to manipulate the quality of data in a counting assay. FIG. 58 shows qualitative assay data that is representative of the type of data that can be used to determine the presence of an analyte using such assay methods as described within this example. It is also understood that while dsDNA was used as a label in this particular example, other labels, such as the label described in Example 12 and/or Example 26 can also be utilized, including, but not limited to nanobeads, dendrimers and the like. Such constructs as needed to generate appropriate reagents can be synthesized through various examples herein this application or otherwise via methods known to those skilled in the art.

### Example 29

### Quantitative Analysis

The following example describes a method for conducting a quantitative assay. Basically, in this example, and as an extension of Example 28, a standard curve was generated so as to demonstrate that increased amounts of counting label, in this case with the countable label being a dsDNA molecule, correlated on a standard curve to the amount of specific binding agent that has been bound (which it turn correlates to the amount of analyte existing in the original sample) in an assay (binding) step. The standard curve for this particular experiment can be found in FIGS. 61, 62, and 64 based on various different methods of data analysis or FIG. 64, which relies up flux to generate a standard curve. In the latter case, the measurement method shown in FIG. 64 based based upon the events/time (flux of counting events) but it is understood that other measurement methods can also be used to generate a standard curve correlating to the amount of analyte concentration being measured in a given sample. The details of the experiments conducted are as follows.

Nanopore Fabrication:

Nanopore fabrication was achieved using controlled dielectric breakdown (CBD) of a 10 nm thick SiNₓ membrane embedded in a TEM window (0.05 μm×0.05 μm) (Norcada NT0052, low stress SiNₓ) as this method is capable of producing small diameter solid-state pores with high precision and minimal cost. The TEM-SiNₓ membrane was placed in a polytetrafluoroethylene (PTFE) fluidic cell containing two buffer chambers, and sealed using two silicone elastomer gaskets. The fluidic cell contained a 16 μl volume channel in the bottom of the cell, which connected the salt solution in the upper chamber to the nanopore membrane. For nanopore fabrication, the fluidic cell was first filled with degassed ethanol, exchanged with degassed deionized water and then filled with degassed 0.5 M KCl, buffered to pH 10 with sodium bicarbonate in 18 MΩ deionized water. Fabrication was performed using an amplifier using a bias voltage of 8V. The two sides of the fluid cell were connected using silver/silver chloride wires. As described in Kwok et al, while setting a fixed voltage of 8V, the current exhibits a capacitance (reduction of current) in real time. When the current increases, the power is removed from the cell. The sampling rate for the fabrication was 25 KHz. An abrupt increase of the leakage current indicated formation of a pore, whereby the voltage was turned off. The 0.5 M KCl buffer was replaced with 3.6 M LiCl (pH=8.3).

The pore diameter was determined from the following conductance-based equation:

$$G = {σ ( \frac{4 L}{π {d}^{2}} + \frac{1}{d} )}^{- 1} ,$$

where G=conductance, σ=bulk conductivity (16.06 S/m measured for LiCl), L=thickness of the membrane (10 nm), and d=pore diameter (S. Kowalczyk, A. Grosberg, Y. Rabin, C. Dekker, Nanotech., 22, 2011). The nanopore was checked for ohmic behavior by generating an I-V curve. The measured diameter of the nanopore was determined to be 4.8 nm, and was subsequently used for detection of the DNA calibration standards.

DNA Dose-Response:

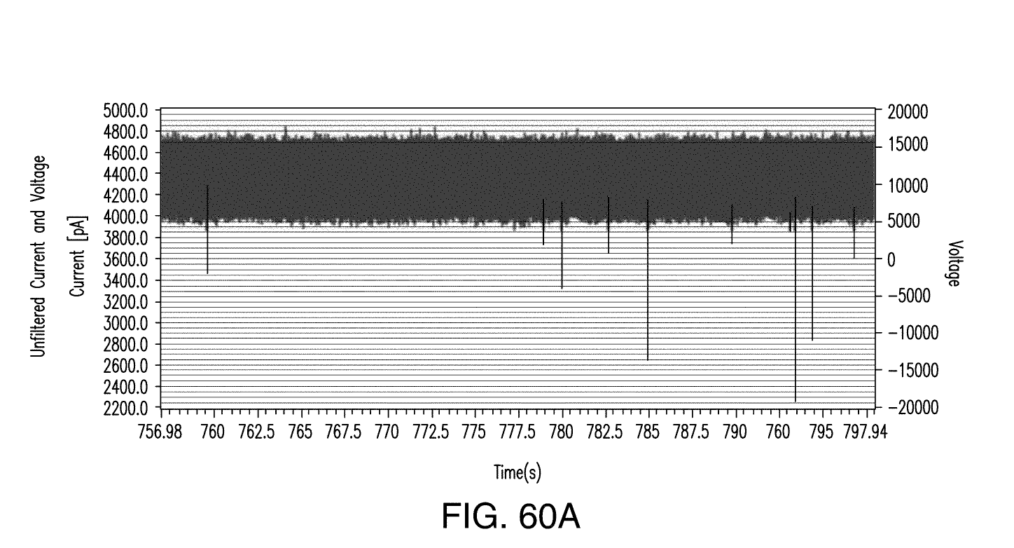

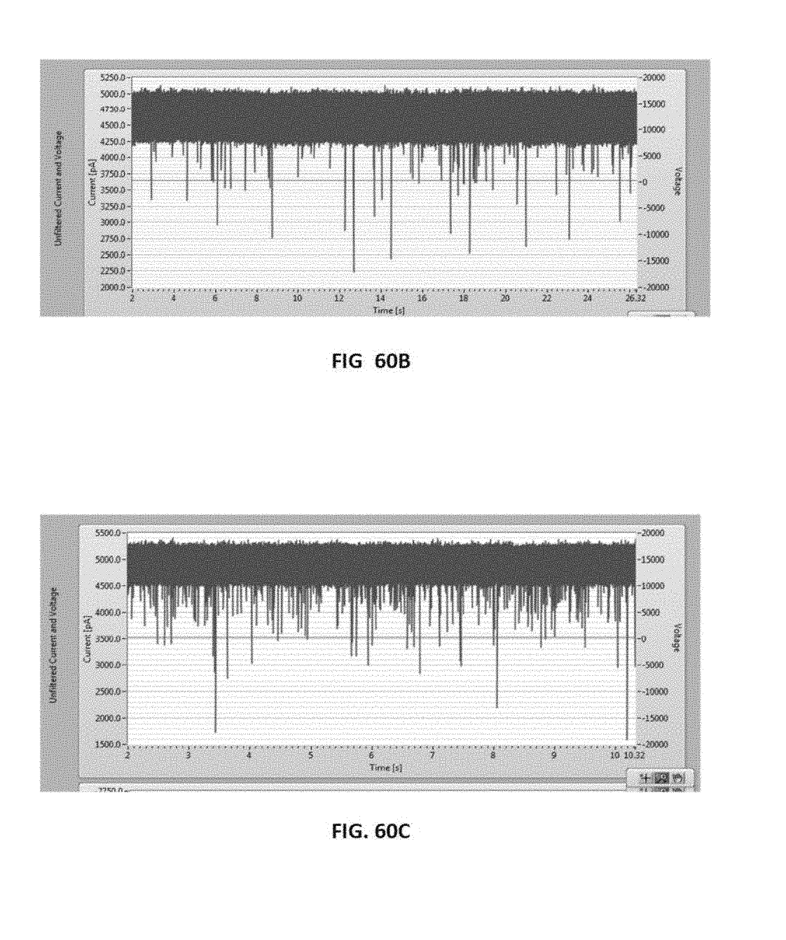

DNA standards were used as calibrators to observe a dose-response curve by determining the change in nanopore flux rate with increasing concentrations of DNA. This generated a standard curve, which can be used for quantitation of a cleaved DNA label in an immunoassay. Two μl of a 1.5 μM 100 bp DNA standard (ThermoScientific) was pipetted into the PTFE fluidic cell containing 30 μl of 3.6 M LiCl salt solution, to give a final concentration of 94 nM DNA. The reagent was mixed by pipetting the solution up and down several times prior to nanopore analysis. The cell was subjected to a DC bias of +200 mV and monitored for current blockades over 60 minutes. CUSUM analysis software was used to characterize electrical signals and count rates. This procedure was repeated two times to give two additional points on the standard curve, 182 nM and 266 nM. Current blockades over different time periods are shown for all three standards—41 seconds for 94 nM (FIG. 60A); 24 seconds for 182 nM (FIG. 60B); 8 seconds for 266 nM (FIG. 60C). Baseline noise was empirically estimated to be approximately 900 pA, 900 pA and 1,000 pA for FIG. 60A, FIG. 60B and FIG. 60C, respectively.

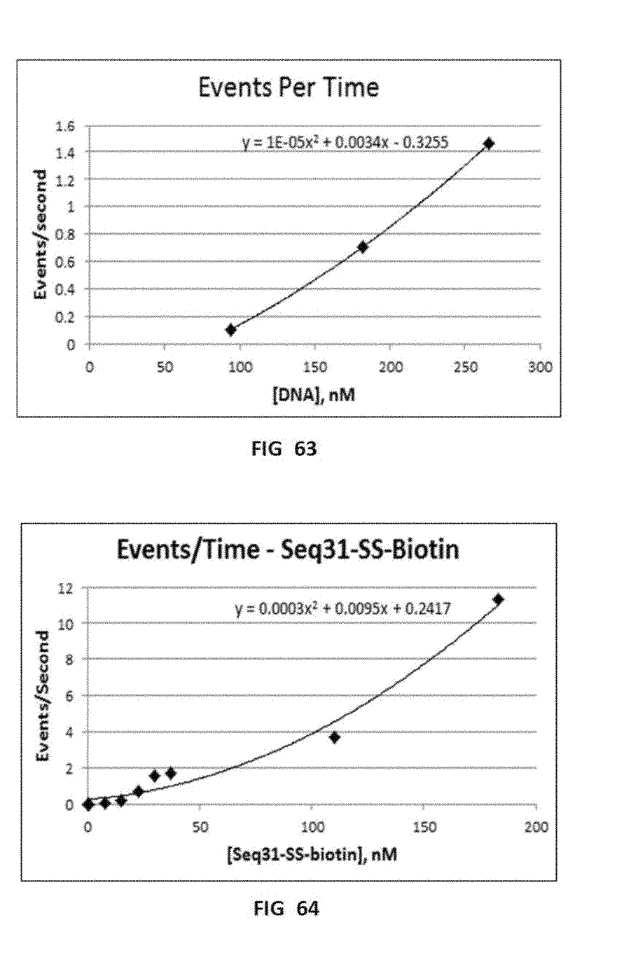

Data from the run was used to generate three different types of dose-response curves—number of events over a fixed amount of time (5 minutes) (FIG. 61); time required for fixed number of events (200 events) (FIG. 62); and events per unit time (FIG. 63). Each of these curves may be used as a standard curve for a quantitative nanopore-based immunoassay, using DNA as the label. Similarly, other labels may be used to quantitate various analytes, such as dendrimers, polymers, nanoparticles, and the like.

Seq31-SS-Biotin DNA Dose-Response:

The synthetic DNA construct, Seq31-SS-biotin, was used as the source material to generate a dose-response curve (FIG. 64). This target can be used to quantitate the cleaved label NP-Seq31-SS-biotin, which was cleaved from the streptavidin beads in the qualitative assay. Since this material has approximately the same MW and charge density as the cleaved label Seq31-SS-biotin, it may be used in a calibration curve to quantitate the cleaved target from streptavidin microparticles using TCEP and/or DTT.

Data Analysis:

The number of translocation events were determined by first calculating the anticipated current change found in a double stranded DNA translocation event under experimental test conditions using the equation:

$$Δ G = \frac{σπ {d}_{DNA}^{2}}{4 L} \qquad ( S1 )$$

as referenced in Kwok et al., “Nanopore Fabrication by controlled Dielectric Breakdown” Supplementary Information Section 8 and Kwok, H.; Briggs, K.; and Tabard-Cossa, V.; “Nanopore Fabrication by Controlled Dielectric Breakdown”—PLoS ONE 9(3): e92880 (2014). Using this anticipated current blockage value, the binary file data of the experimental nanopore output was visually manually scanned for acceptable anticipated current blockage events. Using these events, the Threshold and Hysteresis parameters required for the CUSUM nanopore software were applied and executed. The output from this software was further analyzed using the cusumtools readevents.py software and filtering blockage events greater than 1000 pA (as determined from the first calculation). The flux events, time between events and other calculations were determined from the readevents.py analysis tool. Additional calculations were made on the CUSUM generated data using JMP software (SAS Institute, Cary, N.C.). It is understood that this method of threshold setting is one approach to data analysis and that the present invention is not limited to this method but other such methods as known to those skilled in the art can also be used.

Summary:

This example describes a quantitative assay by conducting the process of steps as described herein. A direct assay was conducted using the cleavable linker conjugate, as described in Example 15, with a thiol based cleavage step, and as shown in FIG. 55. It is understood that other cleavable linker approaches to conducting such an assay may also include, but are not limited to, various other methods of cleavage of a linker so as to allow for counting of various tags using a nanopore, as described herein. For example, such other cleavage methods in addition to the method described in Example 15 can include, but is not limited to, those described in Example 16, Example 17, Example 18, and Example 19, in addition to other methods described herein and known to those skilled in the art. It is also understood that while the assay format demonstrated in this Example (Example 29) represents a direct assay, other formats such as sandwich immunoassay formats and/or various competitive assay formats, such as are known to those skilled in the art, can be implemented as well to conduct an assay.

For example, the sandwich immunoassay format for the detection of TSH (thyroid stimulating hormone), as described in Example 3, demonstrated the ability to conduct such an assay on a low cost DMF chip. Additionally, a number of various bioconjugation reagents useful for the generation of immunoconjugate or other active specific binding members having cleavable linkers can be synthesized by those skilled in the art using various heterobifunctional cleavable linkers and conjugates synthesized by methods such as those described in Example 8, Example 9, Example 10, Example 11, Example 12, and Example 13, in addition to other cleavable linkers or conjugates that could be synthesized by methods that are known to those skilled in the art. Additionally, Example 2 shows the functionality of various fluidic droplet manipulations on a low cost chip that can facilitate various steps needed to carry out various assay formats including sandwich and competitive assay formats as well as other variations thereof known to those skilled in the art. Example 14 also represents another construct useful for the conduct of an assay where a cleavage is effected, thus leading to a countable label being released so as to be countable using the nanopore counting method as described within this example.

Example 26 shows generally how counting can be performed so as to be able to measure translocation events relating to the presence of a label traversing the nanopore. FIG. 59 shows the concept of thresholding of the signal so as to be able to manipulate the quality of data in a counting assay. FIGS. 61, 62 and 63 show quantitative assay data output that is representative of the type of data that can be used to determine the amount of an analyte using such assay methods as described within this example. FIG. 64 shows a standard curve generated from a construct that has been cleaved using a chemical method. It is also understood that while dsDNA was used as a label in this particular example, other labels, such as the label described in Example 12, can also be utilized, including, but not limited to, nanobeads, dendrimers and the like. Such constructs can be synthesized via methods known to those skilled in the art.

### Example 30

### Nanopore Electrical Field Simulations

A series of COMSOL simulation runs were performed on the proposed nanopore membrane design used in the silicon module, to study the influence of the size of the SiO₂ via on the counter ion concentration and electroosmotic flow rate through a theoretical 10 nm diameter nanopore. A top layer of SiO₂ served multiple purposes—1) provide an insulating layer to the SiNₓ membrane and, thereby, reduce the capacitive noise of the nanopore; 2) to increase the robustness and strength of the SiNₓ membrane within the silicon substrate; 3) to decrease the size of the SiNx area exposed to solution, thereby improving positioning of the pore on the membrane from the controlled dielectric breakdown (CBD) process. Electrical field simulations were used to determine interference of the SiO2 layer on localized counter ion concentration and electroosmotic flow through the pore.

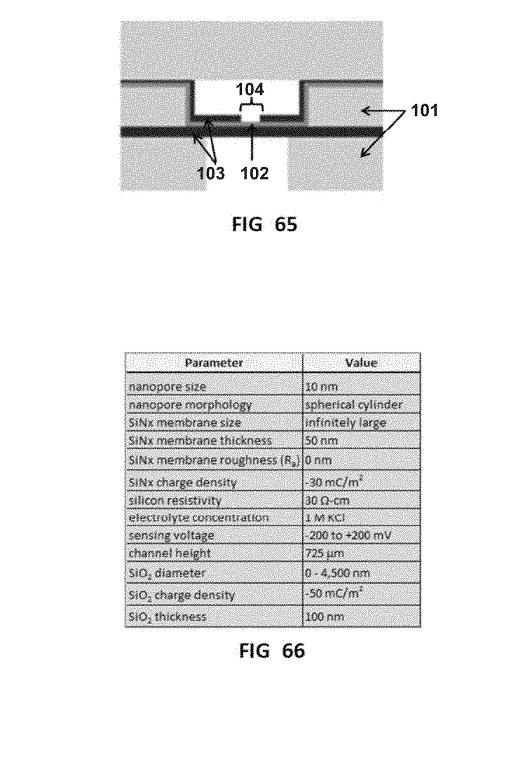

With reference to FIG. 65, the silicon substrate (101) was etched to give cis and trans chambers, situated above and below the SiNₓ membrane. The SiNₓ membrane (50 μm×50 μm) (102) was layered between a 300 μm thick bottom layer of SiO₂ and a 300 μm thick top layer of SiO₂ (103). The top layer was fabricated to form a SiO₂ via (104), which allowed formation of the nanopore during CBD. The optimal diameter of the SiO2 via was determined by the simulation.

COMSOL Simulation Results:

COMSOL electrical field simulations used physical models based on materials, electrostatics, molecular transport and Laminar flow properties. Electric potential was based on Poisson equation; ionic flux was based on Nernst-Planck equation; fluid velocity was based on Stokes equation. Physical parameters used for the simulation are defined in Table 1, shown in FIG. 66.

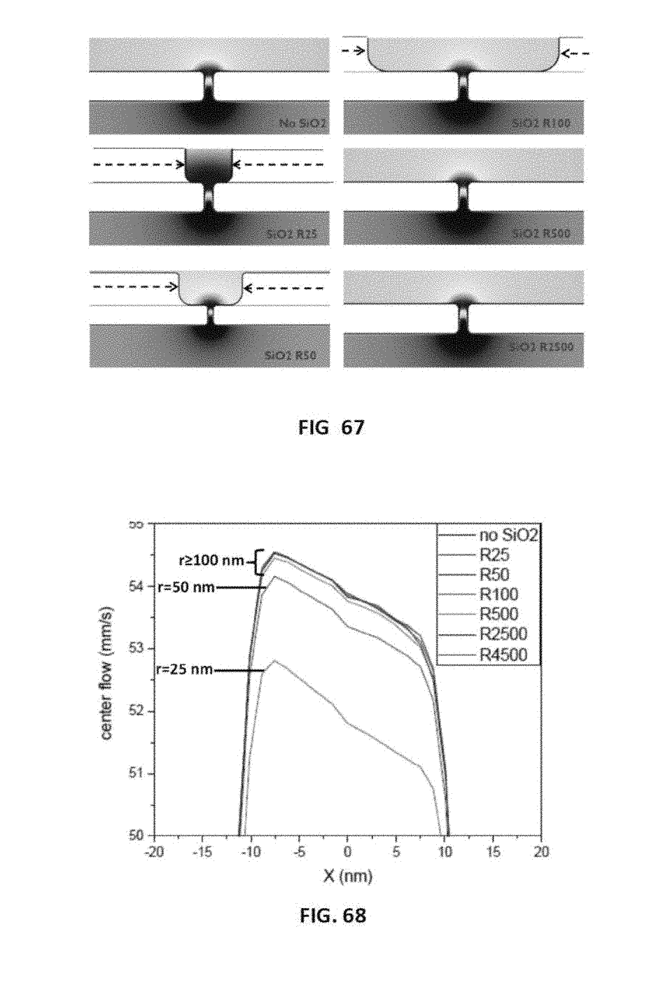

COMSOL results for counter ion concentration gradients near the pore are shown in FIG. 67, and show little to no influence of the ionic concentration when the SiO₂ via diameter was >50 nm in diameter. Below 50 nm, an accumulation of net charge near the mouth of the pore resulted. The most severe effect was observed at a diameter of 25 nm, where a large ionic gradient formed near the pore. The results showed a fairly large influence of the SiO₂ surface when the nanopore was less than 25-50 nm away from the SiO₂ wall.

Electroosmotic flow rates of counter ions through the pore were simulated as a way to determine any influence the SiO₂ layer may have on nanopore sensing (FIG. 68). The highest rate of electroosmotic flow occurred with the larger via diameters (100-4,500 nm). A reduction in flow rate through the pore was observed for a 50 nm SiO₂ via, followed by a significant reduction for a 25 nm via.

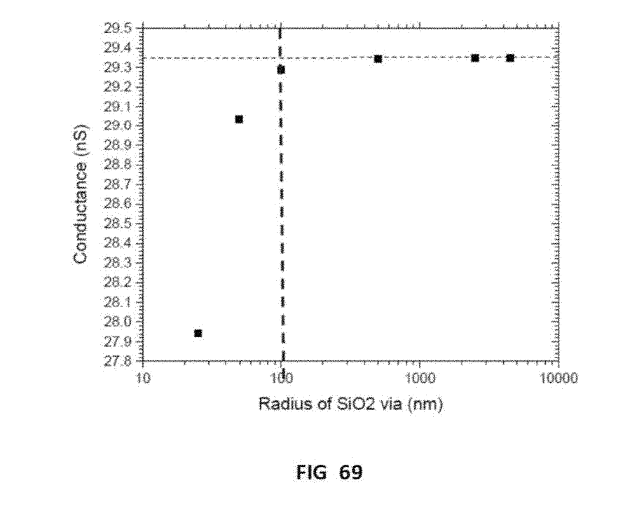

As shown in FIG. 69, measurement of conductance through the pore vs. via diameters showed a saturation curve above 100 nm, with diminishing conductance as the via diameter was reduced in size from 100 nm to 25 nm.

### Example 31

### Integrating a Nanopore Module into a Digital Microfluidic (DMF) Module

The nanopore module was located on one side of the DMF module. A hole was present in the DMF module to allow liquid transport from the DMF module to the nanopore module for pore creation and analyte detection (e.g., see FIG. 70).

One electrode from the nanopore module terminated within the fluid volume in the nanopore module. The other electrode terminated within the fluid volume in the DMF module. This electrode was routed through a second hole in the DMF module. To demonstrate that liquid was able to move through the hole within the DMF module, a flat piece of paper was pushed over the exterior surface of the chip after liquid was moved in place. The wetting of this paper showed that the liquid was able to move from the DMF module to another module located above this hole via capillary forces (FIG. 71).

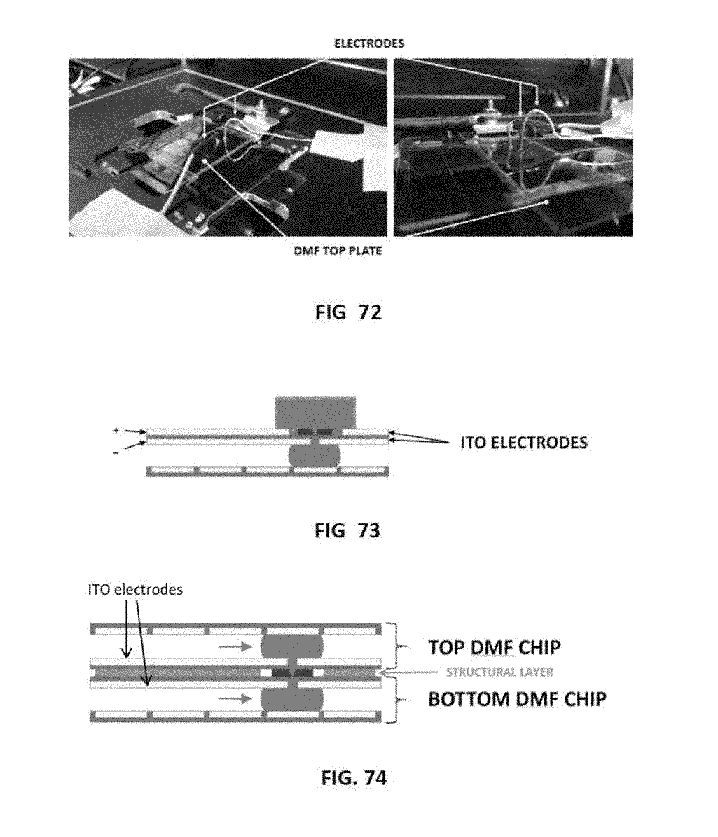

With reference to FIG. 72, the DMF module was equipped with Ag/AgCl electrodes for control of the nanopore fabrication. In this setup, the liquid volume on the nanopore module was an open-air droplet of LiCl. This liquid was dispensed directly onto the nanopore module and the electrode terminal was suspended within this droplet.

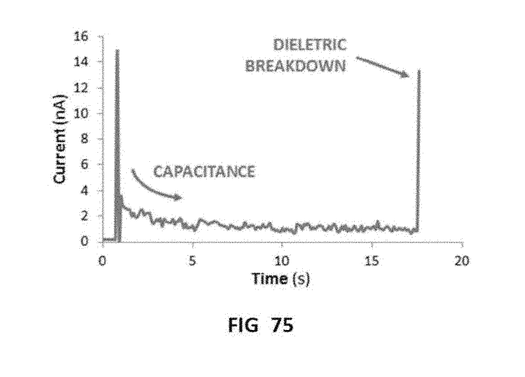

The sample was moved to the hole in the DMF module using DMF technology. The sample passively migrated through the hole to become exposed to the nanopore module for nanopore creation. The nanopore module is sealed to the DMF module (e.g. using PDMS, pressure, wax, etc.), isolating the liquid volumes held within each module. FIG. 75 shows the current as a function of time during the fabrication of the nanopore.

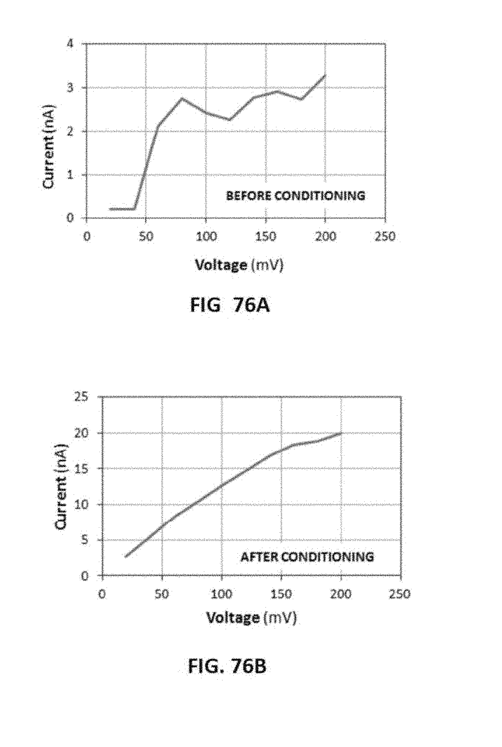

Once the nanopore was created, a conditioning process (varying voltage over time) was used to physically modify the nanopore and clean the signal. This process improved symmetry in the I-V curve. The before and after I-V curves are shown in FIGS. 76A and 76B, respectively.

### Example 32

### Counting Labels and Pore Size Analysis

A set of experiments were run using double stranded DNA under various sets of conditions to analyze and demonstrate certain attributes relative to pore size and counting label size. In these experiments, various parameters were explored including detection voltage, DNA length, DNA concentration, salt concentration and salt composition, membrane material, membrane thickness, nanopore diameter and other factors.

The data set was then analyzed relative to signal to noise ratio and compared that factor to various pore size relative to counting label size (estimated molecular diameter). Certain factors such as membrane material and thickness, for example, were held constant in this set of experiments, while other factors were varied.

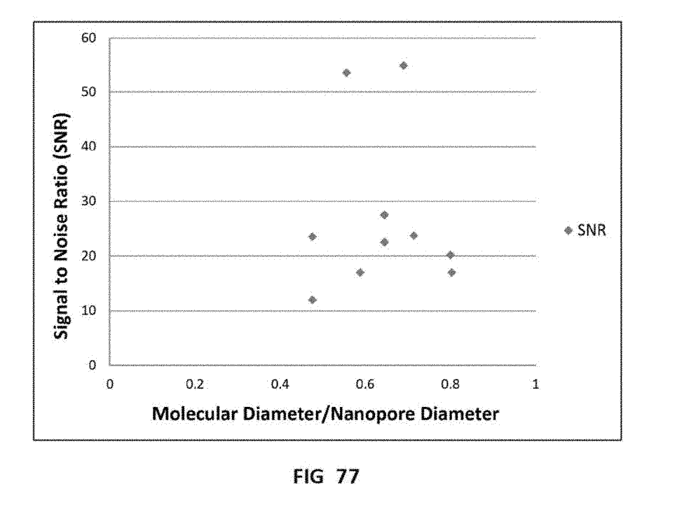

From an aggregate data set analysis, the averages of ratios were plotted between counting label average diameter and nanopore size to the SNR (signal to noise ratio) determined in the experiment (FIG. 77). FIG. 77 demonstrates generally that useful counting data can be obtained from a range of such ratios, in this particular data set from between around 0.4 to 0.8 in such ratio—assuming a molecular diameter of a dsDNA of around 2.0 nm approximately. Linear dsDNA is known from the literature to be about that molecular diameter and the analysis assumes the DNA threads through the pore in its linear conformation. Table 4 shows the calculated data.

TABLE 4

| AVERAGE PORE LABEL MOLECULAR | |
|---|---|
| DIAMETER TO PORE RATIO | SNR |
| 0.645 | 27.5 |
| 0.556 | 53.7 |
| 0.476 | 12 |
| 0.714 | 23.7 |
| 0.803 | 17 |
| 0.645 | 22.5 |
| 0.8 | 20.2 |
| 0.588 | 17 |
| 0.69 | 55 |
| 0.476 | 23.5 |

While conditions varied, as previously mentioned in this example, the general range in this data set shows that counting data with reasonable signal to noise can be obtained within this range. Furthermore, it should be noted that one skilled in the art would recognize that other counting label molecular diameter to nanopore diameter ratios could be utilized to achieve reasonable SNR. Additionally, it would be recognized by one skilled in the art that generally a label should have at least one dimension of its molecular diameter that is less than the size of the nanopore so as to be able to pass through the pore, or in other words, this ratio of label molecular diameter to nanopore diameter should generally be less than one for the label to be able to pass through the pore, except in cases perhaps where conditions such as are described in a technology called nanopore force spectroscopy is used, wherein energy is added to the system to facilitate conformational changes to occur in the label and thus allow it to pass through the pore after deformation to a level that would allow such a translocation event to occur.

It should also be understood to one skilled in the art that other labels can be utilized for counting other than dsDNA as described in this example, and that they may have different behaviors than that shown in this graph. Furthermore, it should also be understood that it is possible to also obtain acceptable SNR from other molecular diameter to nanopore ratios to enable molecular counting of such labels, and that current blockage can be related to molecular diameter of such a counting label as described in the equation below

$$Δ G = \frac{σπ {d}_{DNA}^{2}}{4 L} \qquad ( S1 )$$

which can be found in the following references: Kwok et al., “Nanopore Fabrication by controlled Dielectric Breakdown” Supplementary Information Section 8 and/or Kwok, H.; Briggs, K.; and Tabard-Cossa, V.; “Nanopore Fabrication by Controlled Dielectric Breakdown”—PLoS ONE 9(3): e92880 (2014). This equation can be used in order to gate or threshold signal as described in Examples 28 and 29 in this document.

Certain specific conditions varied within this aggregate set of nanopore counting experiments included:
Ionic Strength—either 3 or 3.6 M
DNA length—10 kbp, 50 bp or 1 kbp
Ionic Salt Used—either LiCl or KCl
Membrane Material—SiNx (constant throughout data set)
Membrane Thickness—10 nm (constant throughout data set)
DNA Concentration(s)—varied between 3 nM and around 306 nM
Voltages—varied including increments between 50 and 600 mV
Nanopore Diameter—a variety of pore sizes including 8.0, 1.1, 3.6, 4.2, 2.8, 2.5, 7.7, 3.1, 2.7, 2.6, 2.9 and 4.2 (all in nanometers).

Conclusions can be drawn that various conditions, including but not limited to these, show that one can obtain in situations where the countable label is smaller than the diameter of the pore can cause a blockage of the flux of ion current across the pore when a voltage is applied as per the amount as calculable but this equation of Kwok et al as referenced in this example [Kwok et al., “Nanopore Fabrication by controlled Dielectric Breakdown” Supplementary Information Section 8 and/or Kwok, H.; Briggs, K.; and Tabard-Cossa, V.; “Nanopore Fabrication by Controlled Dielectric Breakdown”—PLoS ONE 9(3): e92880 (2014)].

It is also understood that these conditions can be applied to show counting label molecular diameters to pore diameters that will function with reasonable signal to noise for other labels besides dsDNA, including but not limited to dendrimers, hemi-dendrimers, nanobeads, anionic or cationic polymers, denatured linearized aptamers, negatively or positively charged poly peptides or other charged polymers or countable molecular entities and the like.

Finally, although the various aspects and features of the invention have been described with respect to various embodiments and specific examples herein, all of which may be made or carried out conventionally, it will be understood that the invention is entitled to protection within the full scope of the appended claims.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A digital microfluidic and analyte detection device, comprising:
a first substrate and a second substrate aligned generally parallel to each other to define a gap therebetween, the first substrate comprising a plurality of electrodes to generate electrical actuation forces on a liquid droplet disposed in the gap;
at least one reagent disposed on at least one of the first substrate or the second substrate and configured to be carried by the liquid droplet; and
an analyte detection device in fluid communication with the gap, wherein the plurality of electrodes are configured to move the liquid droplet towards the analyte detection device.

2. The device of claim 1, wherein the reagent is disposed within a reservoir on at least one of the first substrate or the second substrate.

3. The device of claim 1, wherein the reagent is configured to be hydrated when contacted with the liquid droplet.

4. The device of claim 1, wherein the reagent further comprises a solid support.

5. The device of claim 1, wherein the analyte detection device is configured for single molecule counting.

6. The device of claim 5, wherein the analyte detection device comprises an array of wells dimensioned to hold a portion of the liquid droplet.

7. The device of claim 6, wherein the array of wells is positioned between the gap and the plurality of electrodes.

8. The device of claim 6, wherein the array of wells is positioned on the second substrate.

9. The device of claim 6, wherein the first substrate comprises a first portion at which the liquid droplet is introduced and a second portion comprising the array of wells.

10. The device of claim 5, wherein the analyte detection device is a nanopore module.

11. The device of claim 10, wherein the reagent comprises a detectable label having a cleavable tag.

12. The device of claim 10, wherein at least two electrodes of the plurality of electrodes are positioned across a nanopore layer in the nanopore module, wherein the two electrodes form an anode and a cathode and drive current through a nanopore in the nanopore layer when the liquid droplet is positioned across the nanopore layer.

13. The device of claim 12, further comprising a capillary portion comprising a hydrophilic material to facilitate movement of the liquid droplet to the nanopore module.

14. The device of claim 13, wherein the capillary portion comprises:
a first capillary channel; and
a second capillary channel;
wherein the first capillary channel intersects the second capillary channel with a nanopore layer positioned between the first and second capillary channels.

15. A method of detecting an analyte of interest, comprising:
introducing a liquid droplet comprising an analyte of interest into a device comprising:
a first substrate and a second substrate aligned generally parallel to each other to define a gap therebetween, the first substrate comprising a plurality of electrodes to generate electrical actuation forces on a liquid droplet disposed in the gap;
at least one reagent disposed on at least one of the first substrate or the second substrate and configured to be carried by the liquid droplet; and
an analyte detection device in fluid communication with the gap, wherein the plurality of electrodes are configured to move the liquid droplet towards the analyte detection device;
actuating at least one electrode to move the liquid droplet towards the analyte detection device;
labeling the analyte of interest with a detectable label; and
detecting the detectable label.

16. The method of claim 15, wherein the method comprises single molecule counting.

17. The method of claim 15, wherein the reagent comprises the detectable label.

18. The method of claim 17, wherein the detectable label comprises a binding member and a cleavable tag.

19. The method of claim 15, further comprising introducing a second liquid droplet containing at least one solid support with a specific binding member to bind to the analyte of interest.

20. The method of claim 15, further comprising manipulating the liquid droplet to facilitate mixing of the liquid droplet and the reagent.
